(19) United States
(12) Patent Application Publication
(10) Pub. No.: US 2024/0390293 A1
(43) Pub. Date: Nov. 28, 2024

(54) DERIVATIVES OF THE PROTECTIN 10S,17S-DIHDA (PDX) AND USE THEREOF AS ANTIVIRAL, ANTI-INFLAMMATORY, AND ANTI-DIABETIC AGENTS

(71) Applicant: UNIVERSITÉ LAVAL, Québec (CA)

(72) Inventors: René MALTAIS, Québec (CA); André MARETTE, Lévis (CA); Donald POIRIER, L'Ancienne-Lorette (CA); Jean-Yves SANCÉAU, Québec (CA); Guy BOIVIN, Québec (CA)

(73) Assignee: UNIVERSITÉ LAVAL, Québec, QC (CA)

(21) Appl. No.: 18/572,676

(22) PCT Filed: Jun. 23, 2022
(86) PCT No.: PCT/CA22/51007

§ 371 Date: Dec. 20, 2023

Related U.S. Application Data
(60) Provisional application No. 63/202,822, filed on Jun. 25, 2021.

(51) Int. Cl. A61K 31/085; A61K 31/047; A61K 31/133; A61K 31/202; A61K 31/4453; A61P 31/16

(52) U.S. Cl. CPC A61K 31/085; A61K 31/047; A61K 31/133; A61K 31/202; A61K 31/4453; A61P 31/16

(57) ABSTRACT

Derivatives of 10S, 17S-diHIDA (protectin DX, PDX) and medicinal uses thereof are disclosed. These derivatives are specialized pro-resolving mediators (SPRMs). In specific embodiments, said derivatives are used in to prevent or treat viral infections (including influenza and coronavirus infections), insulin resistance, diabetes, and inflammatory disorders.

### CROSS REFERENCE TO RELATED APPLICATIONS

This application claims the benefit of U.S. Provisional Application 63/202,822, filed Jun. 25, 2021. The contents of the referenced application are incorporated into the present application by reference.

### BACKGROUND

### 1. Field

This disclosure relates to the field of medicine, pharmacology, antiviral activity, endocrinology, inflammation, pharmacology, chemistry and biochemistry. In particular compositions comprising a specialized pro-resolving mediator, structural isomer thereof, and/or structural analog thereof, and methods of treatment using such compositions are disclosed. In particular, but not exclusively, the present disclosure broadly relates to methods of preventing, delaying the onset or reducing the severity of, preventing or reversing the progression of, or treating a viral infection or disease in a subject using a specialized pro-resolving mediator, structural isomer thereof, and/or structural analog thereof. In particular, but not exclusively, the present disclosure broadly relates to methods of preventing, delaying the onset or reducing the severity of, preventing or reversing the progression of, or treating insulin resistance or diabetes in a subject using a specialized pro-resolving mediator, structural isomer thereof, and/or structural analog thereof. In particular, but not exclusively, the present disclosure broadly relates to methods of lowering blood glucose levels in a subject, using a specialized pro-resolving mediator, structural isomer thereof, and/or structural analog thereof. In particular, but not exclusively, the present disclosure broadly relates to methods of preventing, delaying the onset or reducing the severity of, preventing or reversing the progression of, or treating an inflammatory disease, disorder, condition, or symptom in a subject using a specialized pro-resolving mediator, structural isomer thereof, and/or structural analog thereof. In particular, but not exclusively, the present disclosure broadly relates to methods of preventing, delaying the onset or reducing the severity of, preventing or reversing the progression of, or treating an inflammatory disease, disorder, condition, or symptom that is secondary to another condition in a subject using a specialized pro-resolving mediator, structural isomer thereof, and/or structural analog thereof.

The present disclosure also relates to methods of preventing, delaying the onset or reducing the severity of, preventing or reversing the progression of, or treating a viral infection or disease in a subject using a specialized pro-resolving mediator, structural isomer thereof, and/or structural analog thereof and a second agent. The present disclosure also relates to methods of preventing, delaying the onset or reducing the severity of, preventing or reversing the progression of, or treating insulin resistance or diabetes in a subject using a specialized pro-resolving mediator, structural isomer thereof, and/or structural analog thereof and a second agent. The present disclosure also relates to methods of lowering blood glucose levels in a subject, using a specialized pro-resolving mediator, structural isomer thereof, and/or structural analog thereof and a second agent. The present disclosure also relates to methods of preventing, delaying the onset or reducing the severity of, preventing or reversing the progression of, or treating an inflammatory disease, disorder, condition, or symptom in a subject using a specialized pro-resolving mediator, structural isomer thereof, and/or structural analog thereof and a second agent. The present disclosure also relates to methods of preventing, delaying the onset or reducing the severity of, preventing or reversing the progression of, or treating an inflammatory disease, disorder, condition, or symptom that is secondary to another condition in a subject using a specialized pro-resolving mediator, structural isomer thereof, and/or structural analog thereof and a second agent.

### 2. Related Art

Eicosapentaenoic acid (EPA) and docosahexaenoic acid (DHA) are the most physiologically important members of the natural omega-3 fatty acid class, both being key precursors in the biosynthesis of hydroxylated metabolites that mediate resolution of the inflammation process. Such lipid mediators, named specialized pro-resolving lipid mediators (SPMs), include the E-series (RvEs) and D-series (RvDs) resolvins, protectins (PDs) and maresins (MaRs).^{[1,2]} Structurally, some of the SPMs, including members of the resolvins (RvE1 and RvD3), maresins (MRS1) and protectins (PD1 and PDX) are characterized by a conjugated trienic system flanked by two allylic alcohols, including members of resolving (RvE1 and RvD3), maresins (MRS1) and protectins (PD1 and PDX). Interestingly, protectin D1 (PD1), also known as neuroprotectin D1 (NPD1) has a significant role as an anti-inflammatory, immunoregulatory, anti-apoptotic and neuroprotective molecule.^{[3]} PD1 is derived from DHA and proceeds through the action of 15-lipoxygenase (15-LO-1) on DHA, leading to the formation of the (17S)-hydro (peroxy)-DHA intermediate. This intermediate is rapidly processed to form a 16 (17)-epoxide-containing molecule which undergoes enzymatic hydrolysis to form PD1 (FIG. 1A). The molecular structure of PD1 is characterized by the presence of two hydroxy groups and a conjugated triene system comprising one cis-olefin (FIG. 1B).^{[4]}

An additional protectin, derived from DHA trough the sequential action of a pair of lipoxygenases, was reported by Hong et al. in 2003.^{[5]} The structure of this double-oxygenated protectin was partially elucidated by Butovich in 2005.^{[6]} However, its complete stereochemistry was established by Serhan et al. in 2006 as 10S, 17S-diHDA (PDX) (FIG. 1B).^{[3e]} The structural and configurational assignment of 10S, 17S-diHDA was subsequently confirmed by Guichardant et al.^{[7]} Interestingly, the structure of PDX differs from PD1 in the geometry of the conjugated triene system; PD1 exhibiting an E,E,Z-triene system, and PDX exhibiting an E,Z,E-triene system. Moreover, the pair of hydroxy groups in PD1 have the (10R, 17S) configuration, different from the (10S, 17S) configuration of PDX (FIG. 1).

PDX and PD1 have both been shown to exert immunoresolving actions in a number of in vitro and in vivo inflammation models, including acute lung injury and osteoarthritis.^{[8,9]} In addition, PDX has been shown to inhibit human platelet aggregation responses, inhibit the replication of the influenza virus, and confer protection against sepsis in mice.^{[10-12]} PDX has also been shown to reverse the fibrotic process in mice with lung fibrosis, and improve hepatic steatosis.^{[13,14]} The potential use of protectins for the treatment of COVID-19 has also been suggested.^{[15-18]}

The glucoregulatory activity of PDX has also recently been investigated.^{[19]} PDX was shown to induce the release of the prototypic myokine interleukin-6 (IL-6), at submicromolar concentrations, for activation of AMP-activated kinase (AMPK) and for the prevention of lipid-induced and obesity-linked insulin-resistance in mouse models. These finding suggest that PDX could potentially be used in methods for alleviating type-2 diabetes through both anti-inflammatory and insulin-sensitizing actions. Interestingly, PD1 was shown to be ineffective in inducing IL-6 release from skeletal muscle cells.

SPMs are involved in host responses to microorganisms, including bacterial, viral, fungal and parasitic pathogens. They also stimulate the resolution of inflammation in non-infectious diseases and tissue injury.^{[20,21]} These unique properties of SPMs have incited the development of new approaches for treating inflammation-associated diseases and for stimulating tissue regeneration via resolution pharmacology.^{[22]}

Methods of preventing, delaying the onset or reducing the severity of, preventing or reversing the progression of, or treating a viral infection or disease in a subject using a specialized pro-resolving mediator, structural isomer thereof, and/or structural analog are of commercial interest. Moreover, methods of preventing, delaying the onset or reducing the severity of, preventing or reversing the progression of, or treating insulin resistance or diabetes in a subject using a specialized pro-resolving mediator, structural isomer thereof, and/or structural analog thereof are of commercial interest. Moreover, methods of lowering blood glucose levels in a subject, using a specialized pro-resolving mediator, structural isomer thereof, and/or structural analog thereof are of commercial interest. Moreover, methods of preventing, delaying the onset or reducing the severity of, preventing or reversing the progression of, or treating an inflammatory disease, disorder, condition, or symptom in a subject using a specialized pro-resolving mediator, structural isomer thereof, and/or structural analog thereof are of commercial interest. Yet moreover, methods of preventing, delaying the onset or reducing the severity of, preventing or reversing the progression of, or treating an inflammatory disease, disorder, condition, or symptom that is secondary to another condition in a subject using a specialized pro-resolving mediator, structural isomer thereof, and/or structural analog thereof and a second agent are of commercial interest.

### SUMMARY

The present disclosure broadly relates to the use of pro-resolving mediators, structural isomers thereof, and/or structural analogs thereof for treating a viral infection, for treating insulin resistance or diabetes, for lowering blood glucose levels, and for treating an inflammatory disease in a subject.

In an aspect, the present disclosure relates to methods of preventing, delaying the onset or reducing the severity of, preventing or reversing the progression of, or treating a viral infection or disease in a subject using a specialized pro-resolving mediator, structural isomer thereof, and/or structural analog thereof.

In an aspect, the present disclosure relates to methods of preventing, delaying the onset or reducing the severity of, preventing or reversing the progression of, or treating insulin resistance or diabetes in a subject using a specialized pro-resolving mediator, structural isomer thereof, and/or structural analog thereof.

In an aspect, the present disclosure relates to methods of lowering blood glucose levels in a subject, using a specialized pro-resolving mediator, structural isomer thereof, and/or structural analog thereof.

In an aspect, the present disclosure relates to methods of preventing, delaying the onset or reducing the severity of, preventing or reversing the progression of, or treating an inflammatory disease, disorder, condition, or symptom in a subject using a specialized pro-resolving mediator, structural isomer thereof, and/or structural analog thereof.

In an aspect, the present disclosure relates to methods of preventing, delaying the onset or reducing the severity of, preventing or reversing the progression of, or treating an inflammatory disease, disorder, condition, or symptom that is secondary to another condition in a subject using a specialized pro-resolving mediator, structural isomer thereof, and/or structural analog thereof.

In an aspect, the present disclosure relates to methods of preventing, delaying the onset or reducing the severity of, preventing or reversing the progression of, or treating an inflammatory disease, disorder, condition, or symptom that is secondary to another condition in a subject using a specialized pro-resolving mediator, structural isomer thereof, and/or structural analog thereof.

In an aspect, the present disclosure relates to methods of preventing, delaying the onset or reducing the severity of, preventing or reversing the progression of, or treating a viral infection or disease in a subject using a specialized pro-resolving mediator, structural isomer thereof, and/or structural analog thereof and a second agent.

In an aspect, the present disclosure relates to methods of preventing, delaying the onset or reducing the severity of, preventing or reversing the progression of, or treating insulin resistance or diabetes in a subject using a specialized pro-resolving mediator, structural isomer thereof, and/or structural analog thereof and a second agent.

In an aspect, the present disclosure relates to methods of lowering blood glucose levels in a subject, using a specialized pro-resolving mediator, structural isomer thereof, and/or structural analog thereof and a second agent.

In an aspect, the present disclosure relates to methods of preventing, delaying the onset or reducing the severity of, preventing or reversing the progression of, or treating an inflammatory disease, disorder, condition, or symptom in a subject using a specialized pro-resolving mediator, structural isomer thereof, and/or structural analog thereof and a second agent.

In an aspect, the present disclosure relates to methods of preventing, delaying the onset or reducing the severity of, preventing or reversing the progression of, or treating an inflammatory disease, disorder, condition, or symptom that is secondary to another condition in a subject using a specialized pro-resolving mediator, structural isomer thereof, and/or structural analog thereof and a second agent.

In an aspect, the present disclosure relates to methods of medical treatment comprising the use of a specialized pro-resolving mediator, structural isomer thereof, and/or structural analog thereof, having the structure:

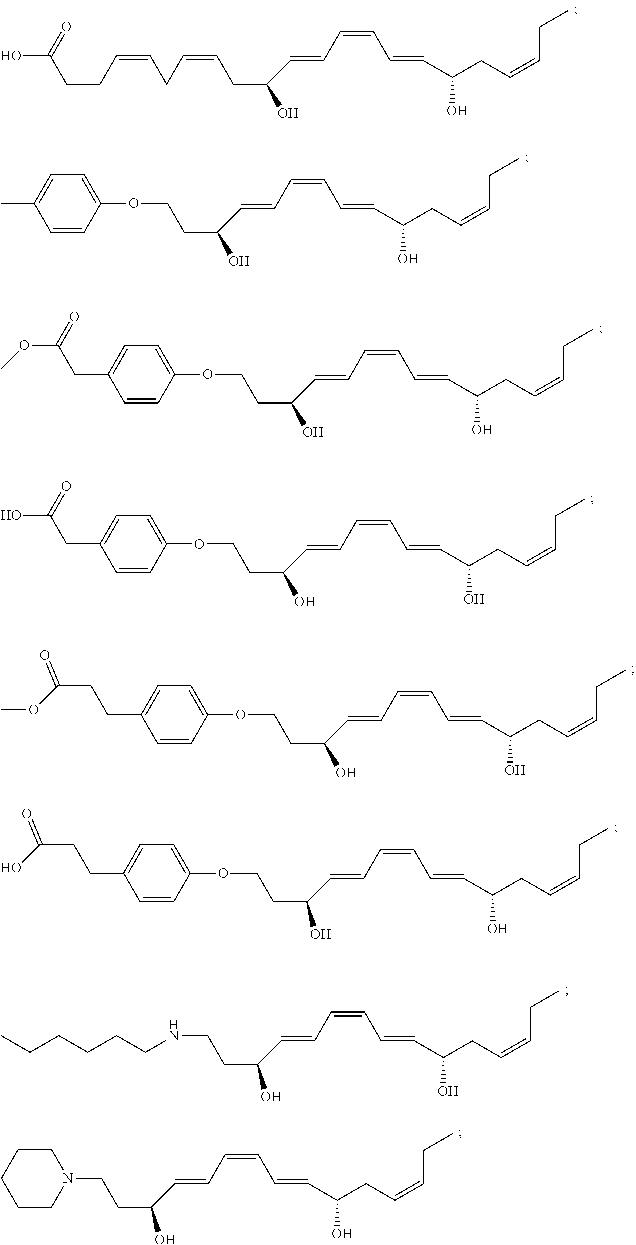

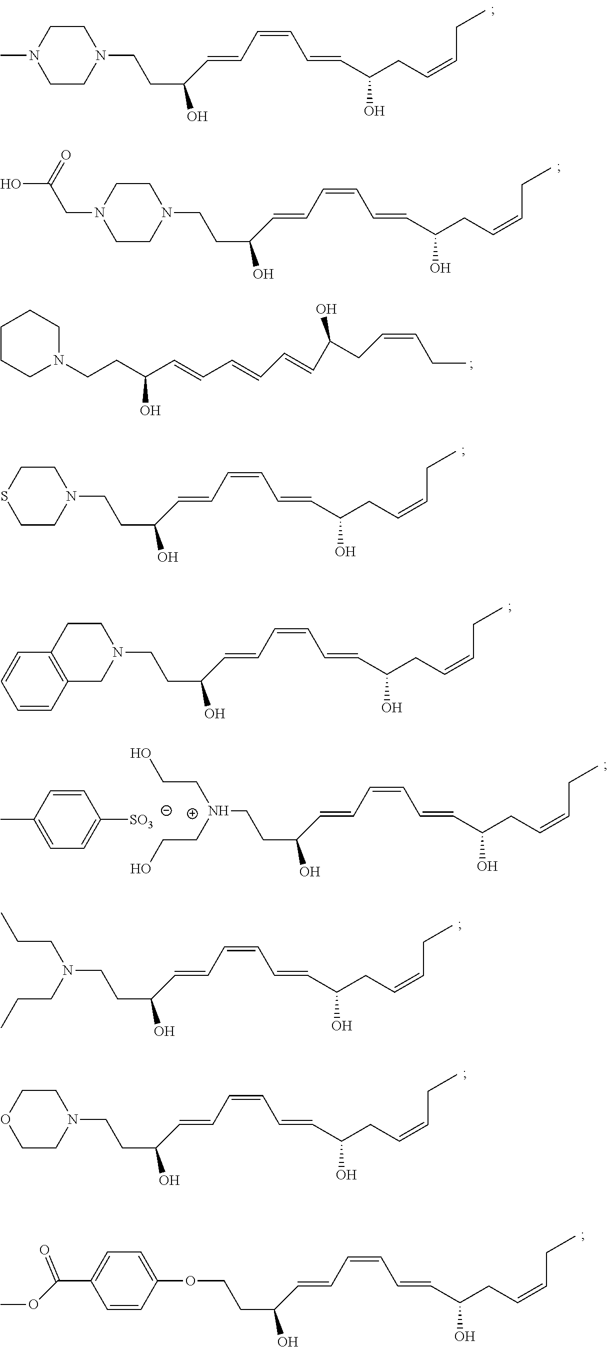

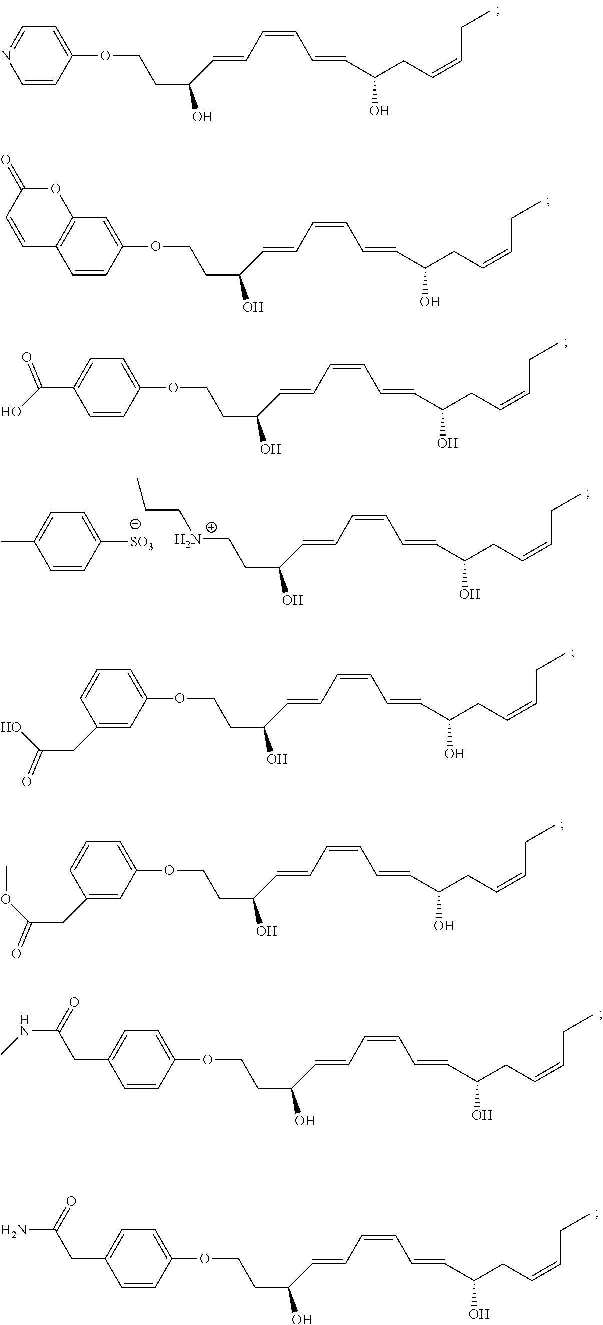

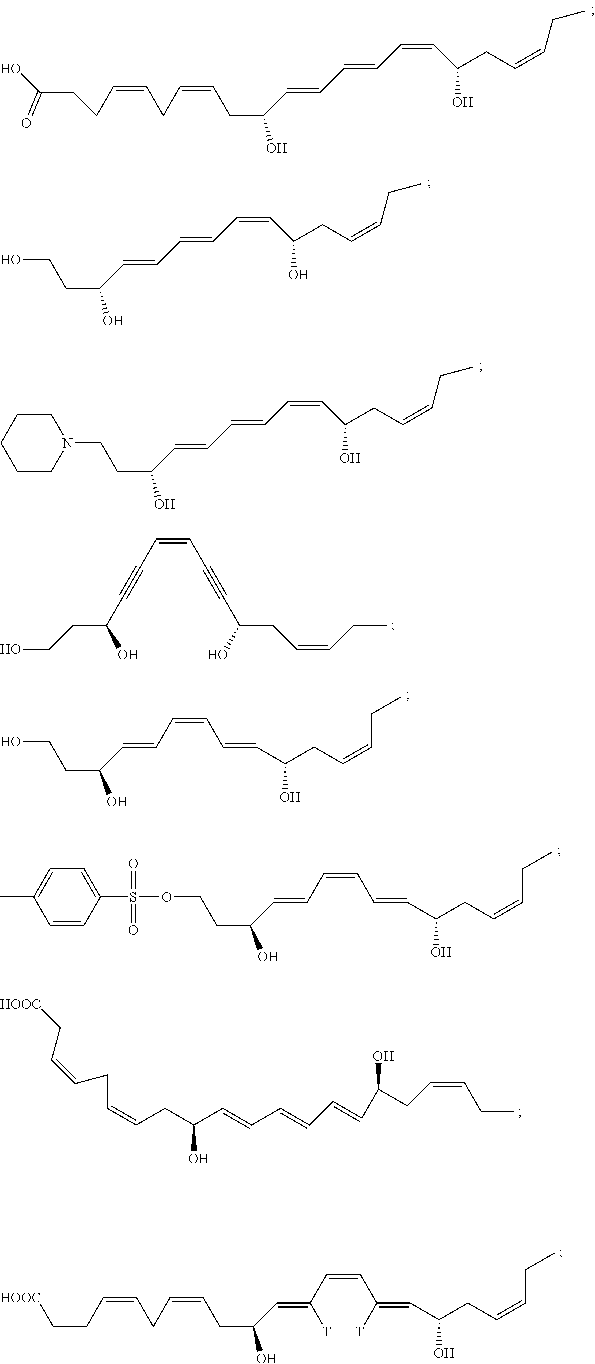

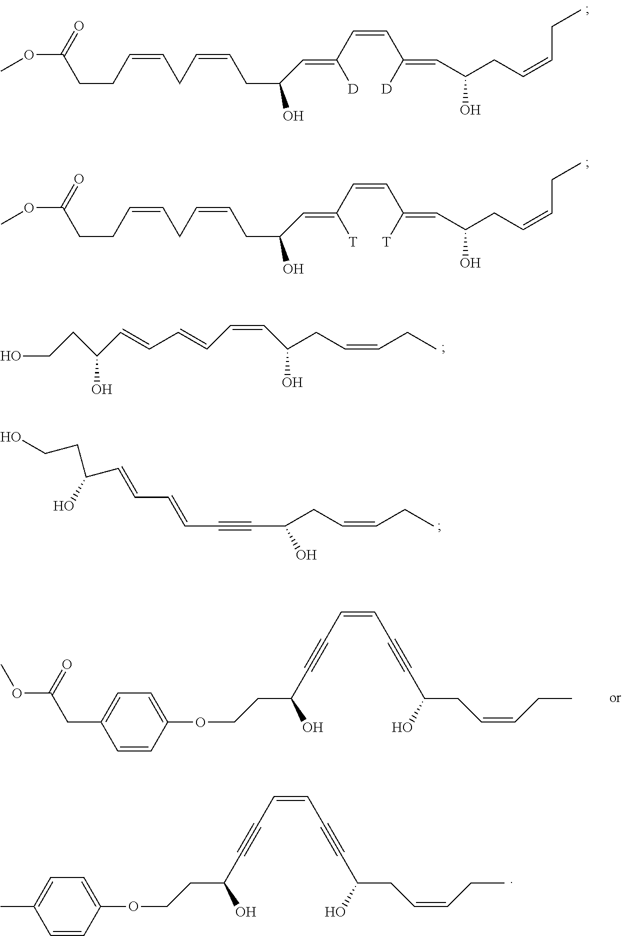

Disclosed in the context of the present disclosure are embodiments 1 to 96: Embodiment 1 is a method of preventing, delaying the onset or reducing the severity of, preventing or reversing the progression of, or treating a viral infection or disease in a subject, said method comprising administering a protectin, protectin analog, structural isomer, or a pharmaceutically acceptable salt thereof, to the subject.

Embodiment 2 is the method of embodiment 1, wherein the viral infection is a coronavirus infection.

Embodiment 3 is the method of embodiment 1 or 2, wherein the viral disease is a viral respiratory disease.

Embodiment 4 is the method of any one of embodiments 1 to 3, wherein the viral infection is a SARS viral infection.

Embodiment 5 is the method of embodiment 4, wherein the viral infection is a SARS-CoV viral infection.

Embodiment 6 is the method of embodiment 5, wherein the viral infection is a SARS-CoV-2 viral infection.

Embodiment 7 is the method of any one of embodiments 1 to 6, wherein the viral disease is COVID-19.

Embodiment 8 is the method of any one of embodiments 1 to 7, wherein the subject suffers from insulin resistance, diabetes, or obesity.

Embodiment 9 is the method of embodiment 1, wherein the viral infection is influenza.

Embodiment 10 is the method of any one of embodiments 1 to 9, wherein the protectin, protectin analog, structural isomer, or a pharmaceutically acceptable salt thereof is at least one of:

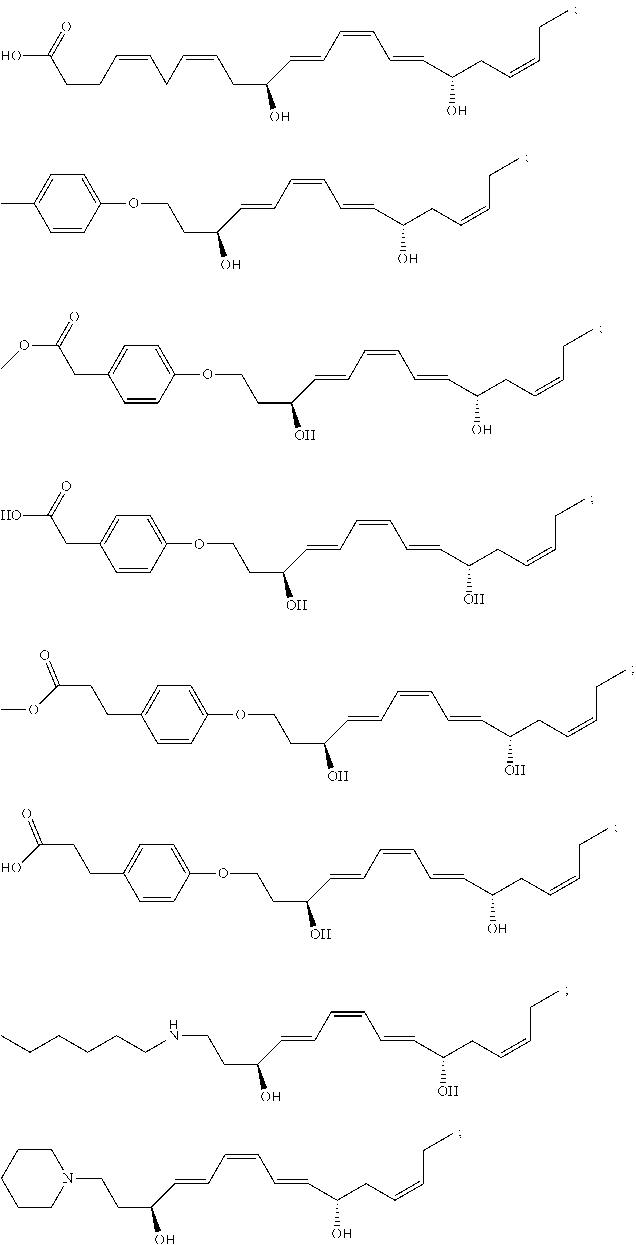

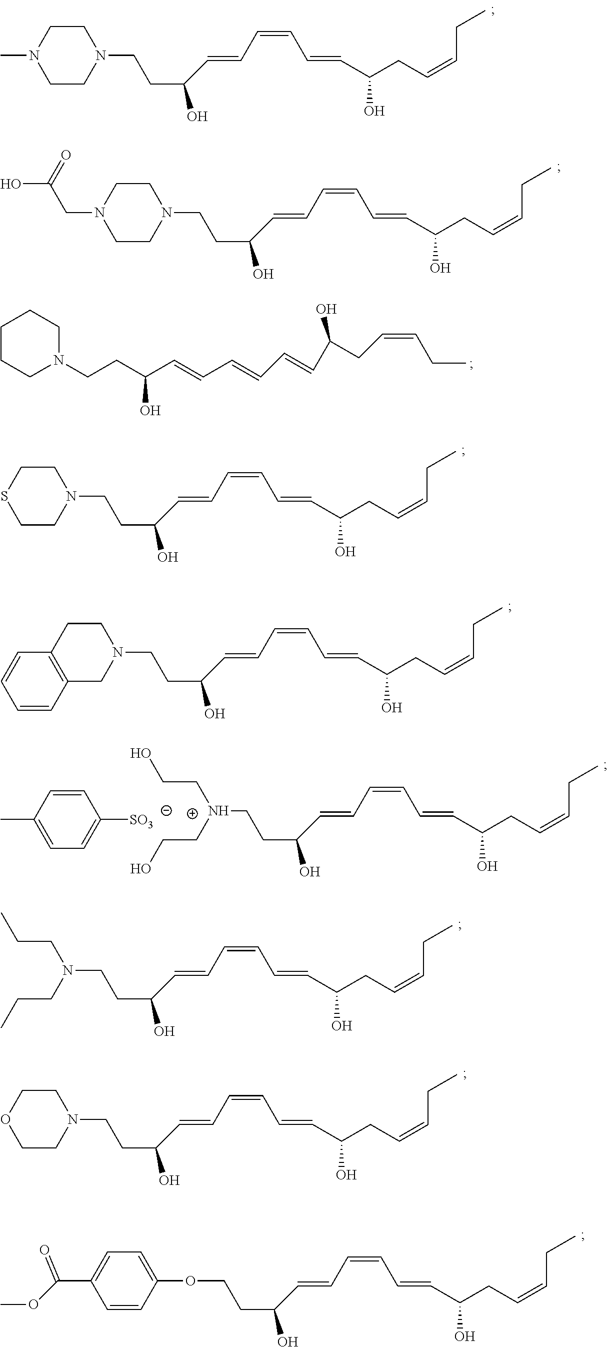

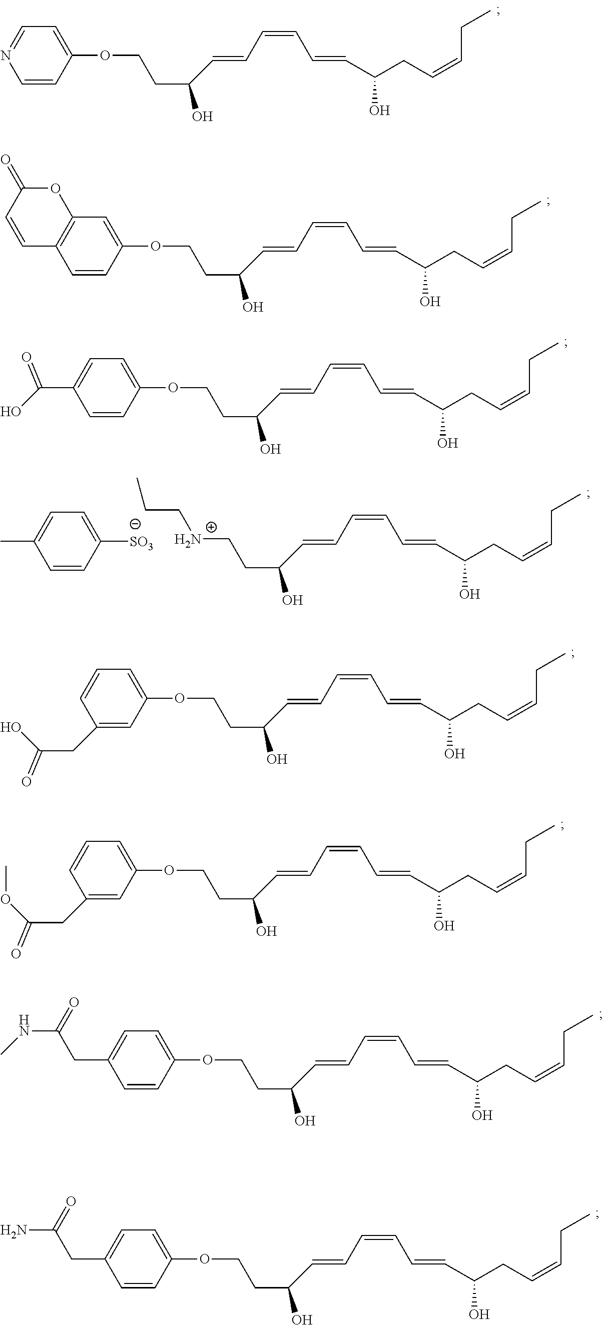

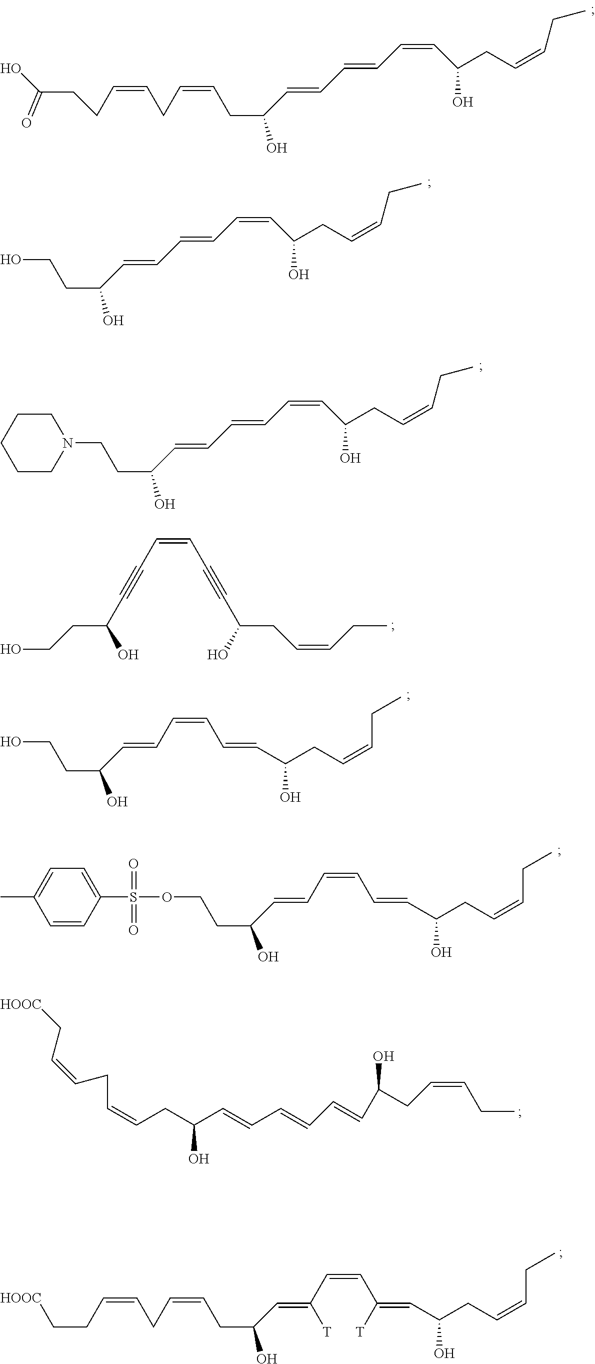

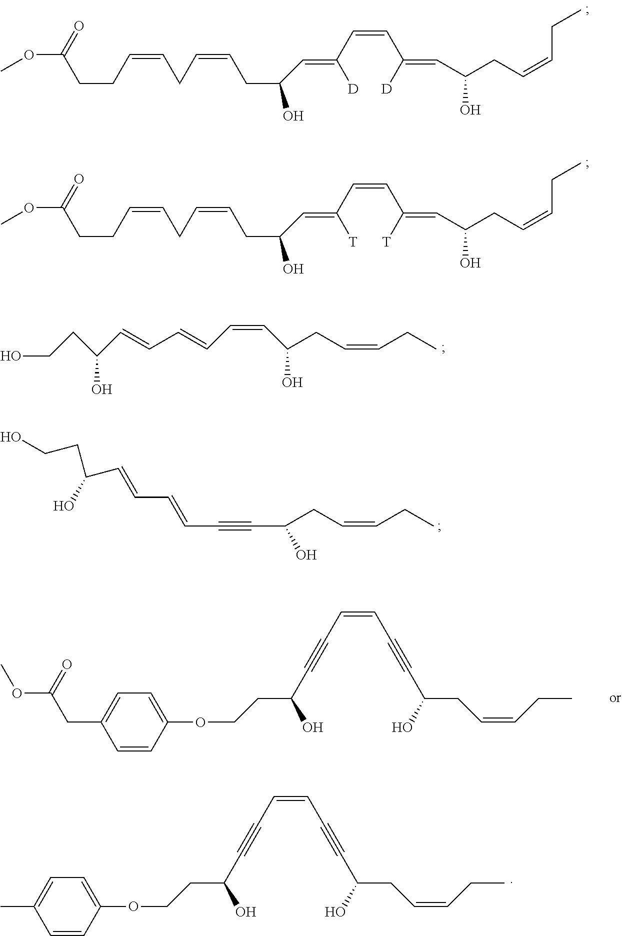

Embodiment 11 is the use of a protectin, protectin analog, structural isomer, or a pharmaceutically acceptable salt thereof for preventing, delaying the onset or reducing the severity of, preventing or reversing the progression of, or treating a viral infection or disease in a subject.

Embodiment 12 is the use of a protectin, protectin analog, structural isomer, or a pharmaceutically acceptable salt thereof for the preparation of a medicament for preventing, delaying the onset or reducing the severity of, preventing or reversing the progression of, or treating a viral infection or disease in a subject.

Embodiment 13 is the use of embodiment 11 or 12, wherein the viral infection is a coronavirus infection.

Embodiment 14 is the use of any one of embodiments 11 to 13, wherein the viral disease is a viral respiratory disease.

Embodiment 15 is the use of any one of embodiments 11 to 14, wherein the viral infection is a SARS viral infection.

Embodiment 16 is the use of embodiment 15, wherein the viral infection is a SARS-CoV viral infection.

Embodiment 17 is the use of embodiment 16, wherein the viral infection is a SARS-CoV-2 viral infection.

Embodiment 18 is the use of any one of embodiments 11 to 17, wherein the viral disease is COVID-19.

Embodiment 19 is the use of any one of embodiments 11 to 18, wherein the subject suffers from insulin resistance, diabetes, or obesity.

Embodiment 20 is the use of embodiment 11 or 12, wherein the viral infection is influenza.

Embodiment 21 is the use of any one of embodiments 11 to 20, wherein the protectin, protectin analog, structural isomer, or a pharmaceutically acceptable salt thereof is at least one of:

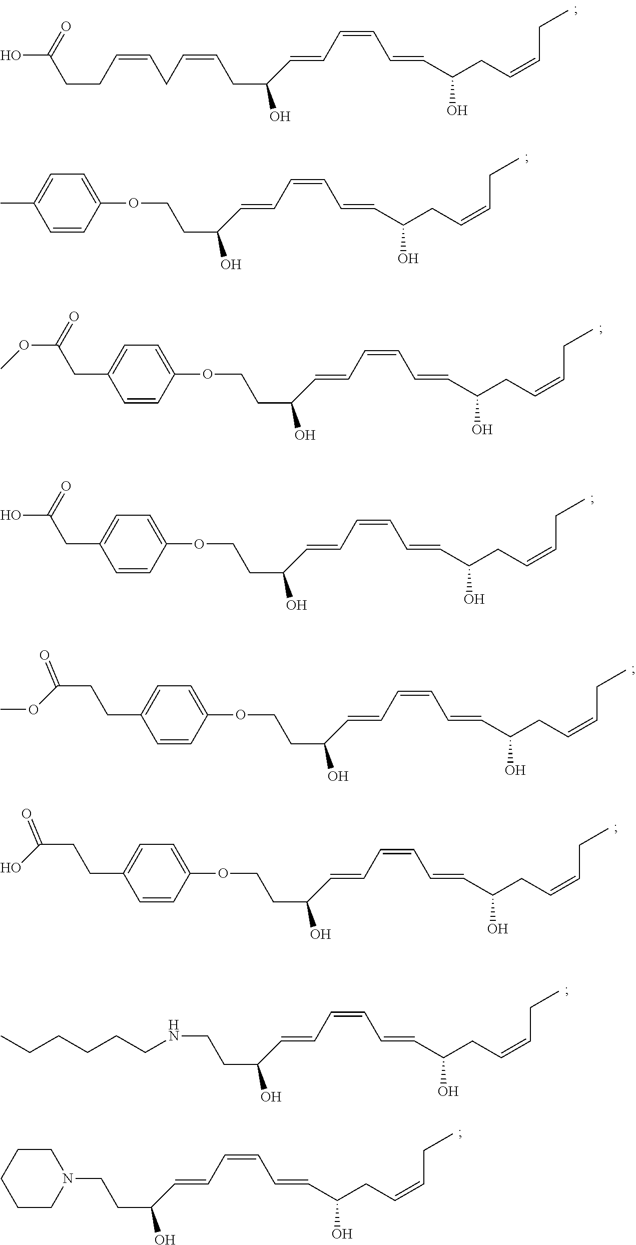

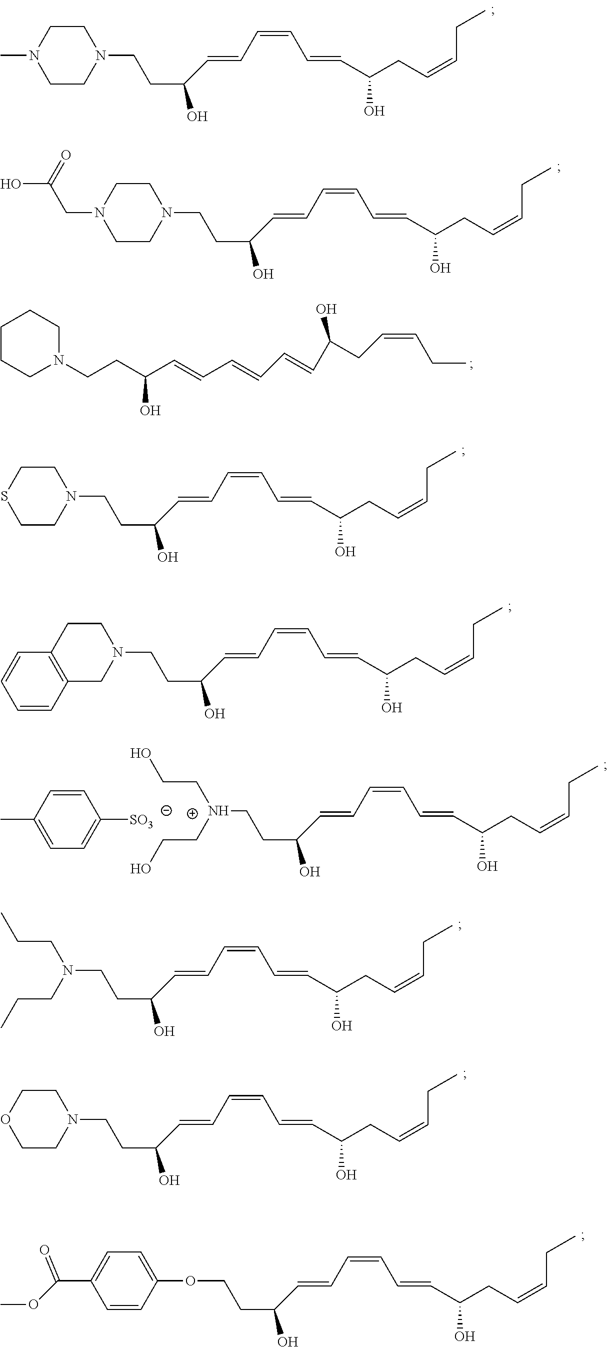

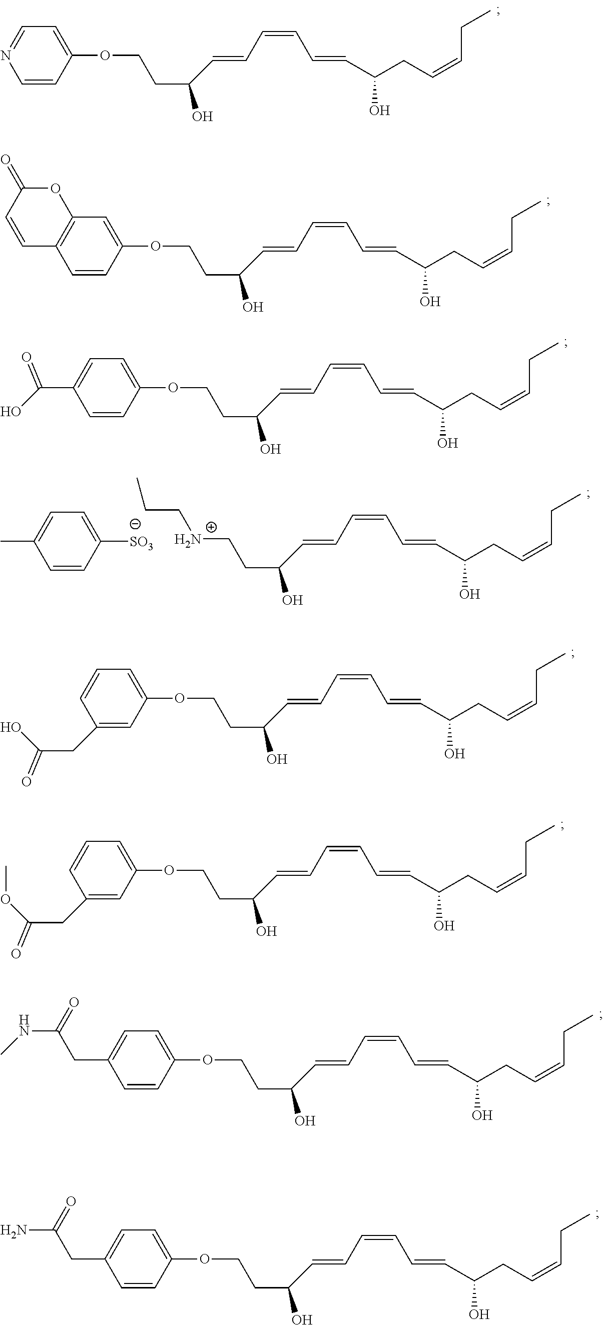

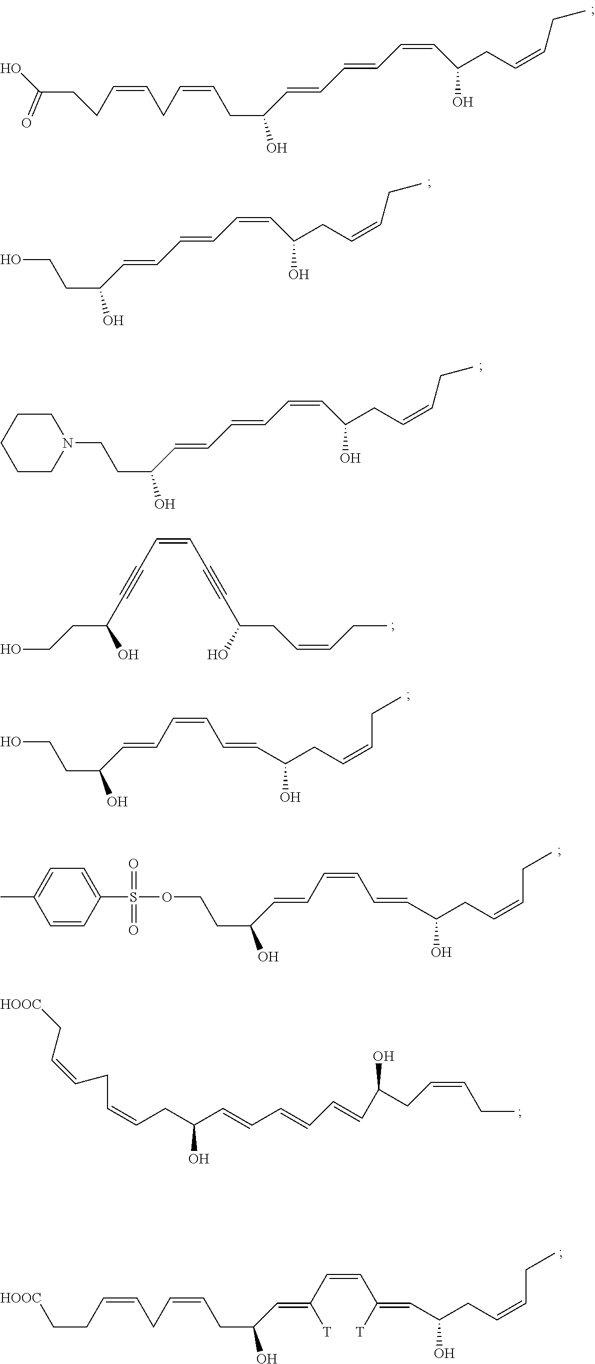

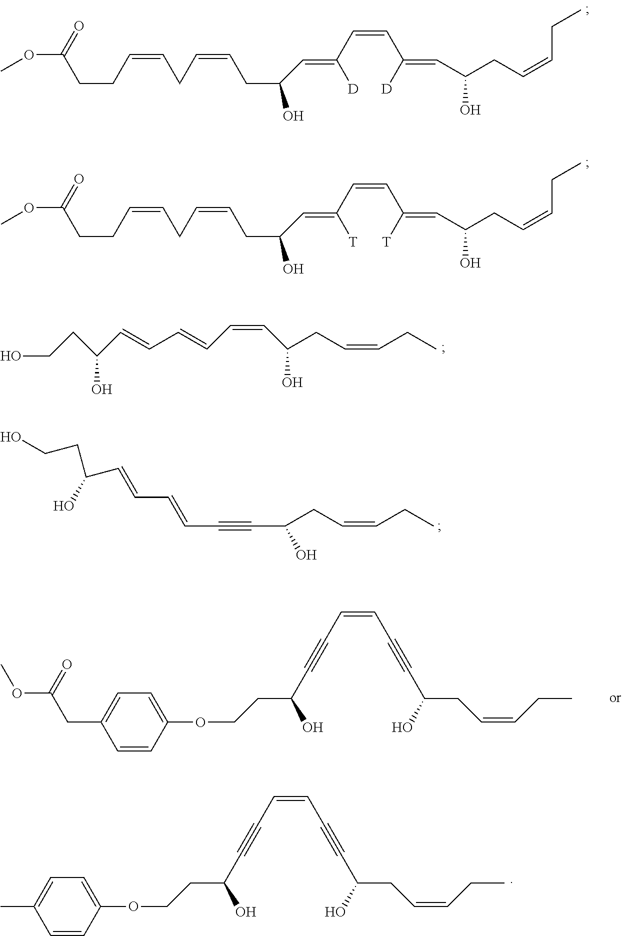

Embodiment 22 is a protectin, protectin analog, structural isomer, or a pharmaceutically acceptable salt thereof for use in preventing, delaying the onset or reducing the severity of, preventing or reversing the progression of, or treating a viral infection or disease in a subject.

Embodiment 23 is the protectin, protectin analog, structural isomer, or pharmaceutically acceptable salt thereof for use of embodiment 22, wherein the viral infection is a coronavirus infection.

Embodiment 24 is the protectin, protectin analog, structural isomer, or pharmaceutically acceptable salt thereof for use of embodiment 22 or 23, wherein the viral disease is a viral respiratory disease.

Embodiment 25 is the protectin, protectin analog, structural isomer, or pharmaceutically acceptable salt thereof for use of any one of embodiments 22 to 24, wherein the viral infection is a SARS viral infection.

Embodiment 26 is the protectin, protectin analog, structural isomer, or pharmaceutically acceptable salt thereof for use of embodiment 25, wherein the viral infection is a SARS-CoV viral infection.

Embodiment 27 is the protectin, protectin analog, structural isomer, or pharmaceutically acceptable salt thereof for use of embodiment 26, wherein the viral infection is a SARS-CoV-2 viral infection.

Embodiment 28 is the protectin, protectin analog, structural isomer, or pharmaceutically acceptable salt thereof for use of any one of embodiments 22 to 27, wherein the viral disease is COVID-19.

Embodiment 29 is the protectin, protectin analog, structural isomer, or pharmaceutically acceptable salt thereof for use of any one of embodiments 22 to 28, wherein the subject suffers from insulin resistance, diabetes, or obesity.

Embodiment 30 is the protectin, protectin analog, structural isomer, or pharmaceutically acceptable salt thereof for use of embodiment 22 or 23, wherein the viral infection is influenza.

Embodiment 31 is the protectin, protectin analog, structural isomer, or pharmaceutically acceptable salt thereof for use of any one of embodiments 22 to 30, wherein the protectin, protectin analog, structural isomer, or a pharmaceutically acceptable salt thereof is at least one of:

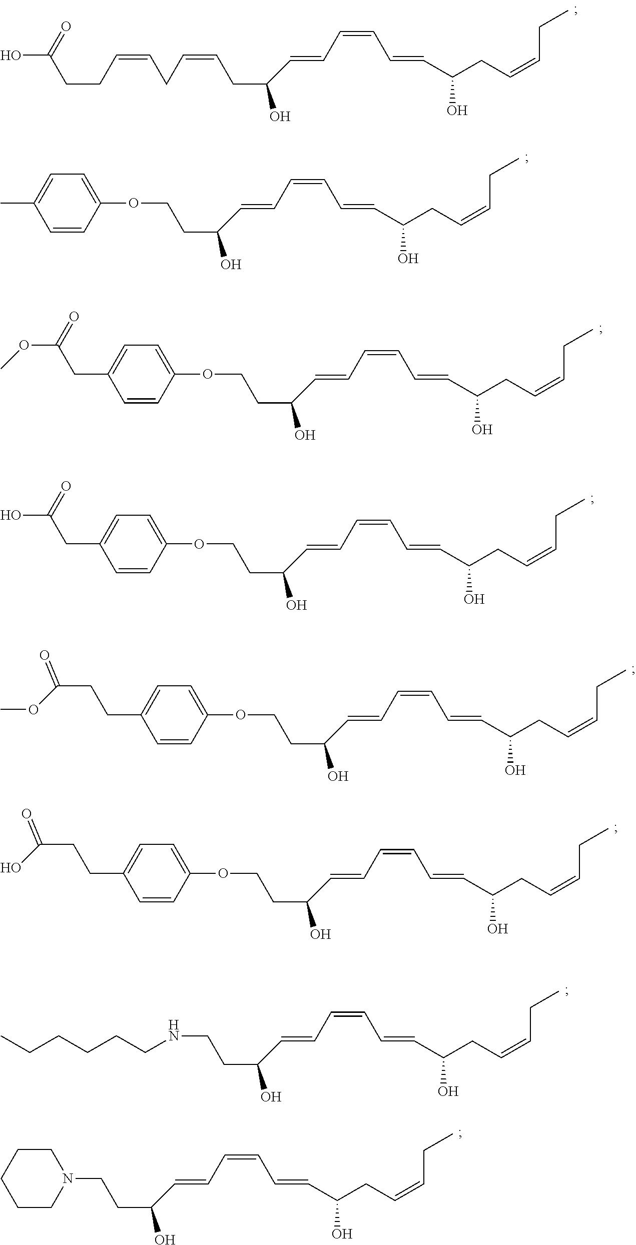

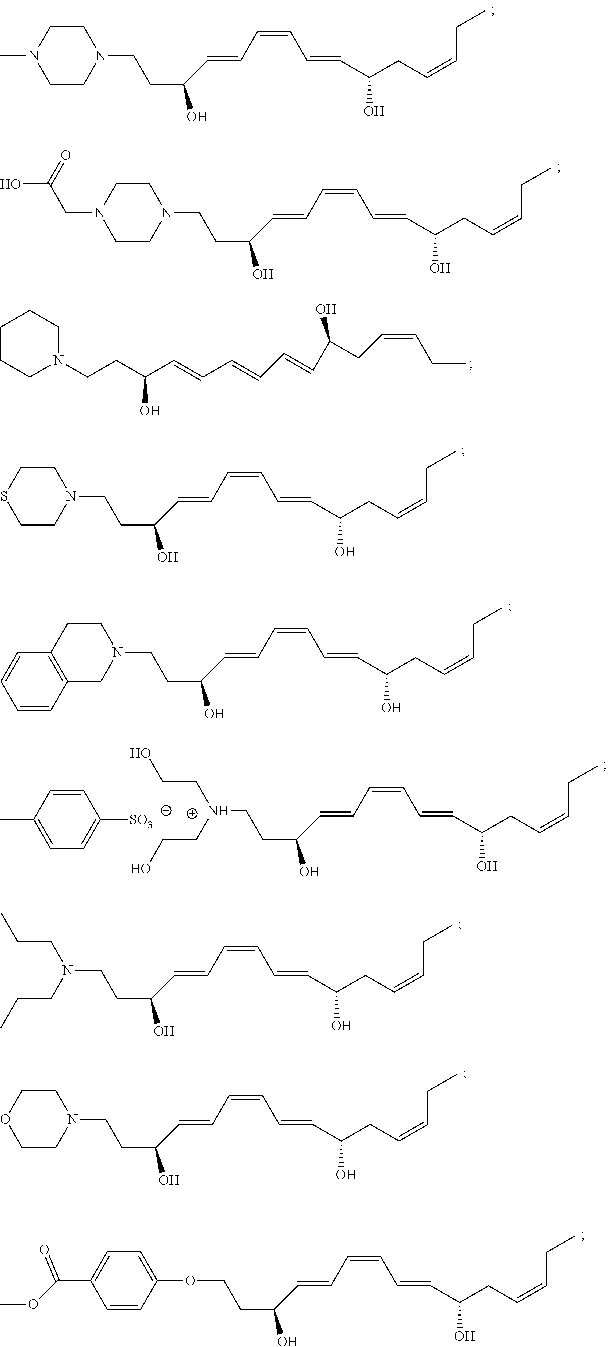

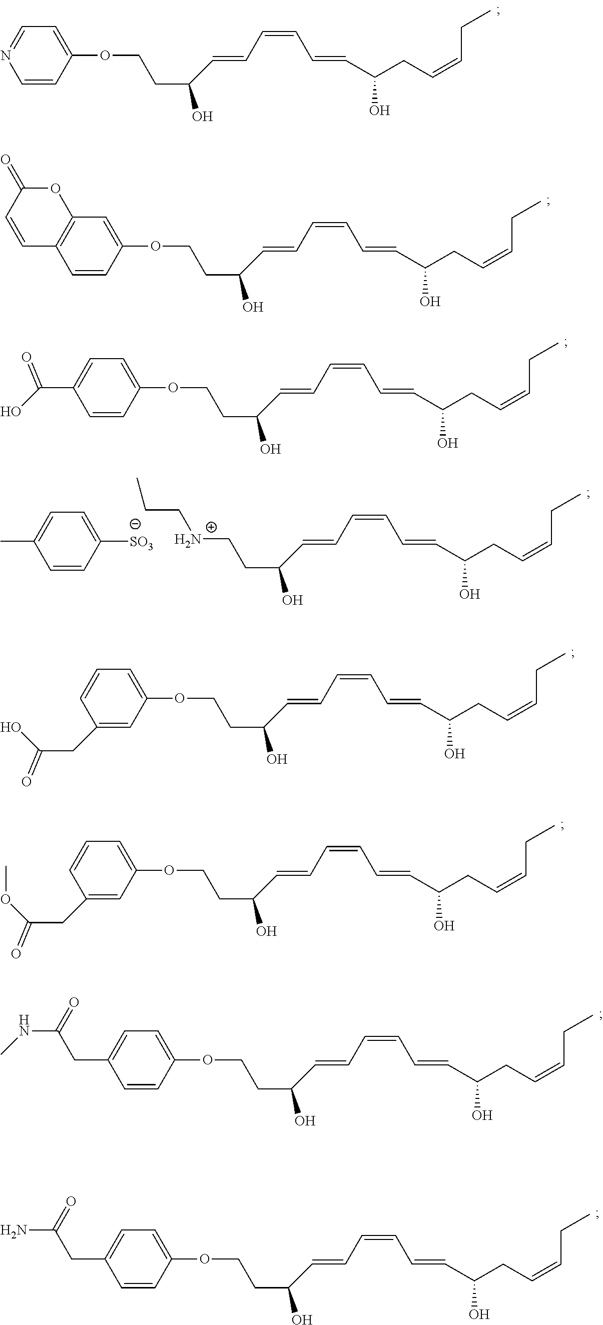

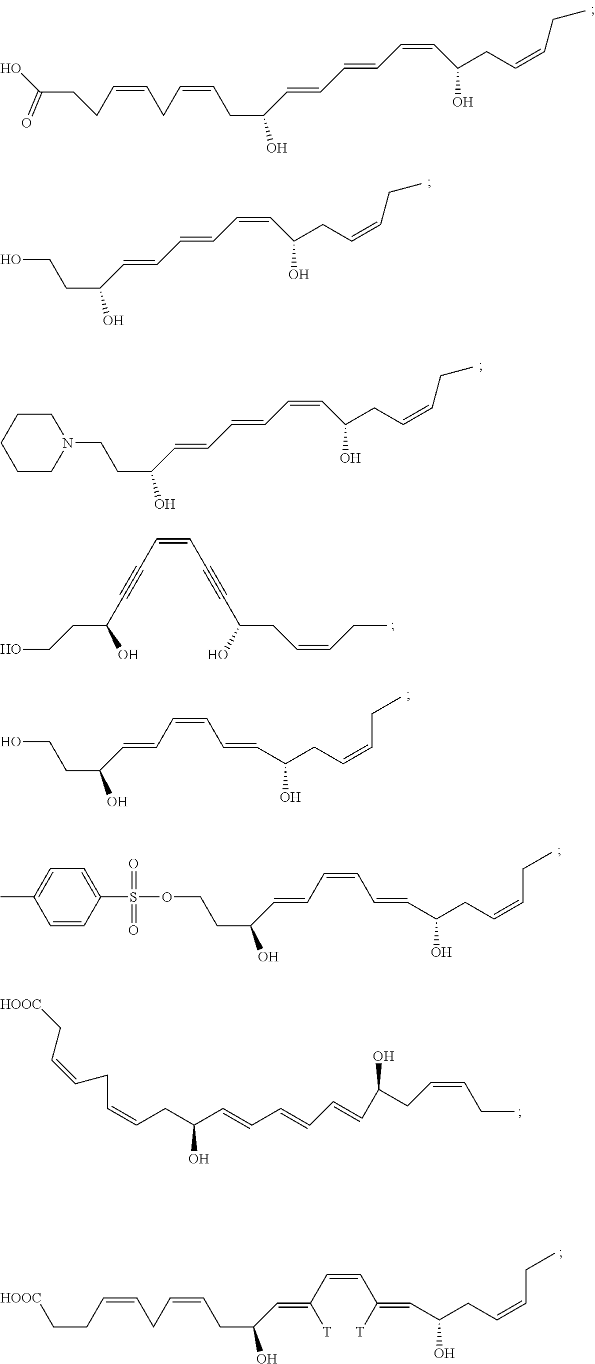

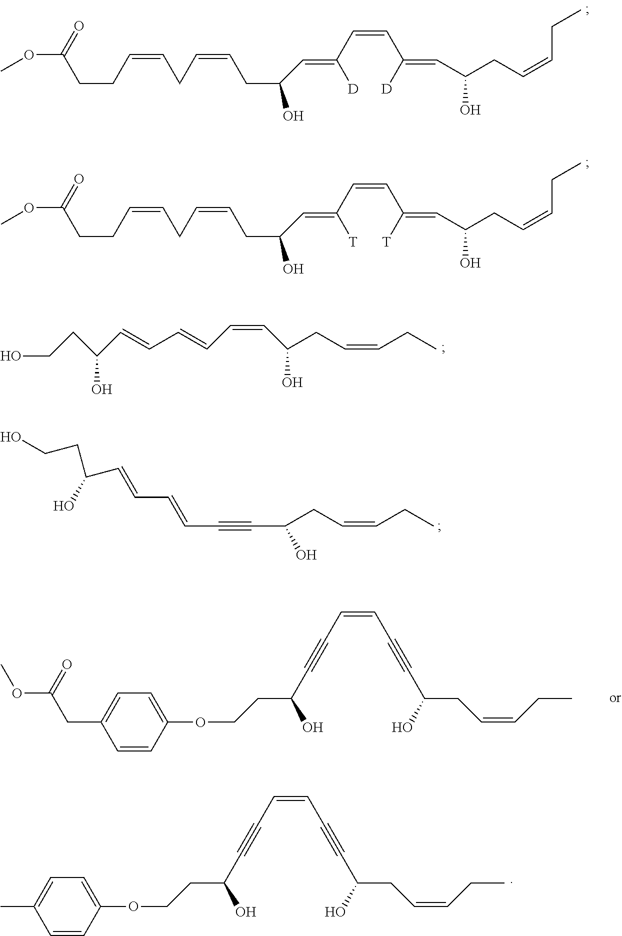

Embodiment 32 is a method of preventing, delaying the onset or reducing the severity of, preventing or reversing the progression of, or treating insulin resistance or diabetes in a subject, said method comprising administering a protectin, protectin analog, structural isomer, or a pharmaceutically acceptable salt thereof, to the subject.

Embodiment 33, the method of embodiment 32, wherein insulin resistance is diagnosed by measuring a patient's insulin level.

Embodiment 34 is the method of embodiment 32, wherein diabetes is diagnosed by measuring a patient's glucose level.

Embodiment 35 is the method of any one of embodiments 32 to 34, wherein diabetes is type 2 diabetes.

Embodiment 36 is the method of any one of embodiments 32 to 35, wherein the subject suffers from obesity.

Embodiment 37 is the method of any one of embodiments 32 to 36, wherein the protectin, protectin analog, structural isomer, or a pharmaceutically acceptable salt thereof is at least one of:

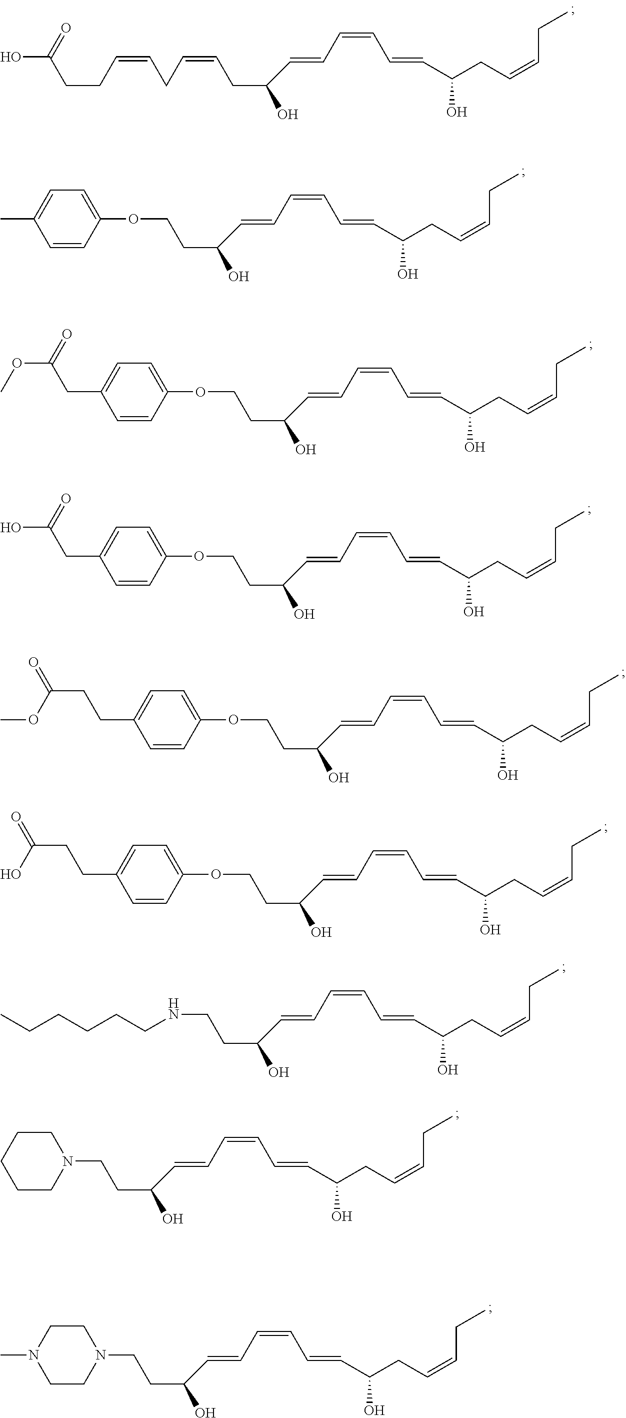

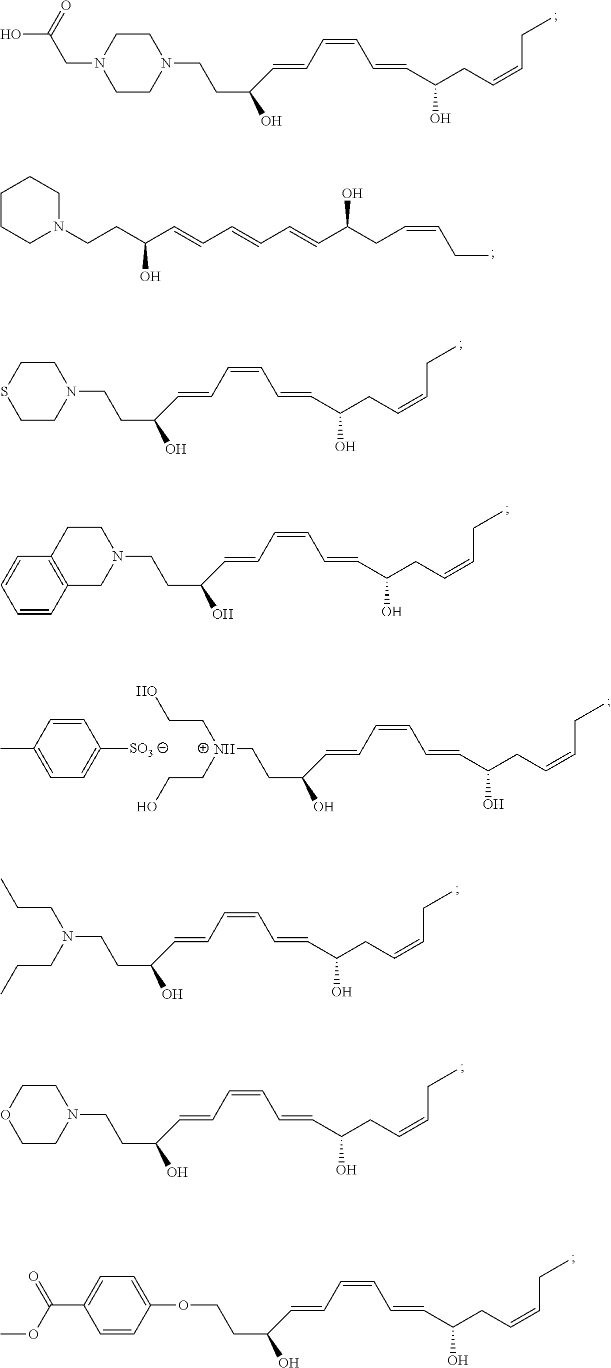

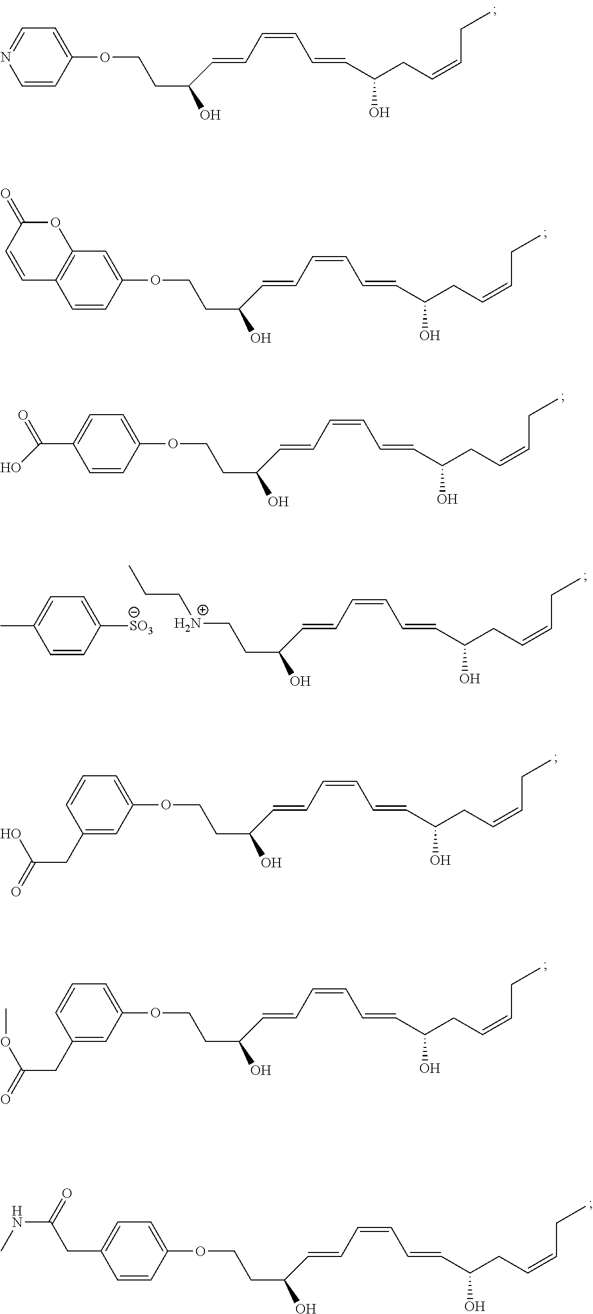

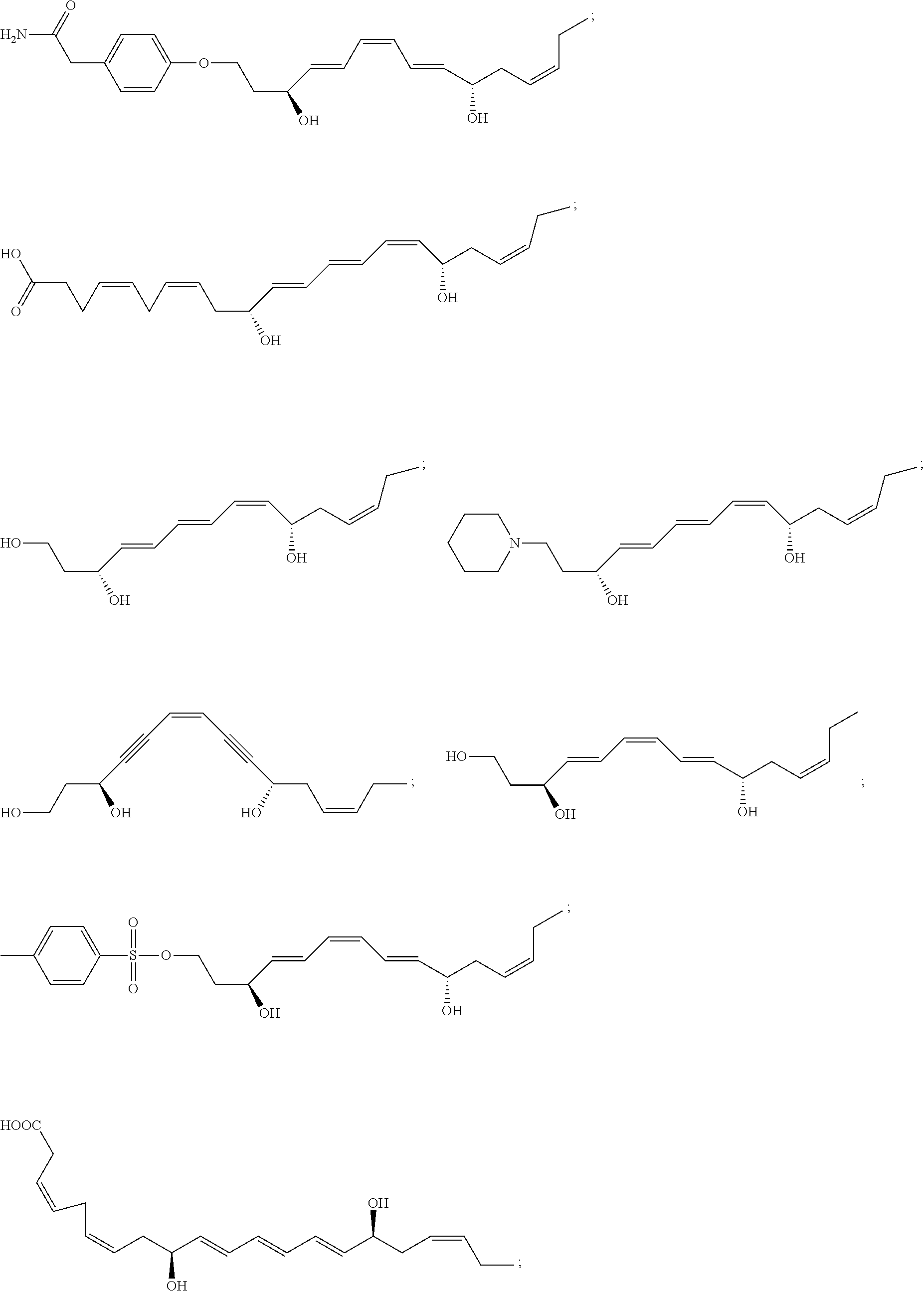

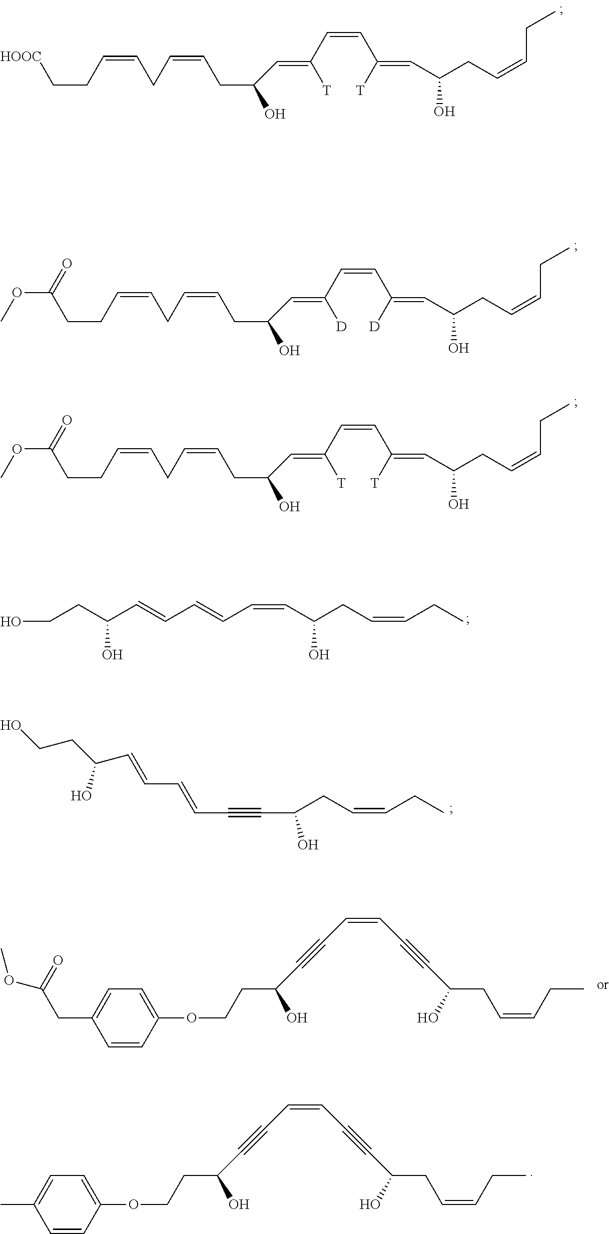

Embodiment 38 is the use of a protectin, protectin analog, structural isomer, or a pharmaceutically acceptable salt thereof for preventing, delaying the onset or reducing the severity of, preventing or reversing the progression of, or treating insulin resistance or diabetes in a subject.

Embodiment 39 is the use of a protectin, protectin analog, structural isomer, or a pharmaceutically acceptable salt thereof for the preparation of a medicament for preventing, delaying the onset or reducing the severity of, preventing or reversing the progression of, or treating insulin resistance or diabetes in a subject.

Embodiment 40 is the use of embodiment 38 or 39, wherein insulin resistance is diagnosed by measuring a patient's insulin level.

Embodiment 41 is the use of embodiment 38 or 39, wherein diabetes is diagnosed by measuring a patient's glucose level.

Embodiment 42 is the use of any one of embodiments 38 to 41, wherein diabetes is type 2 diabetes.

Embodiment 43 is the use of any one of embodiments 38 to 42, wherein the subject suffers from obesity.

Embodiment 44 is the use of any one of embodiments 38 to 43, wherein the protectin, protectin analog, structural isomer, or a pharmaceutically acceptable salt thereof is at least one of:

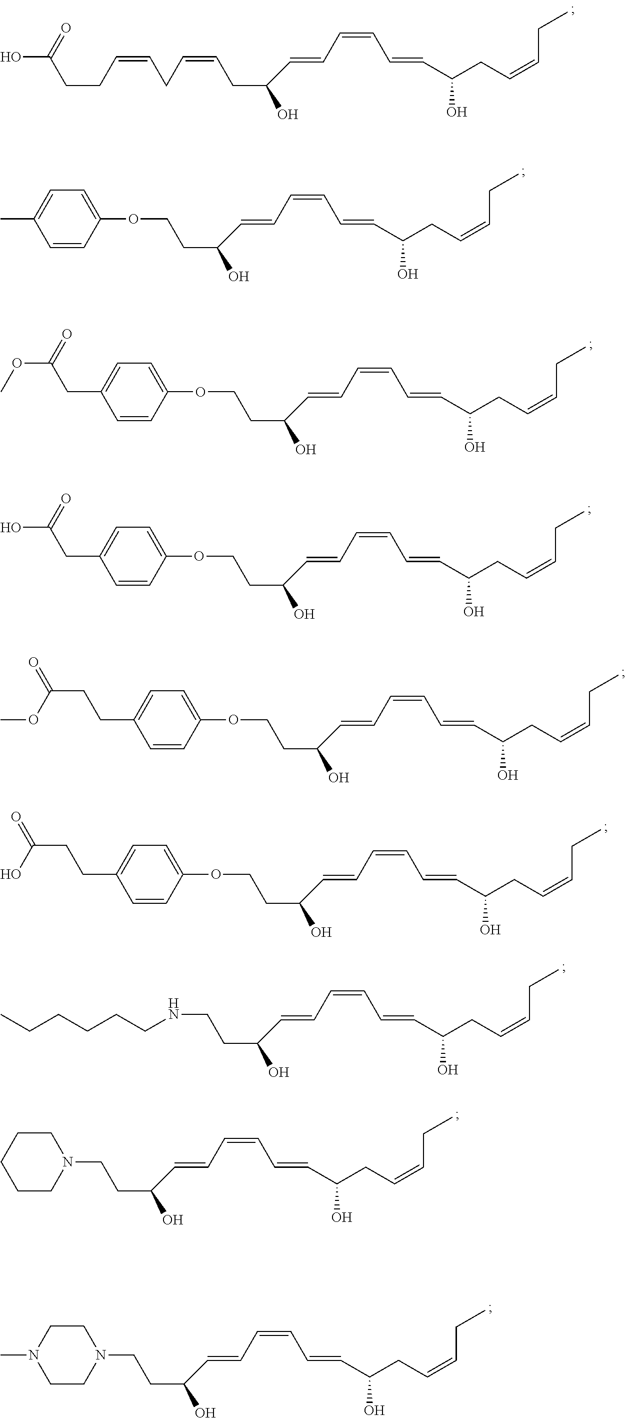

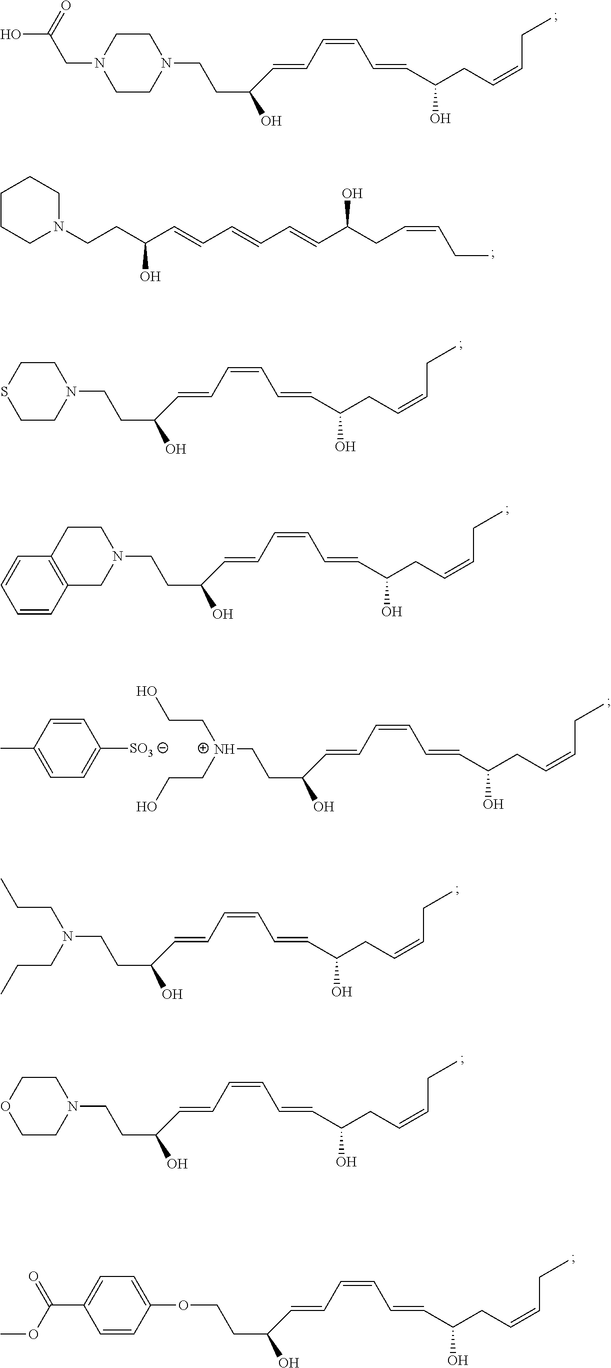

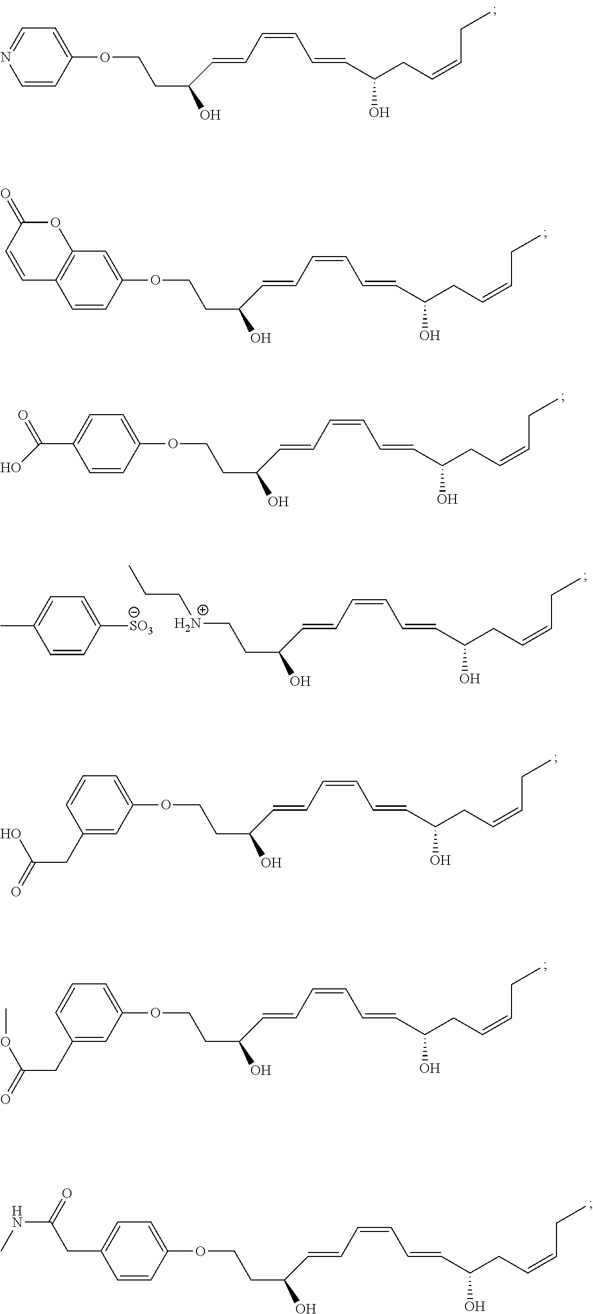

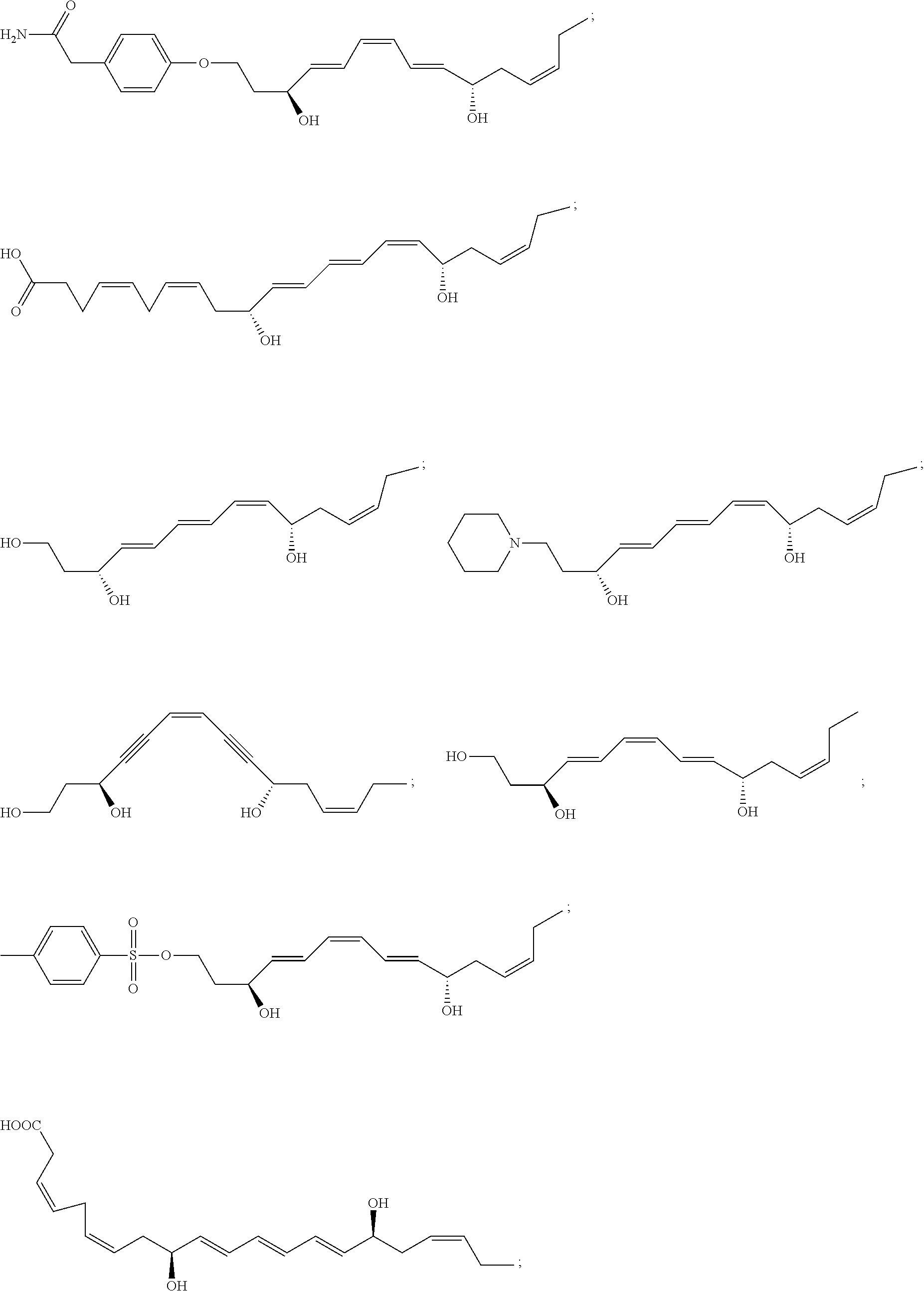

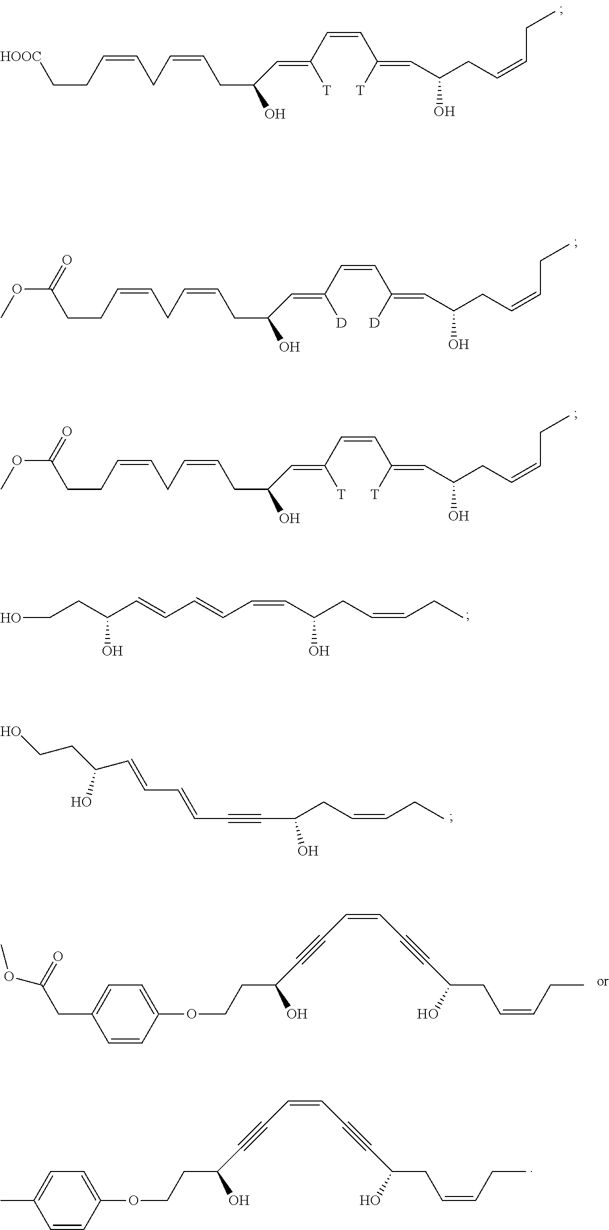

Embodiment 45 is a protectin, protectin analog, structural isomer, or a pharmaceutically acceptable salt thereof for use in preventing, delaying the onset or reducing the severity of, preventing or reversing the progression of, or treating insulin resistance or diabetes in a subject.

Embodiment 46 is the protectin, protectin analog, structural isomer, or pharmaceutically acceptable salt thereof for use of embodiment 45, wherein insulin resistance is diagnosed by measuring a patient's insulin level.

Embodiment 47 is the protectin, protectin analog, structural isomer, or pharmaceutically acceptable salt thereof for use of embodiment 45, wherein diabetes is diagnosed by measuring a patient's glucose level.

Embodiment 48 is the protectin, protectin analog, structural isomer, or pharmaceutically acceptable salt thereof for use of any one of embodiments 45 to 47, wherein diabetes is type 2 diabetes.

Embodiment 49 is the protectin, protectin analog, structural isomer, or pharmaceutically acceptable salt thereof for use of any one of embodiments 45 to 48, wherein the subject suffers from obesity.

Embodiment 50 is the protectin, protectin analog, structural isomer, or pharmaceutically acceptable salt thereof for use of any one of embodiments 45 to 49, wherein the protectin, protectin analog, structural isomer, or a pharmaceutically acceptable salt thereof is at least one of:

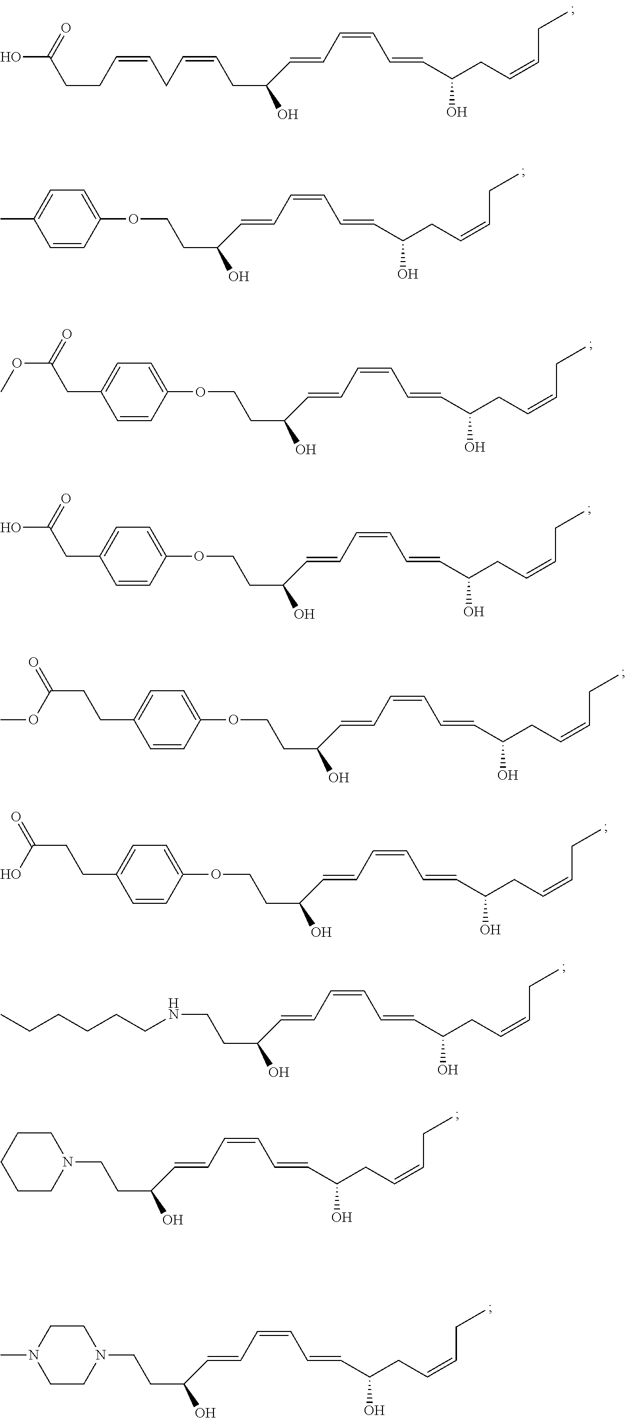

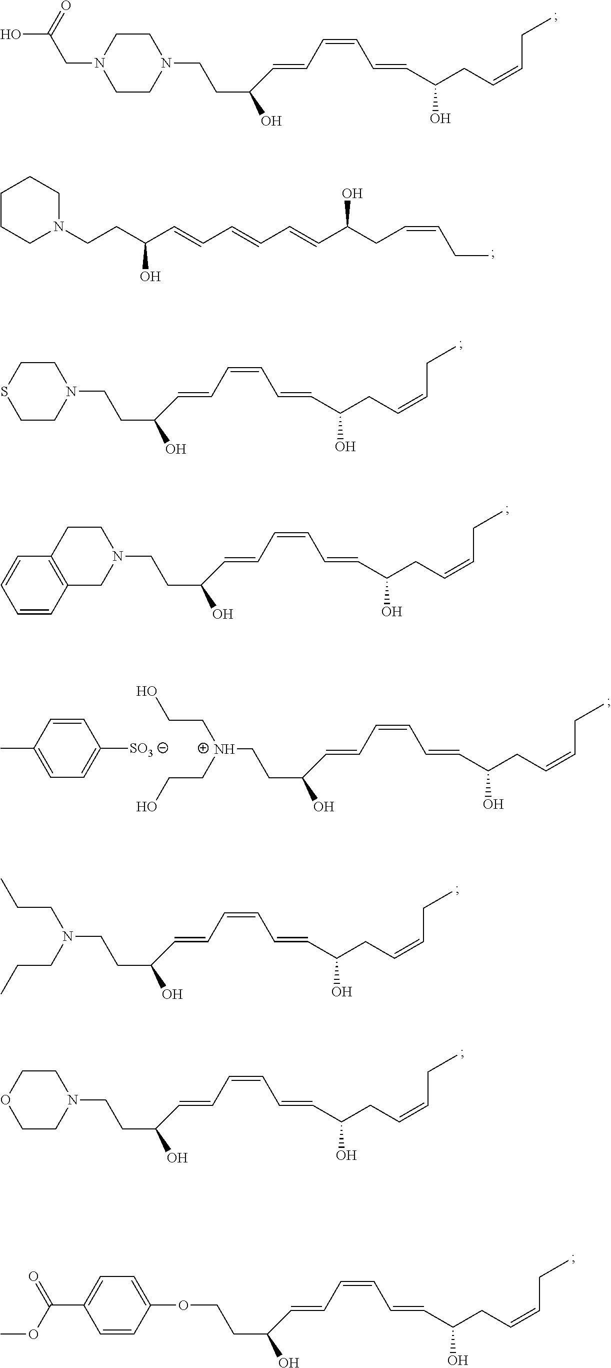

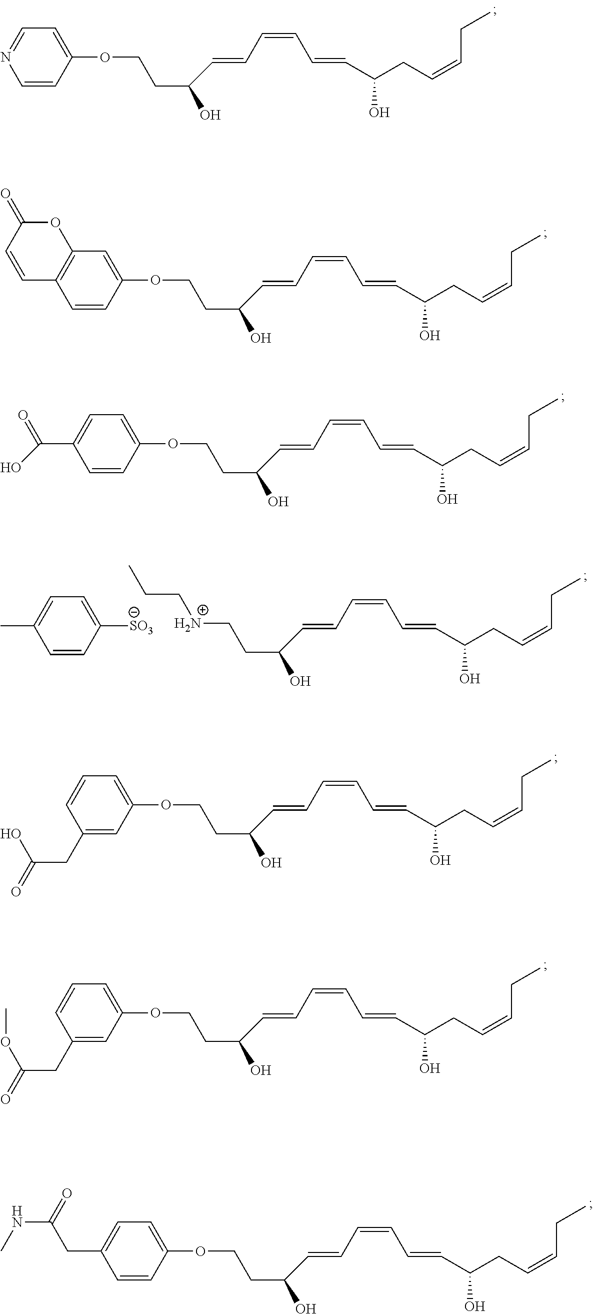

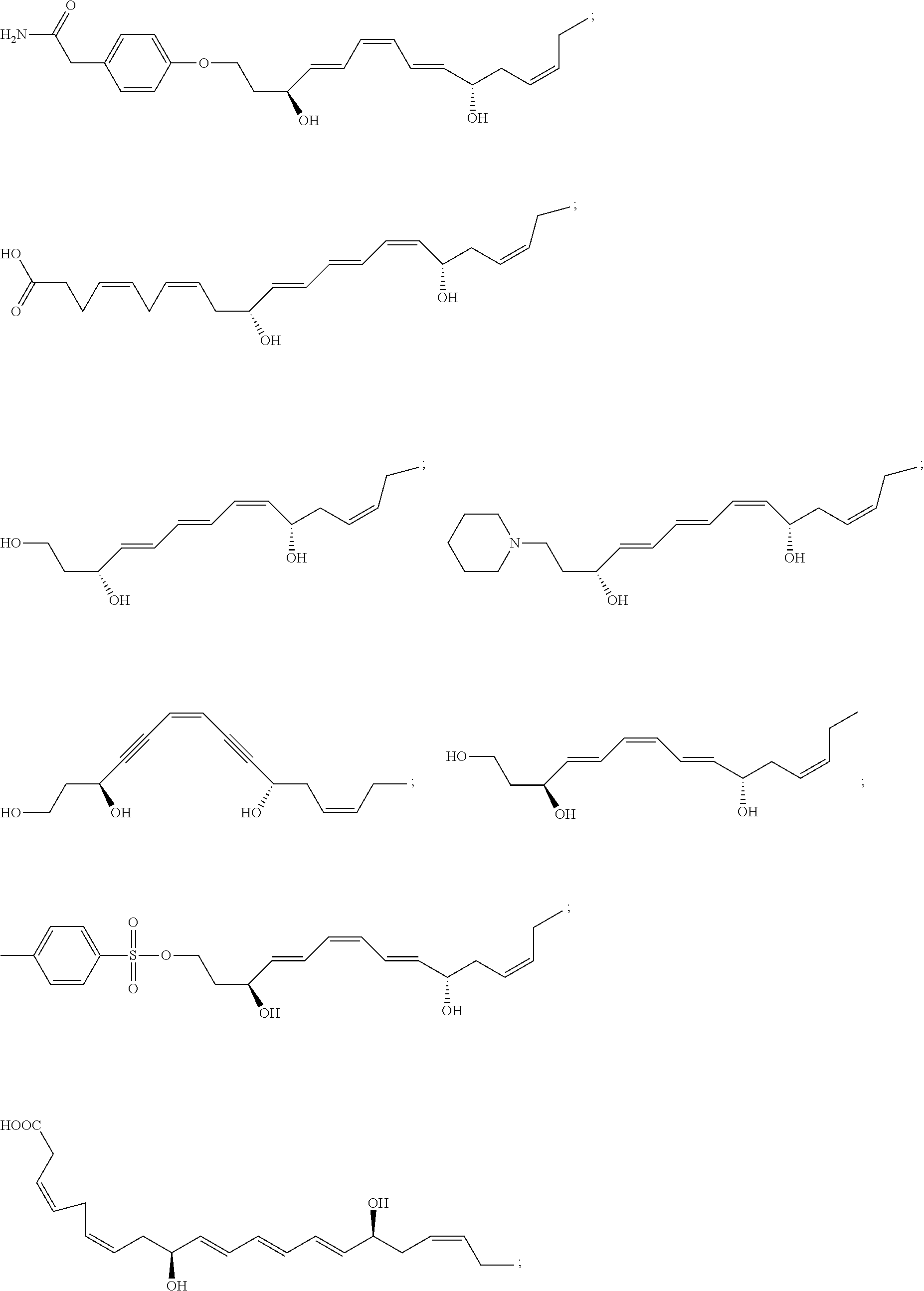

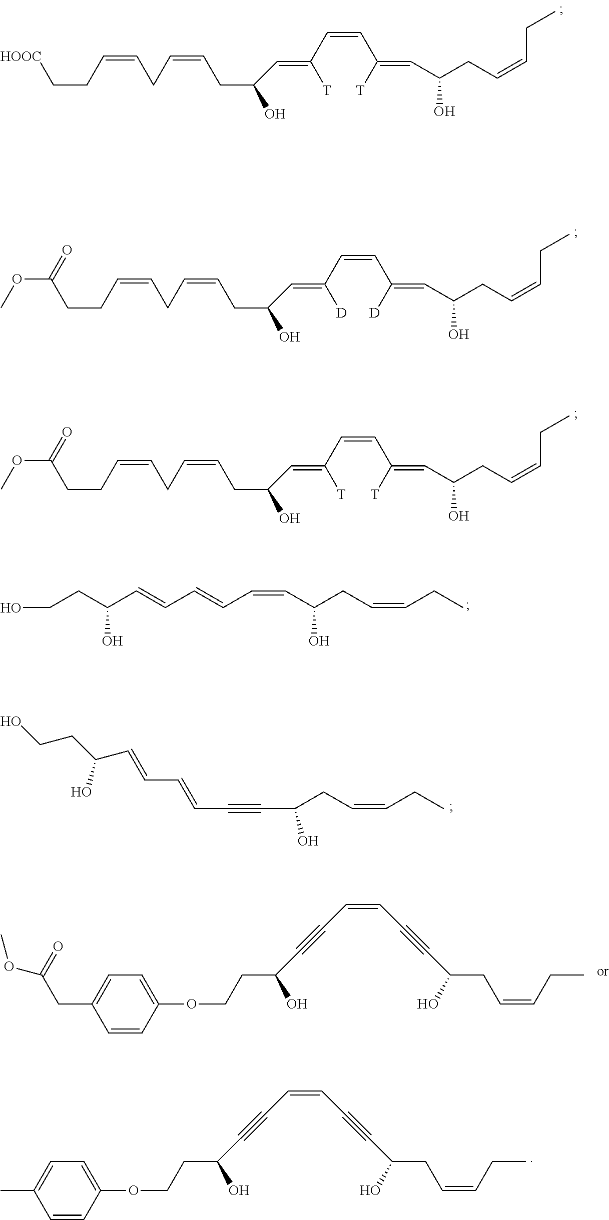

Embodiment 51 is a method of lowering blood glucose levels in a subject, said method comprising administering a protectin, protectin analog, structural isomer, or a pharmaceutically acceptable salt thereof, to the subject.

Embodiment 52 is the method of embodiment 51, wherein the subject suffers from obesity.

Embodiment 53 is the method of embodiment 51 or 52, wherein the protectin, protectin analog, structural isomer, or a pharmaceutically acceptable salt thereof is at least one of:

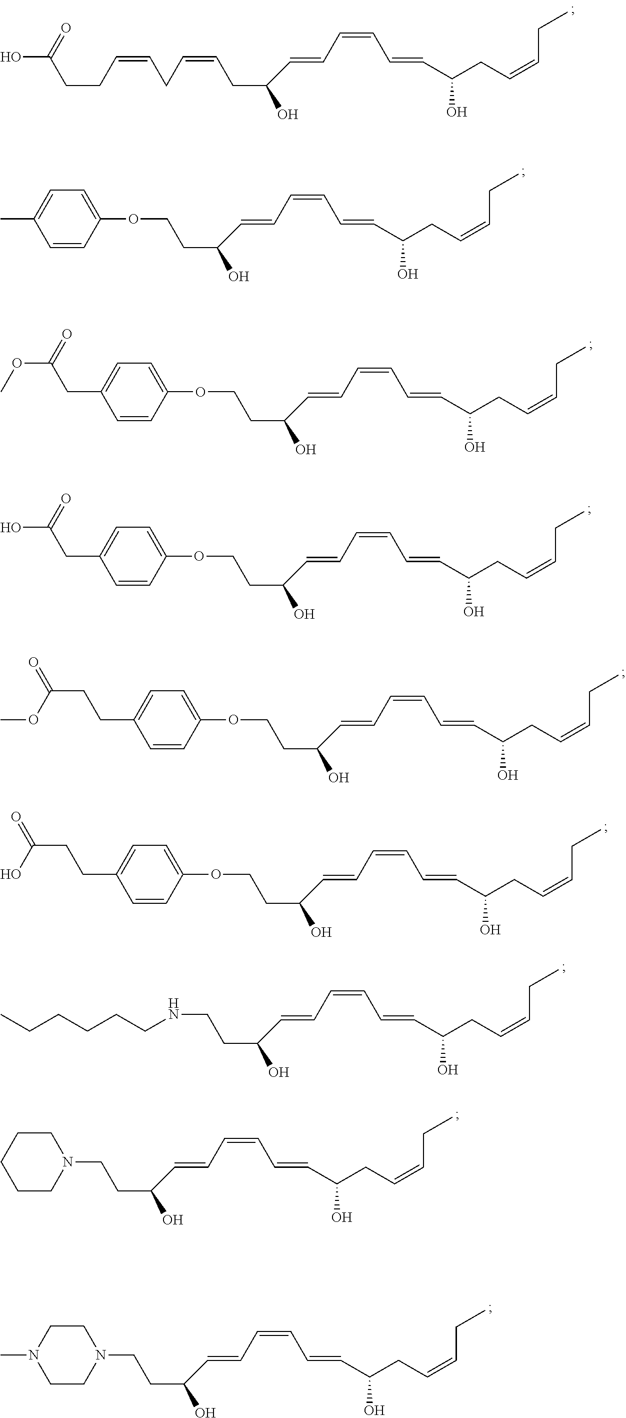

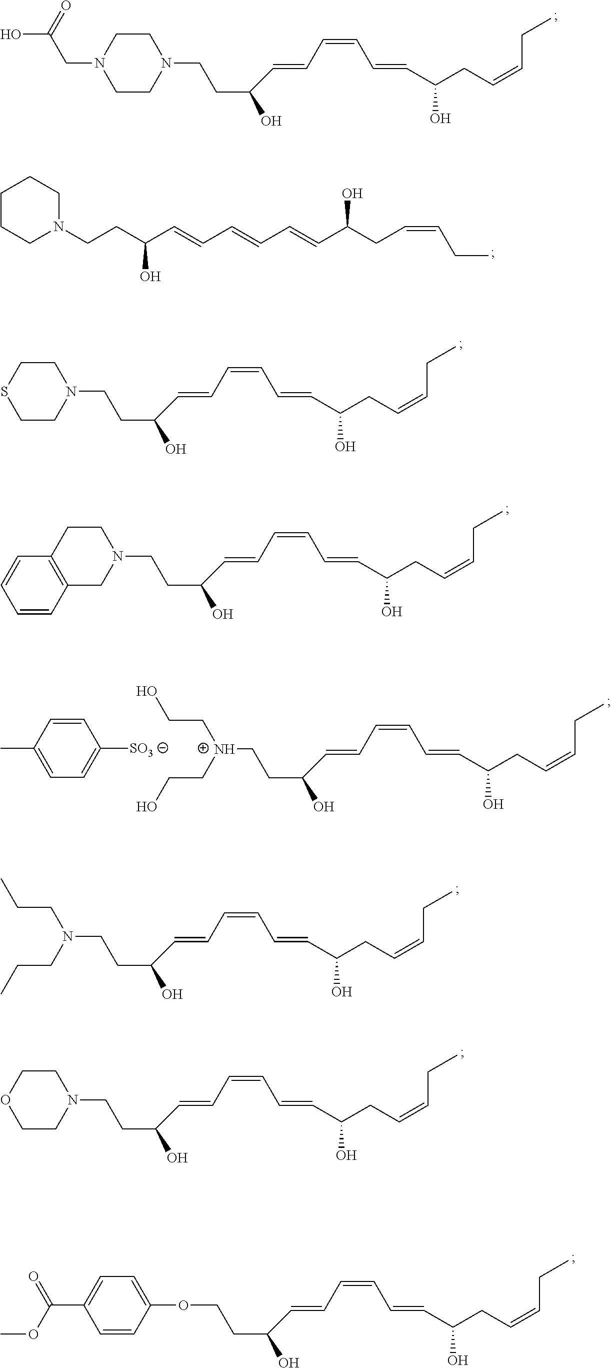

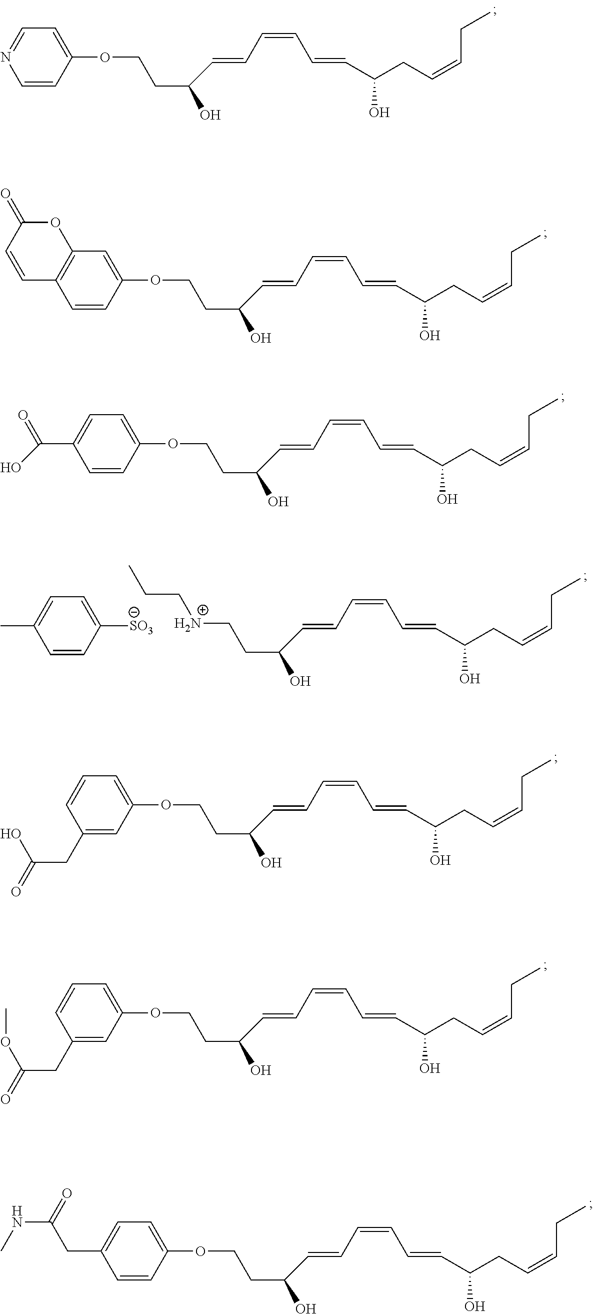

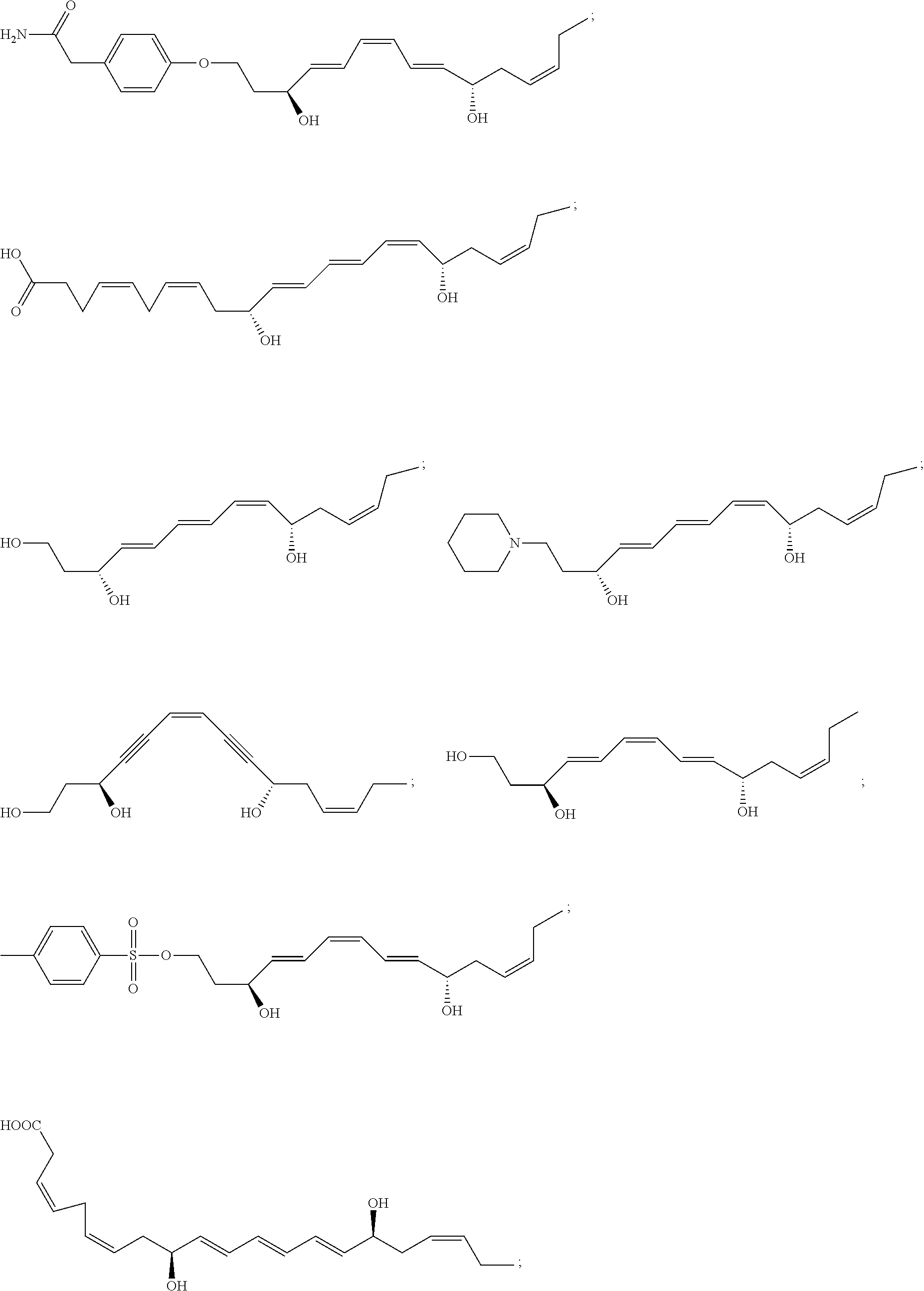

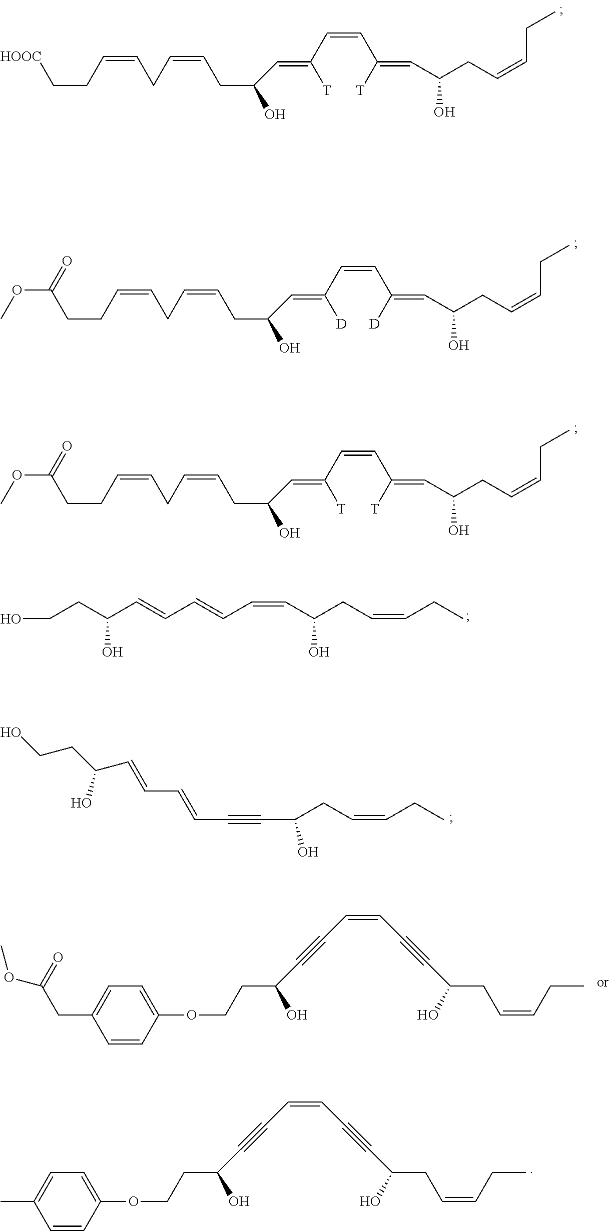

Embodiment 54 is the use of a protectin, protectin analog, structural isomer, or a pharmaceutically acceptable salt thereof for lowering blood glucose levels in a subject.

Embodiment 55 is the use of a protectin, protectin analog, structural isomer, or a pharmaceutically acceptable salt thereof for the preparation of a medicament for lowering blood glucose levels in a subject.

Embodiment 56 is the use of embodiment 54 or 55, wherein the subject suffers from obesity.

Embodiment 57 is the use of any one of embodiments 54 to 56, wherein the protectin, protectin analog, structural isomer, or a pharmaceutically acceptable salt thereof is at least one of:

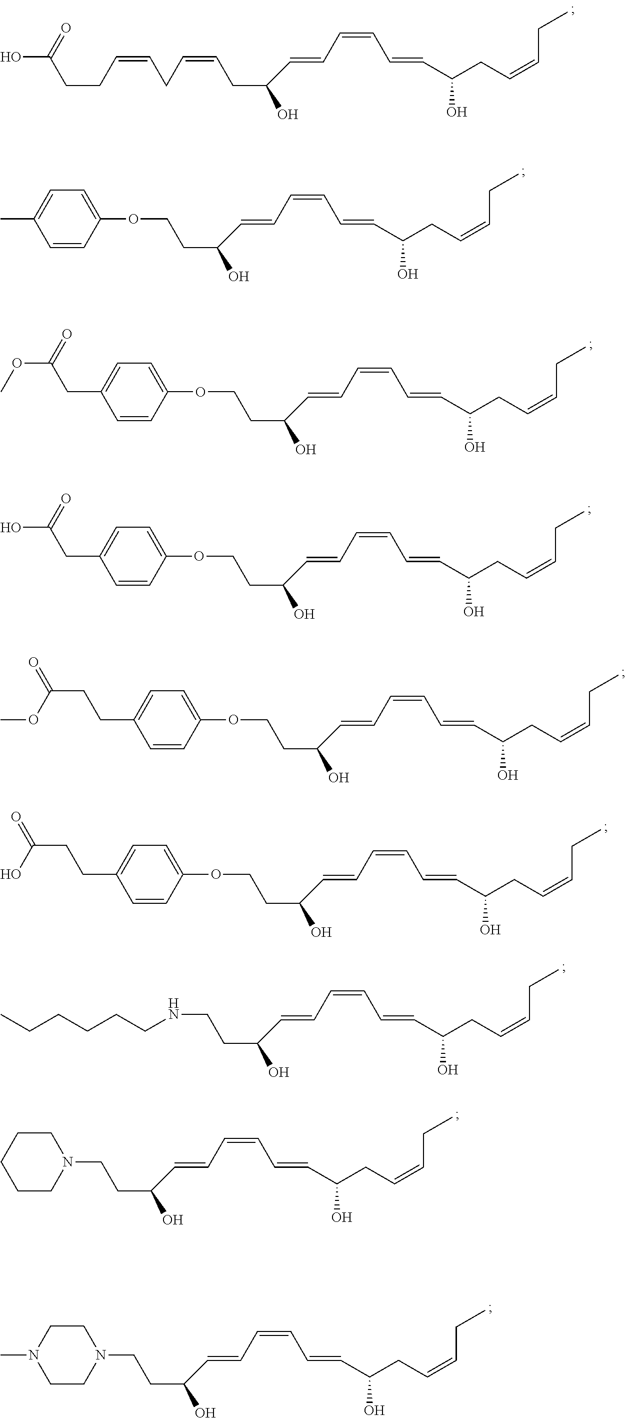

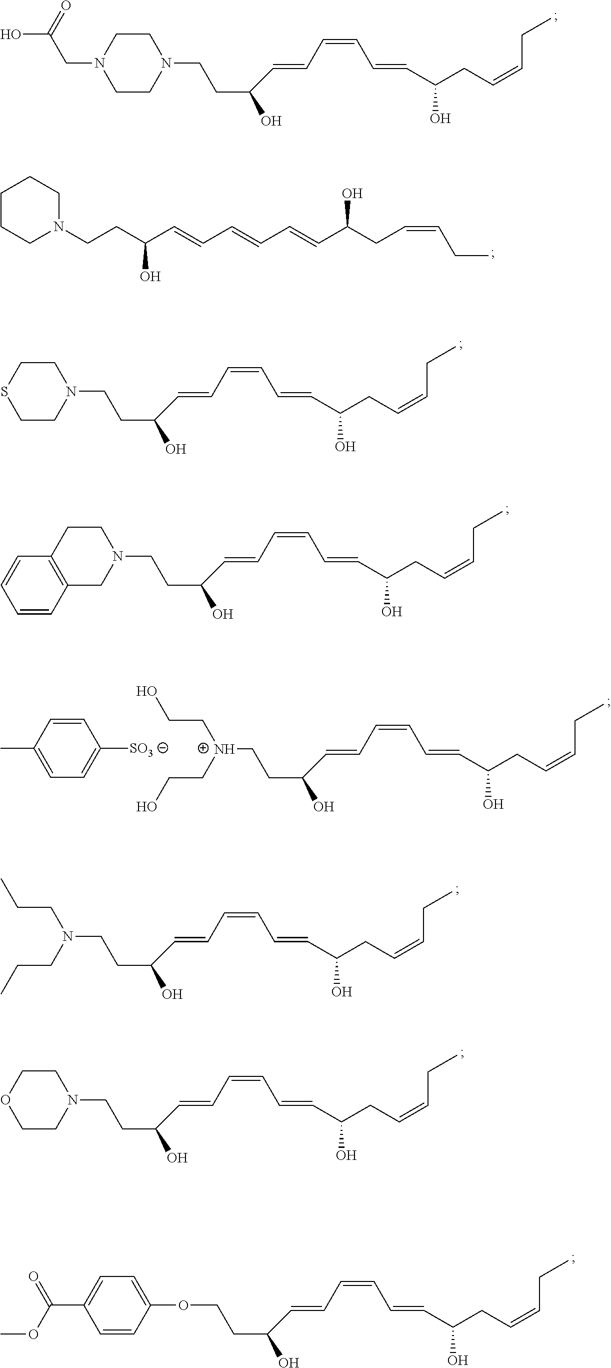

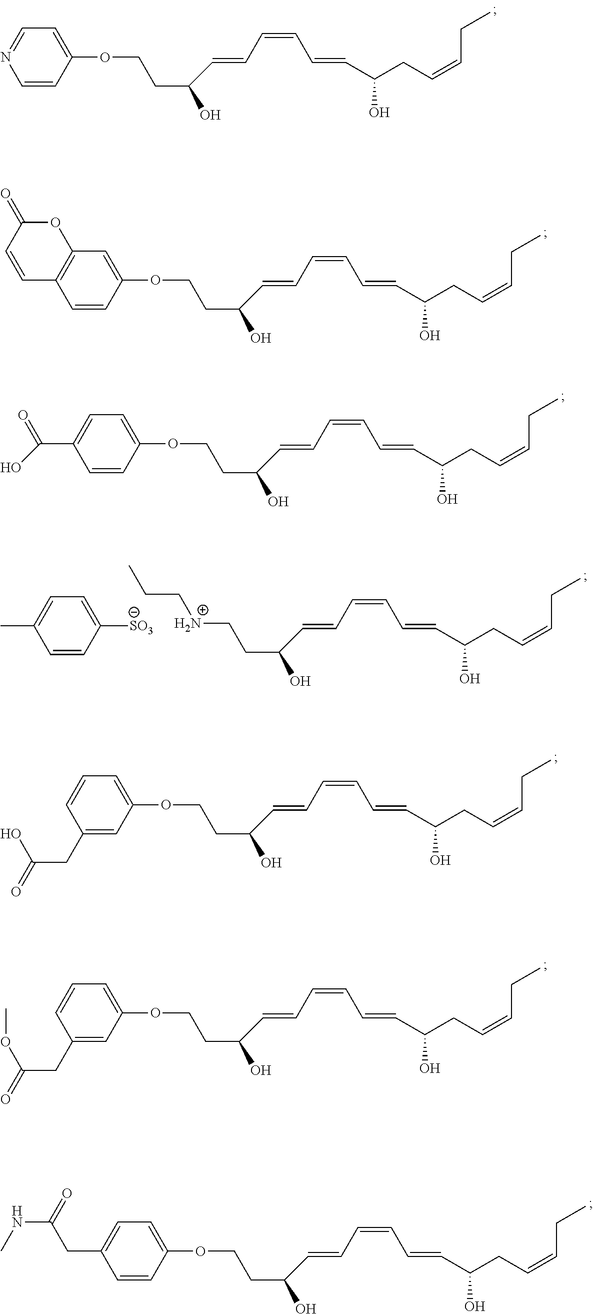

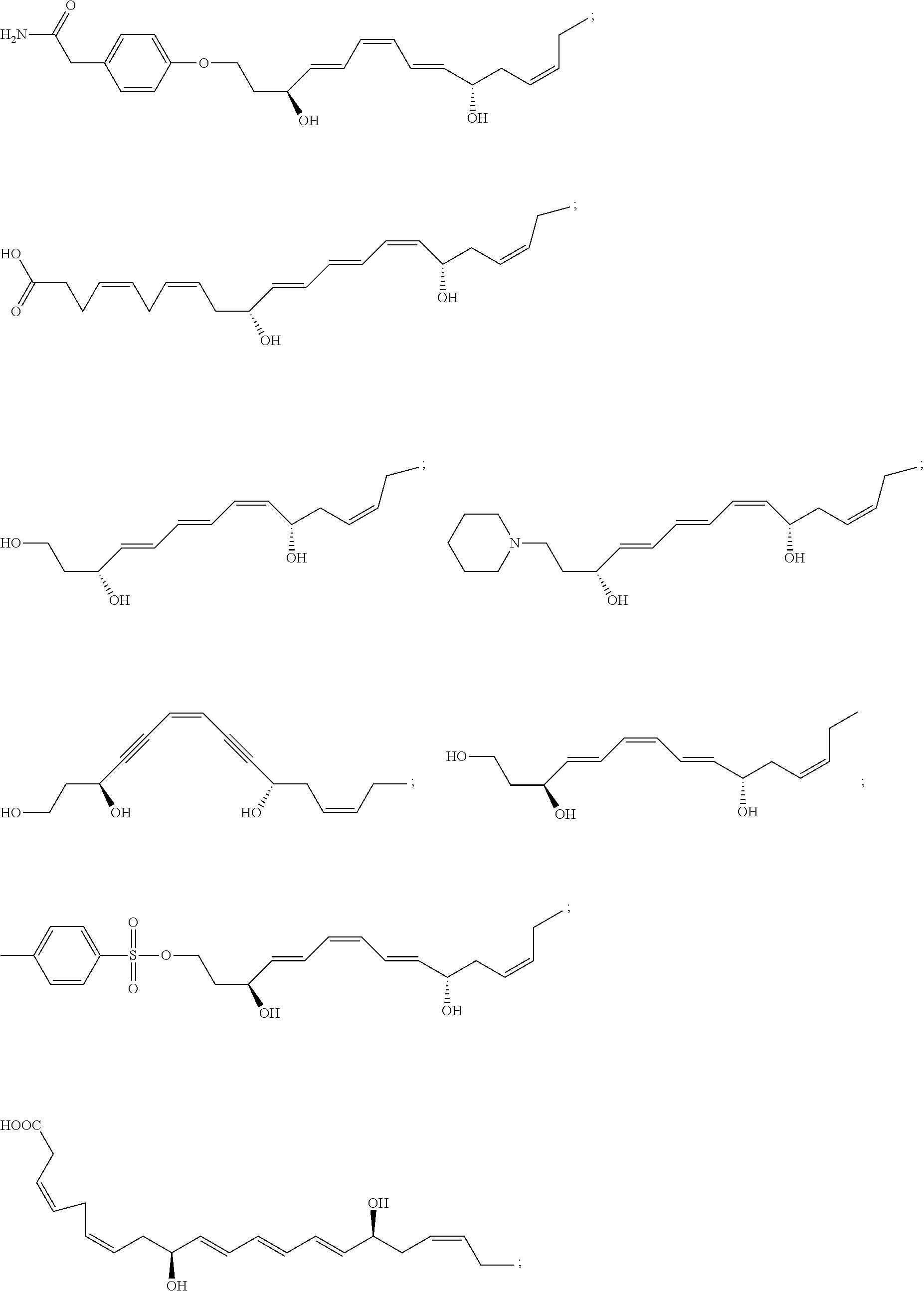

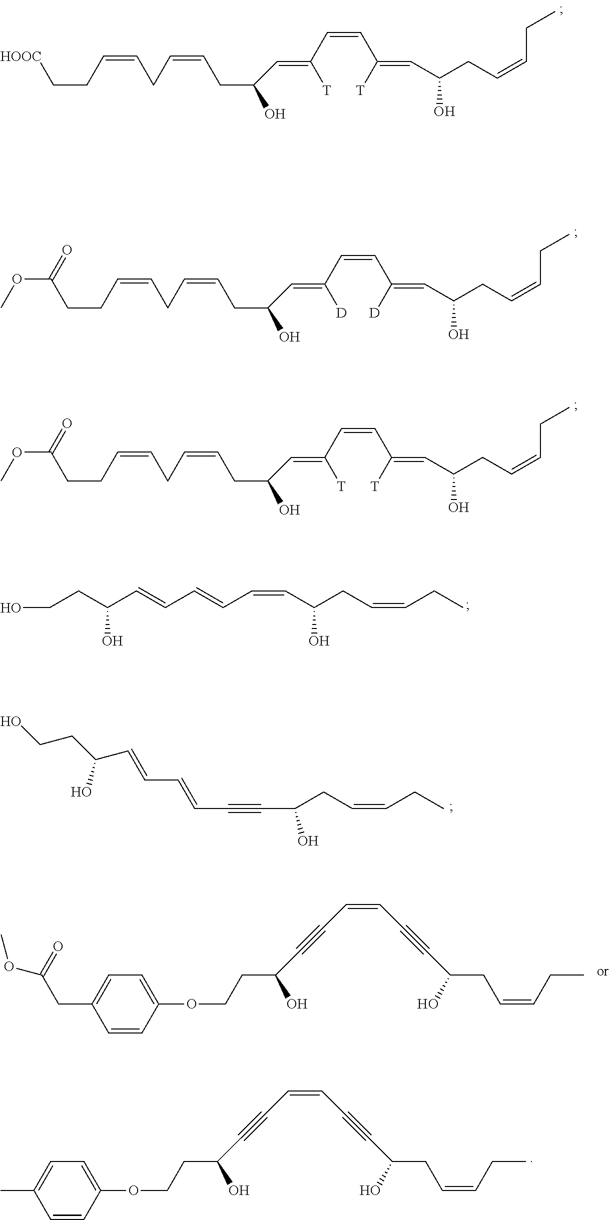

Embodiment 58 is a protectin, protectin analog, structural isomer, or a pharmaceutically acceptable salt thereof for use in lowering blood glucose levels in a subject.

Embodiment 59 is the protectin, protectin analog, structural isomer, or pharmaceutically acceptable salt thereof for use of embodiment 58, wherein the subject suffers from obesity

Embodiment 60 is the protectin, protectin analog, structural isomer, or pharmaceutically acceptable salt thereof for use of embodiment 58 or 59, wherein the protectin, protectin analog, structural isomer, or a pharmaceutically acceptable salt thereof is at least one of:

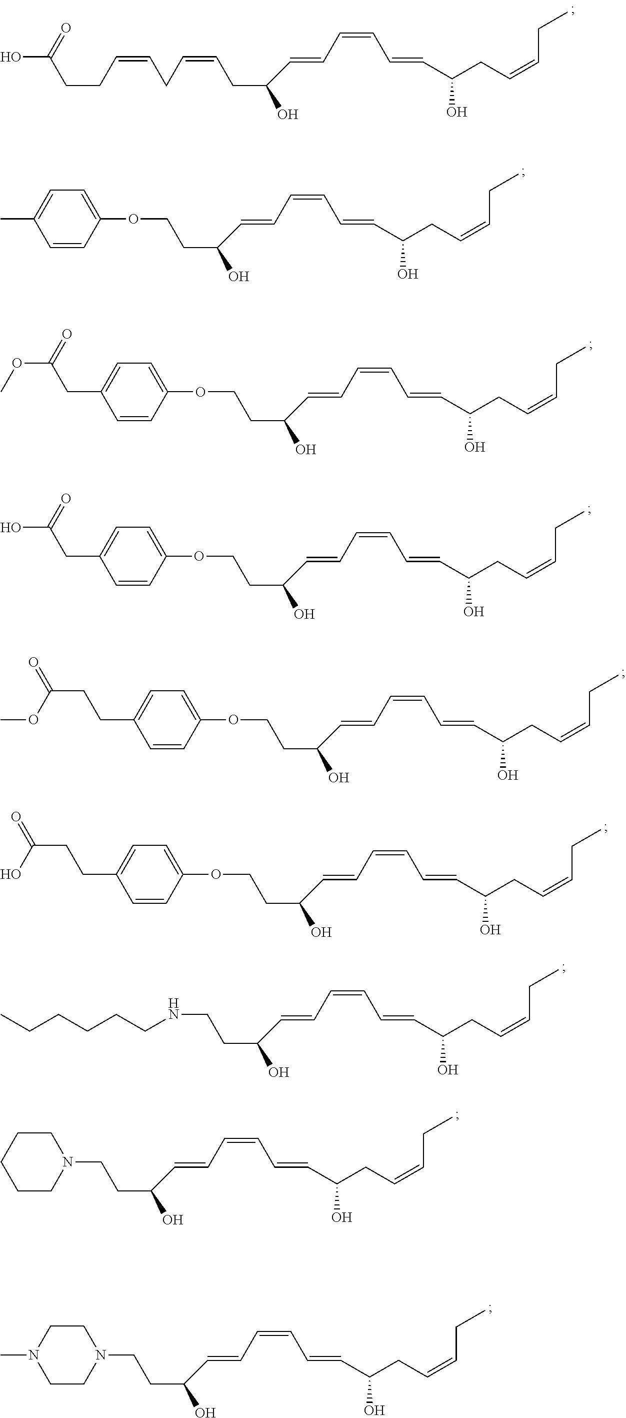

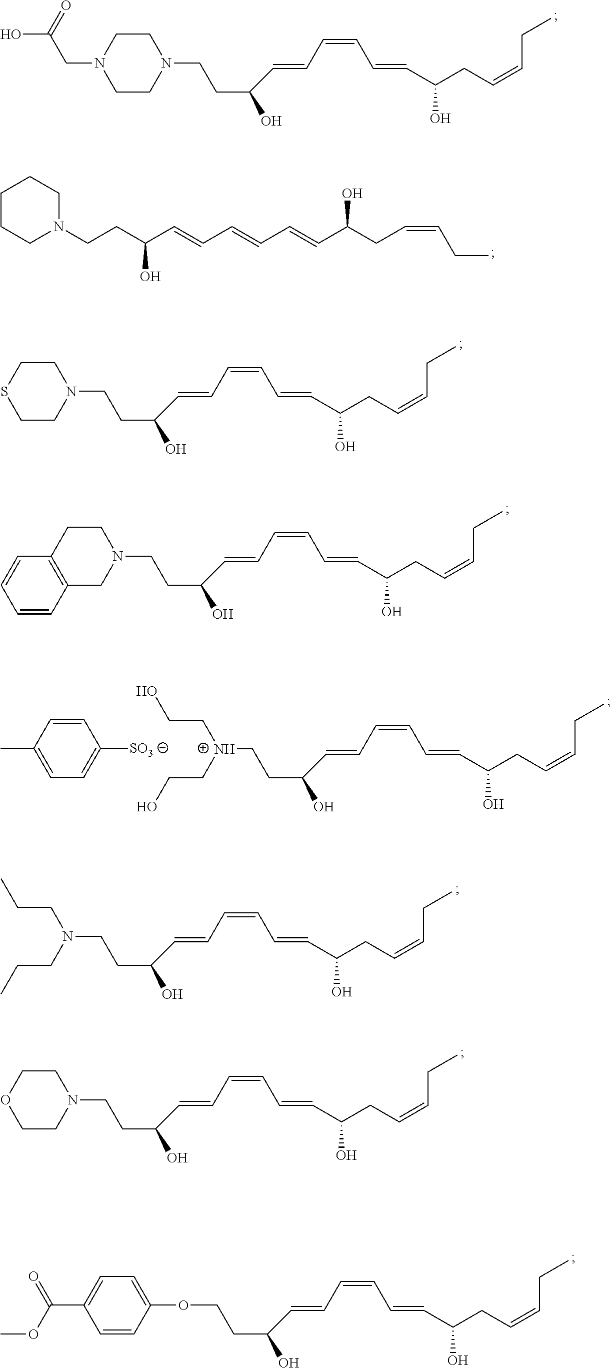

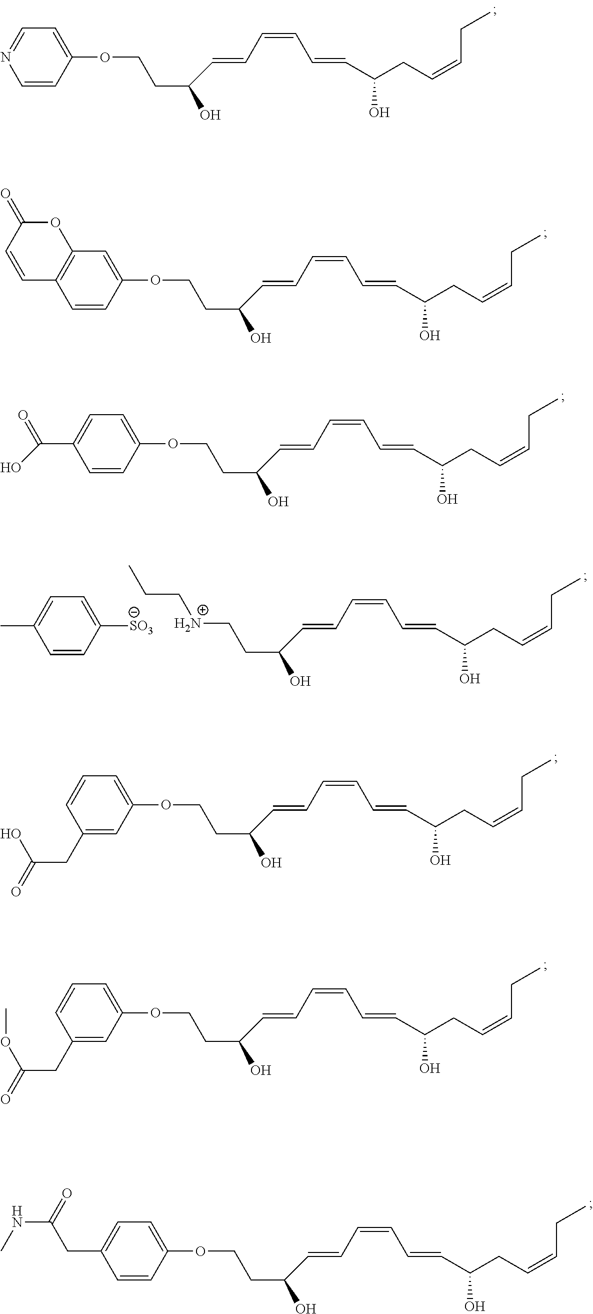

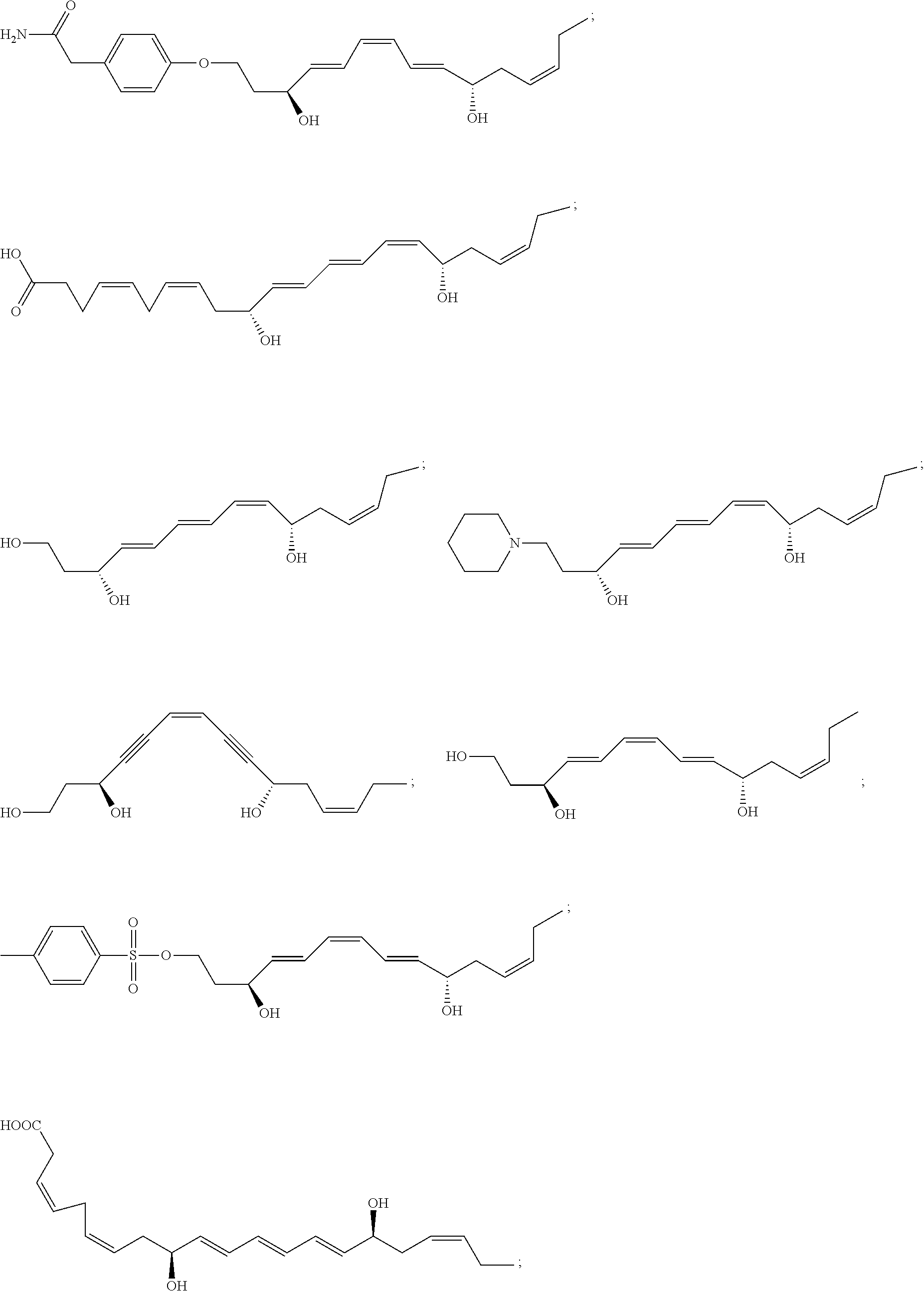

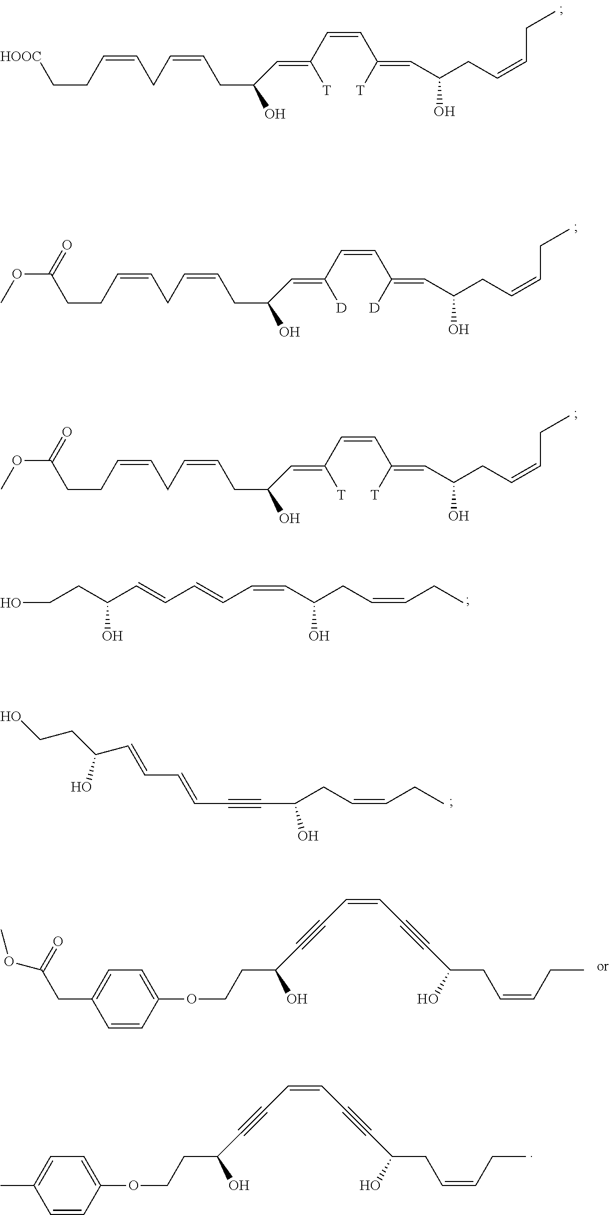

Embodiment 61 is a method of preventing, delaying the onset or reducing the severity of, preventing or reversing the progression of, or treating insulin resistance in a subject, said method comprising administering a protectin, protectin analog, structural isomer, or a pharmaceutically acceptable salt thereof, to the subject.

Embodiment 62 is the method of embodiment 61, wherein the subject suffers from obesity.

Embodiment 63 is the method of embodiment 61 or 62, wherein the protectin, protectin analog, structural isomer, or a pharmaceutically acceptable salt thereof is at least one of:

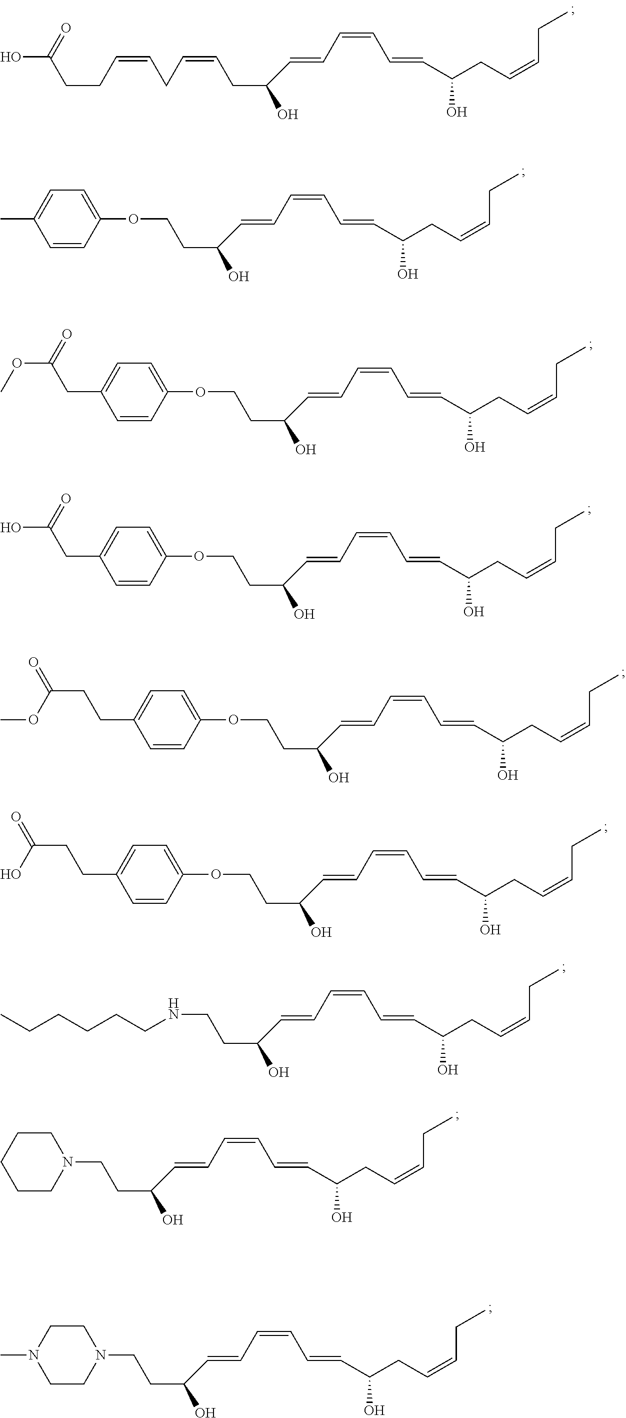

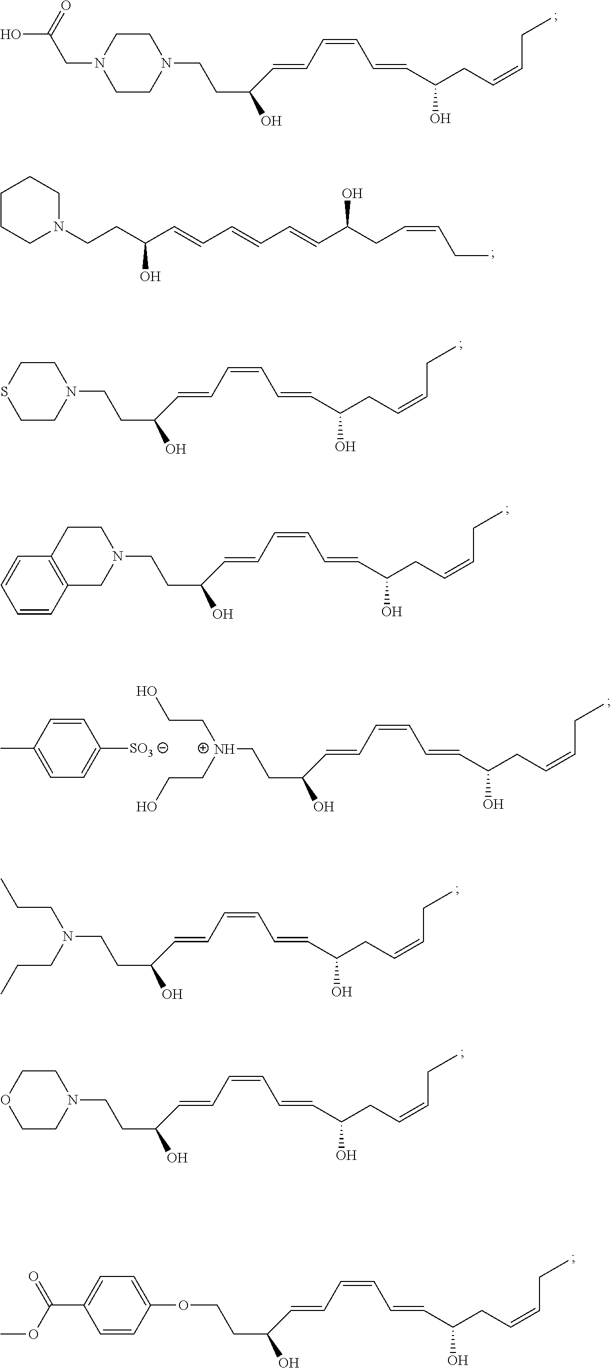

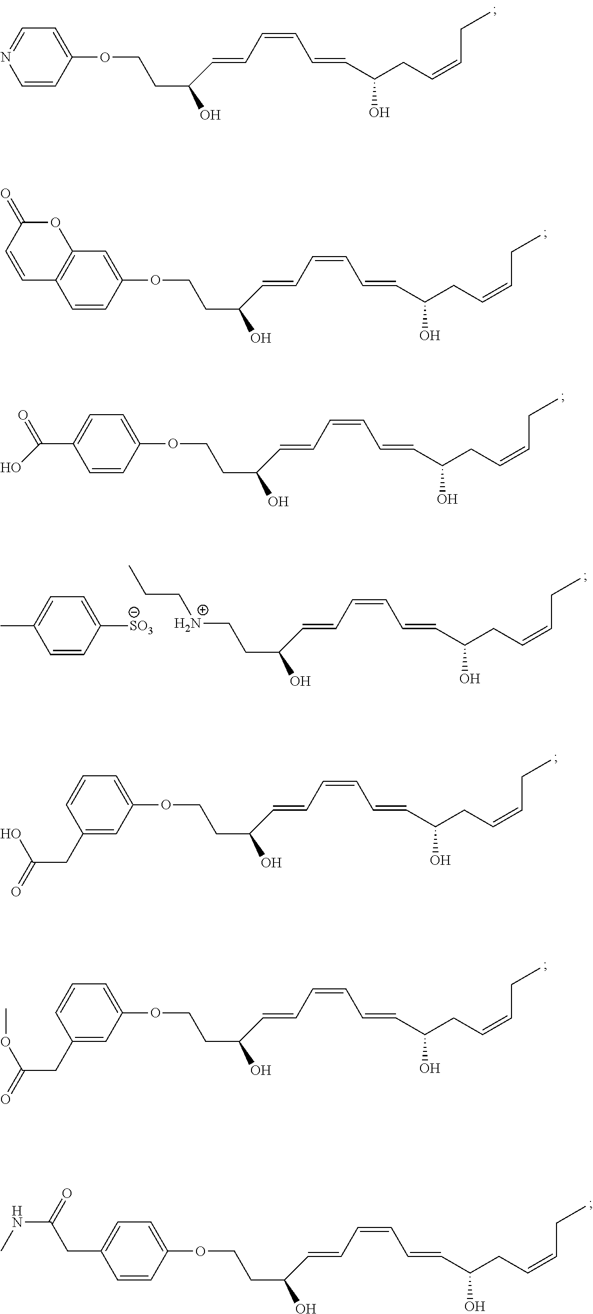

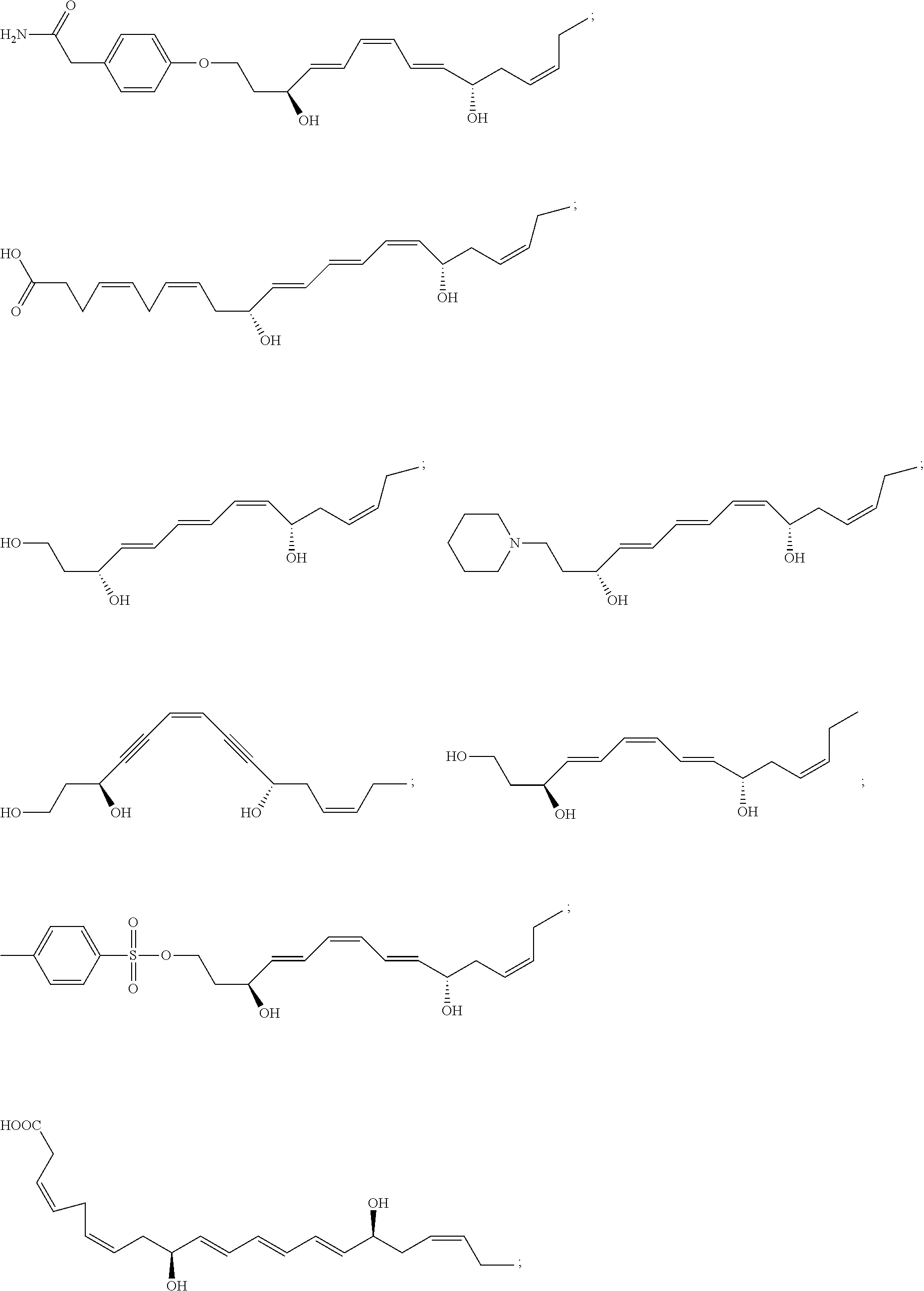

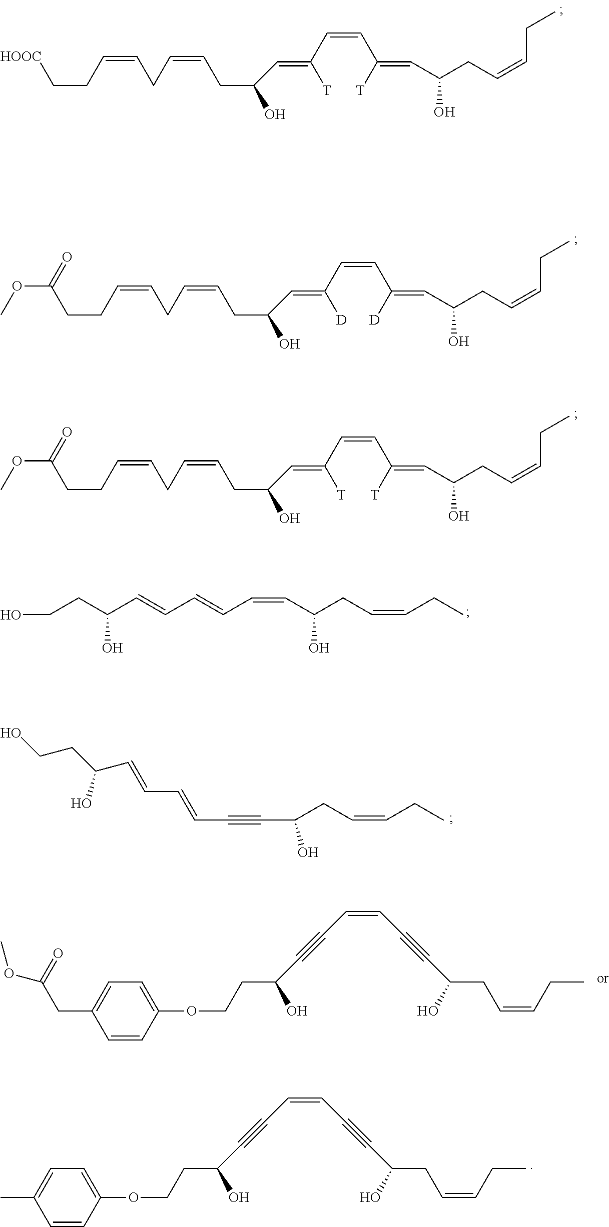

Embodiment 64 is the use of a protectin, protectin analog, structural isomer, or a pharmaceutically acceptable salt thereof for delaying the onset or reducing the severity of, preventing or reversing the progression of, or treating insulin resistance in a subject.

Embodiment 65 is the use of a protectin, protectin analog, structural isomer, or a pharmaceutically acceptable salt thereof for the preparation of a medicament for delaying the onset or reducing the severity of, preventing or reversing the progression of, or treating insulin resistance in a subject.

Embodiment 66 is the use of embodiment 64 or 65, wherein the subject suffers from obesity.

Embodiment 67 is the use of any one of embodiments 64 to 66, wherein the protectin, protectin analog, structural isomer, or a pharmaceutically acceptable salt thereof is at least one of:

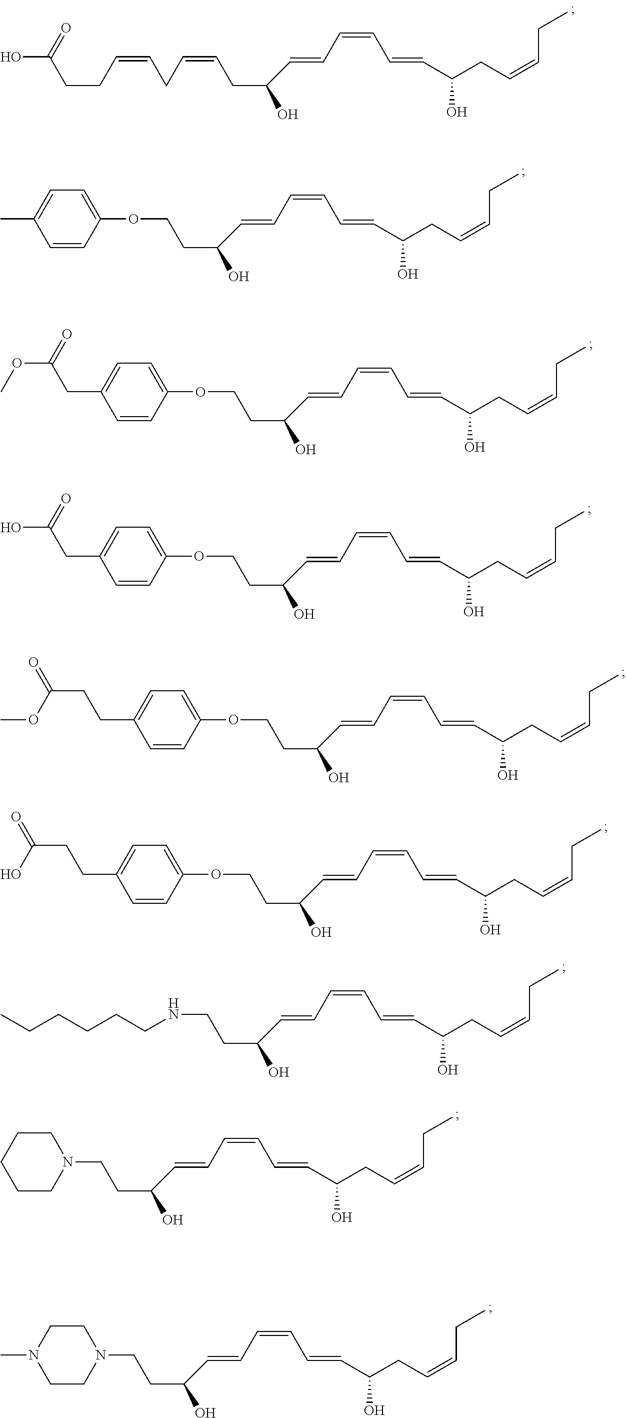

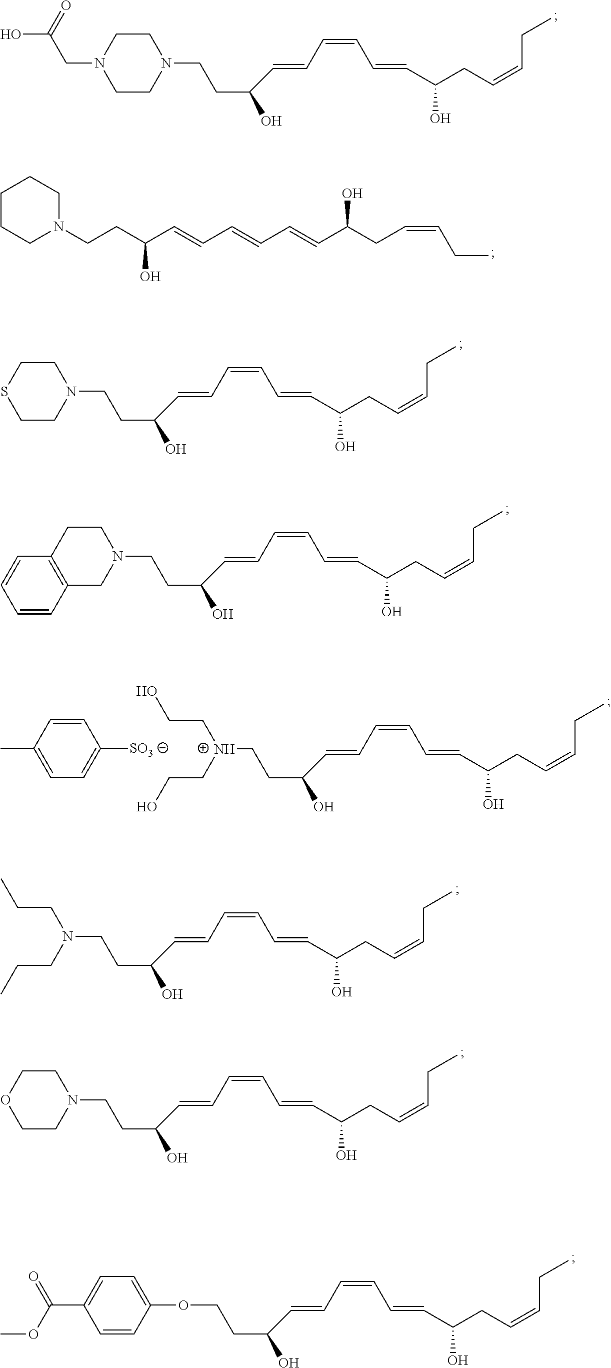

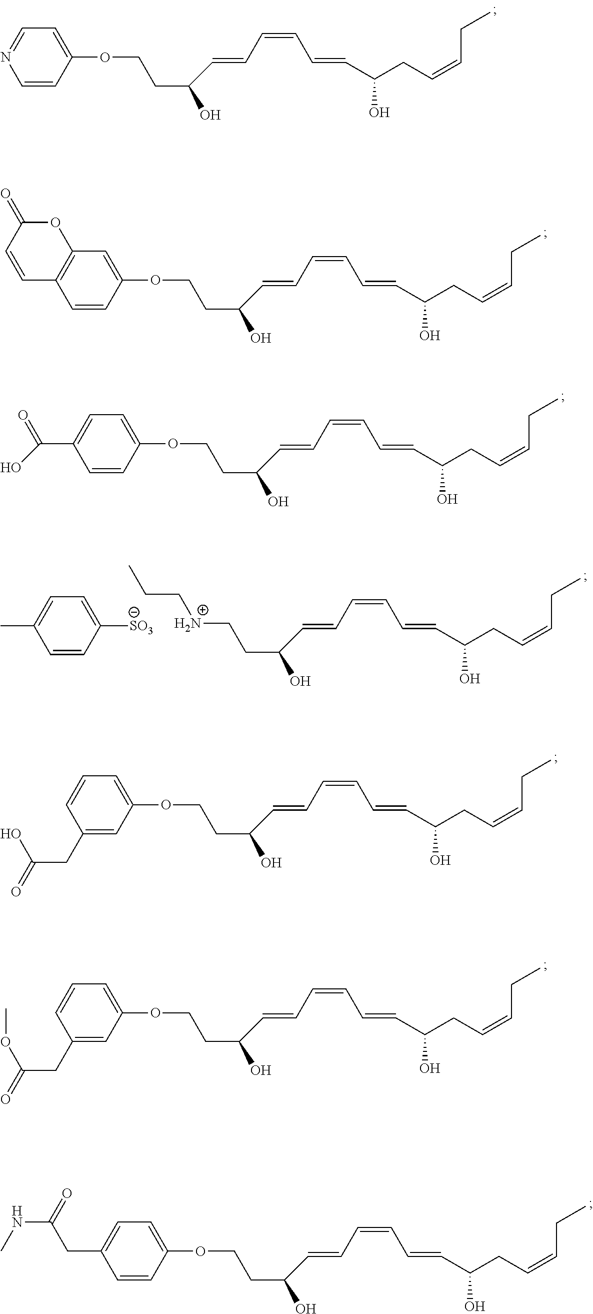

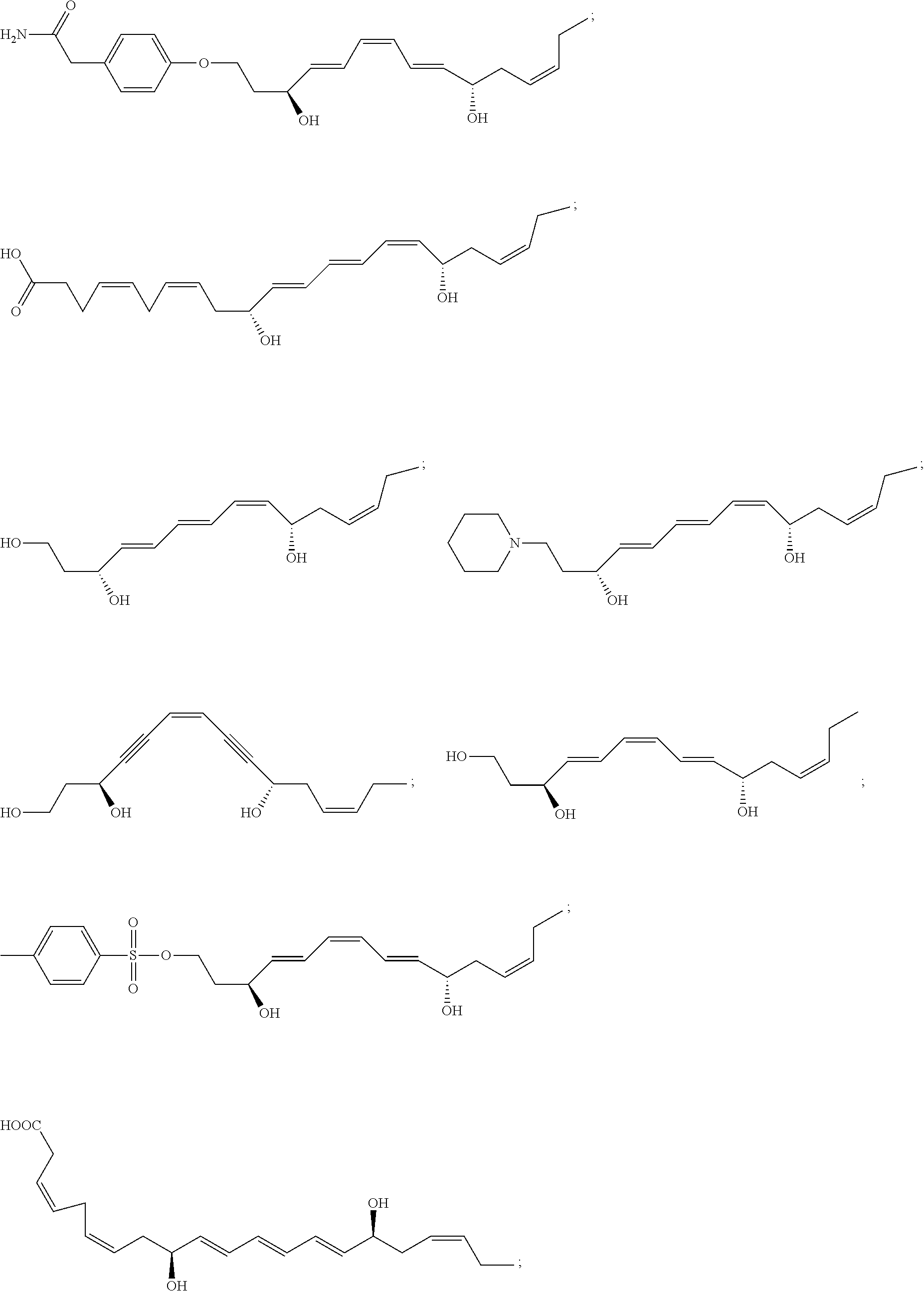

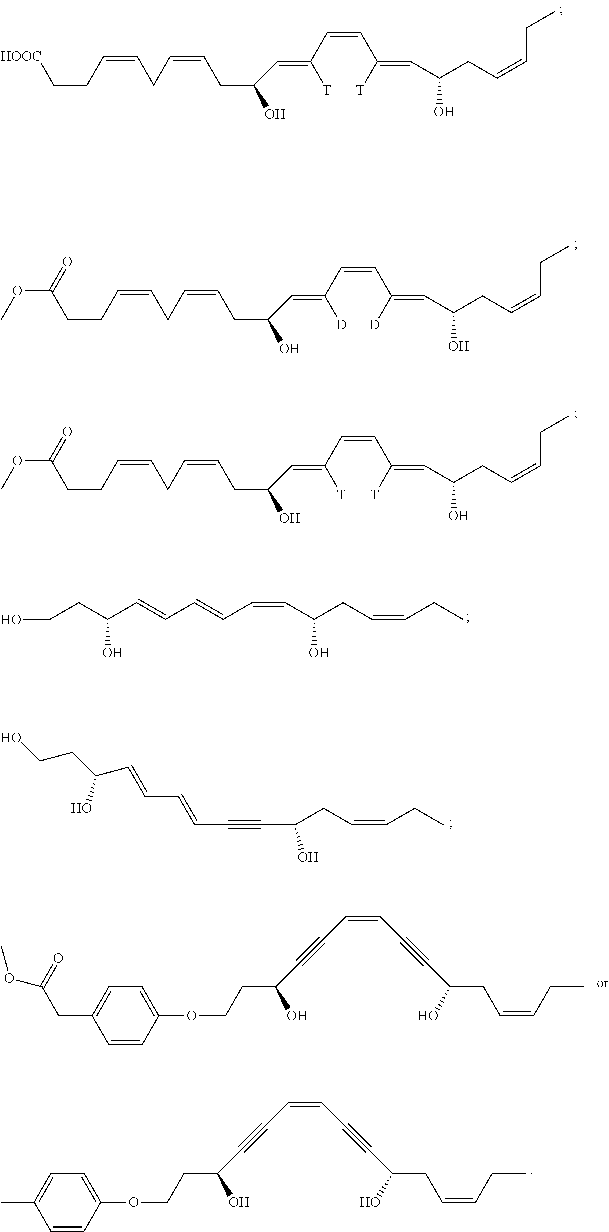

Embodiment 68 is a protectin, protectin analog, structural isomer, or a pharmaceutically acceptable salt thereof for use in delaying the onset or reducing the severity of, preventing or reversing the progression of, or treating insulin resistance in a subject.

Embodiment 69 is the protectin, protectin analog, structural isomer, or pharmaceutically acceptable salt thereof for use of embodiment 68, wherein the subject suffers from obesity.

Embodiment 70 is the protectin, protectin analog, structural isomer, or pharmaceutically acceptable salt thereof for use of embodiment 68 or 69, wherein the protectin, protectin analog, structural isomer, or a pharmaceutically acceptable salt thereof is at least one of:

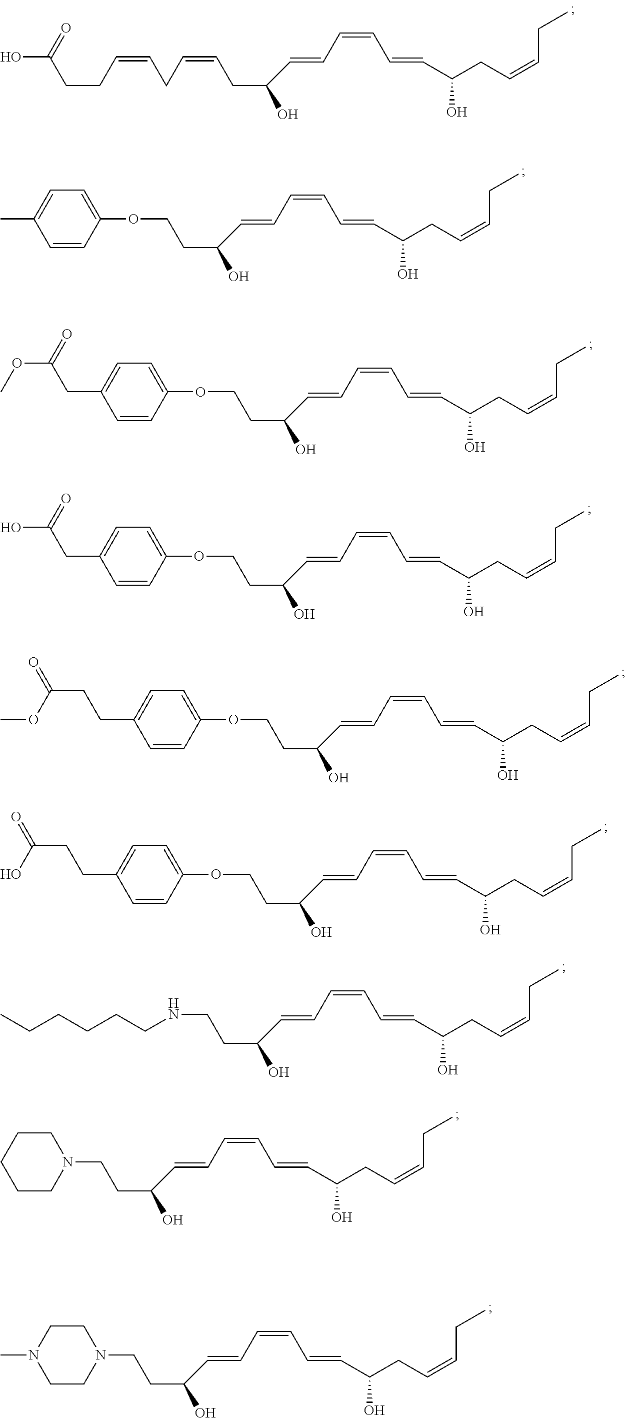

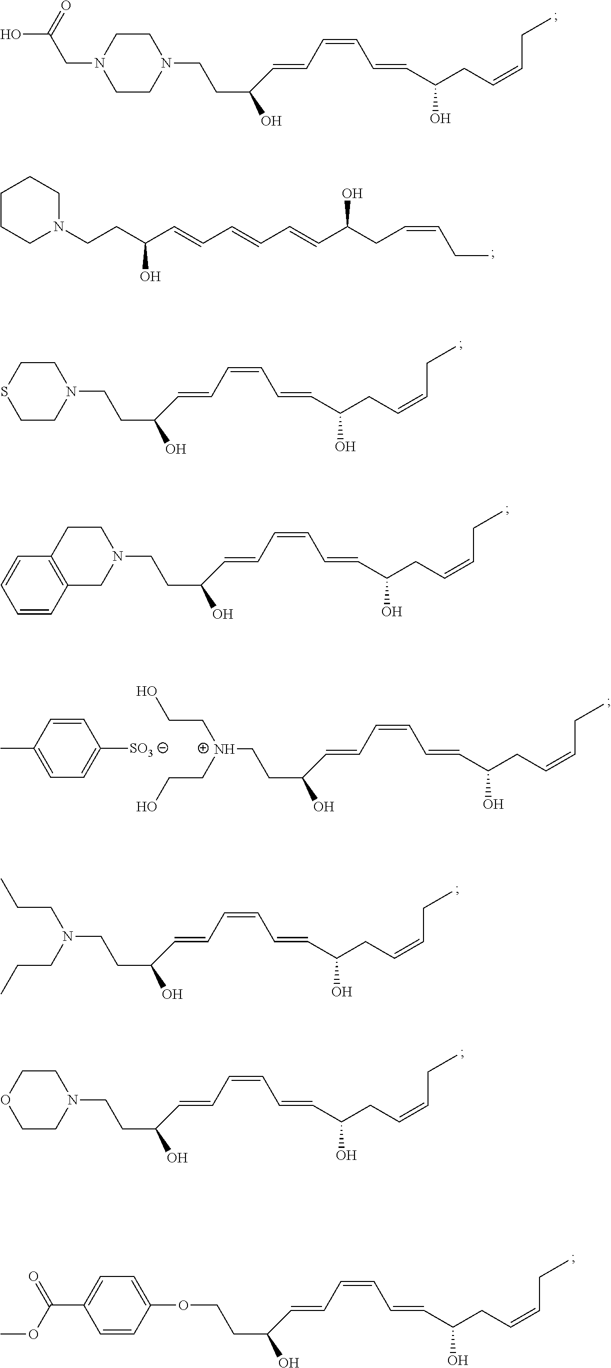

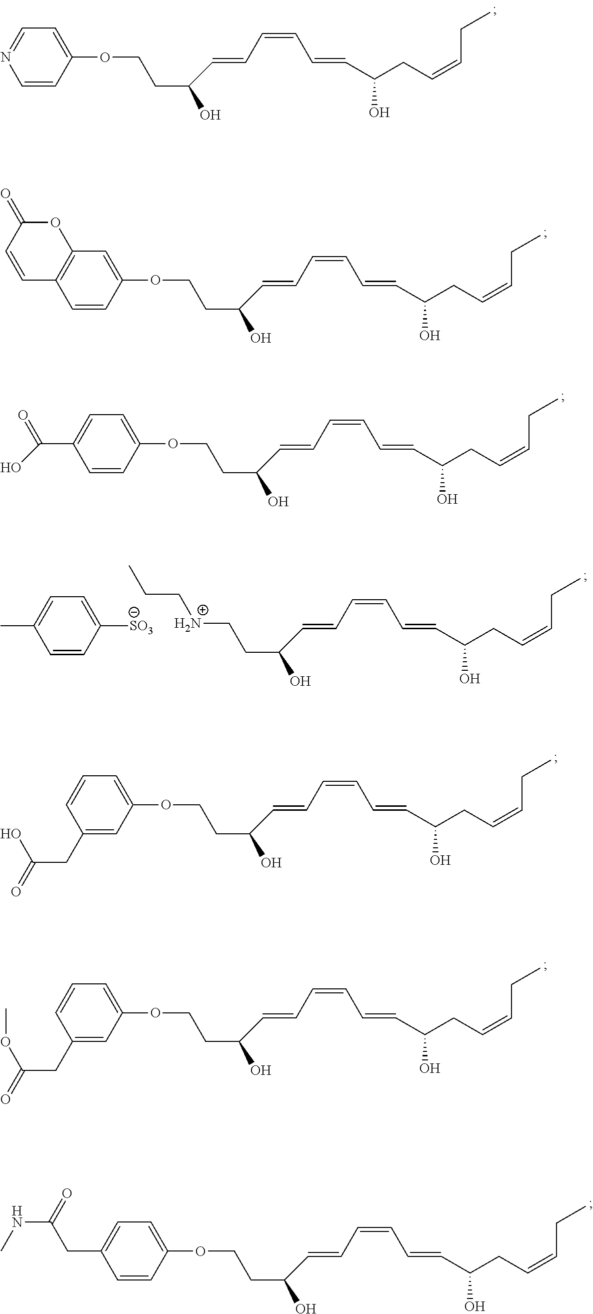

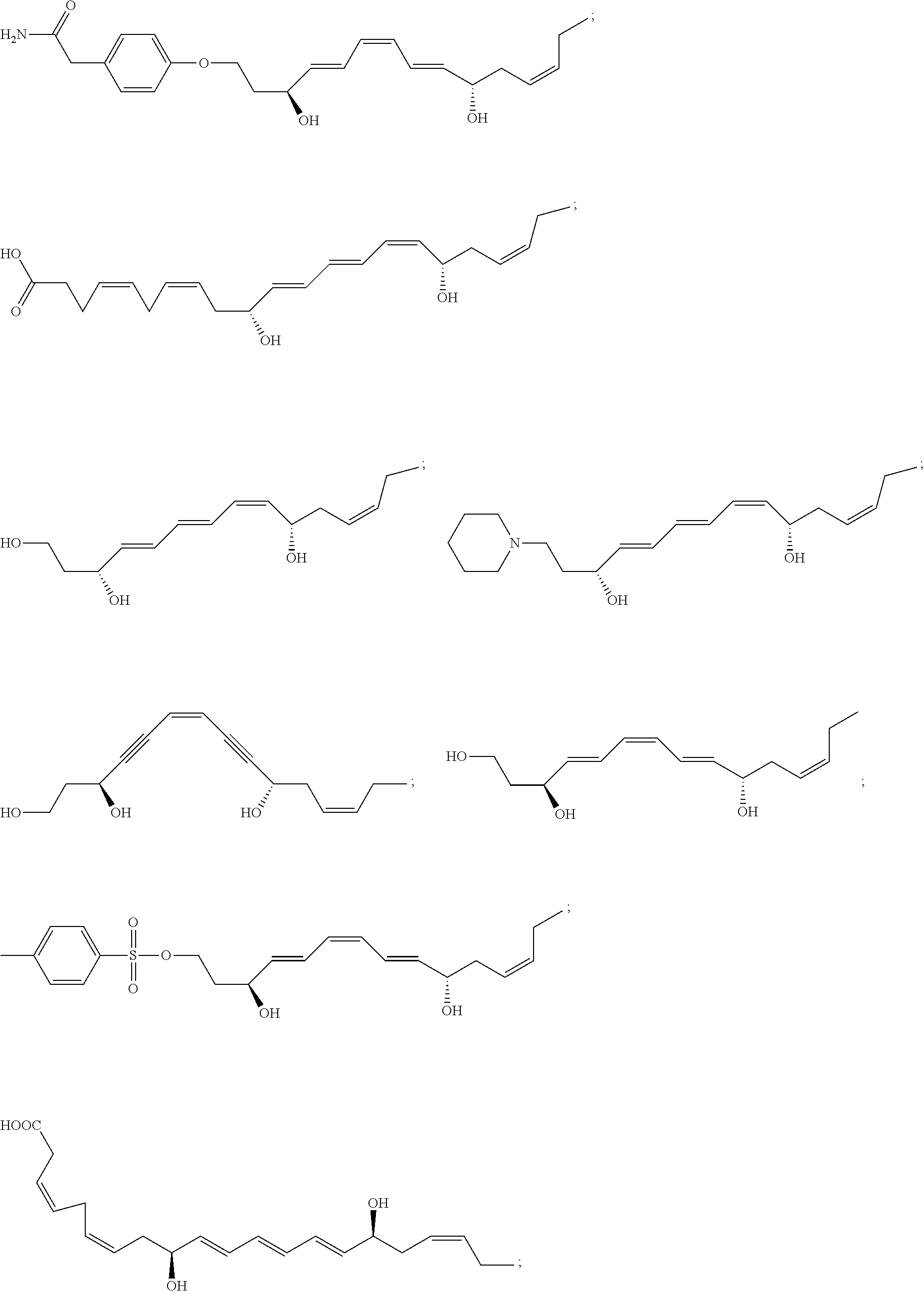

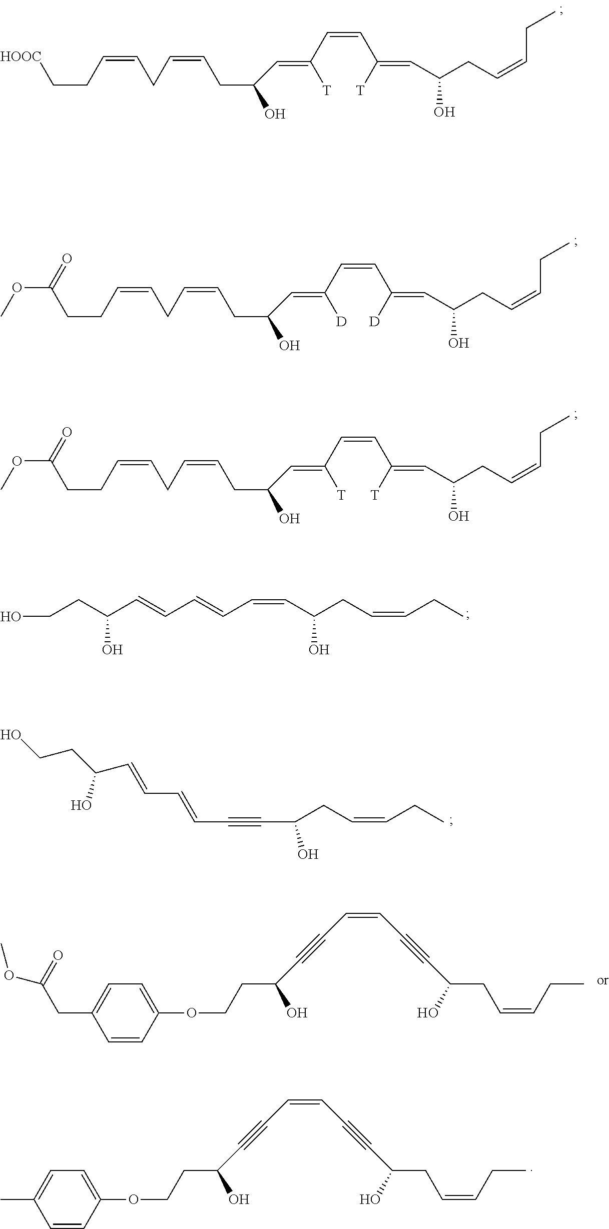

Embodiment 71 is a method of preventing, delaying the onset or reducing the severity of, preventing or reversing the progression of, or treating an inflammatory disease, disorder, condition, or symptom in a subject, said method comprising administering a protectin, protectin analog, structural isomer, or a pharmaceutically acceptable salt thereof, to the subject.

Embodiment 72 is the method of embodiment 71, wherein the subject suffers from obesity. Embodiment 73 is the method of embodiment 71 or 72, wherein the protectin, protectin analog, structural isomer, or a pharmaceutically acceptable salt thereof is at least one of:

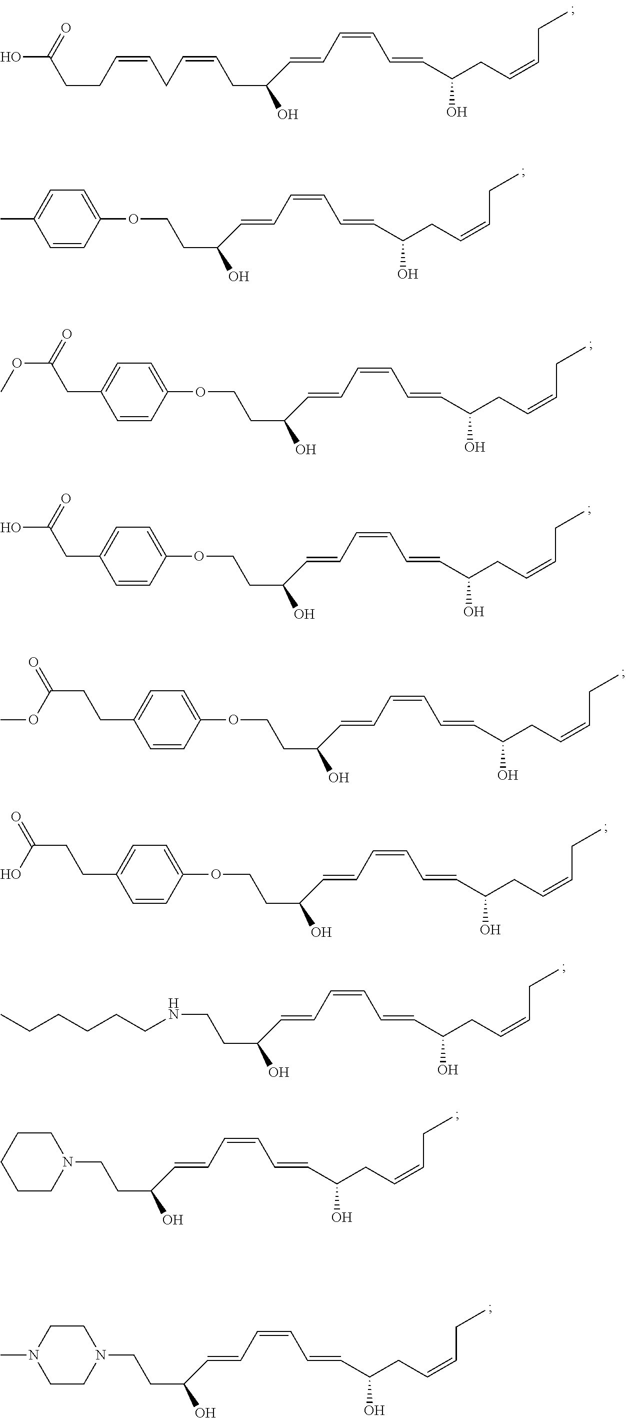

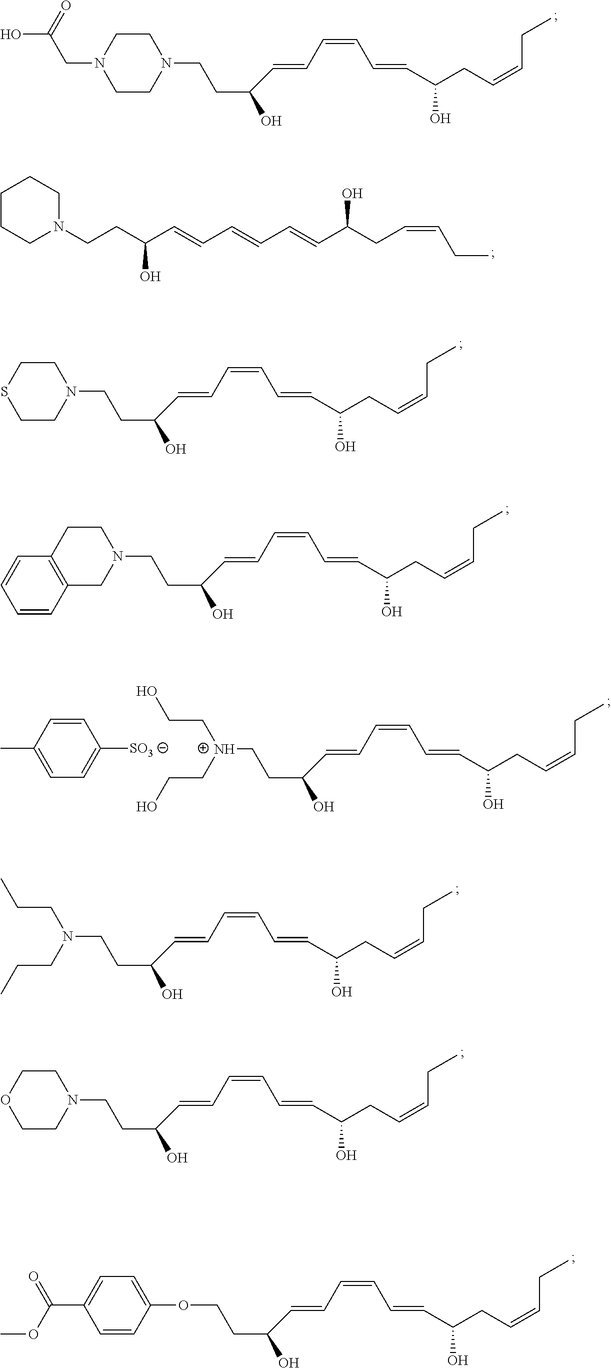

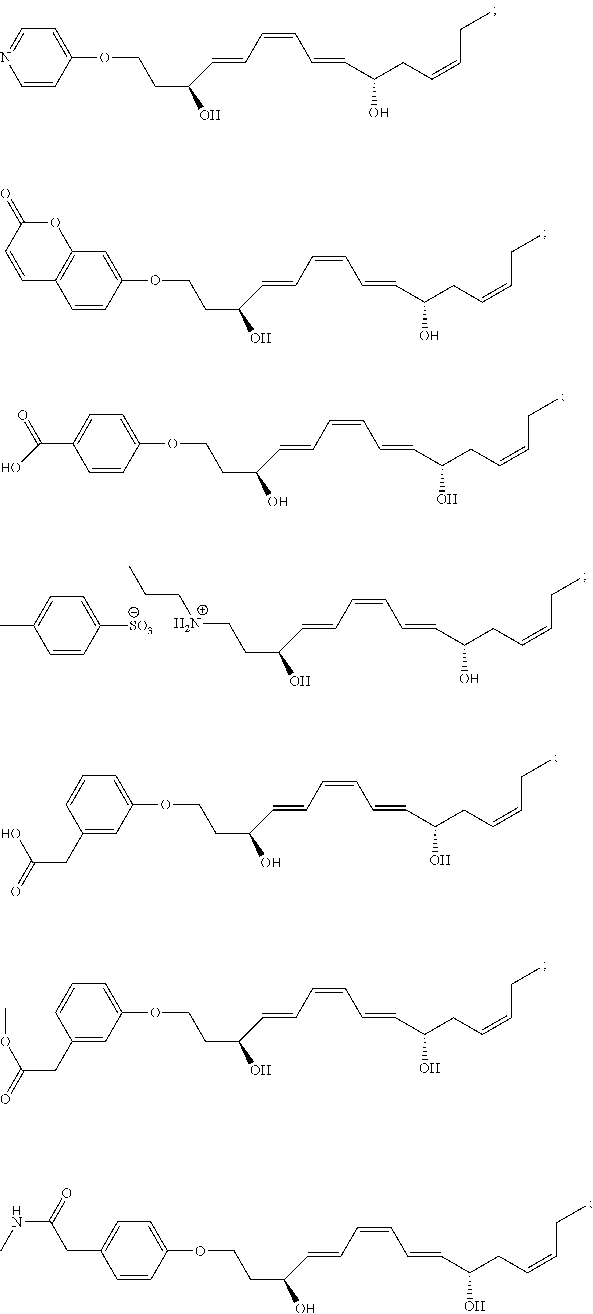

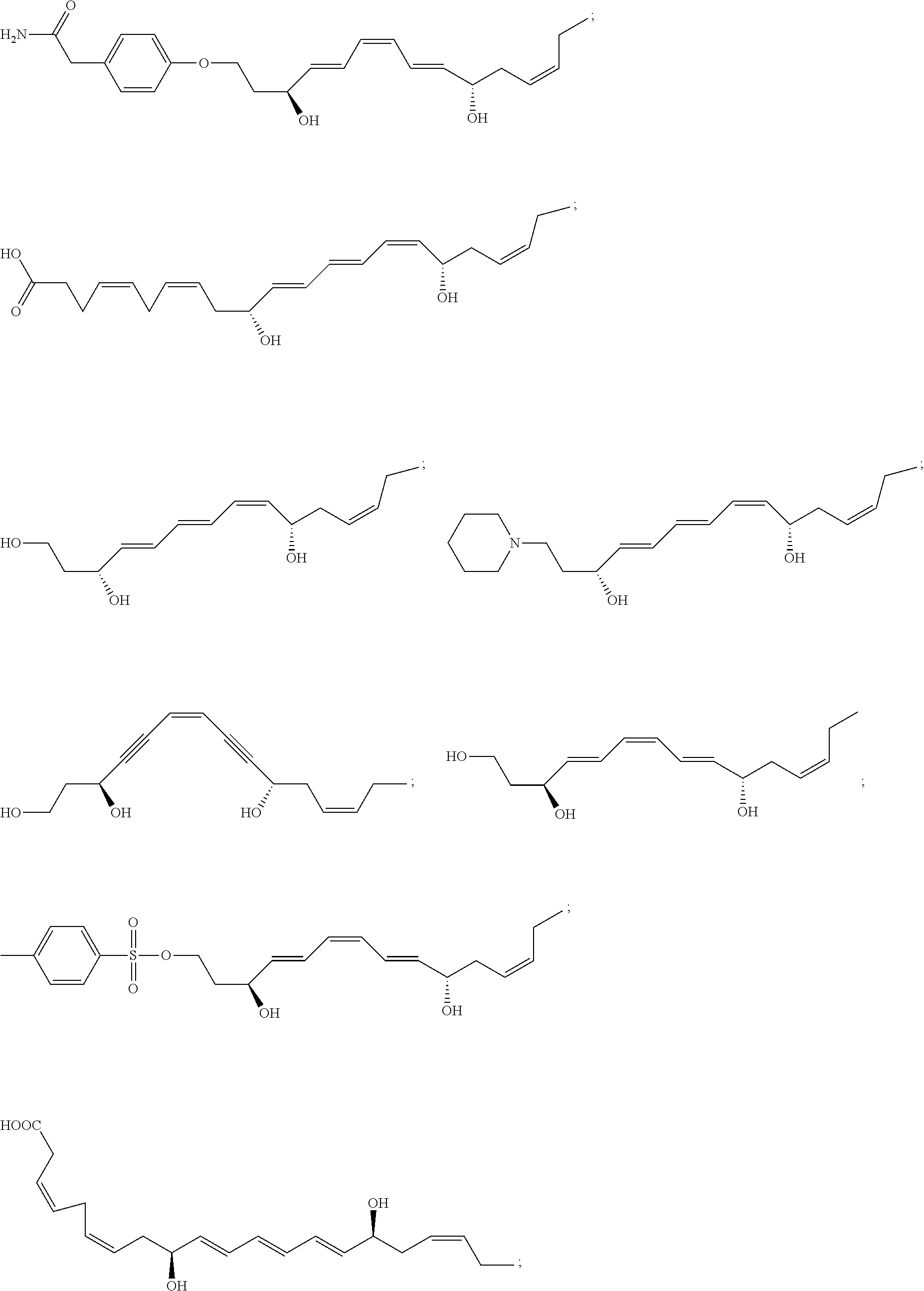

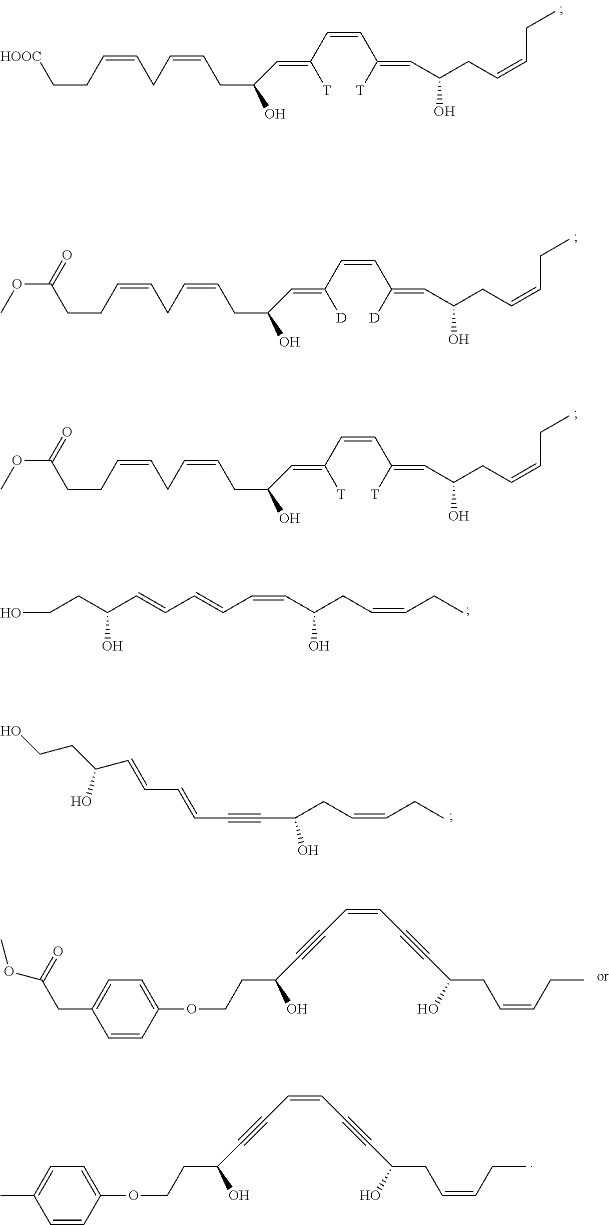

Embodiment 74 is the use of a protectin, protectin analog, structural isomer, or a pharmaceutically acceptable salt thereof for delaying the onset or reducing the severity of, preventing or reversing the progression of, or treating an inflammatory disease, disorder, condition, or symptom in a subject.

Embodiment 75 is the use of a protectin, protectin analog, structural isomer, or a pharmaceutically acceptable salt thereof for the preparation of a medicament for delaying the onset or reducing the severity of, preventing or reversing the progression of, or treating an inflammatory disease, disorder, condition, or symptom in a subject.

Embodiment 76 is the use of embodiment 74 or 75, wherein the subject suffers from obesity.

Embodiment 77 is the use of any one of embodiments 74 to 76, wherein the protectin, protectin analog, structural isomer, or a pharmaceutically acceptable salt thereof is at least one of:

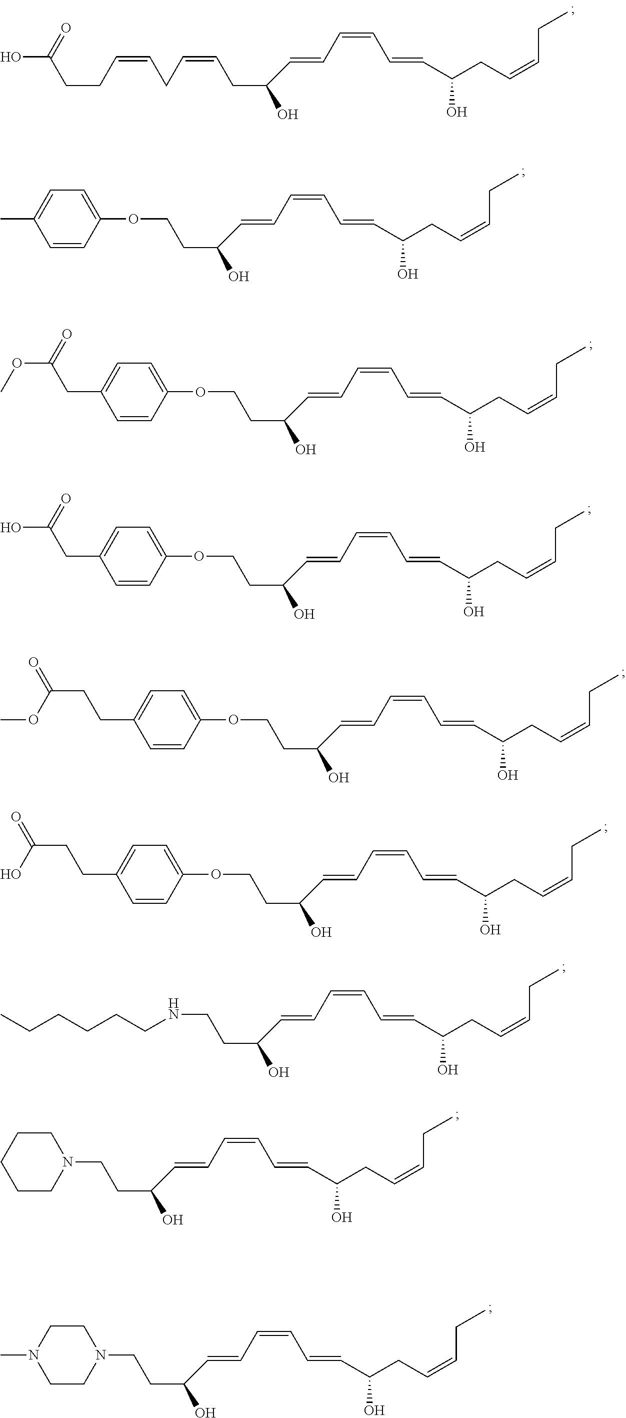

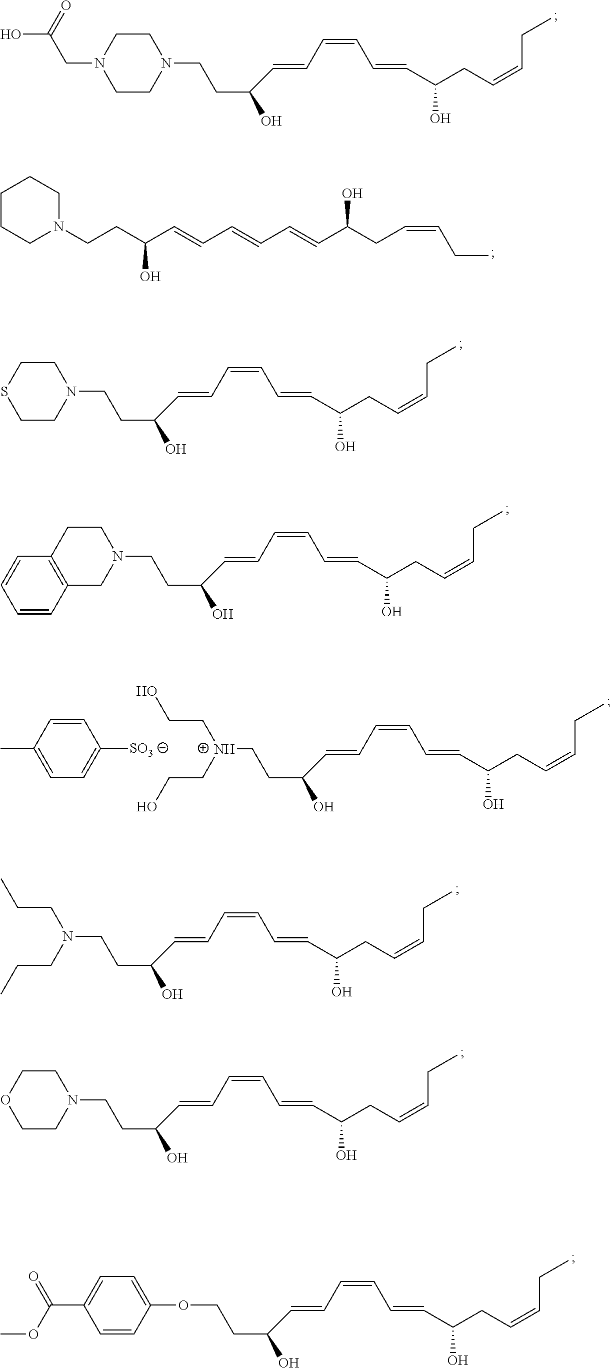

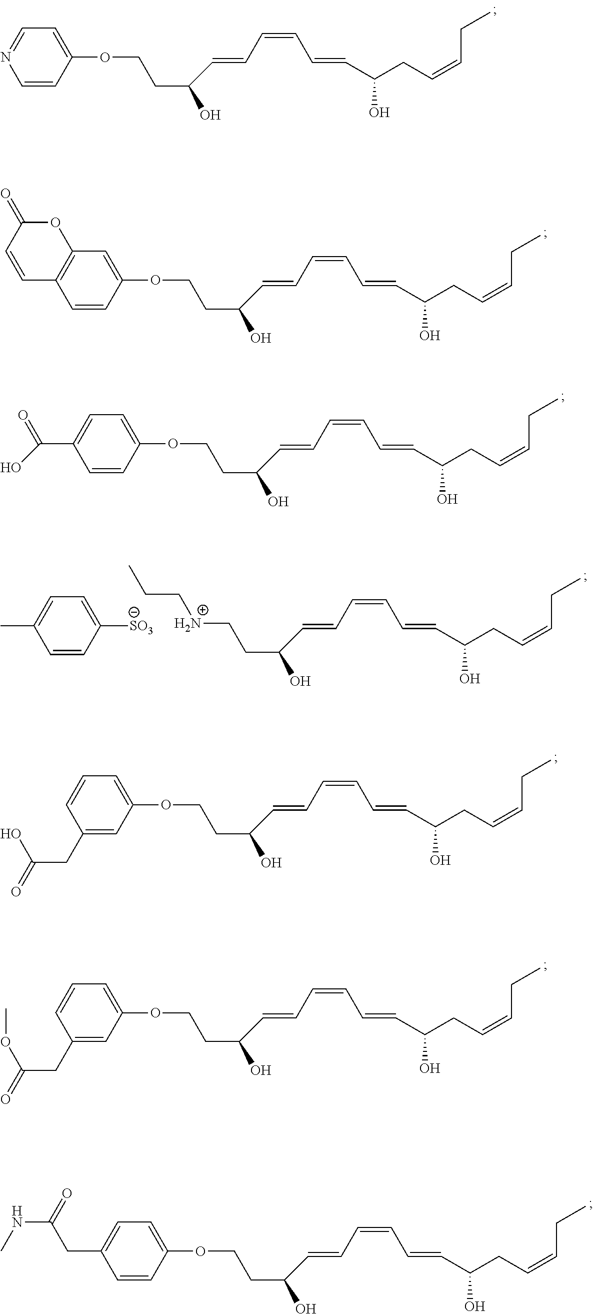

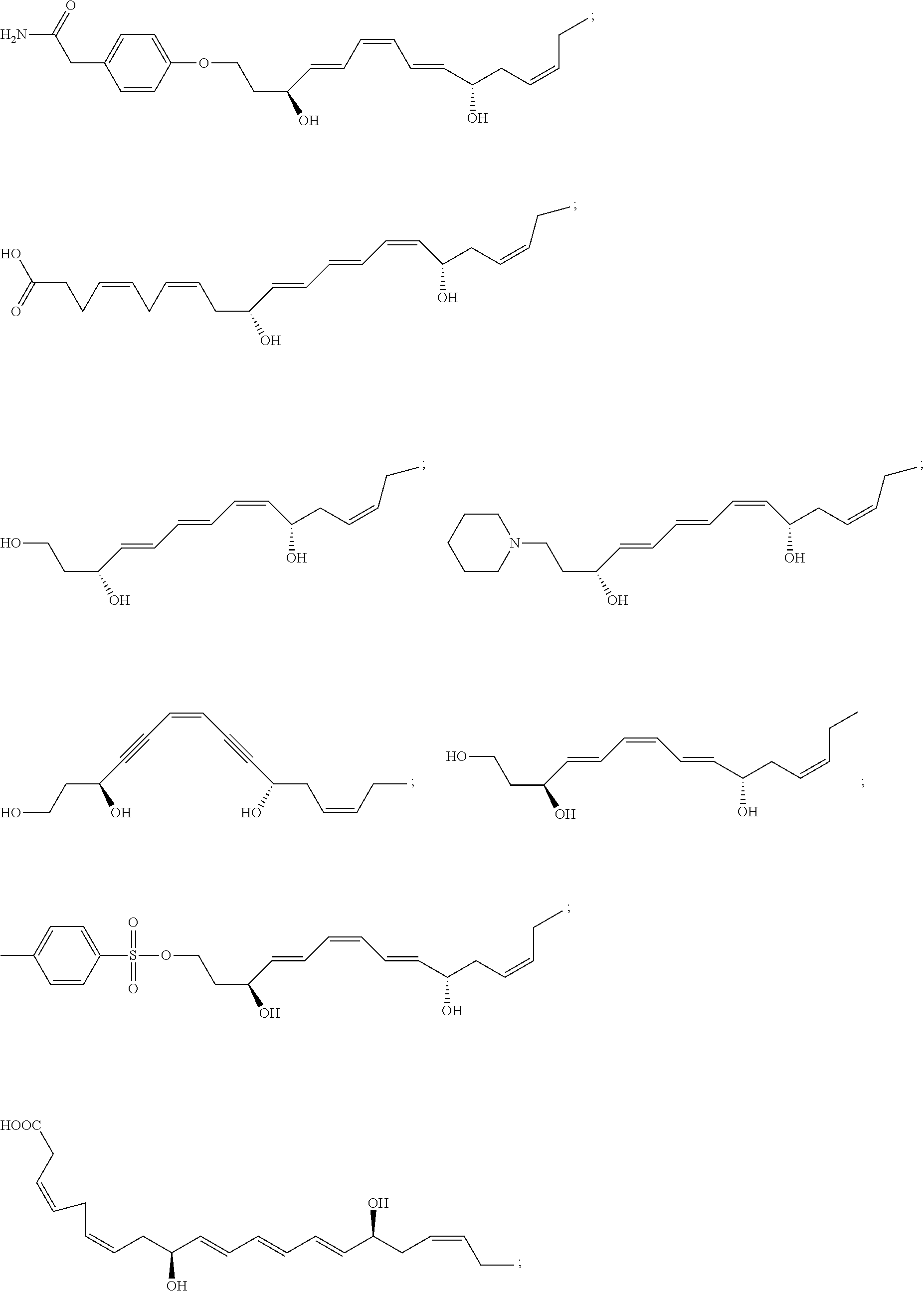

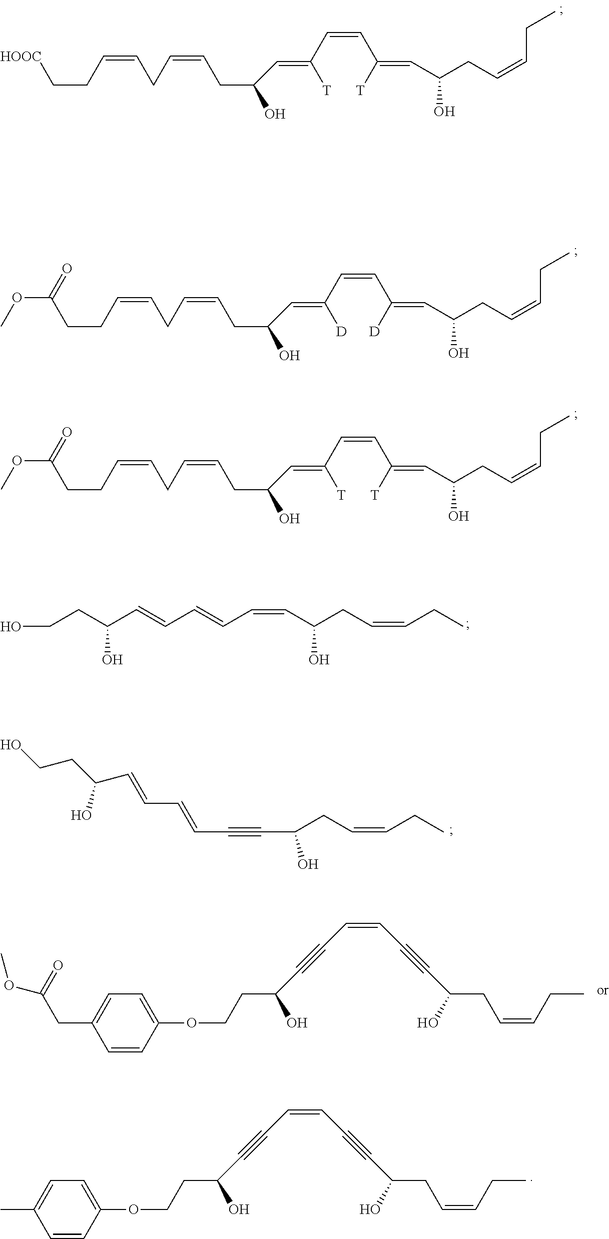

Embodiment 78 is a protectin, protectin analog, structural isomer, or a pharmaceutically acceptable salt thereof for use in delaying the onset or reducing the severity of, preventing or reversing the progression of, or treating an inflammatory disease, disorder, condition, or symptom in a subject.

Embodiment 79 is the protectin, protectin analog, structural isomer, or pharmaceutically acceptable salt thereof for use of embodiment 78, wherein the subject suffers from obesity.

Embodiment 80 is the protectin, protectin analog, structural isomer, or pharmaceutically acceptable salt thereof for use of embodiment 78 or 79, wherein the protectin, protectin analog, structural isomer, or a pharmaceutically acceptable salt thereof is at least one of:

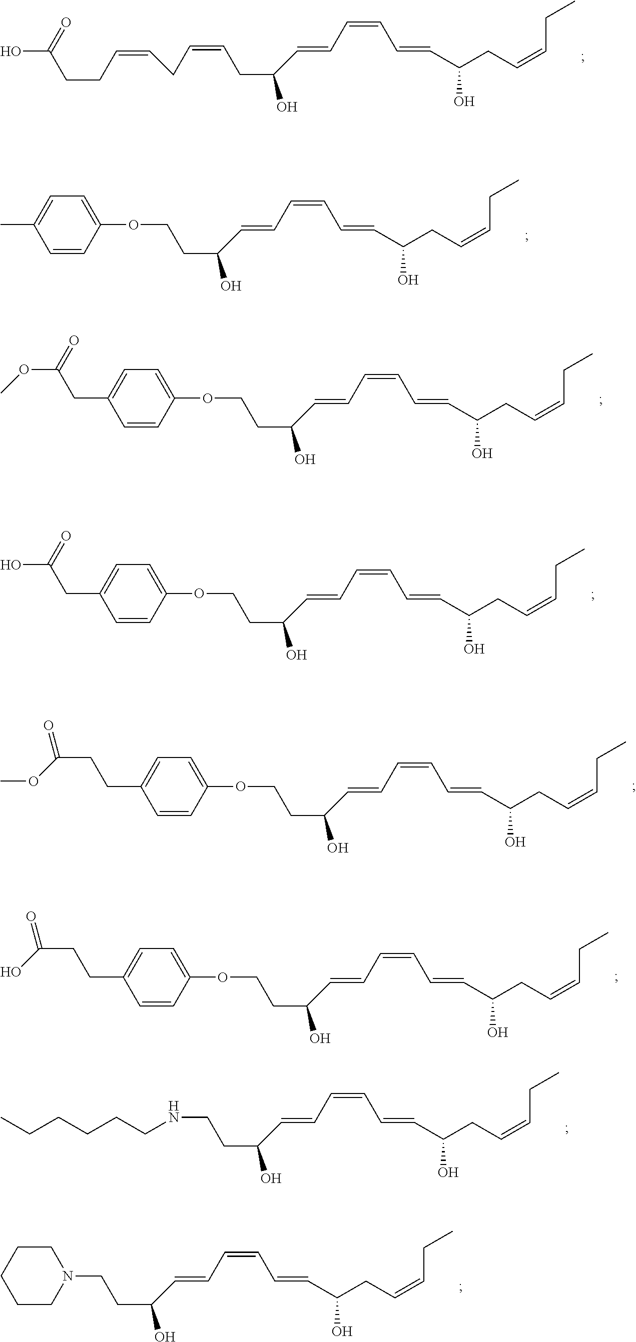

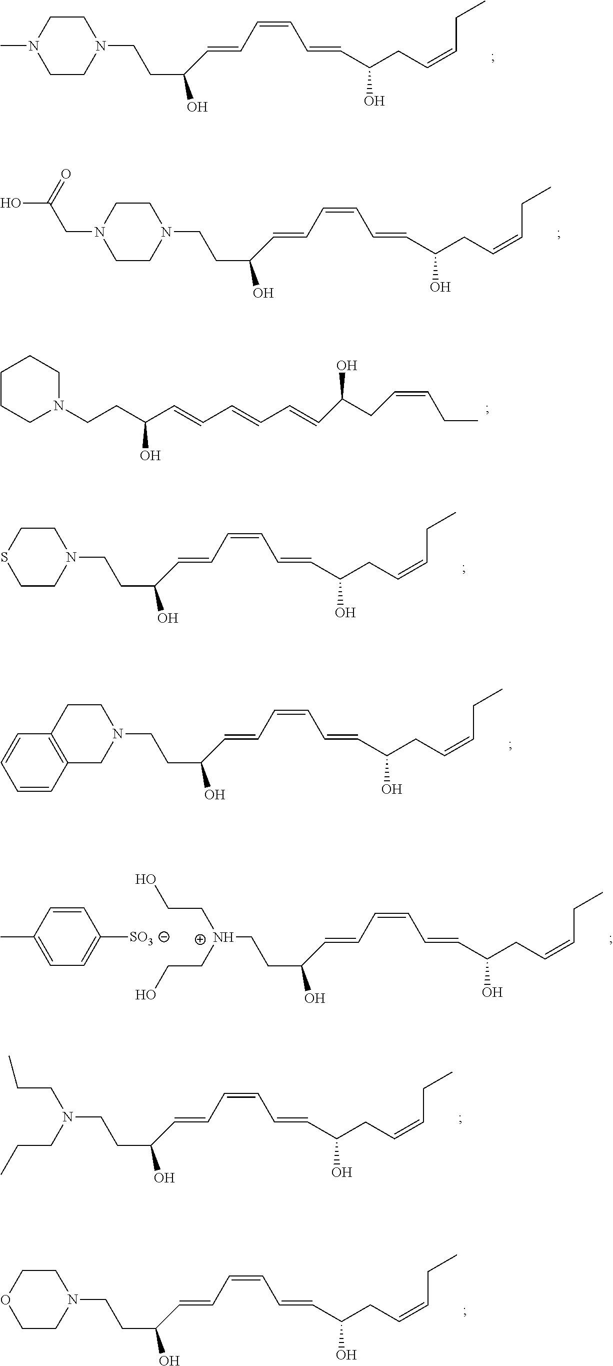

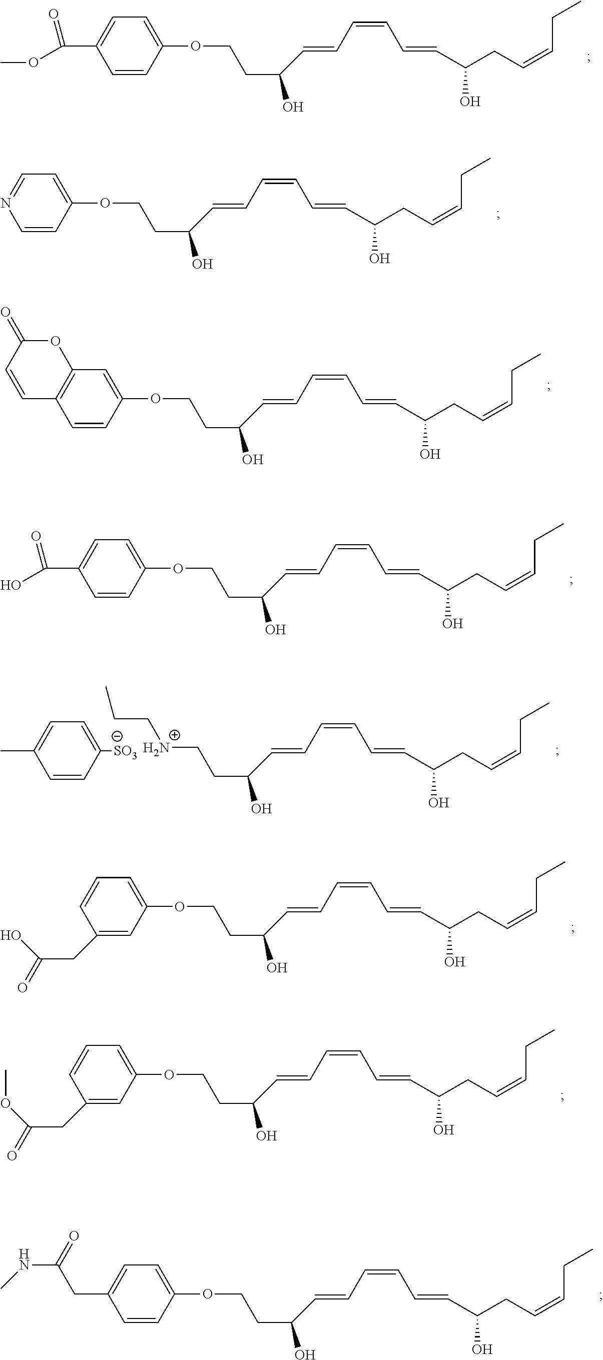

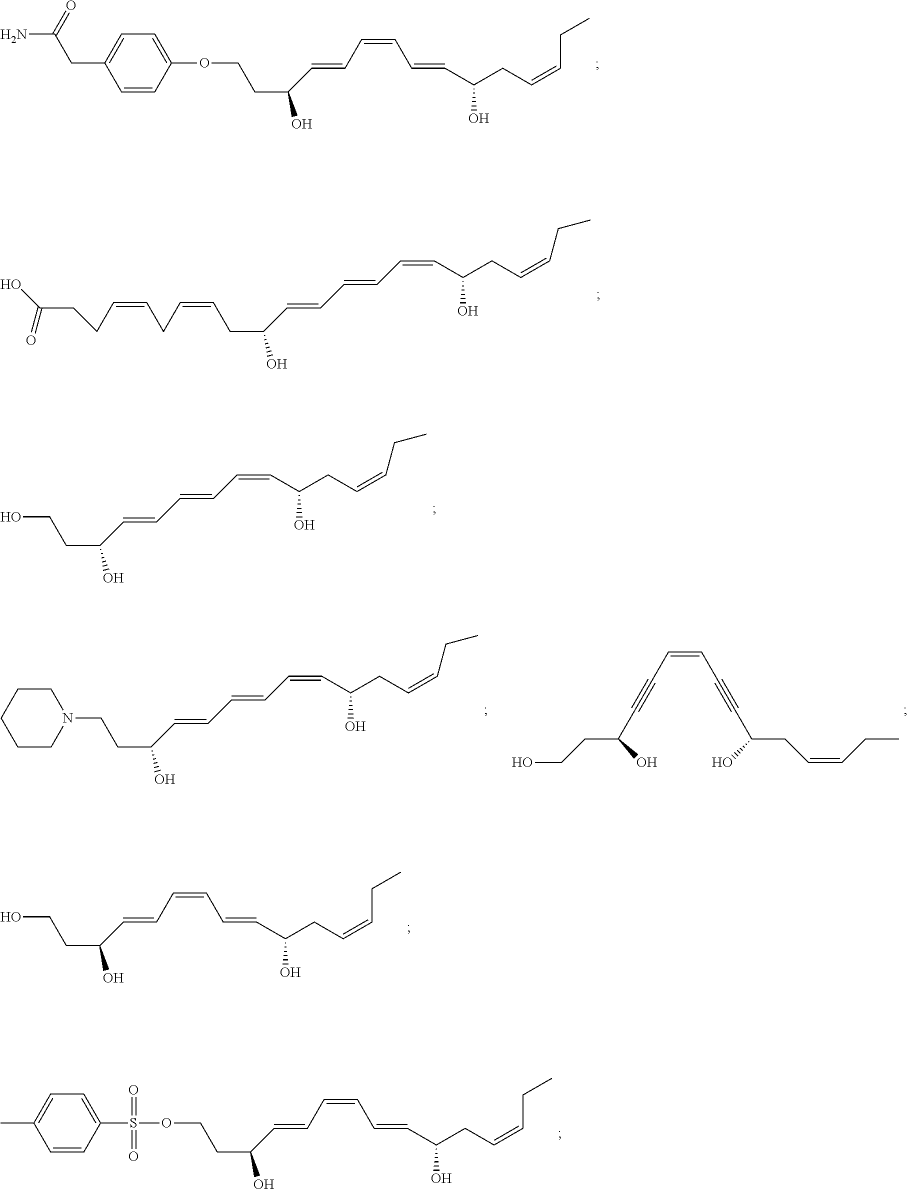

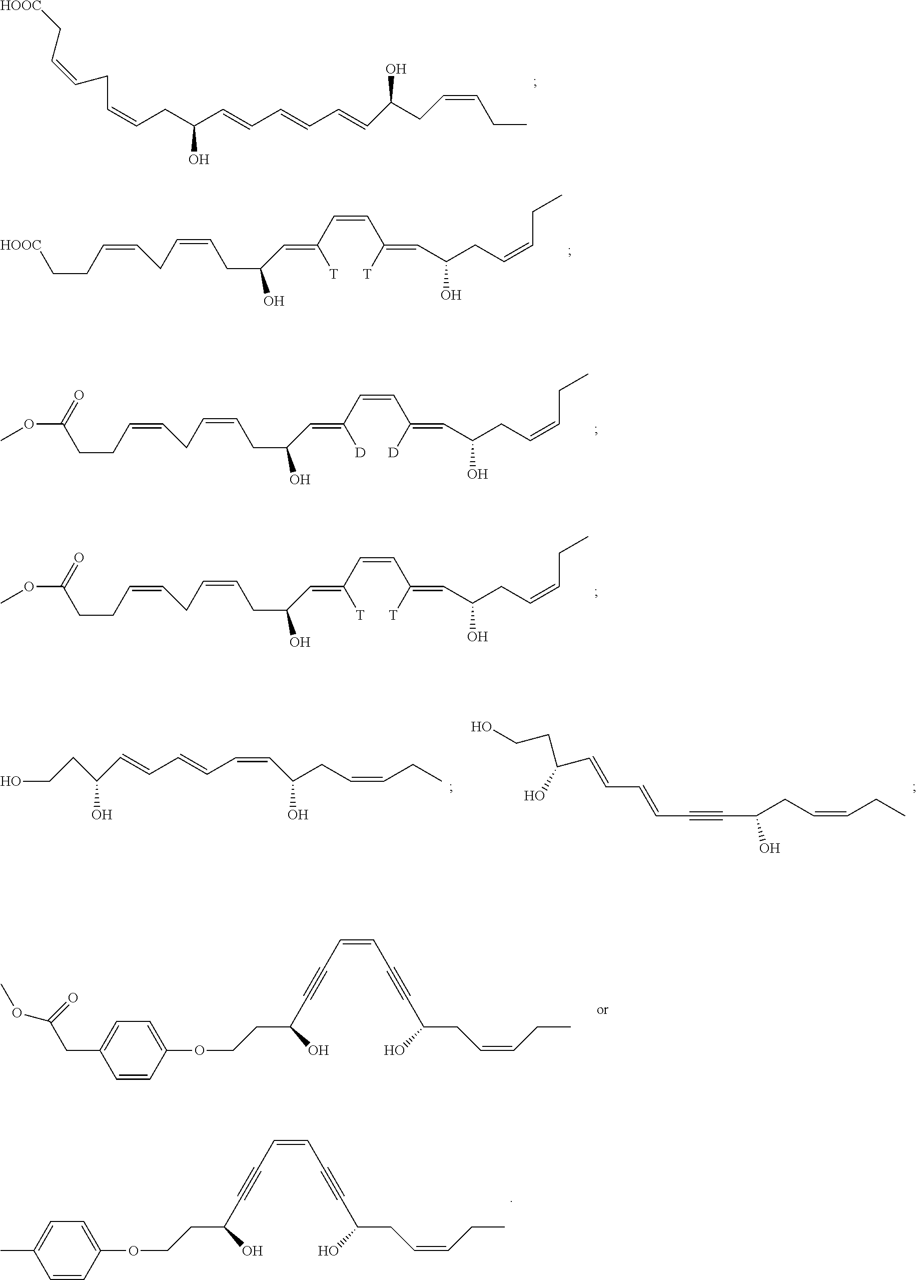

Embodiment 81 is the method of any one of embodiments 1 to 10, further comprising a second agent for delaying the onset or reducing the severity of, preventing or reversing the progression of, or treating a viral infection or disease in a subject.

Embodiment 82 is the method of any one of embodiments 32 to 37, further comprising a second agent for delaying the onset or reducing the severity of, preventing or reversing the progression of, or treating diabetes in a subject.

Embodiment 83 is the method of any one of embodiments 51 to 53, further comprising a second agent for lowering blood glucose levels in a subject.

Embodiment 84 is the method of any one of embodiments 61 to 63, further comprising a second agent for delaying the onset or reducing the severity of, preventing or reversing the progression of, or treating insulin resistance in a subject.

Embodiment 85 is the method of any one of embodiments 71 to 73, further comprising a second agent for delaying the onset or reducing the severity of, preventing or reversing the progression of, or treating an inflammatory disease, disorder, condition, or symptom in a subject.

Embodiment 86 is a method of preventing, delaying the onset or reducing the severity of, preventing or reversing the progression of, or treating an inflammatory disease, disorder, condition, or symptom that is secondary to another condition in a subject, said method comprising administering a protectin, protectin analog, structural isomer, or a pharmaceutically acceptable salt thereof, to the subject.

Embodiment 87 is the method of embodiment 86, wherein the protectin, protectin analog, structural isomer, or a pharmaceutically acceptable salt thereof is at least one of:

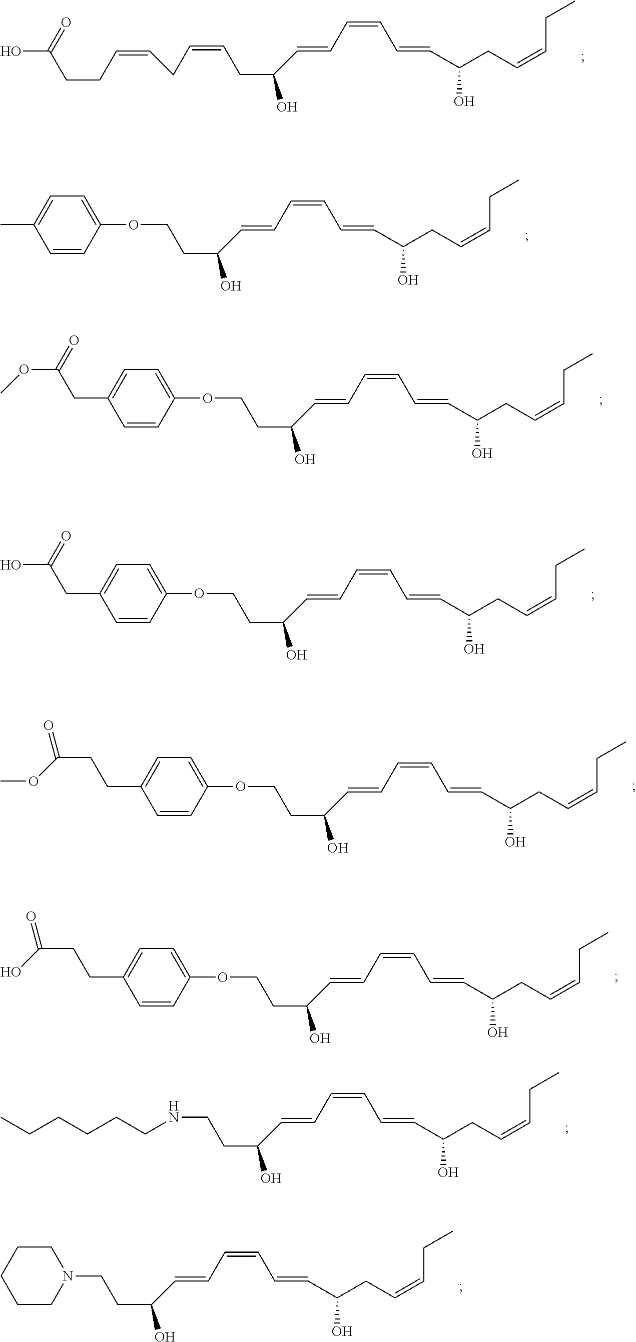

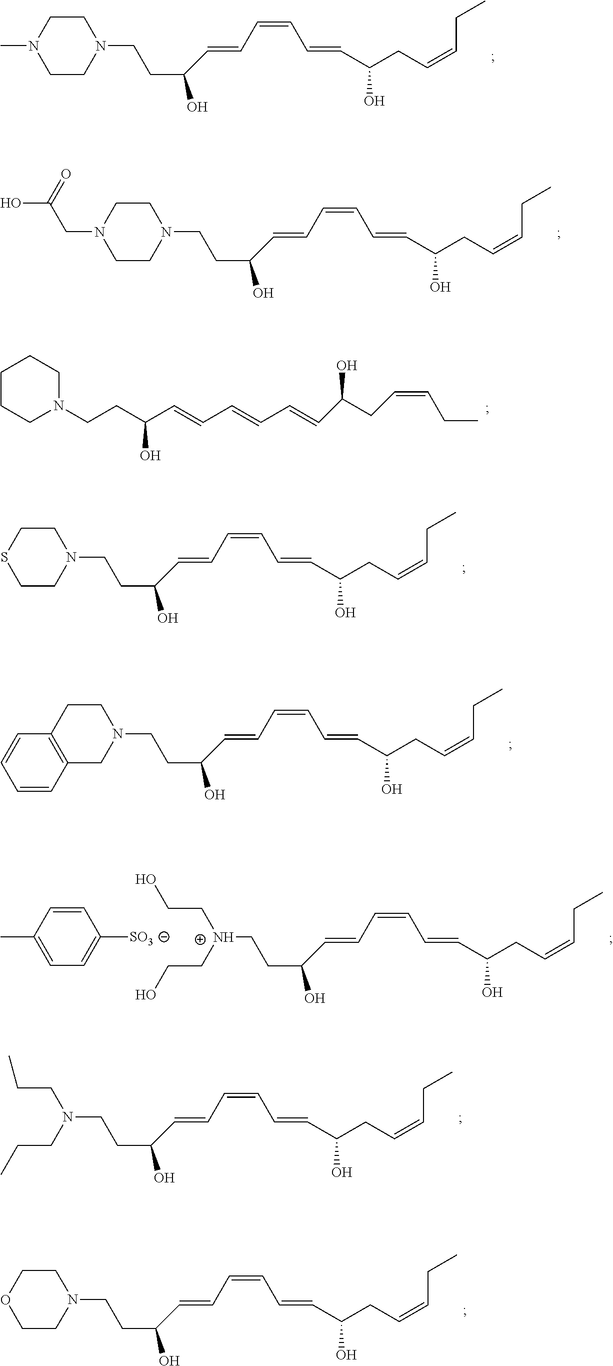

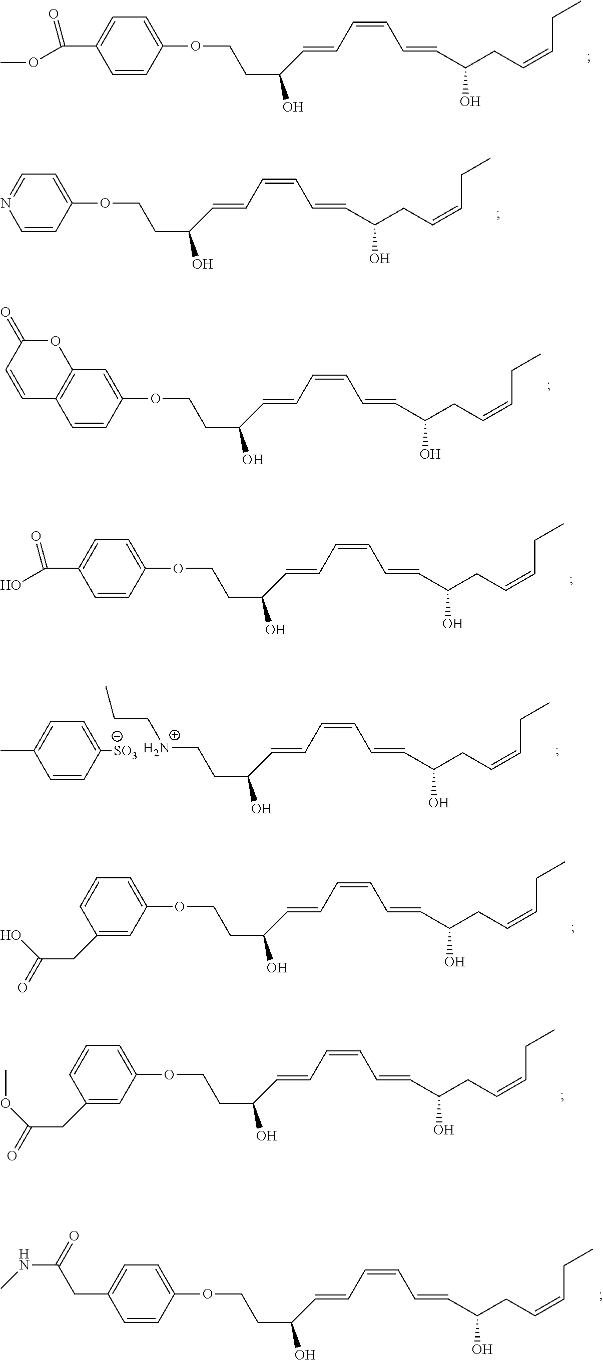

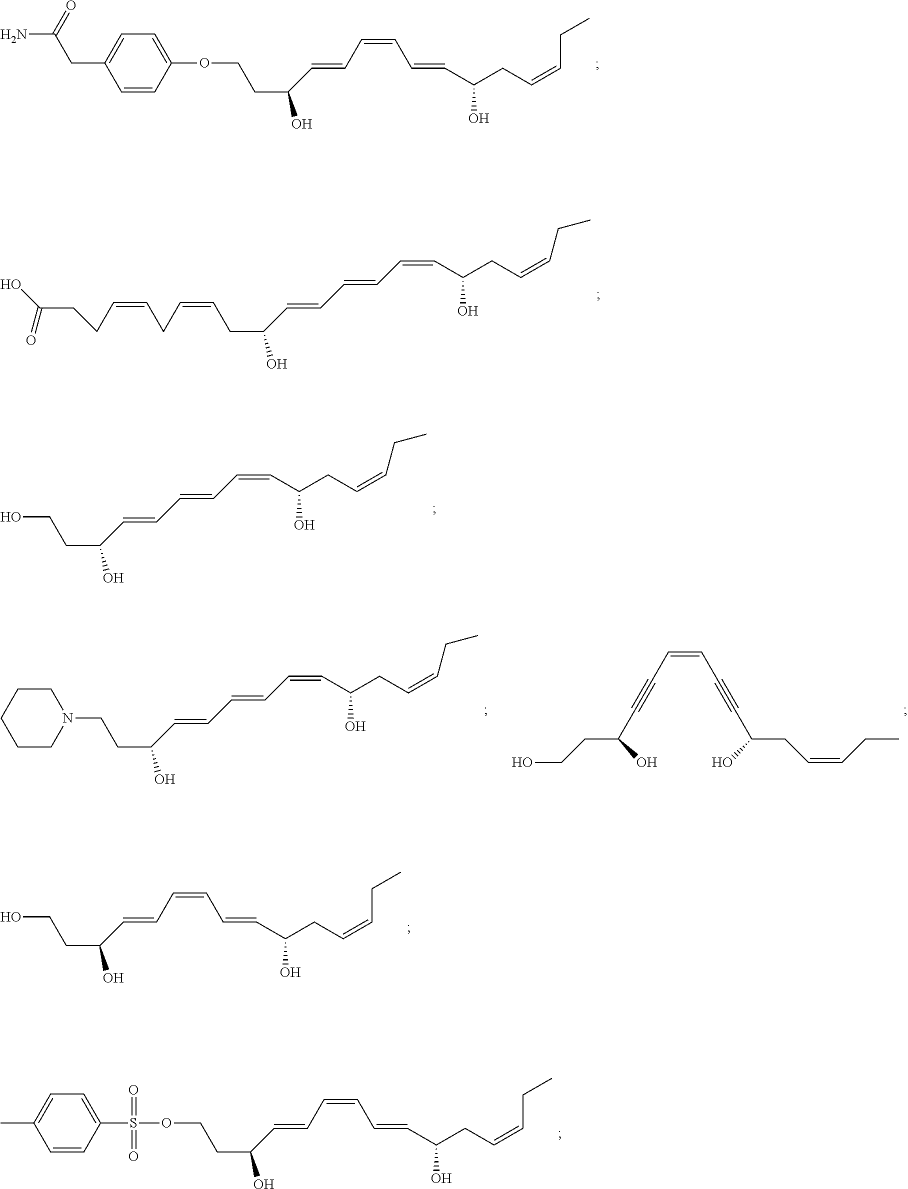

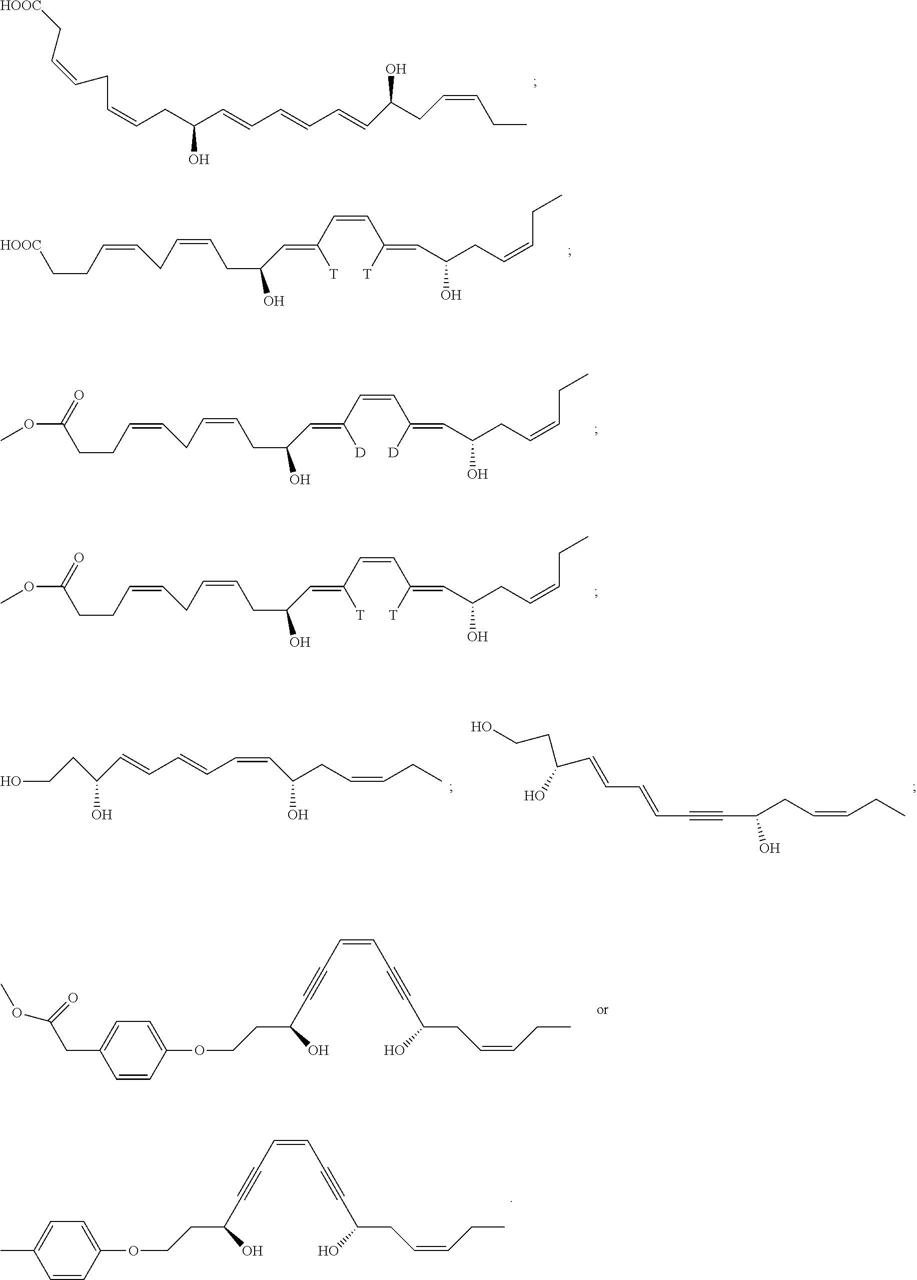

Embodiment 88 is the use of a protectin, protectin analog, structural isomer, or a pharmaceutically acceptable salt thereof for delaying the onset or reducing the severity of, preventing or reversing the progression of, or treating an inflammatory disease, disorder, condition, or symptom that is secondary to another condition in a subject.

Embodiment 89 is the use of a protectin, protectin analog, structural isomer, or a pharmaceutically acceptable salt thereof for the preparation of a medicament for delaying the onset or reducing the severity of, preventing or reversing the progression of, or treating an inflammatory disease, disorder, condition, or symptom that is secondary to another condition in a subject.

Embodiment 90 is the use of embodiment 88 or 89, wherein the protectin, protectin analog, structural isomer, or a pharmaceutically acceptable salt thereof is at least one of:

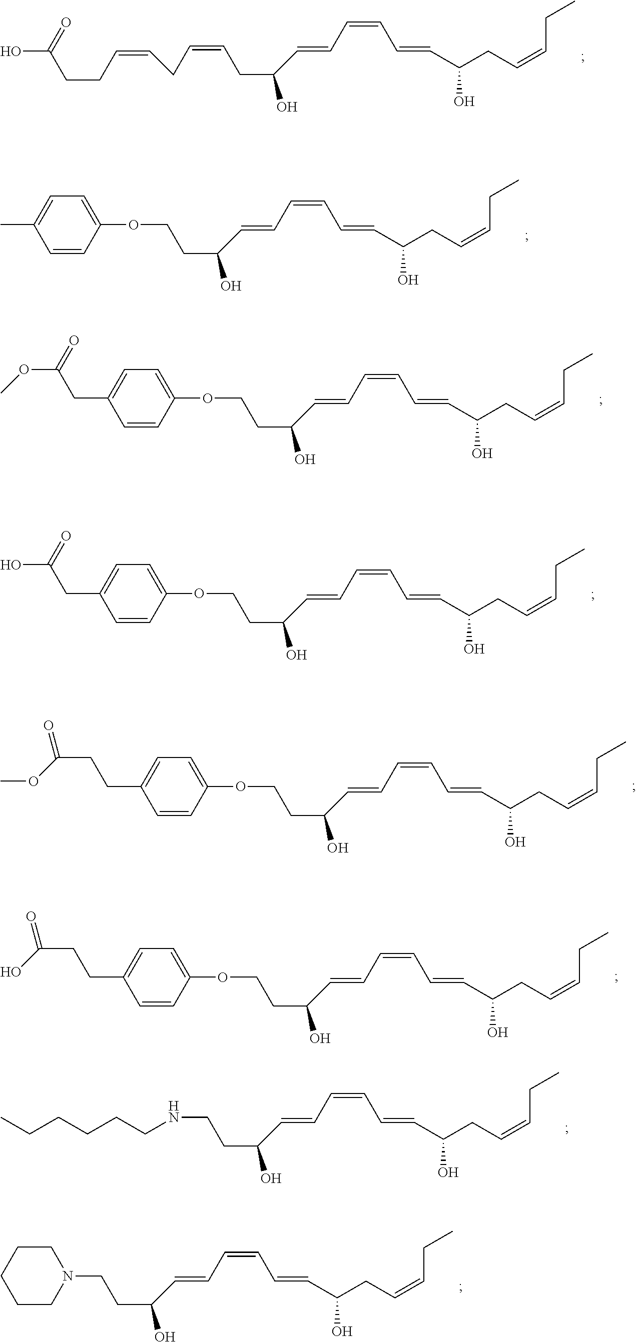

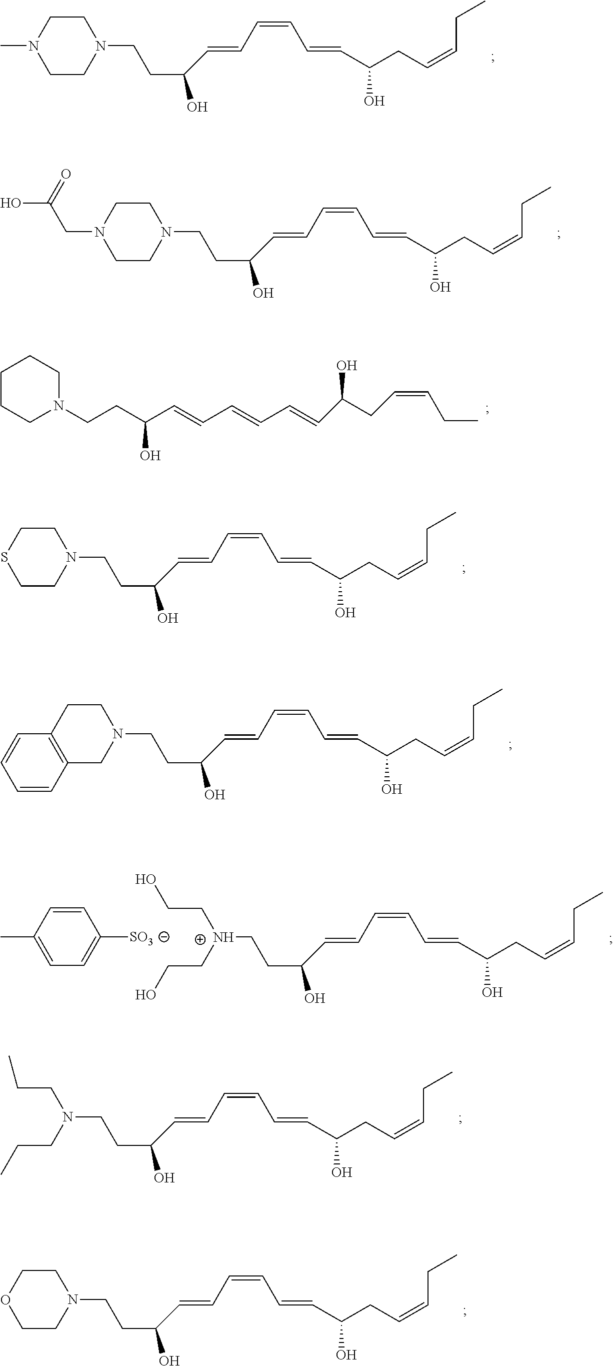

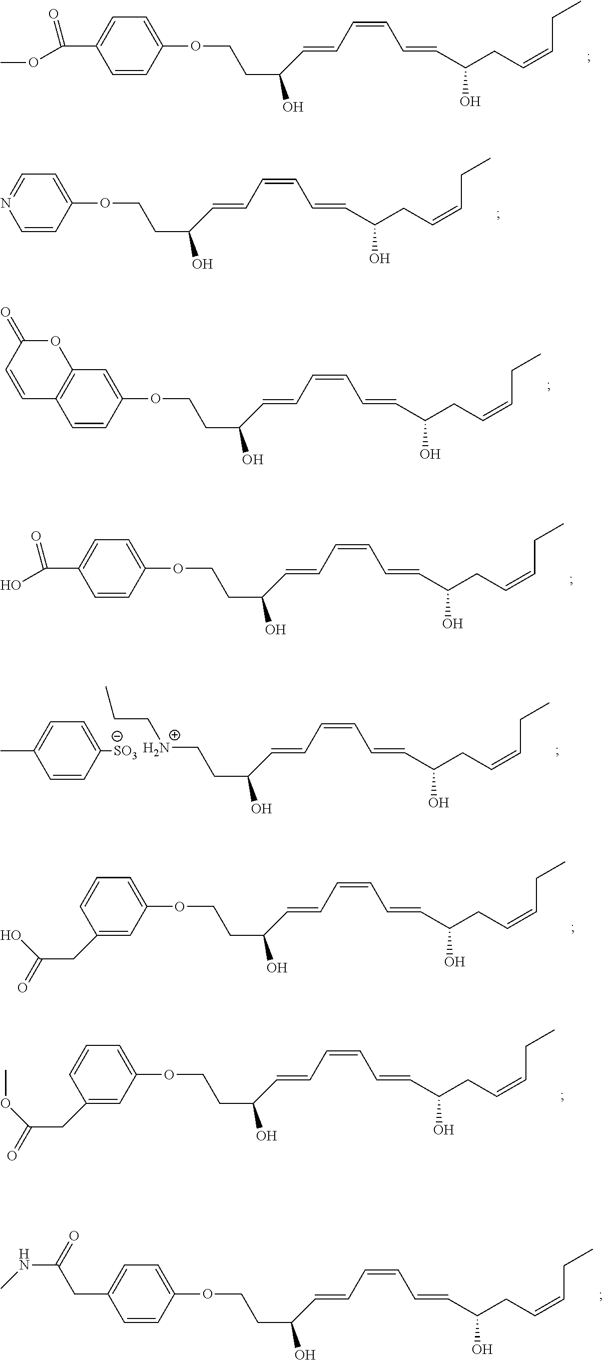

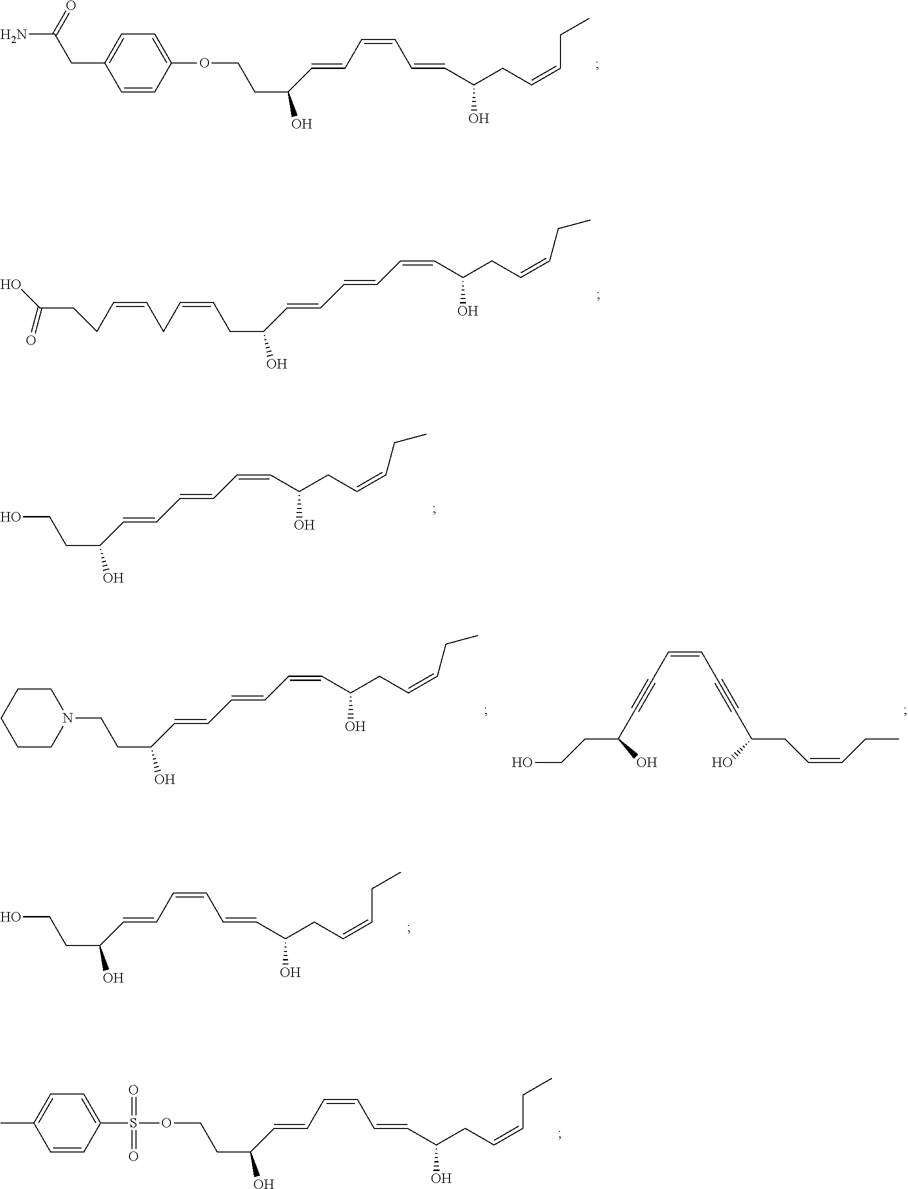

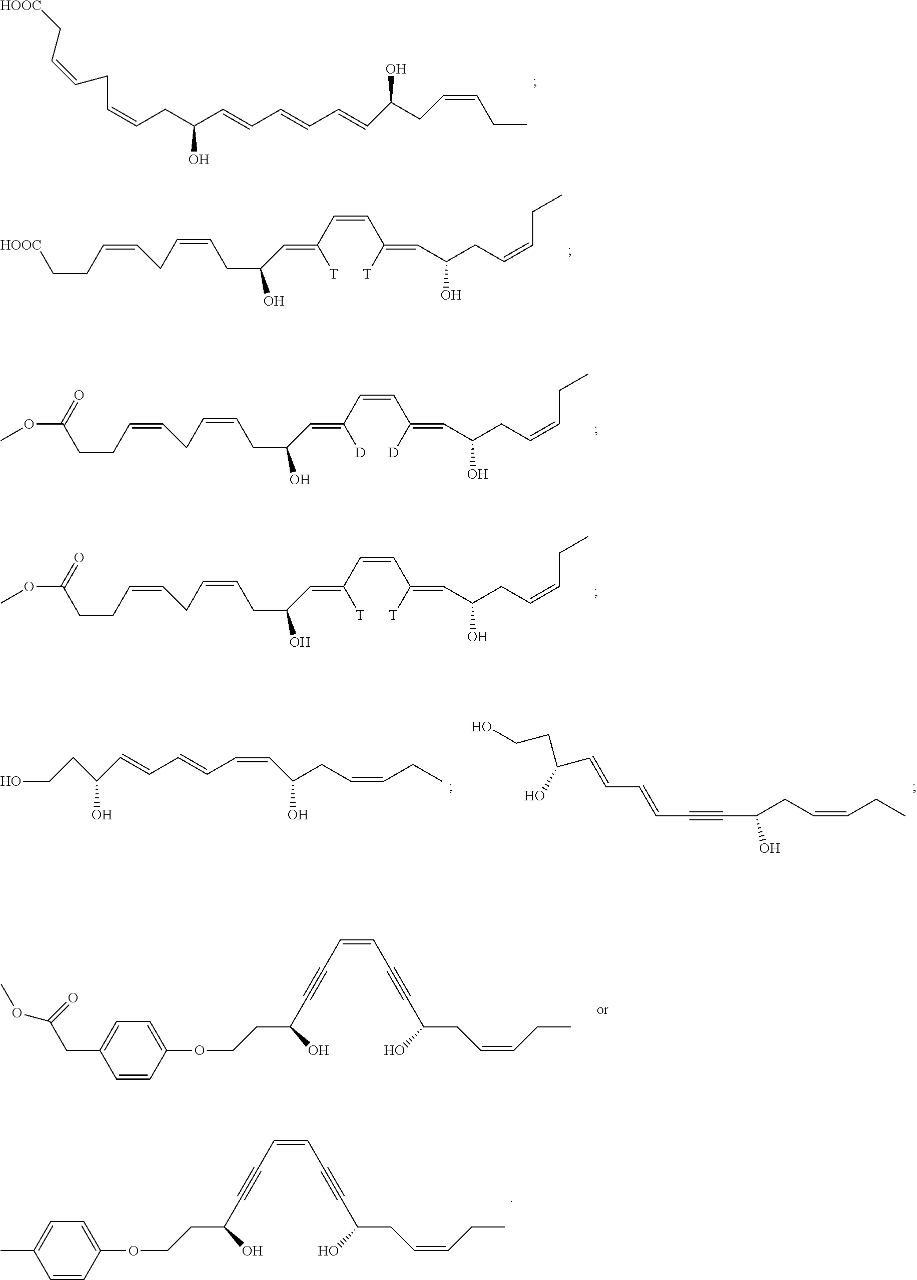

Embodiment 91 is a protectin, protectin analog, structural isomer, or a pharmaceutically acceptable salt thereof for use in delaying the onset or reducing the severity of, preventing or reversing the progression of, or treating an inflammatory disease, disorder, condition, or symptom that is secondary to another condition in a subject.

Embodiment 92 is the protectin, protectin analog, structural isomer, or pharmaceutically acceptable salt thereof for use of embodiment 91, wherein the protectin, protectin analog, structural isomer, or a pharmaceutically acceptable salt thereof is at least one of:

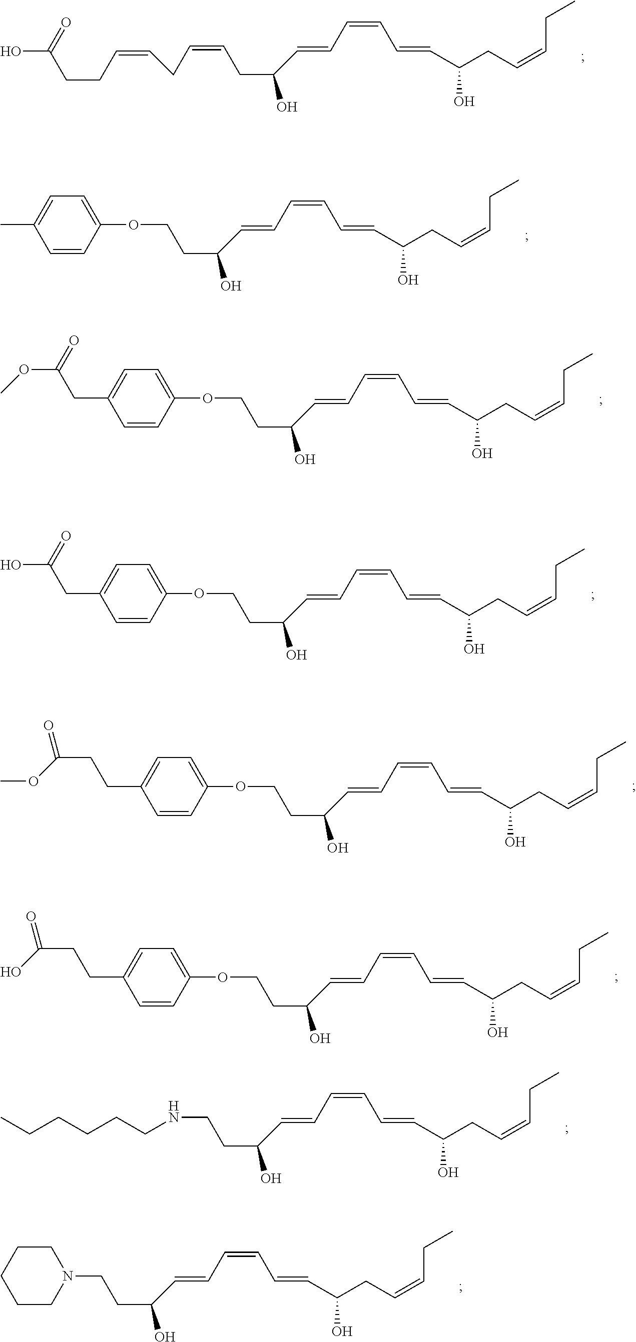

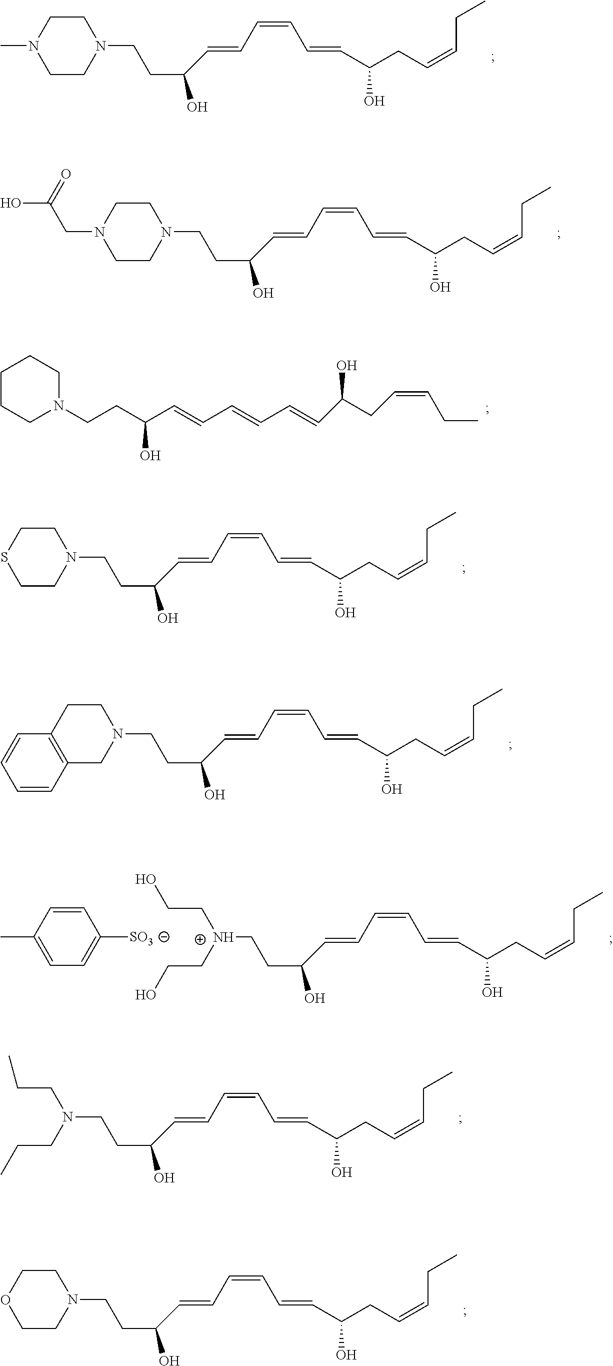

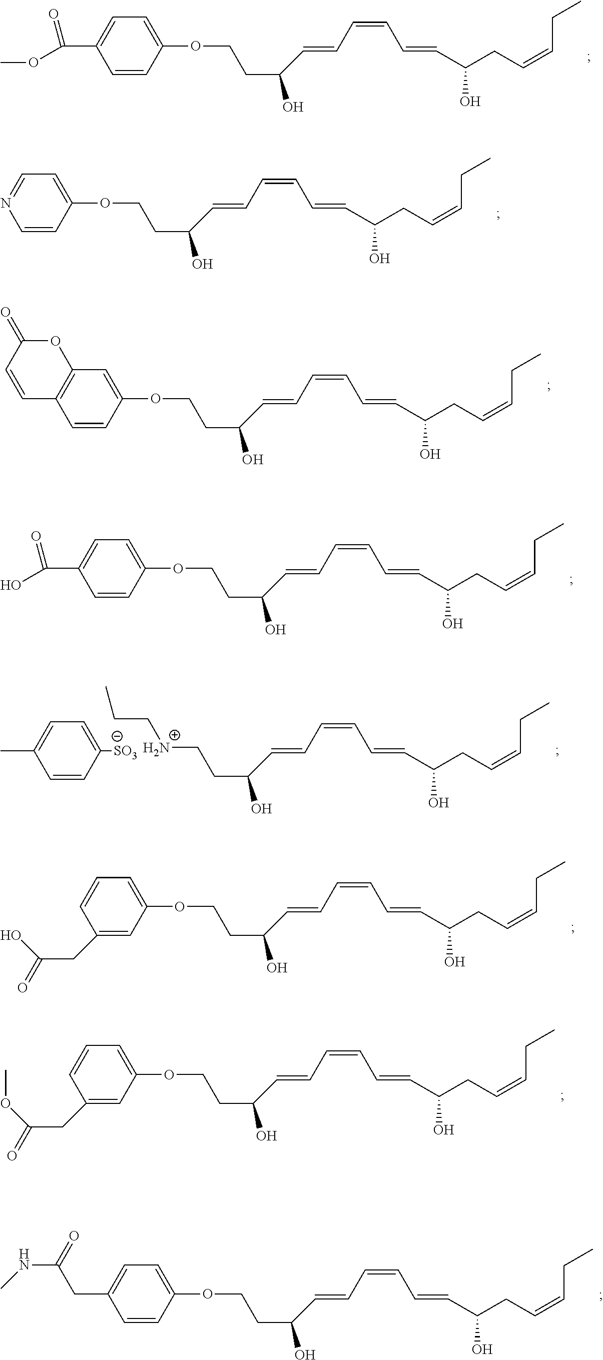

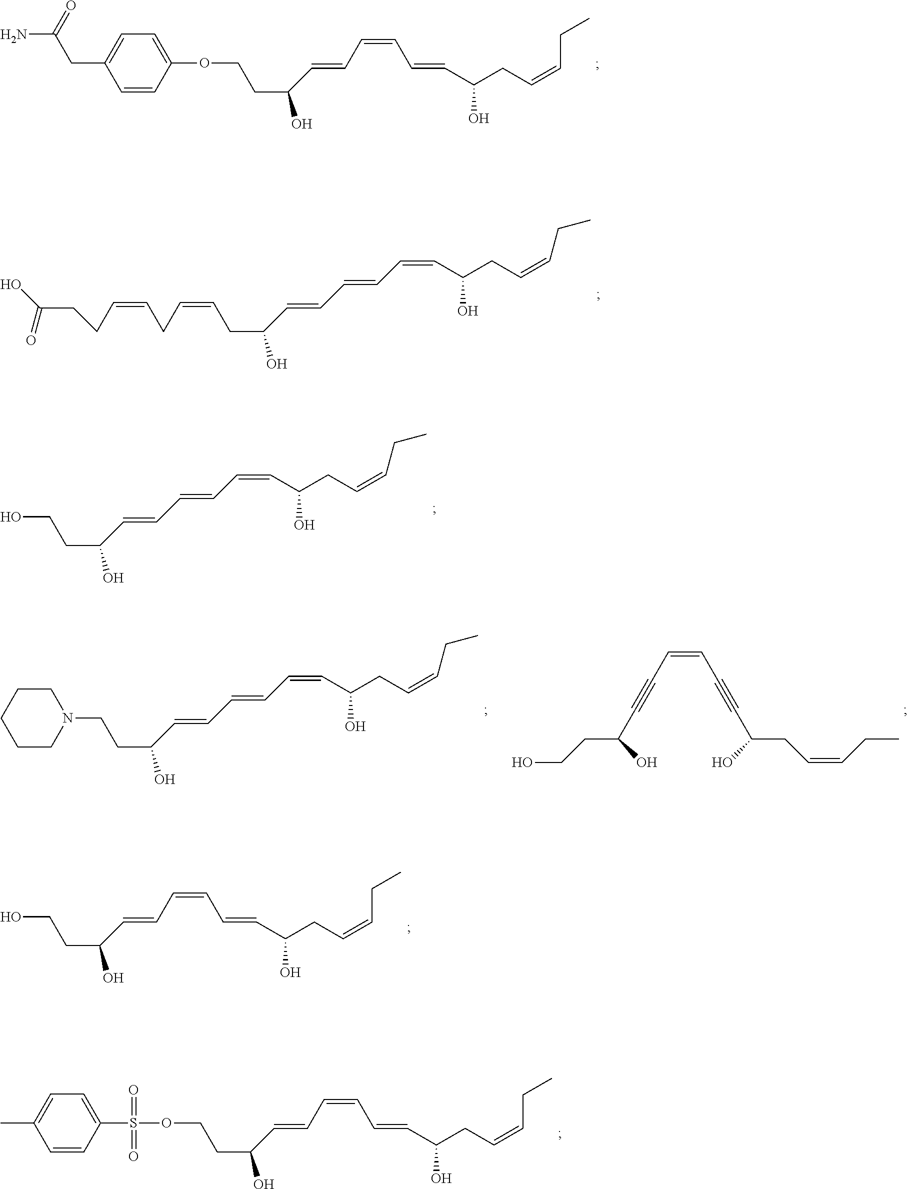

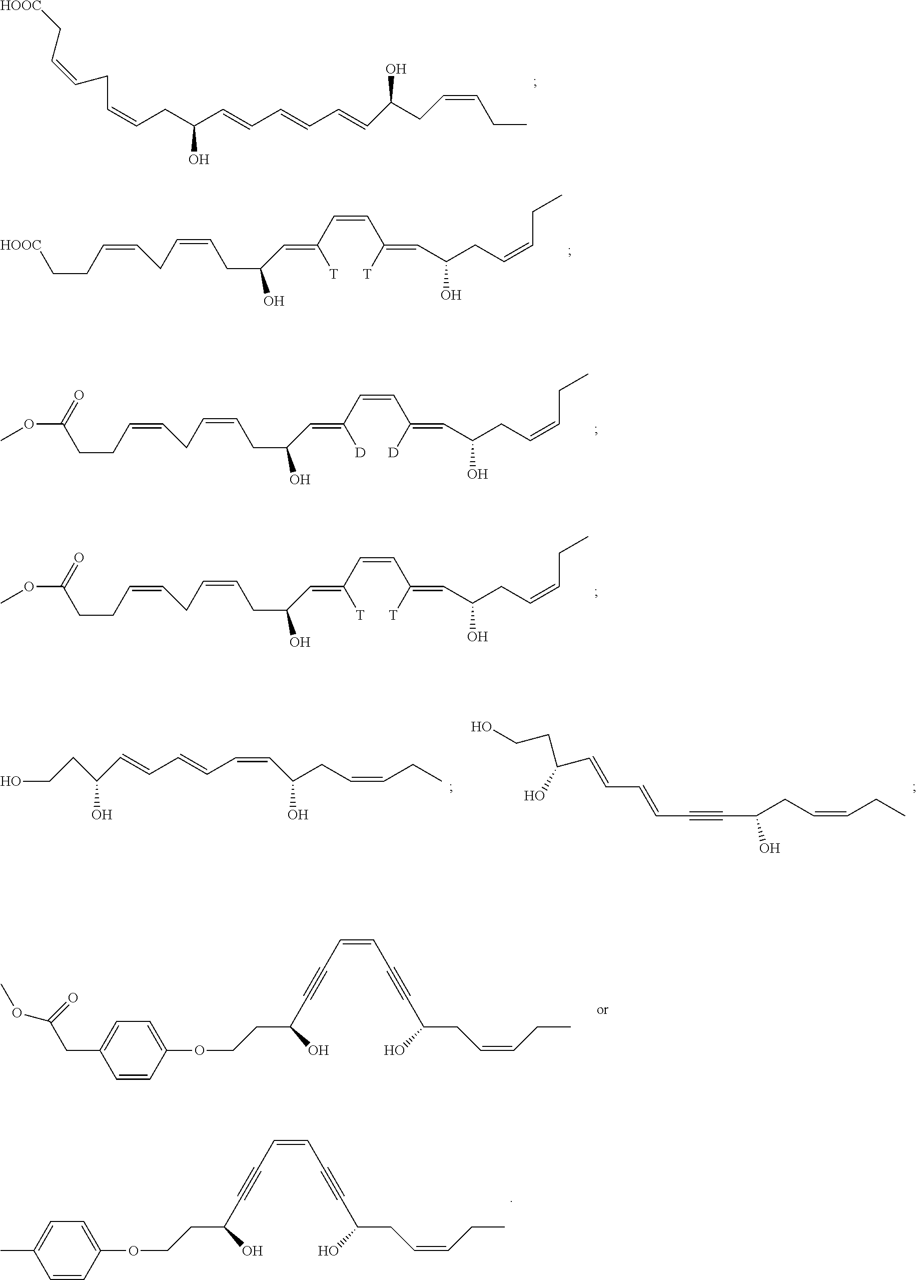

Embodiment 93 is a method of preventing, delaying the onset or reducing the severity of, preventing or reversing the progression of, or treating chronic low-level inflammation associated with chronic conditions in a subject, said method comprising administering a protectin, protectin analog, structural isomer, or a pharmaceutically acceptable salt thereof, to the subject.

Embodiment 94 is the method of embodiment 93, wherein the chronic conditions comprise neurological disorders, ocular disorders, autoimmune disorders, metabolic disorders, gastrointestinal disorders, and skin disorders.

Embodiment 95 is the method of embodiment 93 or 94, wherein reduction of inflammation is through concomitantly reducing the initiation phase and enhancing the resolution phase of the inflammation.

Embodiment 96 is the method of any one of embodiments 93 to 95, wherein the protectin, protectin analog, structural isomer, or a pharmaceutically acceptable salt thereof is at least one of:

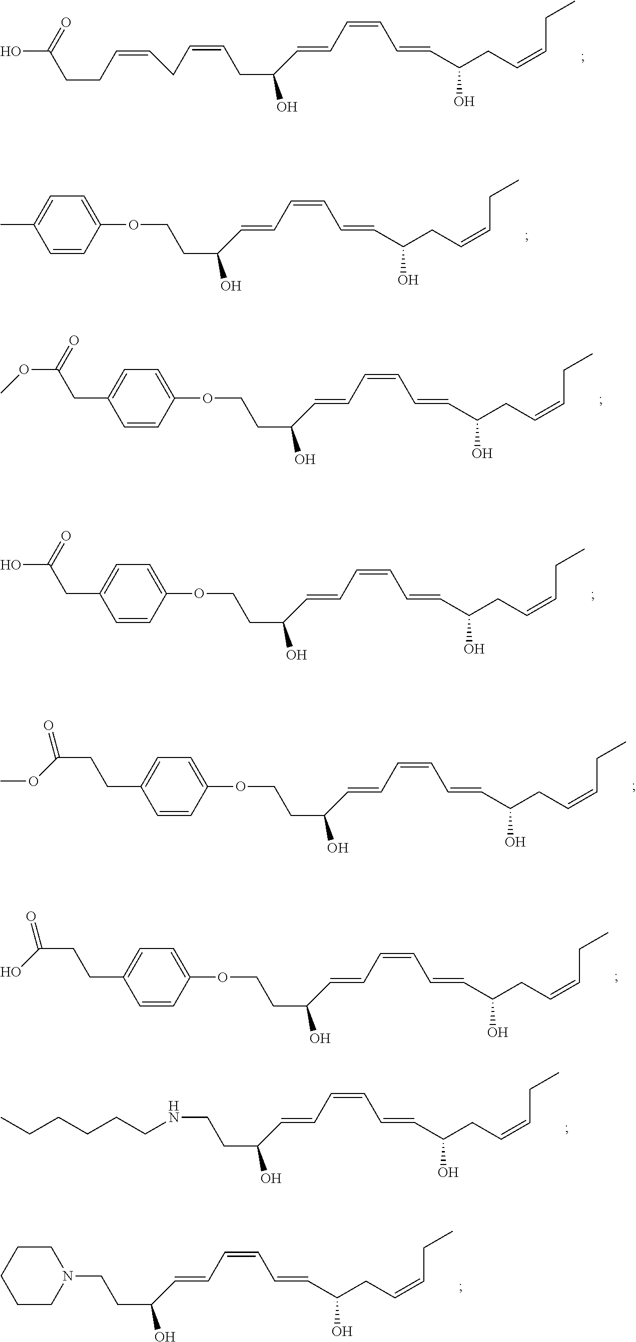

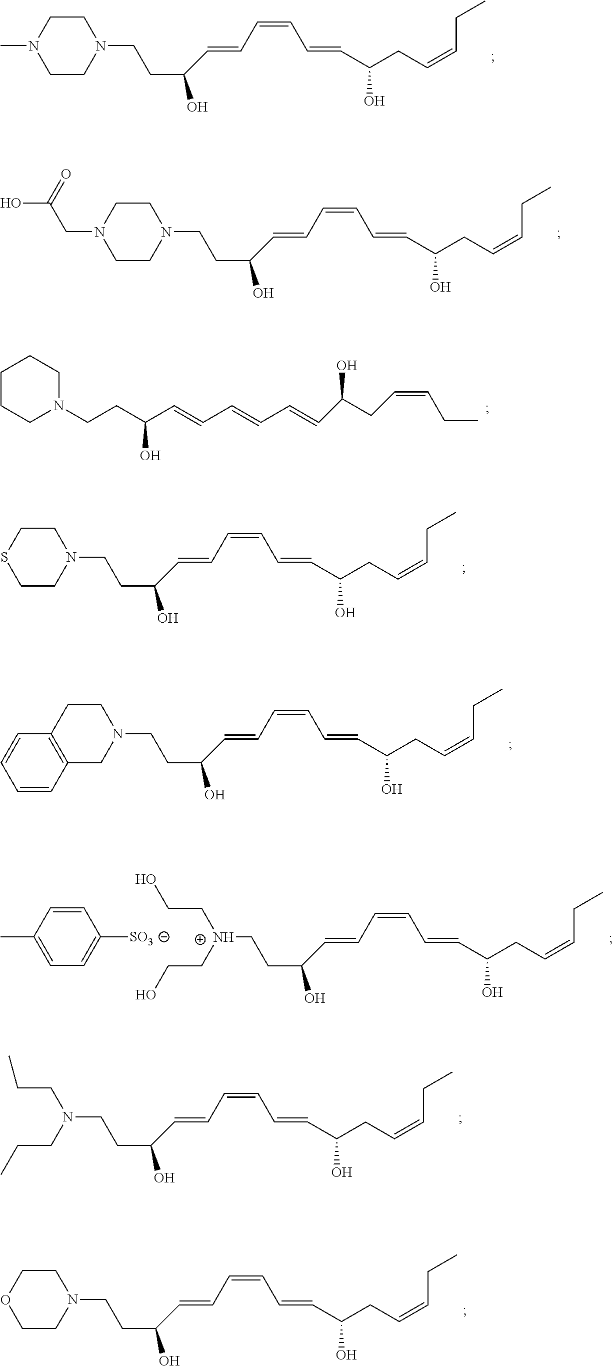

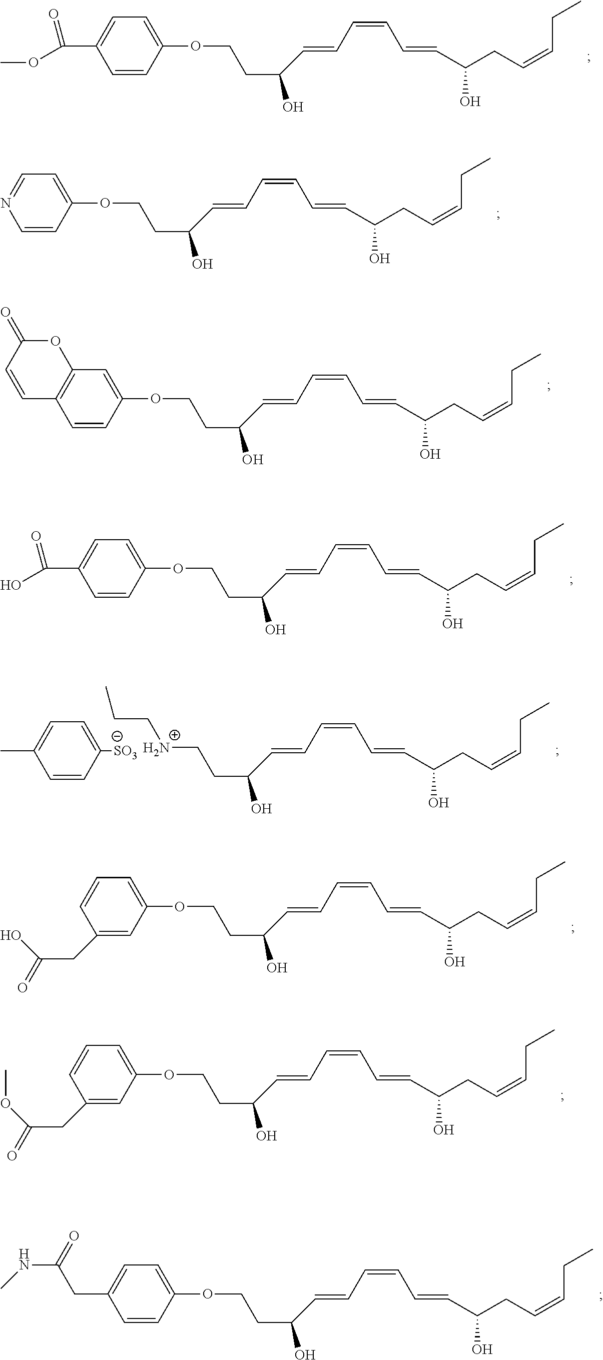

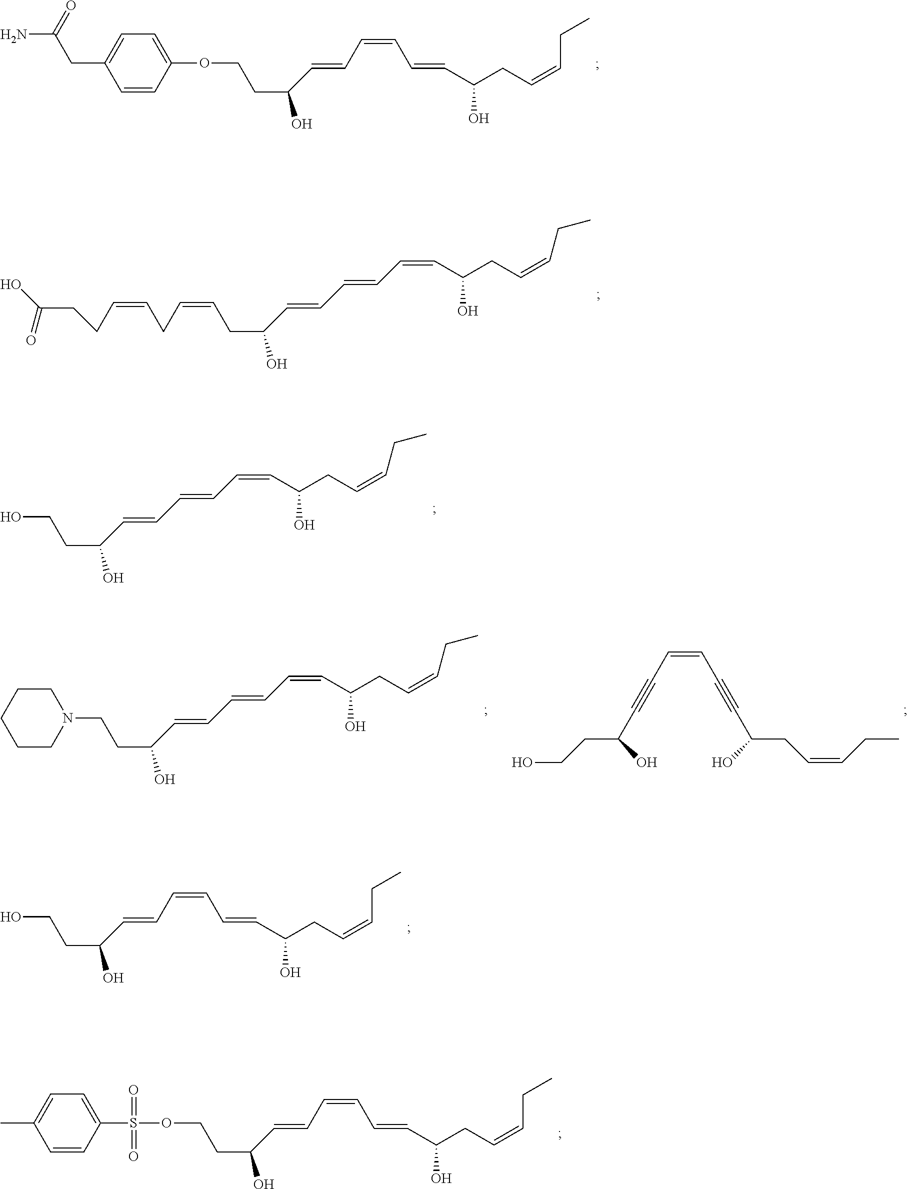

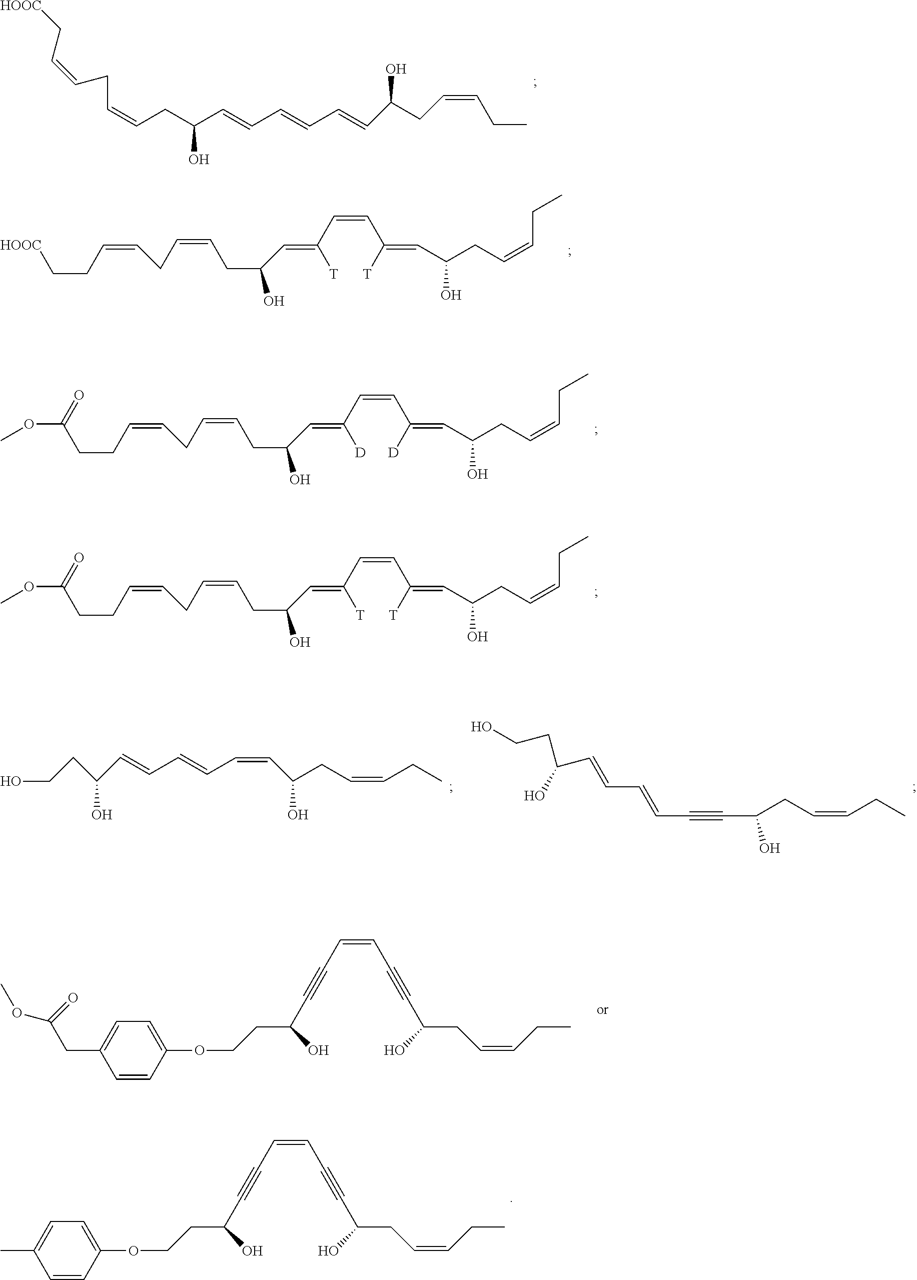

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “derivative” as used herein, is understood as being a substance which comprises the same basic carbon skeleton and carbon functionality in its structure as a given compound, but can also bear one or more substituents or rings.

The term “analog” as used herein, is understood as being a substance similar in structure to another compound but differing in some structural detail.

The term “structural isomer” as used herein refers to any of two or more chemical compounds, having the same molecular formula but different structural formulas.

The term “geometric isomer” or “stereoisomer” as used herein refers to two or more compounds which contain the same number and types of atoms, and bonds (i.e., the connectivity between atoms is the same), but which have different spatial arrangements of the atoms, for example cis and trans isomers of a double bond, enantiomers, and diastereomers.

In the context of the methods described herein, the term “treating” or “effective to treat” may refer to the amelioration or stabilization of one or more symptoms associated with the disease or disorder being treated. The term “treating” may also encompass the management of a disease or disorder, referring to the beneficial effects that a subject derives from a therapy which does not result in a cure of the underlying disease or disorder. The compositions of the present disclosure can also be used in the prevention of certain diseases, disorders, and conditions. In this context, the term “prevention” refers to preventing the recurrence, development, progression or onset of one or more symptoms of the disease, disorder, or condition.

The specialized pro-resolving mediators, structural isomers thereof, and/or structural analogs thereof or compositions comprising same can also be used in combination therapy. As used herein, “combination therapy” or “co-therapy” includes the administration of a therapeutically effective amount of one or more of the pro-resolving mediators, structural isomers thereof, and/or structural analogs thereof and an additional active agent.

The foregoing and other advantages and features of the present disclosure will become more apparent upon reading of the following non-restrictive detailed description of illustrative embodiments thereof, with reference to the accompanying drawings/figures. It should be understood, however, that the detailed description and the illustrative embodiments, while indicating specific embodiments of the disclosure, are given by way of illustration only, since various changes and modifications within the spirit and scope of the disclosure will become apparent to those skilled in the art from this description.

### BRIEF DESCRIPTION OF THE DRAWINGS/FIGURES

The following figures/drawings form part of the present specification and are included to further demonstrate certain aspects of the present specification. The present specification may be better understood by reference to one or more of these figures/drawings in combination with the detailed description. In the appended drawings/figures:

FIG. 1—Illustration of the structure of specialized pro-resolving lipid mediators (SPMs), including the E-series (RvEs) and D-series (RvDs) resolvins, protectins (PDs) and maresins (MaRs) (A); and the molecular structures of protectin D1 (PD1) and protectin DX (PDX) (B).

FIG. 2—Illustration of the effects of PDX analogs on baseline and insulin-induced glucose uptake in C2C12 myocytes in accordance with an embodiment of the present disclosure; glucose uptake without insulin stimulation (blue), and glucose uptake with insulin stimulation in C2C12 cells treated with analogs at 25 μM (orange) (n=6, mean±SEM); 2-deoxy-glucose uptake of C2C12 muscle cells treated for 2 hours with PDX or analogs (100 nM) in the basal and insulin stimulated state (100 nM, 45 minutes). #P<0.05 vs corresponding control (+insulin) condition.

FIG. 3—Illustration of the antiviral effect (SARS-CoV-2) of representative compounds in accordance with an embodiment of the present disclosure. #: p<0.01; *: p<0.05 vs control;!!: cytotoxicity observed. Antiviral effect against SARS-CoV-2 of PDX and/or structural PDX analogs in VERO E6 cells by plaque reduction assay (PRA).

FIG. 4—Illustration of the comparative inhibition of LPS-induced iNOS activity in macrophages by PDX analogue RM-598-54-2 versus quercetin. Data show nitrite production in LPS-induced J774 mice macrophages. Vehicle alone (e.g., ethanol or DMSO) also partially reduced iNOS. Nitrite production in culture media after inflammation induction by LPS. Results are means±SEM of 4-6 separate experiments. LPS-stimulated J774 macrophages (LPS at 2.5 ng/ml) treated with RM-598-54-2 or Quercetin at the indicated concentration for 18 h hours. Nitrite concentration was measured in the media by the Griess method.

FIG. 5—Illustration of the dose-dependent inhibition of LPS-induced iNOS activity in macrophages in accordance with an embodiment of the present disclosure. PDX analogs (RM-598-44; RM-598-54-2; RM-598-53 and RM-598-94) reduced nitrite production in LPS-induced J774 mice macrophages. Nitrite production in culture media after inflammation induction by LPS. Results are means±SEM of 4-6 separate experiments. LPS-stimulated J774 macrophages (2.5 ng/ml) treated with PDX or analogs at the indicated concentration for 18 h hours. Nitrite concentration was measured in the media by the Griess method.

FIG. 6—Illustration of the dose-dependent inhibition of LPS-induced iNOS activity in macrophages in accordance with an embodiment of the present disclosure. PDX analogs (RM-598-44; RM-598-53; and RM-598-54-2) reduced nitrite production in LPS-induced J774 mice macrophages. Nitrite production in culture media after inflammation induction by LPS. Results are means±SEM of 4 separate experiments. LPS-stimulated J774 macrophages (2.5 ng/ml) treated with PDX or analogs at the indicated concentration for 18 h hours. Nitrite concentration was measured in the media by the Griess method.

FIG. 7—Illustration of the dose-dependent inhibition of LPS-induced iNOS activity in macrophages in accordance with an embodiment of the present disclosure. PDX analogs (RM-598-48; RM-598-49; and Quercetin) reduced nitrite production in LPS-induced J774 mice macrophages. Nitrite production in culture media after inflammation induction by LPS. Results are means±SEM of 4 separate experiments. LPS-stimulated J774 macrophages (2.5 ng/ml) treated with PDX or analogs at the indicated concentration for 18 h hours. Nitrite concentration was measured in the media by the Griess method.

FIG. 8—Illustration of the effects of a single dose (25 μM) of several PDX analogs (Upper panel) and the dose-dependent inhibition of LPS-induced iNOS activity by RM-598-94 (lower panel) in macrophages in accordance with an embodiment of the present disclosure. LPS-stimulated J774 macrophages (2.5 ng/ml) were treated with analogs of PDX for 18 h hours. Nitrite concentration was measured in the media by the Griess method.

FIG. 9—Illustration of the dose-dependent inhibition of LPS-induced iNOS activity in macrophages in accordance with an embodiment of the present disclosure. PDX analogs (RM-598-132A; RM-598-132B; RM-598-132C; RM-598-133; RM-598-134; RM-598-135A; RM-598-135B; RM-598-137A; RM-598-137B) reduced nitrite production in LPS-induced J774 mice macrophages. Nitrite production in culture media after inflammation induction by LPS. Results are means±SEM of 4 separate experiments. LPS-stimulated J774 macrophages (2.5 ng/ml) treated with analogs of PDX at the indicated concentration for 18 h hours. Nitrite concentration was measured in the media by the Griess method.

FIG. 10—Illustration of the effects of PDX on 2-deoxyglucose uptake in skeletal muscle cells (C2C12 and L6). 2-Deoxy-glucose uptake of C2C12 and L6 muscle cells treated for 2 hours with PDX at the indicated concentrations in the basal and insulin stimulated state (100 nM, 45 minutes).

FIG. 11—Illustration of the effects of PDX analog RM-598-53 on 2-deoxyglucose uptake in skeletal muscle cells (C2C12 and L6). 2-Deoxy-glucose uptake of C2C12 and L6 muscle cells treated for 2 hours with analogue RM-598-53 at the indicated concentrations in the basal and insulin stimulated state (100 nM, 45 minutes).

FIG. 12—Illustration of the effects of PDX analog RM-598-54-2 on 2-deoxyglucose uptake in skeletal muscle cells (C2C12 and L6). 2-Deoxy-glucose uptake of C2C12 and L6 muscle cells treated for 2 hours with analogue RM-598-54-2 at the indicated concentrations in the basal and insulin stimulated state (100 nM, 45 minutes).

FIG. 13—Illustration of the effects of PDX analog RM-598-48 on 2-deoxyglucose uptake in skeletal muscle cells (C2C12 and L6). 2-Deoxy-glucose uptake of C2C12 and L6 muscle cells treated for 2 hours with analogue RM-598-48 at the indicated concentrations in the basal and insulin stimulated state (100 nM, 45 minutes).

FIG. 14—Illustration of the effects of PDX analog RM-598-44 on 2-deoxyglucose uptake in skeletal muscle cells (C2C12 and L6). 2-Deoxy-glucose uptake of C2C12 and L6 muscle cells treated for 2 hours with analogue RM-598-44 at the indicated concentrations in the basal and insulin stimulated state (100 nM, 45 minutes).

FIG. 15—Illustration of the effects of metformin on 2-deoxyglucose uptake in skeletal muscle cells (C2C12 and L6). 2-Deoxy-glucose uptake of C2C12 and L6 muscle cells treated for 2 hours with Metformin at the indicated concentrations in the basal and insulin stimulated state (100 nM, 45 minutes).

FIG. 16—Illustration of the effects of rosiglitazone on 2-deoxyglucose uptake in skeletal muscle cells (C2C12 and L6). 2-Deoxy-glucose uptake of C2C12 and L6 muscle cells treated for 2 hours with Rosiglitazone at the indicated concentrations in the basal and insulin stimulated state (100 nM, 45 minutes).

FIG. 17—Illustration of the enhanced antiviral effects of the combination of PDX-2 (RM-598-48; 40 μM) and Remdesivir on SARS-CoV-2, showing a higher potency of such a combination over Remdesivir when used alone in the VERO E6 plaque reduction assay.

FIG. 18—Illustration of the anti-inflammatory effect of selected PDX analogs (RM-598-44 and RM-598-48) on obese hamsters stimulated with LPS. The hamsters were given an intraperitoneal (IP) injection of 0.6 mg/kg LPS followed by an IP injection of either Control (vehicle 92:8 propylene glycol: DMSO) or PDX analogue (50 mg/kg RM-598-44 or RM-598-48 in vehicle). Plasma TNF-alpha was measured by ELISA. Data=mean±SEM of 4 female hamsters. One-way ANOVA with Dunnett's multiple comparison correction *** p<0.001. **** p<0.0001.

FIG. 19—Illustration of the anti-inflammatory effect of selected PDX analogs (RM-598-140, RM-598-141, RM-598-188, and RM-598-190). LPS-stimulated J774 macrophages (5 ng/mL) were treated with analogues at the indicated concentrations for 18 hours. Nitrite concentrations were subsequently measured in the media by the Griess method. Data are mean±SEM for 8 independent experiments performed in triplicate. #p<0.05 versus LPS alone.

FIG. 20—Illustration of the effect of selected PDX analogs (RM-598-140, RM-598-141, RM-598-188, and RM-598-190) on 2-deoxyglucose uptake in C2C12 skeletal muscle cells. 2-Deoxy-glucose uptake of C2C12 muscle cells treated for 2 hours with analogues RM-598-140, RM-598-141, RM-598-188, and RM-598-190 at the indicated concentrations in the basal and insulin stimulated state (100 nM, 45 minutes). Data are mean±SEM for 5-8 independent experiments. *p<0.05 vs basal values, #p<0.05 vs insulin values.

FIG. 21—Illustration of the antiviral effect of the PDX analogue RM-598-137B (50 μM), PDX (50 μM), PD1 (50 M), and Baloxavir acid (BXA) (5 μM) on the influenza (H1N1 A/PR/8/34) viral charge in infected human lung epithelial cells (A549). A decrease in the viral charge of 39% (P=0.0058) could be observed for RM-598-137B; a decrease in the viral charge of 10% (P=0.038) could be observed for PD1; and a decrease in the viral charge of 88% (P=<0.0001) could be observed for BXA.

FIG. 22—Illustration of the antiviral effect of the PDX analogue RM-598-48 (50 mg/kg) on SARS-Cov-2 infected hamsters (viral titers in nasal turbinates (NT) and lung viral titers). Hamsters were infected with 4.5 log PFU of original SARS-CoV-2 and treated on days 1, 2 and 3 post-infection. Viral titers were determined on day 4 post-infection in nasal turbinates and lungs. The viral loads were reported in TCID₅₀ per gram of organ. *P<0.05 compared to infected none-treated hamsters, based on t-test (A). The decrease of the viral loads in nasal turbinates induced by RM-598-48 (50 mg/kg) was found to be significantly different from the untreated hamsters. Viral loads remained unchanged for the combination of RM-598-48 (50 mg/kg) with Remdesivir (15 mg/kg).

FIG. 23—Illustration of the plasma concentration of the PDX analogue RM-598-48 after intraperitoneal (IP) injection in a hamster at various concentrations (5 mg/kg; 25 mg/kg; and 50 mg/kg) over time; after 1 and 2 hours respectively (A), and over the course of 24 hours (B).

FIG. 24—Illustration of the plasma concentration of the PDX analogue RM-598-43 (metabolite) after intraperitoneal (IP) injection of the PDX analogue RM-598-48 in a hamster at various concentrations (5 mg/kg; 25 mg/kg; and 50 mg/kg) over time; after 1 and 2 hours respectively (A), and over the course of 24 hours (B).

FIG. 25—Illustration of the plasma concentration of the PDX analogue RM-598-44 after intraperitoneal (IP) injection in a hamster at various concentrations (5 mg/kg; 25 mg/kg; and 50 mg/kg) over time; after 1 and 2 hours respectively.

FIG. 26—Illustration of the weight variation (%) of hamsters following viral infection with SARS-CoV-2. Hamsters were infected with 4.5 log PFU of original SARS-CoV-2 and treated on days 1, 2 and 3 post-infection with Remdesivir (A) or PDX analog RM-598-48 (B). Weight variation was monitored on a daily basis and reported as % of initial weight. ***P<0.001 for Remdesivir-treated hamsters compared to infected and vehicle-treated hamsters. *P<0.05 (days 3, 9, 11, 12 and 14) and **P<0.01 (days 10 and 12) for PDX analogue RM-598-48 treated hamsters compared to infected and vehicle-treated hamsters. RM-598-48 was able to significantly limit weight loss of treated hamsters at days 3, 9, 11, 12 and 14 following infection by SARS-CoV-2; Remdesivir significantly limits weight loss from days 3 to 15.

FIG. 27—Illustration of the weight variation (%) of hamsters following viral infection with SARS-CoV-2. Hamsters were infected with 4.5 log PFU of original SARS-CoV-2 and treated from day 1 until day 5 post-infection with PDX analogs RM-598-44 (A) and RM-598-48 (B). Weight variation was monitored on a daily basis and reported as % of initial weight. *P<0.05 for PDX analog RM-598-48 treated hamsters compared to infected and vehicle-treated hamsters at days 7, 8 and 9, and based on ANOVA. RM-598-48 was able to significantly limit weight loss of treated hamsters following infection by SARS-CoV-2. RM-598-44 shows a tendency to limit weight loss, but not in a significant manner.

FIG. 28—Illustration of an animal study protocol. 5-6 Week-old Golden Syrian hamsters were infected with 4.5 log PFU of original SARS-CoV-2 strain. On days 1, 2 and 3, or 1 to 5 post-infection, the animals were treated intraperitoneally with either Remdesivir (15 mg/kg), analog RM-598-48 (50 mg/kg), Remdesivir (15 mg/kg)+analog RM-598-48 (50 mg/kg), analog RM-598-44 (50 mg/kg), or with vehicle only (FIG. 28). The hamsters were monitored daily over a period of 14 days for clinical symptoms and weight loss. On day 4 post-infection, nasal turbinates and lungs were harvested to assess viral replication.

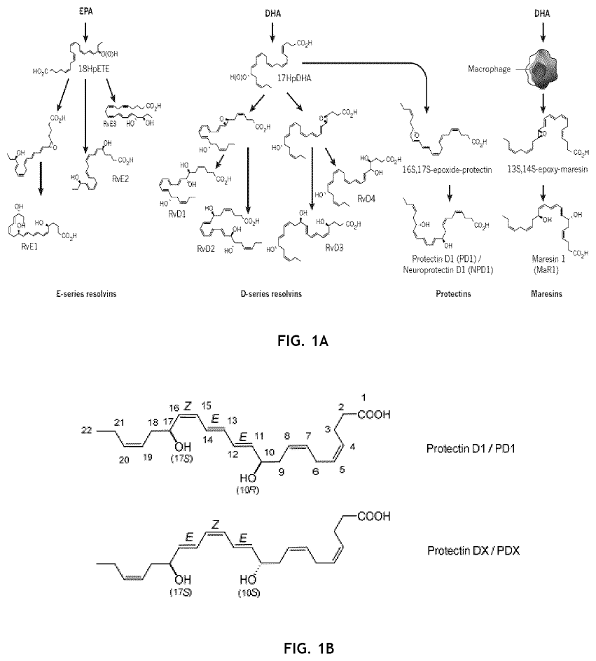

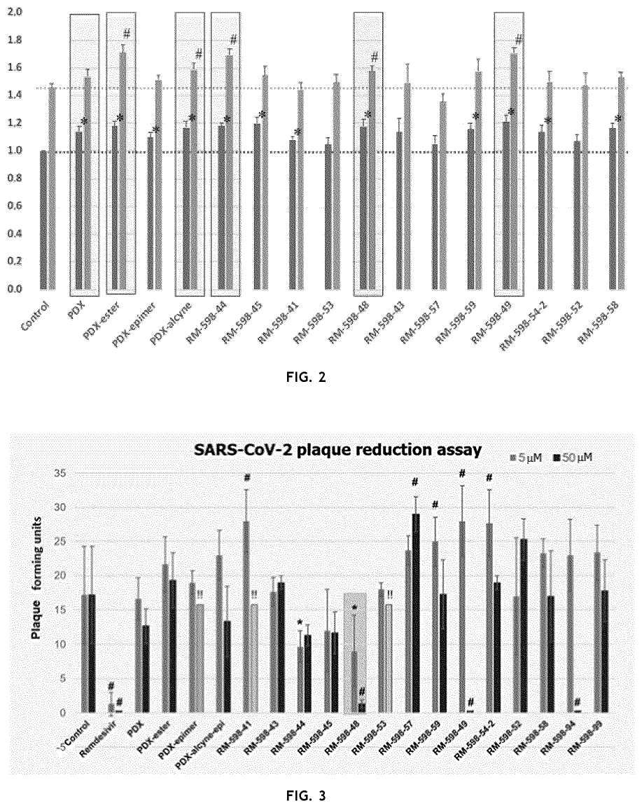

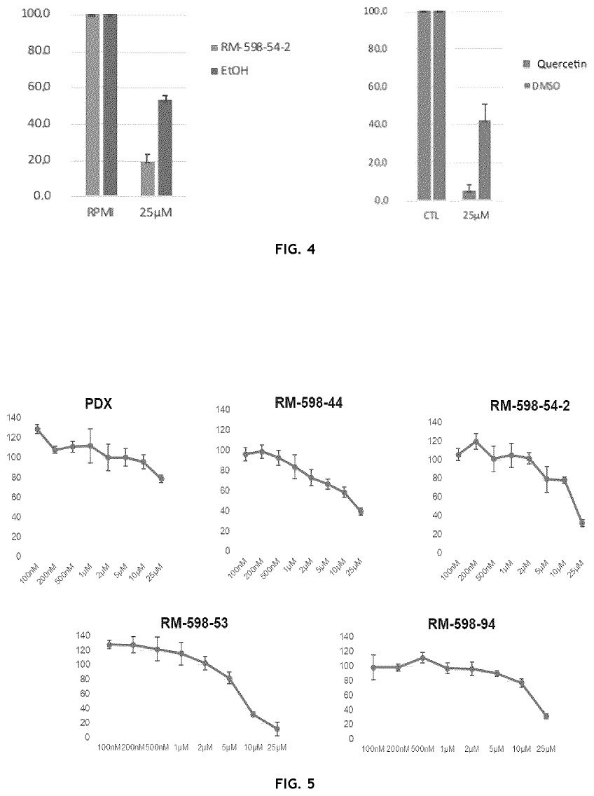

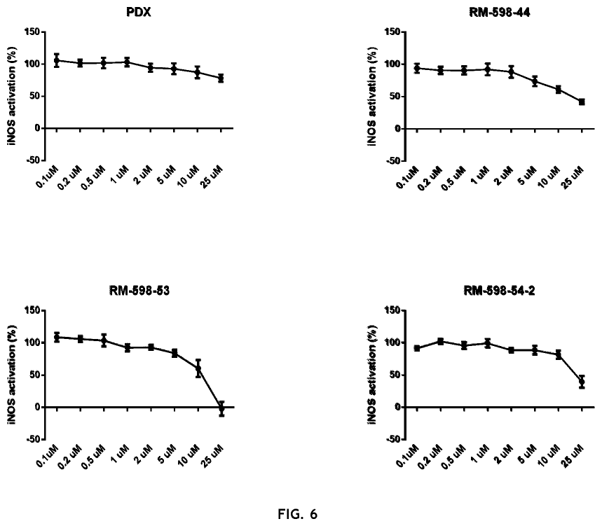

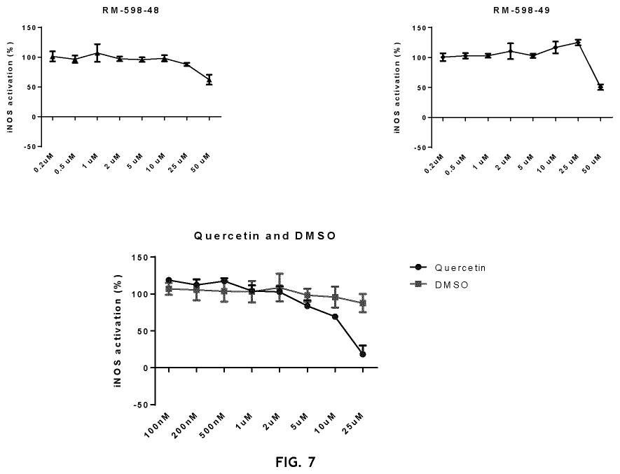

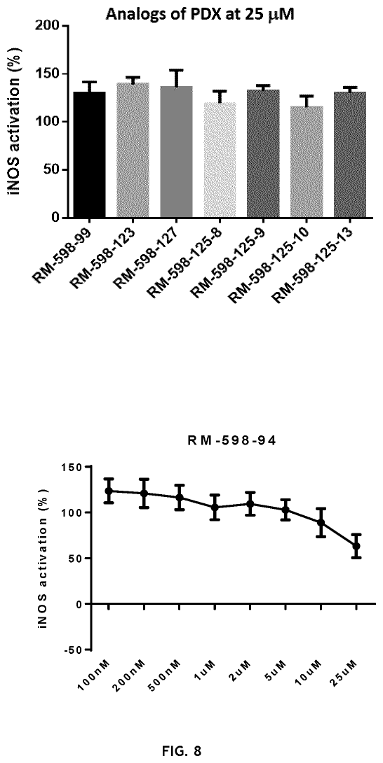

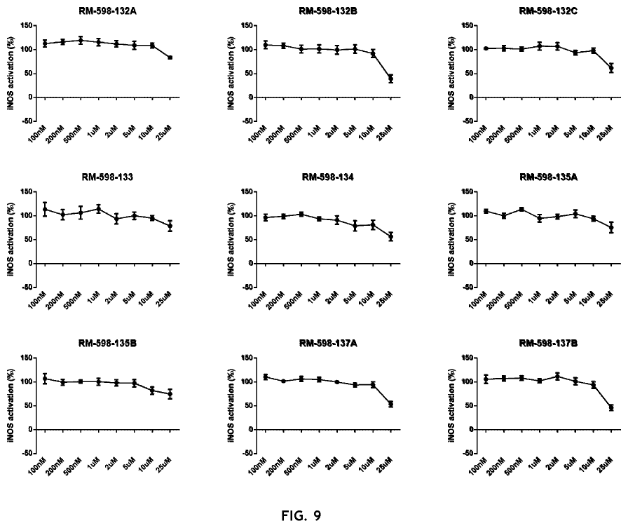

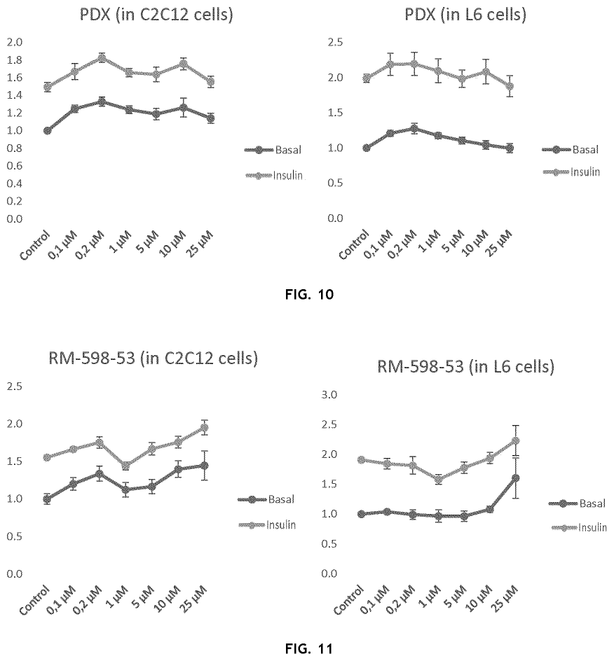

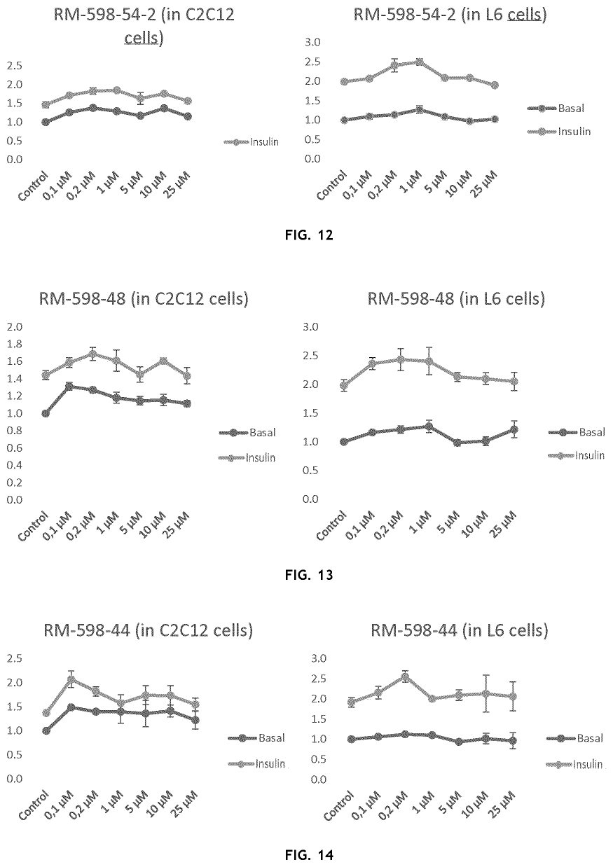

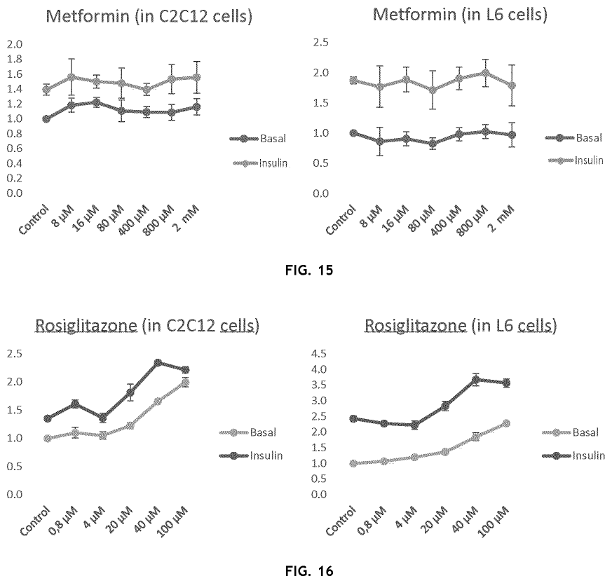

### DETAILED DESCRIPTION OF ILLUSTRATIVE EMBODIMENTS

The present disclosure relates to methods of preventing, delaying the onset or reducing the severity of, preventing or reversing the progression of, or treating a viral infection or disease in a subject using a specialized pro-resolving mediator, structural isomer thereof, and/or structural analog thereof. In another aspect, the present disclosure relates to methods of preventing, delaying the onset or reducing the severity of, preventing or reversing the progression of, or treating insulin resistance or diabetes in a subject using a specialized pro-resolving mediator, structural isomer thereof, and/or structural analog thereof. In yet another aspect, the present disclosure relates to methods of lowering blood glucose levels in a subject, using a specialized pro-resolving mediator, structural isomer thereof, and/or structural analog thereof. In yet another aspect, the present disclosure relates to methods of preventing, delaying the onset or reducing the severity of, preventing or reversing the progression of, or treating an inflammatory disease, disorder, condition, or symptom in a subject using a specialized pro-resolving mediator, structural isomer thereof, and/or structural analog thereof. In yet another aspect, the present disclosure relates to methods of preventing, delaying the onset or reducing the severity of, preventing or reversing the progression of, or treating an inflammatory disease, disorder, condition, or symptom that is secondary to another condition in a subject using a specialized pro-resolving mediator, structural isomer thereof, and/or structural analog thereof. These and other aspects of the disclosure are described in greater detail below.

The present disclosure also relates to methods of preventing, delaying the onset or reducing the severity of, preventing or reversing the progression of, or treating a viral infection or disease in a subject using a specialized pro-resolving mediator, structural isomer thereof, and/or structural analog thereof and a second agent. In another aspect, the present disclosure relates to methods of preventing, delaying the onset or reducing the severity of, preventing or reversing the progression of, or treating insulin resistance or diabetes in a subject using a specialized pro-resolving mediator, structural isomer thereof, and/or structural analog thereof and a second agent. In yet another aspect, the present disclosure relates to methods of lowering blood glucose levels in a subject, using a specialized pro-resolving mediator, structural isomer thereof, and/or structural analog thereof and a second agent. In yet another aspect, the present disclosure relates to methods of preventing, delaying the onset or reducing the severity of, preventing or reversing the progression of, or treating an inflammatory disease, disorder, condition, or symptom in a subject using a specialized pro-resolving mediator, structural isomer thereof, and/or structural analog thereof and a second agent. In yet another aspect, the present disclosure relates to methods of preventing, delaying the onset or reducing the severity of, preventing or reversing the progression of, or treating an inflammatory disease, disorder, condition, or symptom that is secondary to another condition in a subject using a specialized pro-resolving mediator, structural isomer thereof, and/or structural analog thereof and a second agent. These and other aspects of the disclosure are described in greater detail below.

The present disclosure also relates to methods of preventing, delaying the onset or reducing the severity of, preventing or reversing the progression of, or treating chronic low-level inflammation in a subject using a specialized pro-resolving mediator, structural isomer thereof, and/or structural analog thereof. Situations of improperly regulated inflammation may result in chronic low-level inflammation. For example, the initiation phase can be too strong, or the resolution phase may be too weak. Left untreated, this situation will result in chronic low-grade inflammation that can lead to a number of chronic conditions including obesity, metabolic syndrome, diabetes, cardiovascular disease, cancer, auto-immune disorders, and neurological disorders. Conditions that can be improved by reducing chronic low-level inflammation comprise neurological disorders such as brain trauma (mild and severe), depression, attention deficit hyperactivity disorder (ADHD), attention deficit disorder (ADD), Parkinson's disease, Alzheimer's disease and neuropathy; ocular disorders such as age-related macular degeneration (AMD), dry eye syndrome, optic nerve damage and diabetic retinopathy; auto-immune disorders such as type 1 diabetes, rheumatoid arthritis, multiple sclerosis, lupus, Sjogren's syndrome, cancer, heart disease and osteoporosis; metabolic disorders such as type 2 diabetes, metabolic syndrome, non-alcoholic steatohepatitis (NASH), obesity, asthma and allergies; gastrointestinal disorders such as colitis, leaky gut syndrome and Crohn's disease; endometriosis and polycystic ovarian syndrome; emphysema, chronic bronchitis and chronic back pain; and skin disorders such as psoriasis and inflammation caused by intense exercise.

The present disclosure also relates to methods of preventing, delaying the onset or reducing the severity of, preventing or reversing the progression of, or treating acute inflammation.

PDX, PD1, and analogs thereof may contain two or more asymmetrically-substituted carbon atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures, and individual diastereomers. In some embodiments, a single diastereomer is obtained. The chiral centers of the compounds of the present disclosure can have the S- or the R-configuration.

PDX, PD1, and analogs thereof of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure. Prodrugs of PDX, PD1, and analogs thereof employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of PDX, PD1, and analogs thereof provided herein are within the scope of the disclosure. It will also be appreciated by those skilled in organic chemistry that many organic compounds can exist in more than one crystalline form. For example, crystalline forms may vary from solvate to solvate. Thus, all crystalline forms of PDX, PD1, and analogs thereof or the pharmaceutically acceptable solvates thereof are within the scope of the present disclosure.

In aspect, the present disclosure relates to pharmaceutical compositions comprising a therapeutically effective amount of one or more of the specialized pro-resolving mediators, structural isomers thereof, and/or structural analogs thereof, or pharmaceutically acceptable salts, or solvates thereof, as disclosed herein, and at least one pharmaceutically acceptable excipient, non-limiting examples of which are carriers and diluents. The term “therapeutically effective amount” is understood as being an amount of pro-resolving mediator, structural isomer thereof, and/or structural analog thereof, or a pharmaceutically acceptable salt, or solvate thereof, as disclosed herein, required upon administration to a patient in order to treat a condition such as a viral infection, insulin resistance or diabetes, inflammation or for lowering blood glucose levels in the patient. Therapeutic methods comprise the step of treating patients in a pharmaceutically acceptable manner with the specialized pro-resolving mediators, structural isomers thereof, and/or structural analogs thereof, or pharmaceutically acceptable salts, or solvates thereof, as disclosed herein, or with compositions comprising such specialized pro-resolving mediators, structural isomers thereof, and/or structural analogs thereof, or pharmaceutically acceptable salts, or solvates thereof. Such compositions may be in the form of tablets, coated tablets, capsules, caplets, powders, granules, lozenges, suppositories, reconstitutable powders, syrups, liquid preparations such as oral or sterile parenteral solutions or suspensions, as well as injectable formulations and transdermal formulations.

The specialized pro-resolving mediators, structural isomers thereof, and/or structural analogs thereof, or pharmaceutically acceptable salts, or solvates thereof, as disclosed herein, may be administered alone or in combination with pharmaceutically acceptable carriers. The proportion of each carrier is determined by the solubility and chemical nature of the specialized pro-resolving mediator, structural isomer thereof, and/or structural analog thereof, or pharmaceutically acceptable salt, or solvate thereof, the route of administration, and standard pharmaceutical practice. In order to ensure consistency of administration, in an embodiment of the present disclosure, the pharmaceutical composition is in the form of a unit dose. The unit dose presentation forms for oral administration may be tablets, coated tablets and capsules and may contain conventional excipients. Non-limiting examples of conventional excipients include binding agents such as acacia, gelatin, sorbitol, or polyvinylpyrrolidone; fillers such as lactose, dextrose, saccharose, sugar, maize-starch, calcium phosphate, sorbitol or glycine; tableting lubricants such as talc, stearic acid, calcium or magnesium stearate, polyethylene glycols, gums or gels; disintegrants such as starch, polyvinylpyrrolidone, sodium starch glycollate or microcrystalline cellulose; or pharmaceutically acceptable wetting agents such as sodium lauryl sulfate.

The specialized pro-resolving mediators, structural isomers thereof, and/or structural analogs thereof, or pharmaceutically acceptable salts, or solvates thereof, as disclosed herein, may be injected parenterally; this being intramuscularly, intravenously, subcutaneously, or intraperitoneally. For parenteral administration, the specialized pro-resolving mediators, structural isomers thereof, and/or structural analogs thereof, or pharmaceutically acceptable salts, or solvates thereof, as disclosed herein, may be used in the form of sterile solutions containing solutes for example, sufficient saline or glucose to make the solution isotonic.

Further suitable routes of administration of the specialized pro-resolving mediators, structural isomers thereof, and/or structural analogs thereof, or pharmaceutically acceptable salts, or solvates thereof, as disclosed herein include intranasal administration and ophthalmic administration. For intranasal administration, the specialized pro-resolving mediators, structural isomers thereof, and/or structural analogs thereof, or pharmaceutically acceptable salts, or solvates thereof, as disclosed herein may be formulated as an aerosol spray.

The specialized pro-resolving mediators, structural isomers thereof, and/or structural analogs thereof, or pharmaceutically acceptable salts, or solvates thereof, as disclosed herein, may also be administered topically such as via transdermal routes using dermal or skin patches.

The specialized pro-resolving mediators, structural isomers thereof, and/or structural analogs thereof, or pharmaceutically acceptable salts, or solvates thereof, as disclosed herein, may be administered orally in the form of tablets, coated tablets, capsules, or granules, containing suitable excipients non-limiting examples of which are starch, lactose, white sugar, and the like. The specialized pro-resolving mediators, structural isomers thereof, and/or structural analogs thereof, or pharmaceutically acceptable salts, or solvates thereof, as disclosed herein, may be administered orally in the form of solutions which may contain coloring and/or flavoring agents. The specialized pro-resolving mediators, structural isomers thereof, and/or structural analogs thereof, or pharmaceutically acceptable salts, or solvates thereof, as disclosed herein, may also be administered sublingually in the form of tracheas or lozenges in which the active ingredient(s) is/are mixed with sugar or corn syrups, flavoring agents and/or dyes, and then dehydrated sufficiently to make the mixture suitable for pressing into solid form.

The solid oral compositions may be prepared by conventional methods of blending, granulation, compression, coating, filling, tableting, or the like. Repeated blending operations may be used to distribute the active agent throughout those compositions employing large quantities of fillers. Such operations are, of course, conventional in the art. The tablets may be coated according to methods well known in normal pharmaceutical practice, in particular with an enteric coating.

Oral liquid preparations may be in the form of emulsions, suspensions, syrups, or elixirs, or may be presented as a dry product for reconstitution with water or other suitable vehicle before use. Such liquid preparations may or may not contain conventional additives. Non limiting examples of conventional additives include suspending agents such as sorbitol, cyclodextrins, syrup, natural gums, agar, methyl cellulose, gelatin, pectin, sodium alginate, hydroxyethylcellulose, carboxymethylcellulose, aluminum stearate gel, or hydrogenated edible fats; emulsifying agents such as sorbitan monooleate or acaci; non-aqueous vehicles (which may include edible oils) such as almond oil, fractionated coconut oil, oily esters selected from the group consisting of glycerin, propylene glycol, ethylene glycol, and ethyl alcohol; preservatives such as for instance methyl para-hydroxybenzoate, ethyl para-hydroxybenzoate, n-propyl parahydroxybenzoate, n-butyl parahydroxybenzoate, or sorbic acid; and, if desired conventional flavoring such as saccharose, glycerol, mannitol, sorbitol, or coloring agents.

For parenteral administration, fluid unit dosage forms may be prepared by utilizing the specialized pro-resolving mediators, structural isomers thereof, and/or structural analogs thereof, or pharmaceutically acceptable salts, or solvates thereof, as disclosed herein, and a sterile vehicle (i.e., sterile water) and, depending on the concentration employed, the specialized pro-resolving mediators, structural isomers thereof, and/or structural analogs thereof, or pharmaceutically acceptable salts, or solvates thereof, may be either suspended or dissolved in the vehicle. Other suitable vehicles may include olive oil, ethyl oleate, and glycols. If needed, a suitable quantity of lidocaine hydrochloride may also be included. Once in solution, the specialized pro-resolving mediators, structural isomers thereof, and/or structural analogs thereof, or pharmaceutically acceptable salts, or solvates thereof, may be injected and filter sterilized before filling a suitable vial or ampoule followed by subsequently sealing the carrier or storage package. Adjuvants, such as a local anesthetic, a preservative, or a buffering agent, may be dissolved in the vehicle prior to use. Stability of the pharmaceutical composition may be enhanced by freezing the composition after filling the vial and removing the water under vacuum, (e.g., freeze drying).

Parenteral suspensions may be prepared in substantially the same manner, except that the specialized pro-resolving mediators, structural isomers thereof, and/or structural analogs thereof, or pharmaceutically acceptable salts, or solvates thereof, should be suspended in the vehicle rather than being dissolved, and, further, sterilization is not achievable by filtration. The specialized pro-resolving mediators, structural isomers thereof, and/or structural analogs thereof, or pharmaceutically acceptable salts, or solvates thereof, may be sterilized, however, by exposing it to ethylene oxide before suspending it in the sterile vehicle. A surfactant or wetting solution may be advantageously included in the composition to facilitate uniform distribution of the specialized pro-resolving mediators, structural isomers thereof, and/or structural analogs thereof, or pharmaceutically acceptable salts, or solvates thereof.

The specialized pro-resolving mediators, structural isomers thereof, and/or structural analogs thereof, or pharmaceutically acceptable salts, or solvates thereof, may be administered in the form of suppositories. Suppositories may contain pharmaceutically acceptable vehicles such as cocoa butter, polyethylene glycol, sorbitan, esters of fatty acids, lecithin, and the like.

The pharmaceutical compositions of the present disclosure comprise a pharmaceutically effective amount of at least one specialized pro-resolving mediator, structural isomer thereof, and/or structural analog thereof, or a pharmaceutically acceptable salt, or solvate thereof, as disclosed herein, and one or more pharmaceutically acceptable carriers, excipients, or diluents. In an embodiment of the present disclosure, the pharmaceutical compositions contain from about 0.1% to about 99% by weight of a specialized pro-resolving mediator, structural isomer thereof, and/or structural analog thereof, or a pharmaceutically acceptable salt, or solvate thereof. In an embodiment of the present disclosure, the pharmaceutical compositions contain from about 10% to about 60% by weight of a specialized pro-resolving mediator, structural isomer thereof, and/or structural analog thereof, or a pharmaceutically acceptable salt, or solvate thereof, depending on which method of administration is employed.

Physicians will determine the most-suitable dosage of the present therapeutic agents (the specialized pro-resolving mediator, structural isomer thereof, and/or structural analog thereof, or a pharmaceutically acceptable salt, or solvate thereof). Dosages may vary with the mode of administration and the particular specialized pro-resolving mediator, structural isomer thereof, and/or structural analog thereof, or a pharmaceutically acceptable salt, or solvate thereof chosen. In addition, the dosage may vary with the particular patient under treatment. The dosage of the specialized pro-resolving mediator, structural isomer thereof, and/or structural analog thereof, or a pharmaceutically acceptable salt, or solvate thereof used in the treatment may vary, depending on the relative efficacy of the compound and the judgment of the treating physician.

In an embodiment of the present disclosure the pharmaceutical compositions comprise a therapeutically effective amount of one or more of the specialized pro-resolving mediators, structural isomers thereof, and/or structural analogs thereof, or a pharmaceutically acceptable salt, or solvate thereof, and at least one pharmaceutically acceptable excipient, non-limiting examples of which are carriers and diluents.

### EXAMPLES

The following examples are included to demonstrate preferred embodiments of the disclosure. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Selected Examples of Specialized Pro-Resolving Mediators, Structural Isomers, and/or Structural Analogs Thereof

Selected structures of specialized pro-resolving mediators, structural isomers, and/or structural analogs thereof, in accordance with an embodiment of the present disclosure, are illustrated in Table 1.

TABLE 1
Selected structures of specialized pro-resolving mediators, structural
isomers, and/or structural analogs thereof.

| | |
|---|---|
| 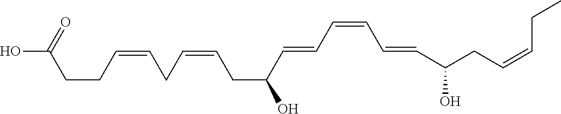 [SMILES: CC/C=C\C[C@H](O)/C=C/C=C\C=C\[C@@H](O)C/C=C\C/C=C\CCC(=O)O] | PDX |
| 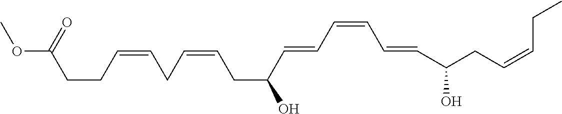 [SMILES: CC/C=C\C[C@H](O)/C=C/C=C\C=C\[C@@H](O)C/C=C\C/C=C\CCC(=O)OC] | PDX-Ester |
| 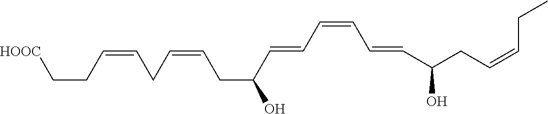 [SMILES: CC/C=C\C[C@@H](O)/C=C/C=C\C=C\[C@@H](O)C/C=C\C/C=C\CCC(=O)O] | PDX-Epimer |
| 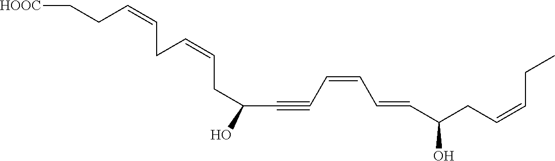 [SMILES: CC/C=C\C[C@@H](O)/C=C/C=C\C#C[C@@H](O)C/C=C\C/C=C\CCC(=O)O] | PDX-Epimer-Alkyne |
| 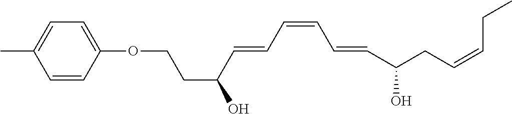 [SMILES: CC/C=C\C[C@H](O)/C=C/C=C\C=C\[C@@H](O)CCOc1ccc(C)cc1] | PDX-1 [RM-598-53] |
| 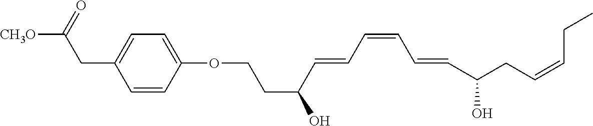 | PDX-2 [RM-598-48] |
| 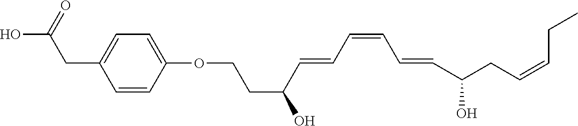 [SMILES: CC/C=C\C[C@H](O)/C=C/C=C\C=C\[C@@H](O)CCOc1ccc(CC(=O)O)cc1] | PDX-3 [RM-598-43] |
| 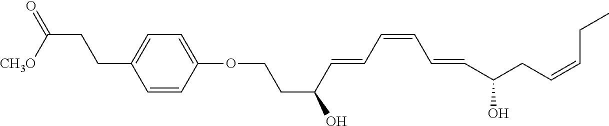 | PDX-4 [RM-598-57] |
| 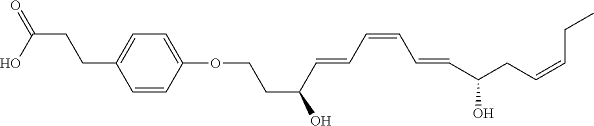 [SMILES: CC/C=C\C[C@H](O)/C=C/C=C\C=C\[C@@H](O)CCOc1ccc(CCC(=O)O)cc1] | PDX-5 [RM-598-59] |
| 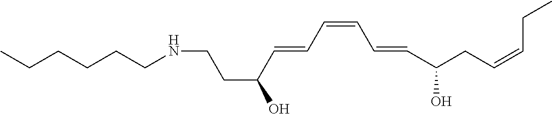 [SMILES: CC/C=C\C[C@H](O)/C=C/C=C\C=C\[C@@H](O)CCNCCCCCC] | PDX-6 [RM-598-49] |
| 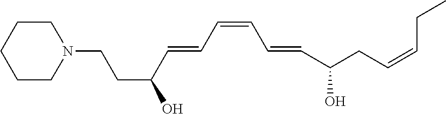 [SMILES: CC/C=C\C[C@H](O)/C=C/C=C\C=C\[C@@H](O)CCN1CCCCC1] | PDX-7 [RM-598-54-2] |
| 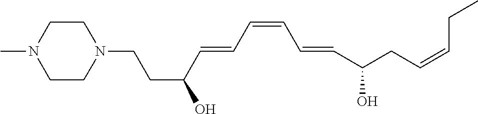 [SMILES: CC/C=C\C[C@H](O)/C=C/C=C\C=C\[C@@H](O)CCN1CCN(C)CC1] | PDX-8 [RM-598-52] |
| 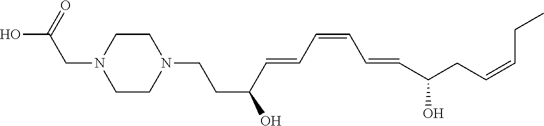 [SMILES: CC/C=C\C[C@H](O)/C=C/C=C\C=C\[C@@H](O)CCN1CCN(CC(=O)O)CC1] | PDX-9 [RM-598-58] |
| 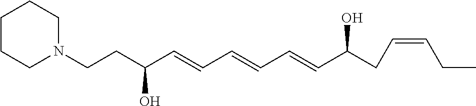 [SMILES: CC/C=C\C[C@H](O)/C=C/C=C/C=C/[C@@H](O)CCN1CCCCC1] | PDX-10 [RM-598-94] |
| 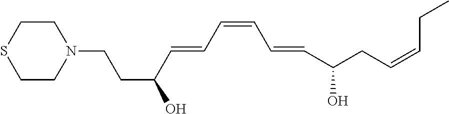 [SMILES: CC/C=C\C[C@H](O)/C=C/C=C\C=C\[C@@H](O)CCN1CCSCC1] | PDX-11 [RM-598-125-8] |
| 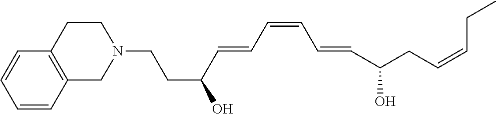 [SMILES: CC/C=C\C[C@H](O)/C=C/C=C\C=C\[C@@H](O)CCN1CCc2ccccc2C1] | PDX-12 [RM-598-125-9] |
| 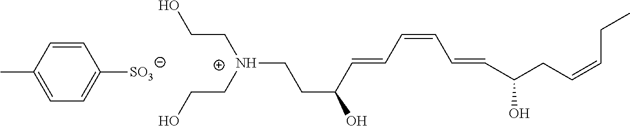 | PDX-13 [RM-598-125-10] |
| 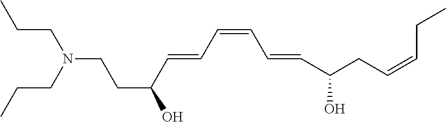 [SMILES: CC/C=C\C[C@H](O)/C=C/C=C\C=C\[C@@H](O)CCN(CCC)CCC] | PDX-14 [RM-598-125-13] |
| 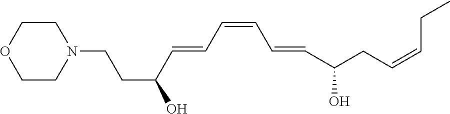 [SMILES: CC/C=C\C[C@H](O)/C=C/C=C\C=C\[C@@H](O)CCN1CCOCC1] | PDX-15 [RM-598-123] |
| 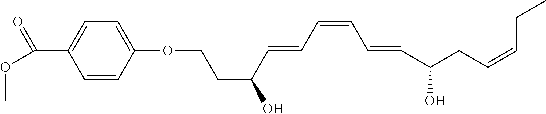 [SMILES: CC/C=C\C[C@H](O)/C=C/C=C\C=C\[C@@H](O)CCOc1ccc(C(=O)OC)cc1] | PDX-16 [RM-598-132-A] |
| 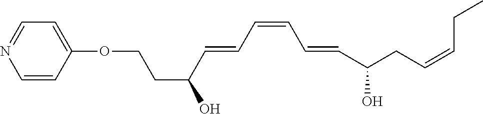 [SMILES: CC/C=C\C[C@H](O)/C=C/C=C\C=C\[C@@H](O)CCOc1ccncc1] | PDX-17 [RM-598-132-B] |
| 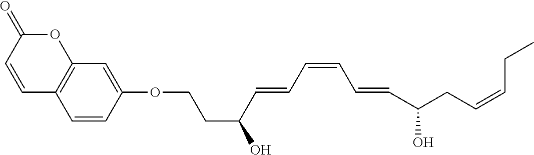 [SMILES: CC/C=C\C[C@H](O)/C=C/C=C\C=C\[C@@H](O)CCOc1ccc2ccc(=O)oc2c1] | PDX-18 [RM-598-132-C] |
| 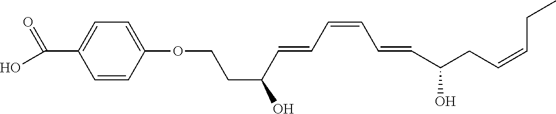 [SMILES: CC/C=C\C[C@H](O)/C=C/C=C\C=C\[C@@H](O)CCOc1ccc(C(=O)O)cc1] | PDX-19 [RM-598-133] |
| 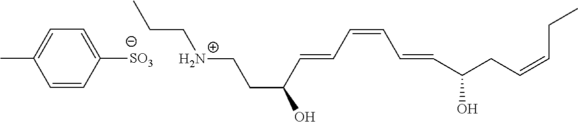 | PDX-20 [RM-598-134] |
| 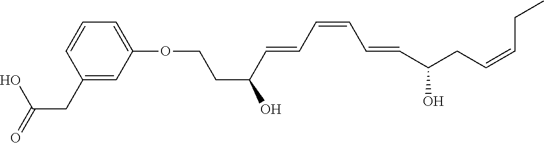 [SMILES: CC/C=C\C[C@H](O)/C=C/C=C\C=C\[C@@H](O)CCOc1cccc(CC(=O)O)c1] | PDX-21 [RM-598-135A] |
| 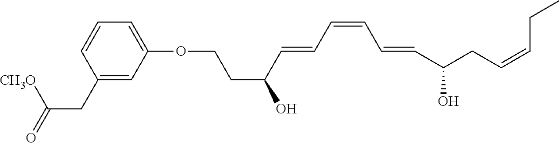 | PDX-22 [RM-598-135B] |
| 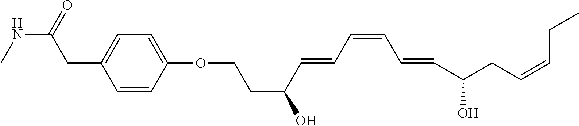 [SMILES: CC/C=C\C[C@H](O)/C=C/C=C\C=C\[C@@H](O)CCOc1ccc(CC(=O)NC)cc1] | PDX-23 [RM-598-137A] |
| 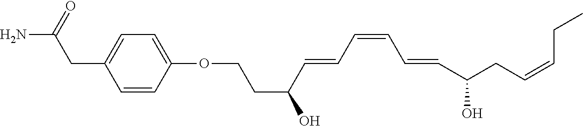 [SMILES: CC/C=C\C[C@H](O)/C=C/C=C\C=C\[C@@H](O)CCOc1ccc(CC(N)=O)cc1] | PDX-24 [RM-598-137B] |
| 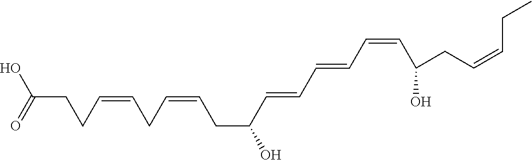 [SMILES: CC/C=C\C[C@H](O)\C=C/C=C/C=C/[C@H](O)C/C=C\C/C=C\CCC(=O)O] | PD1 |
| 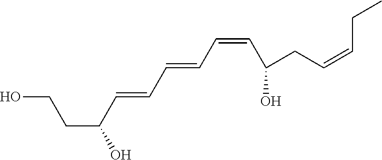 [SMILES: CC/C=C\C[C@H](O)\C=C/C=C/C=C/[C@H](O)CCO] | PD1-2 [RM-598-140] |
| 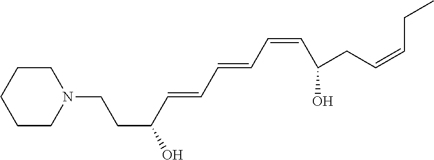 [SMILES: CC/C=C\C[C@H](O)\C=C/C=C/C=C/[C@H](O)CCN1CCCCC1] | PD1-1 [RM-598-127] |
| 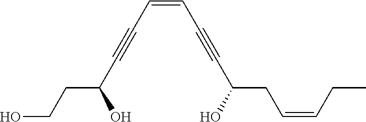 [SMILES: CC/C=C\C[C@H](O)C#C/C=C\C#C[C@@H](O)CCO] | PDX-Al-1 [RM-598-44] |
| 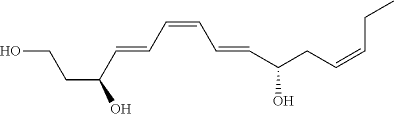 [SMILES: CC/C=C\C[C@H](O)/C=C/C=C\C=C\[C@@H](O)CCO] | PDX-27 [RM-598-45] |
| 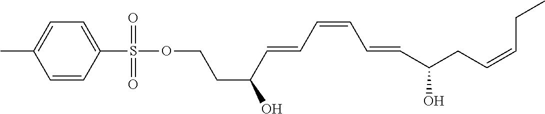 [SMILES: CC/C=C\C[C@H](O)/C=C/C=C\C=C\[C@@H](O)CCOS(=O)(=O)c1ccc(C)cc1] | [PDX-28 [RM-598-41] |
| 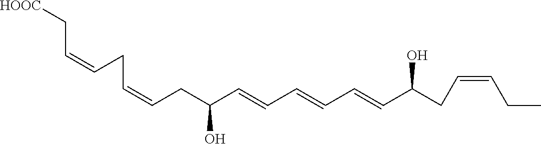 [SMILES: CC/C=C\C[C@H](O)/C=C/C=C/C=C/[C@@H](O)C/C=C\C/C=C\CC(=O)O] | PDX-EEE [RM-598-99] |
| 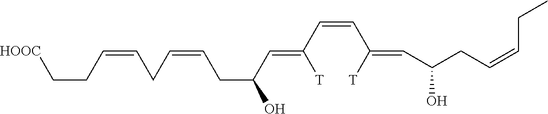 | PDX-T [RM-598-169b] |
| 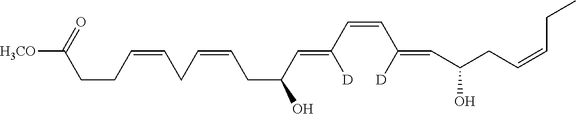 [SMILES: [2H]C(/C=C\C([2H])=C\[C@@H](O)C/C=C\C/C=C\CCC(=O)OC)=C\[C@@H](O)C/C=C\CC] | PDX-D2 [SA-600-118] |
| 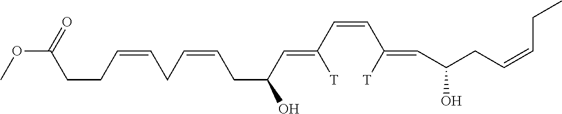 | PDX-Ester-T [RM-598-169a] |
| 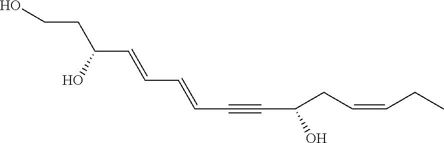 [SMILES: CC/C=C\C[C@H](O)C#C/C=C/C=C/[C@H](O)CCO] | PD1-Al-1 [RM-598-141] |
| 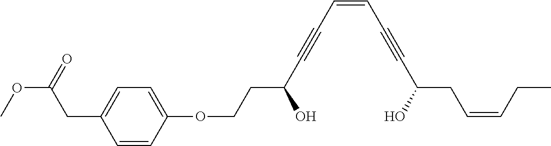 [SMILES: CC/C=C\C[C@H](O)C#C/C=C\C#C[C@@H](O)CCOc1ccc(CC(=O)OC)cc1] | PDX-Al-2 [RM-598-188] |
| 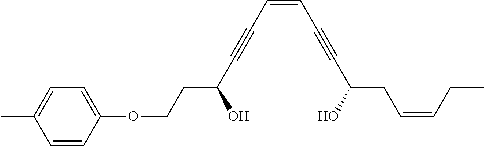 [SMILES: CC/C=C\C[C@H](O)C#C/C=C\C#C[C@@H](O)CCOc1ccc(C)cc1] | [PDX-Al-3 [RM-598-190] |

### Example 2—Biological Activity

A. Combination of a pro-resolving mediator, structural isomer, and/or structural analogs thereof with a known antiviral agent.

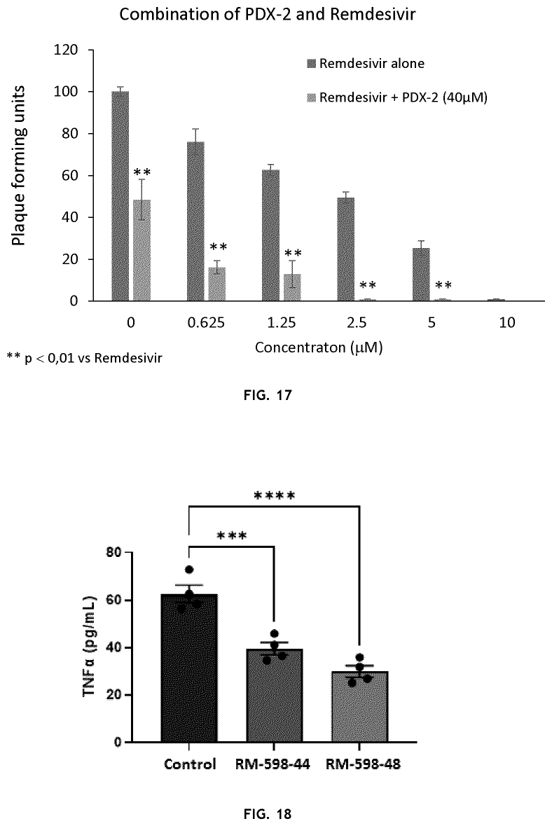

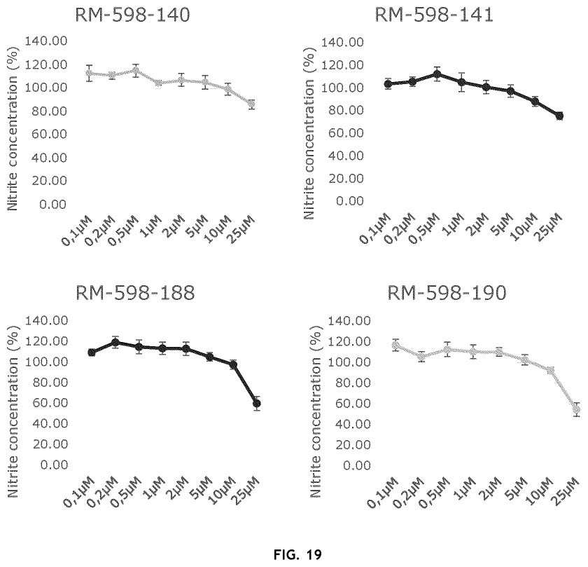

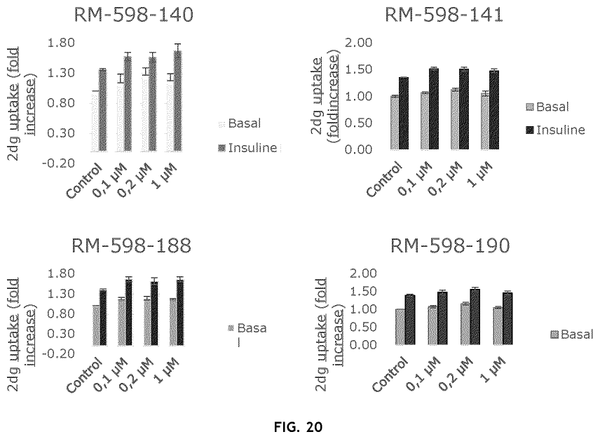

Combination of PDX-2 analog RM-598-48 (40 μM) with Remdesivir increased the antiviral potency of Remdesivir compared to when used alone on SARS-CoV-2. This result suggests an advantage of such combination for treatment of COVID-19 infected patients (FIGS. 17 and 22).

A1. Antiviral effect of selected protectin DX analogs (RM-598-48 and RM-598-44) in SARS-CoV-2 infected hamsters.

Animal Studies. 5-6 Week-old Golden Syrian hamsters were infected with 4.5 log PFU of original SARS-CoV-2 strain. On days 1, 2 and 3, or 1 to 5 post-infection, the animals were treated intraperitoneally with either Remdesivir (15 mg/kg), analog RM-598-48 (50 mg/kg), Remdesivir (15 mg/kg)+analog RM-598-48 (50 mg/kg), analog RM-598-44 (50 mg/kg), or with vehicle only (FIG. 28). The hamsters were monitored daily over a period of 14 days for clinical symptoms and weight loss. On day 4 post-infection, nasal turbinates and lungs were harvested to assess viral replication. All animal studies were approved by the Animal Protection Committee of the Centre Hospitalier Universitaire de Québec according to the Canadian Council on Animal Care guidelines.

Viral titers. Viral titers were determined in cell culture to characterize the presence of live virus. Ten-fold dilutions of nasal turbinates and lung homogenates were performed in infection medium. After one week of infection, titers were determined and reported in TCID₅₀ per gram of organ.

B. Evaluation of the inhibition of SARS-CoV-2 replication by a pro-resolving mediator, structural isomer, and/or structural analog thereof, on VERO E6 cells by Plaque Reduction Assay (PRA).

The antiviral effect against SARS-CoV-2 of PDX analogs and/or structural analogs in VERO E6 cells was evaluated. Remdesivir was used as a positive control.

VERO E6 cell plates (12-well plates) are prepared in a BSL-2 facility in culture media (Minimum essential medium powder (MEM) (#61100-103, Gibco)+1% Hepes (#600-032-LG, Wisent)+10% fetal bovine serum (FBS) (#SH30396.03, GE Healthcare Bio-sciences, now Cytiva). Each well is seeded with 320 000 cells/mL in a final volume of 1 mL. The plates are then placed in an incubator at 37° C. under 5% CO₂ for 3 days (Forma Direct Heat-Thermo Scientific model 310).

On the day of infection, in a BSL-3 facility, a viral suspension of SARS-CoV-2 is prepared in assay media (MEM+1% Hepes+2% FBS+1% antibiotics (gentamicin 1 mg/ml (#15710064, Life Technologies)+2.5 mg/mL vancomycin (#M109D, Hospira)+amphotericin B 0.125 mg/mL (450-105-QL, Wisent)) at 110 pfu/mL. The culture media is removed by aspiration (VACUSIP, Integra) and 250 μL of viral suspension is added in each well. The plates are then placed in an incubator at 37° C. under 5% CO₂ for 1 hour (Forma SteriCycle-Thermo Scientific).

By that time, 2-times final concentrations of each test compound are prepared in 2× medium (MEM 2×(#61100-103 Gibco)+2% Hepes+4% FBS+2% antibiotics as above). After a 1-hour incubation period, each 2-final concentration of the test compounds are diluted 1:1 with 1.2% (p/v in water) of sterilized Sea plaque agarose (#CA12001-898, Lonza), melted and kept at 37° C. The final concentrations of the test compounds are then 1× and the sea plaque agarose 0.6%. The assay media is removed by aspiration (VACUSIP, Integra) and 2 mL of each test compound concentration are added in 12-well plates in triplicate. The plates are left at room temperature to solidify the agarose (10-15 minutes) and then placed in an incubator at 37° C. under 5% CO₂ for 3 days (Forma SteriCycle-Thermo Scientific). After 3 days, the cells are fixed by completely filling all the wells with formalin 4% (formaldehyde 37% [FR-0137, Laboratoire MAT] diluted 1:10 in water) and incubated for 1 hour at room temperature to inactivate the virus and to melt the agarose. The agarose is carefully removed from the well and the cells are stained by adding a few drops of crystal violet 0.8% (p/v) (CA1.01408.0100, Millipore Sigma in 50% ethanol) in each well. The plates are then rinsed with water and dried.

The plaque forming units (pfu) (cytopathic effects) for all the wells are counted under a binocular (Wild Leitz Canada). The average of the 3 wells with no compounds is considered the 100% cytopathic effect. With Microsoft Excel (2013), the decrease of the percentage of the cytopathic effects against the increasing concentrations of compounds is calculated (the average count for one concentration is divided by the count with no compounds and then multiplied by 100). The IC₅₀ can also be calculated if there are at least 6 different concentrations of a same compound by drawing a graph with the logarithm of the concentrations of the compound against the % of cytopathic effect.

The anti-inflammatory and antiviral effects against SARS-CoV-2 of selected PDX analogs and/or structural analogs in accordance with an embodiment of the present disclosure, are illustrated in Table 2.

TABLE 2
Anti-inflammatory and antiviral effects against SARS-COV-2 of selected PDX
analogs and/or structural analogs.

| | | SARS-COV-2 | Anti-inflammatory |
|---|---|---|---|
| | | Inhibition | Activity on iNOS |
| | Structure | IC₅₀ | IC₅₀ |
| PDX-Al-1 [RM-598- 44] | 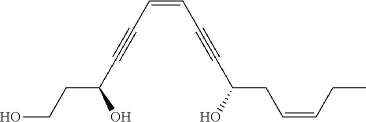 [SMILES: CC/C=C\C[C@H](O)C#C/C=C\C#C[C@@H](O)CCO] | 40% (5 μM) 30% (50 μM) | 18 μM |
| PDX-2 [RM-598- 48] | 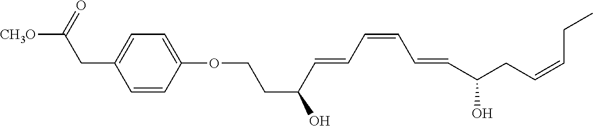 | 33 μM | 64 μM |
| PDX-6 [RM-598- 49] | 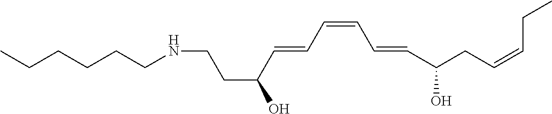 [SMILES: CC/C=C\C[C@H](O)/C=C/C=C\C=C\[C@@H](O)CCNCCCCCC] | 29 μM | 50 μM |
| PDX-1 [RM-598- 53] | 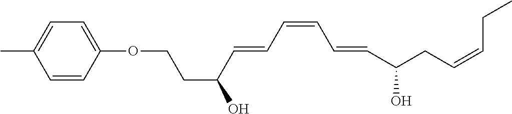 [SMILES: CC/C=C\C[C@H](O)/C=C/C=C\C=C\[C@@H](O)CCOc1ccc(C)cc1] | — | 11 μM |
| PDX-10 [RM-598- 94] | 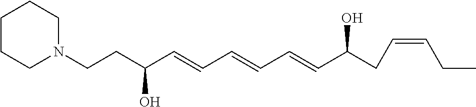 [SMILES: CC/C=C\C[C@H](O)/C=C/C=C/C=C/[C@@H](O)CCN1CCCCC1] | 56 μM | 32 μM |
| PDX | 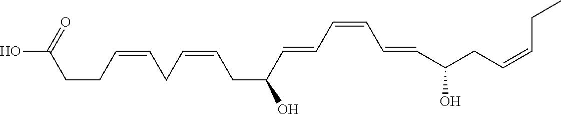 [SMILES: CC/C=C\C[C@H](O)/C=C/C=C\C=C\[C@@H](O)C/C=C\C/C=C\CCC(=O)O] | 0% (5 μM) 18% (50 μM) | 96 μM |
| Remdesivir | | 3.9 μM | — |

C. Evaluation of the activity of PDX and analogs against influenza A/Puerto Rico/8/34 (H1N1) infection, in vitro.

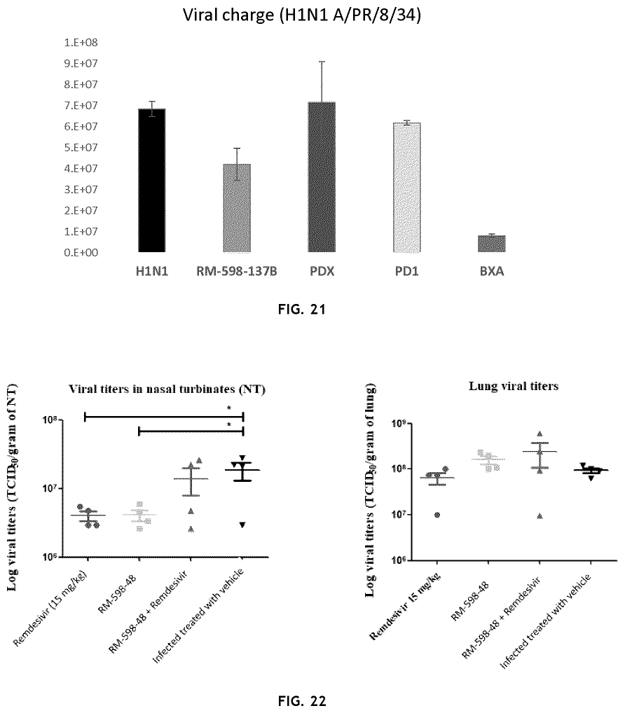

The purpose of this study was to evaluate the antiviral activity of protectin DX, protectin D1 and PDX analogs (e.g., RM-598-137B) against influenza A/Puerto Rico/8/34 (H1N1), in vitro. The influenza polymerase inhibitor Baloxavir acid (BXA) was used as a control (FIG. 21).

Viral infection. Human lung epithelial cells (A549) were grown in a 24-well plate at a confluency of 100% in DMEM+10% FBS+1% antibiotics. On the day of infection, the cells were washed with warm PBS and infected with 200 μL of a viral suspension of influenza A/Puerto Rico/8/34 (H1N1) in infectious medium (DMEM+0.1% bovine serum albumin+0.5 μg/ml of TPCK-trypsin), at a multiplicity of infection (MOI) of 2. After 1 hour of adsorption at 37° C./CO₂, the medium was removed, the cells were washed with PBS, and supplied with 500 μL of fresh infectious medium containing 50 μM of PD1, PDX, analog (e.g., RM-598-137B) or 5 μM of Baloxavir acid (BXA). After 24 hours of incubation at 37° C./CO₂, the supernatants were harvested and frozen at −80° C. The experiment was performed in triplicate.

Quantification of the viral yield. The supernatants were thawed and used for viral RNA isolation (MagNA Pure system (Roche)). Viral titration was evaluated by RT-PCR (LigthCycler system (Roche) (FIG. 21).

### D. Anti-Inflammatory Assays

J774A.1 macrophages are grown in DMEM, supplemented with 10% Bovine Growth Serum (BGS) and 1% penicillin-streptomycin (PS). The J774A.1 macrophages are subsequently stimulated with LPS 2.5 ng/ml in DMEM supplemented with 10% BGS and 1% PS for 16 h-18 h. At the same time, PDX and analogs, at concentrations of 100 nM, 200 nM, 500 nM, 1 μM, 2 μM, 5 μM, 10 μM and 25 μM are added to the cells. After incubation, the level of inflammation is evaluated by measuring the nitrite content in the medium by using the Griess method and normalized by protein concentration.

D1. Evaluation of the Anti-Inflammatory Effect of Selected PDX Analogs (RM-598-44 and RM-598-48) in Obese Hamster Stimulated with LPS

Preparation of LPS. LPS-EB Ultrapure (InvivoGen, E. coli 0111: B4 strain TLR4 ligand). LPS was diluted with sterile 0.9% saline to make a 5 mg/mL stock solution. On the morning of the protocol the working solution was made by diluting the stock solution to 1 mg/ml in sterile 0.9% saline.

Preparation of analog dose. RM-598-44 and RM-598-48 were provided neat. Each analogue was dissolved in 1 mL of 100% ethanol and aliquoted for storage at −80° C. On the day of the protocol the ethanol was dried down under nitrogen and the analog was re-suspended in vehicle (50 mg/mL, 92:8 propylene glycol: DMSO) and then divided into individual vials (1 per animal) to ensure an accurate dose.

Experimental Procedures. Hamsters were administered an intraperitoneal (IP) injection of 0.6 mg/kg LPS followed by an IP injection of either Control (vehicle 92:8 propylene glycol: DMSO) or PDX analog (50 mg/kg RM-598-44 or RM-598-48 in vehicle). Plasma from cardiac puncture was used to measure TNFα(myBioSource MBS7606475) following the manufacturers' instructions (FIG. 18).

### E. Glucose Transport Assays

L6 myoblasts are grown in a MEM medium supplemented with 10% Fetal Bovine Serum (FBS) and differentiated into myotubes in a MEM medium with 2% FBS for 7 days. C2C12 myoblasts are grown in DMEM high glucose medium supplemented with 10% FBS and differentiated into myotubes in a DMEM high glucose medium with 2% Horse Serum for 7 days. The L6 and C2C12 myotubes are serum deprived for 5 h. Two hours prior to glucose transport measurement, PDX and analogs at concentrations of 100 nM, 200 nM, 1 μM, 5 μM, 10 μM and 25 μM are added to the cells. 45 min prior to glucose transport, insulin 100 nM is added to the cells. Both basal and insulin stimulated 2-deoxyglucose uptake is evaluated by incubating cells with 10 μM unlabeled 2-deoxyglucose and D-2-deoxy-[3H] glucose (0.33 μCi/mL) for 8 min. The reaction is terminated by washing three times with ice cold 0.9% NaCl (w/v). Cell-associated radioactivity is determined by lysing the cells with 0.05N NaOH, followed by liquid scintillation counting, and normalized to the protein concentration.

### F. Pharmacokinetic Study of the PDX Analogs RM-598-44 and RM-598-48 in Hamsters.

Animals. Five to six week-old female hamsters weighing approximately 120 g were obtained from Charles-River, Inc (St-Constant, Qc., Canada). The hamsters were housed one per cage and were fed certified commercial rodent food (2018SX, Envigo, USA), and water was available ad libitum. The hamsters were randomized according to their body weight and assigned to 1 to 3 groups of 4 animals each at day 1 of the study. The hamsters were then acclimatized to the environmental conditions (temperature: 22±3° C.; humidity: 50±20%; 12-h light/12-h dark cycles, lights on at 07:15 h) for at least 4 days before starting the experiment. The experiments with the animals were conducted in an animal facility approved by the Canadian Council on Animal Care (CCAC) and the Association for Assessment and Accreditation of Laboratory Animal Care. The study was performed in accordance with the CCAC Guide for Care and Use of Experimental Animals. Institutional approval was obtained.

In vivo treatments with RM-598-44 and RM-598-48. The pharmacokinetic study was carried out following one intraperitoneal injection (IP) of RM-598-44 or RM-598-48 at one concentration (25 mg/kg of body weight in 0.1 mL of vehicle fluid) or at three concentrations (5, 25 and 50 mg/kg of body weight in 0.1 mL of vehicle fluid). The analogs were first dissolved in DMSO, followed by the addition of aqueous propylene glycol to obtain a final concentration of DMSO of 8%. Blood samples for determination of analog plasma concentration were collected by lateral saphenous vein (0.1 mL per animal). At the end of the experiment, the hamsters, under isoflurane anesthesia, were sacrificed by asphyxiation using CO₂. Blood samples were collected in Microvette potassium-EDTA (ethylenediamine tetra-acetic acid)-coated tubes (Sarstedt, Montreal, Canada) and centrifuged at 3200 rpm for 10 minutes at 4° C. The plasma was collected and stored at −80° C. until used for the determination of inhibitor concentrations by liquid chromatography tandem mass spectrometry (LC/MS/MS).

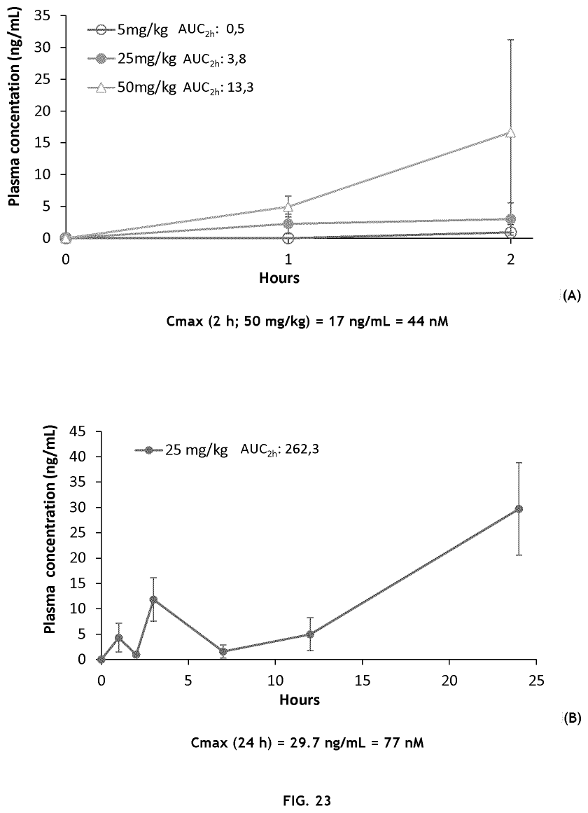

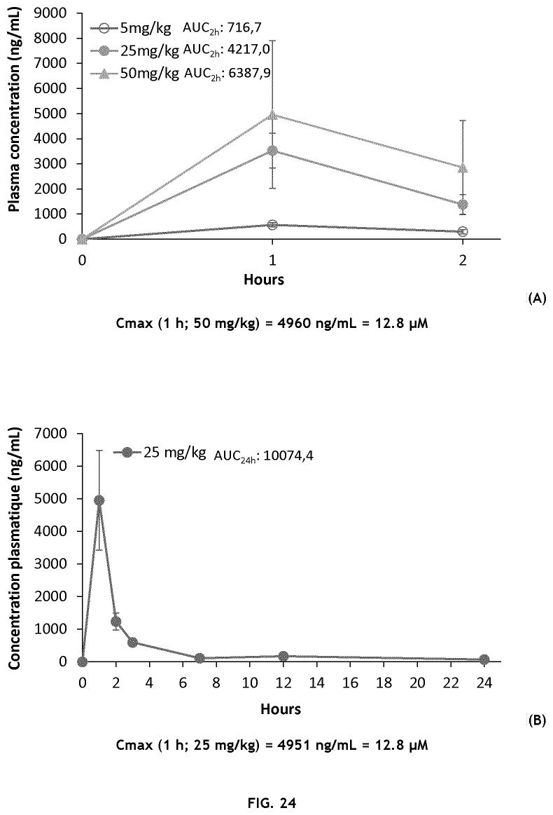

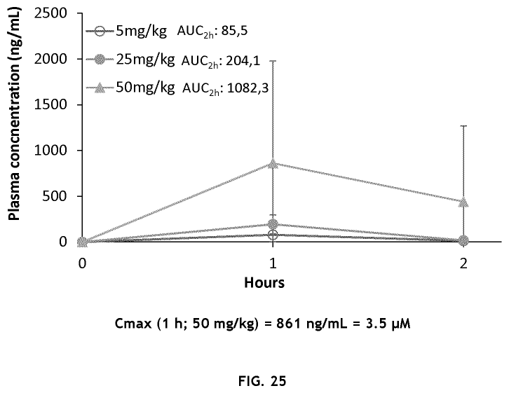

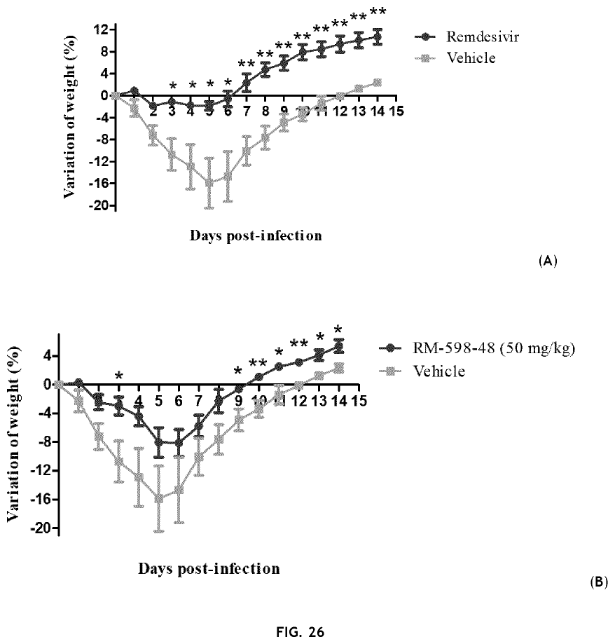

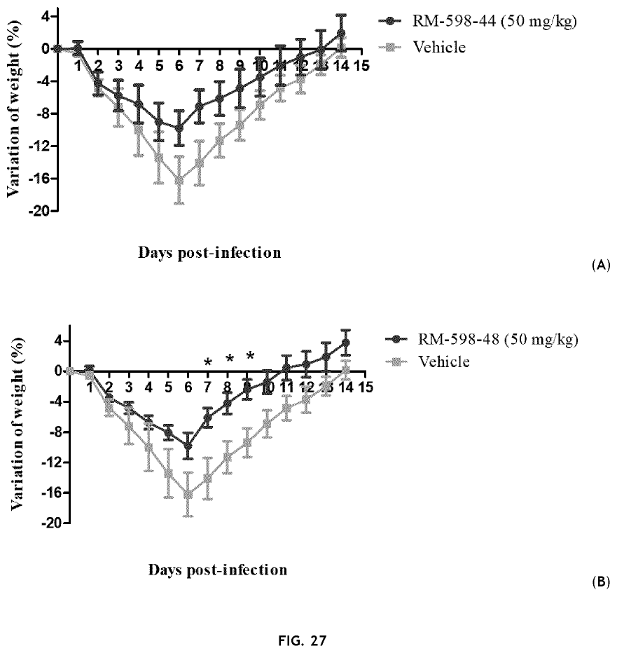

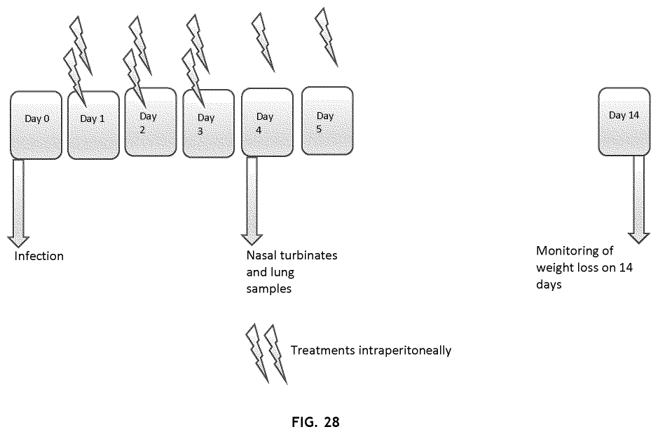

Quantification method of RM-598-44, RM-598-48, and metabolite RM-598-43 from hamster plasma. 25 μL of plasma was diluted in 2 mL of water containing 0.1% formic acid and 0.005% butylated hydroxytoluene (BHT; Sigma, St. Louis, MO), followed by the addition of 50 UL of internal standard (leukotriene B₄-d₄; LTB₄-d₄, 50 ng/ml; and Resolvin D₂-d₅, RvD2-d5, 20 ng/ml; Cayman chemicals, Michigan, USA). Solid phase extraction (SPE) Strata-X 60 mg columns (Phenomenex, Torrance, CA, USA) were conditioned with 2×1 mL methanol and 2×1 mL water 0.1% formic acid. Samples were added to the columns and successively washed with 2×1 mL of H₂O and 2×1 ml of water: methanol (80:20) 0.1% formic acid. Analytes were eluted with 2×1 mL of methyl formate (Sigma, St. Louis, MO). The elutes were completely evaporated at 25° C. under N₂ and reconstituted in 100 μL of water: MeOH (50:50). The same procedure was also applied to the calibration standards which correspond to the dilution of 25 μL of the working solutions (0.5-5 000 ng/ml in ethanol) in 25 μL of stripped plasma (FIGS. 23-25).

The chromatographic separations were achieved with an ultra-high pressure liquid chromatography (UHPLC) Nexera Separations Module (Shimadzu Scientific Instruments Inc., Columbia, MD, USA) using a 150×2.1 mm Poroshell 120 EC-C18 column (2.7 μM particles) (Agilent, Santa Clara, CA) at 40° C. The flow rate was set at 0.3 mL/minute and the following mobile phases were used: 3.5 mM ammonium formate in water 0.01% acetic acid (solvent A), and MeOH 0.01% acetic acid (solvent B; (WVR, Montréal, Québec, Canada)).

10 μL was injected for the analysis of analogs RM-598-43 (metabolite) and RM-598-48. The following chromatographic program was used: initial conditions (57% B) was followed by a linear gradient to 59% B over the next 6 min and a second linear gradient to 80% B over 9 minutes; the column was then flushed with 100% B for 5 min and re-equilibrated to the initial conditions for 7 min.

2 μL was injected for the analysis of analog RM-598-44 and the following chromatographic program was used: initial conditions (45% B) was maintained for 2 minutes followed by a linear gradient to 70% B over the next 8 min; the column was then flushed with 100% B for 5 min and re-equilibrated to the initial conditions for 7 min.

All analytes were quantified by tandem mass spectrometry (MS/MS) using an API6500 instrument (Applied Biosystems, Concord, ON, Canada) in positive mode. The temperature was set at 300° C., the entrance potential (EP) at 10V, and the declustering potential (DP) at 70V. MS/MS parameters (ion transition (MRM), and collision energy (CE)) were as follows: RM-598-43:404.2→369.1, 10V; RM-598-48:418.3→365.2, 12V; RM-598-44:266.3→213.0, 10V; LTB₄-d₄: 358.2→305.2, 12V; and RvD2-d5 399.2→364.1, 10V.

### REFERENCES

1. (a) Dalli, J. Mol. Aspects Med. 2017, 58, 1-130. (b) Kuda, O. Biochimie 2017, 136, 12-20. (c) Duvall, M. G.; Levy, B. D. Eur. J. Pharmacol. 2016, 785, 144-155. (d) Serhan, C. N. Nature 2014, 510, 92-101. (e) Spite, M.; Claria, J.; Serhan, C. N. Cell Metabol. 2014, 19, 21-36.
2. (a) Serhan, C. N.; Hamberg, M.; Samuelsson, B. Biochem. Biophys. Res. Commun. 1984, 3, 943-949.
3. (a) Dalli, J.; Serhan, C. N. Microbiol. Spectr. 2016, 4, 1-23. (b) Serhan, C. N.; Chiang, N.; Dalli, J.; Levy, B. D. Cold Spring Harb Perspect Biol 2015, 7, 1-20. (c) Bazan, N. G. Prostaglandins Leukotrienes Essent. Fatty Acids 2013, 88, 127-129. (d) Serhan, C. N.; Petasis, N. A. Chem. Rev. 2011, 111, 5922-5943. (e) Serhan, C. N.; Gotlinger, K.; Hong, S.; Lu, Y.; Siegelman, J.; Baer, T.; Yang, R.; Colgan, S. P. N.; A. Petasis J. Immunol 2006, 176, 1848-1859.
4. (a) Serhan, C. N.; Hong, S.; Gronert, K.; Colgan, S. P.; Devchand, P. R.; Mirick, G.; Moussignac, R. L. J. Exp. Med. 2002, 196, 1025-1037. (b) Serhan, C. N.; Clish, C. B.; Brannon, J.; Colgan, S. P.; Chiang, N.; Gronert, K. J. Exp. Med. 2000, 192, 1197-1204.
5. Hong, S.; Gronert, K.; Devchand, P. R.; Moussignac, R. L.; Serhan, C. N. J. Biol. Chem 2003, 278, 14677-14687.
6. Butovich, I. A. J. Lipid. Res. 2005, 46, 2311-2314.
7. Chen, P.; Fenet, B.; Michaud, S.; Tomczyk, N.; Vericel, E.; Lagarde, M.; Guichardant, M. FEBS Lett. 2009, 583, 3478-3484.
8. Zhuo, X-J.; Hao, Y.; Cao, F.; Yan, S-F.; Li, H.; Wang, Q.; Cheng, B-H.; Ying, B-Y; Smith, F. G.; Jin, S-W. Exp. Mol. Med. 2018, 50:49.
9. Piao, S.; Du, W.; Wei, Y.; Yang, Y.; Feng, X.; Bai, L. Intern. Immunopharmacology 2020, 106043.
10. Liu, M.; Boussetta, T.; Makni-Maalej, M. K.; Fay, M.; Driss, F.; El-Benna, J. Lipids 2013, 49, 49-57.
11. Morita, M.; Kuba, K.; Nakayama, A. M.; Katahira, J.; Iwamoto, K.; Watanebe, T.; Sakabe, S., Nakamura, T. S.; Kadowaki, A.; Ohto, T.; Nakanishi, H.; Taguchi, R.; Nakaya, T.; Murakami, M.; Yoneda, Y.; Arai, H.; Kawaoka, Y.; Penninger, J. M.; Arita, M.; Imai; Y. Cell 2013, 153, 112-125 (it should be noticed that PDX has been mistaken as PD1 throughout the paper; see Balas, L.; Guichardant, M.; Durand, T.; Lagarde, M. Biochimie 2014, 99, 1-7.
12. Xia, H.; Chen, L.; Liu, H.; Chun, Z.; Yang, W.; Yang, Y.; Cui, S.; Li, S.; Wang, Y.; Song, L.; Abdelgawad, A. F.; Shang, Y.; Yao, S. Nature 2016, 7, 1-11.
13. Li, H.; Hao, Y.; Zhang, H.; Ying, W.; Li, D.; Ge, Y.; Ying, B.; Cheng, B.; Lian, Q.; Jin, S. Sci. Rep. 2017, 7, 46754.
14. Jung, T. W.; Kyung, E. J.; Kim, H-C.; Shin, Y. K.; Lee, S. H.; Park, S. P.; Hacimüftüoglu, A.; Abd El-Aty, A. M. J.; Jeong, J. H. Pharmacol. Exp. Ther. 2018, 365, 485-493.
15. Zhang, L.; Y, Liu. J. Med. Virol. 2020; 92, 479-490.
16. Panigraphy, D. et al. Cancer Metastasis Rev., 2020, 39, 337-340.
17. Regidor, P. A. Med. Hypotheses, 2020, 142, 109813.
18. Shaw, J. Harvard Magazine, April 2020, Available at: https://harvardmagazine.com/2020/04/resolution-of-inflammation (accessed July 2020).
19. (a) Mitchell, P. L.; Nachbar, R.; Lachance, D.; St-Pierre, P.; Trottier, J.; Barbier, O. Marette, A. Diabetes Obes. Metab. 2017, 19, 313-319. (b) U.S. Pat. No. 9,782,379 and EP 2 948 163B1. (c) White, P.-J.; Mitchell, P. L.; Schwab, M.; Trottier, J.; Kang, J. X.; Barbier, O., Marette, A. Transgenic ω-3 PUFA enrichment alters morphology and gene expression profile in adipose tissue of obese mice: Potential role for protectins. Metabol. Clin. Expl. 2015, 64, 666-676. (d) White, P. J.; St-Pierre, P.; Charbonneau, A.; Mitchell, P. L.; St-Amand, E.; Marcotte, B.; Marette, A. Nat. Med. 2014, 20, 664-669. (e) Marette, A.; Liu, Y.; Sweeney, G. Skeletal muscle glucose metabolism and inflammation in the development of the metabolic syndrome. Rev. Endocrine Metabol. Disorders 2014, 15, 299-305.
20. Basil, M. C.; Levy, B. D. Nat Rev Immunol, 2016, 16, 51-67.
21. Chiang, N.; Serhan, C. N. Essays Biochem., 2020, 64, 443-462.
22. Park, J.; Langmead, C. J.; Riddy, D. M. ACS Pharmacology & Translational Science, 2020, 3, 88-106.

1. A method of preventing, delaying the onset or reducing the severity of, preventing, or reversing the progression of, or treating a viral infection or disease in a subject, said method comprising administering a protectin, protectin analog, structural isomer, or a pharmaceutically acceptable salt thereof, to the subject.

2. The method of claim 1, wherein the viral infection is a coronavirus infection.

3. The method of claim 1, wherein the viral disease is a viral respiratory disease.

4. The method of claim 1, wherein the viral infection is a SARS viral infection.

5. The method of claim 4, wherein the viral infection is a SARS-CoV viral infection.

6. The method of claim 5, wherein the viral infection is a SARS-CoV-2 viral infection.

7. The method of claim 1, wherein the viral disease is COVID-19.

8. The method of claim 1, wherein the subject suffers from insulin resistance, diabetes, or obesity.

9. The method of claim 1, wherein the viral infection is influenza.

10. The method of claim 1, wherein the protectin, protectin analog, structural isomer, or a pharmaceutically acceptable salt thereof is at least one of:

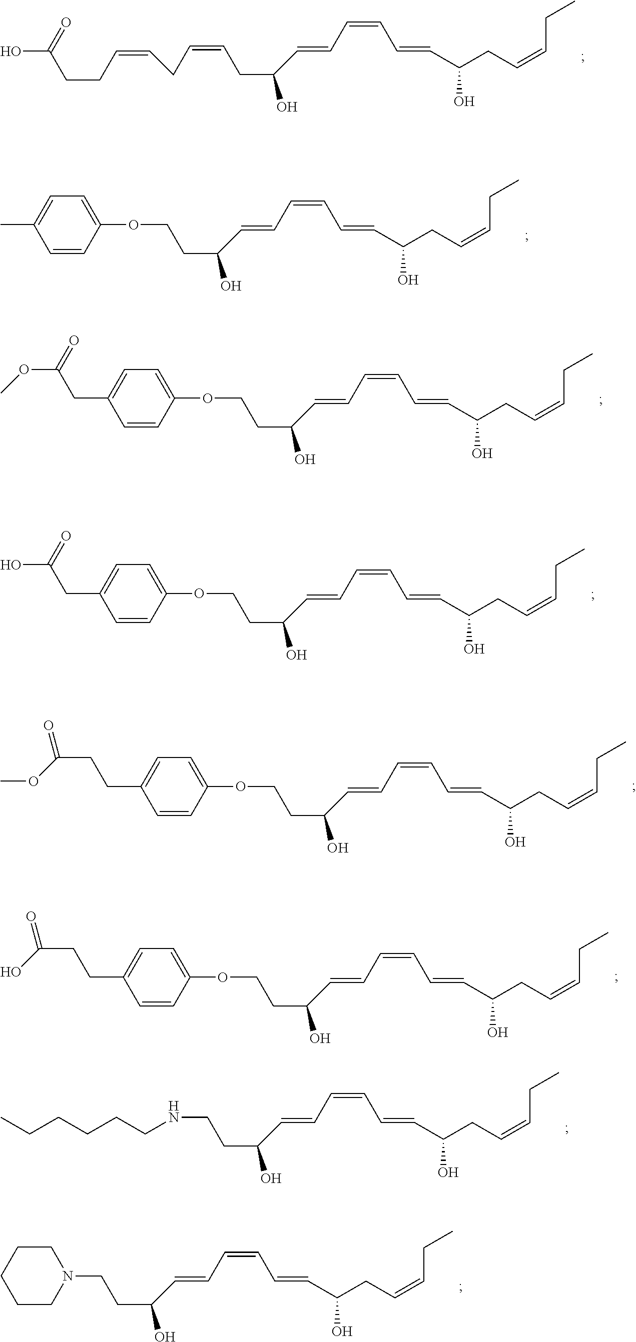

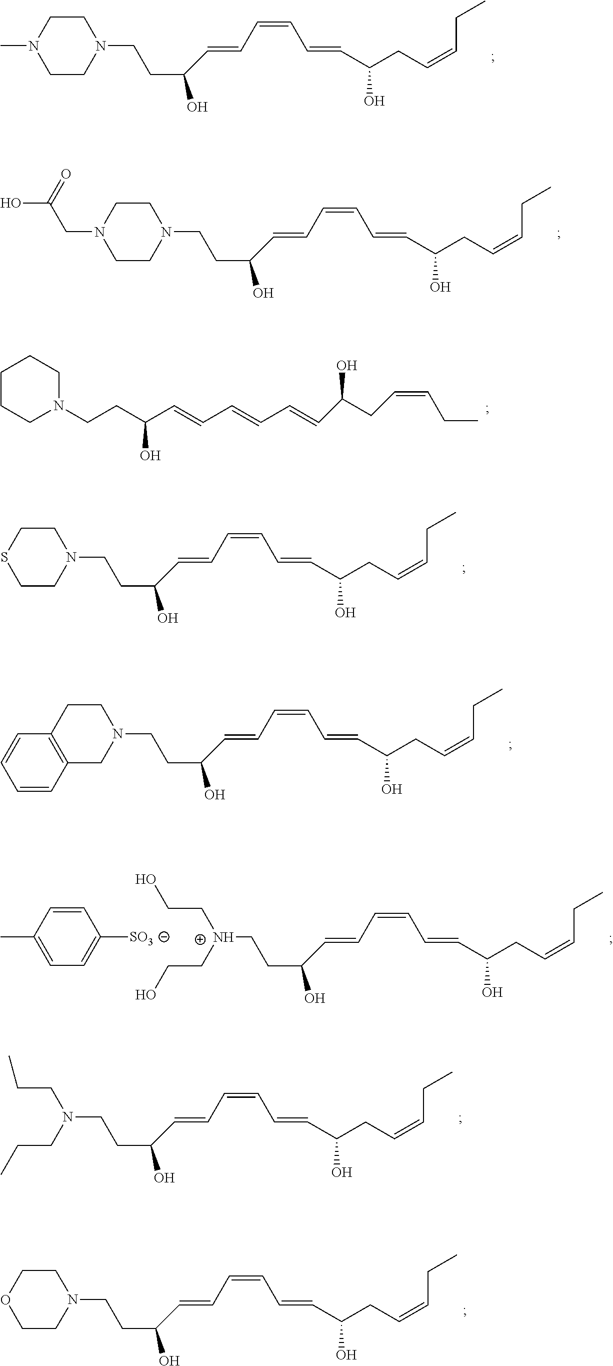

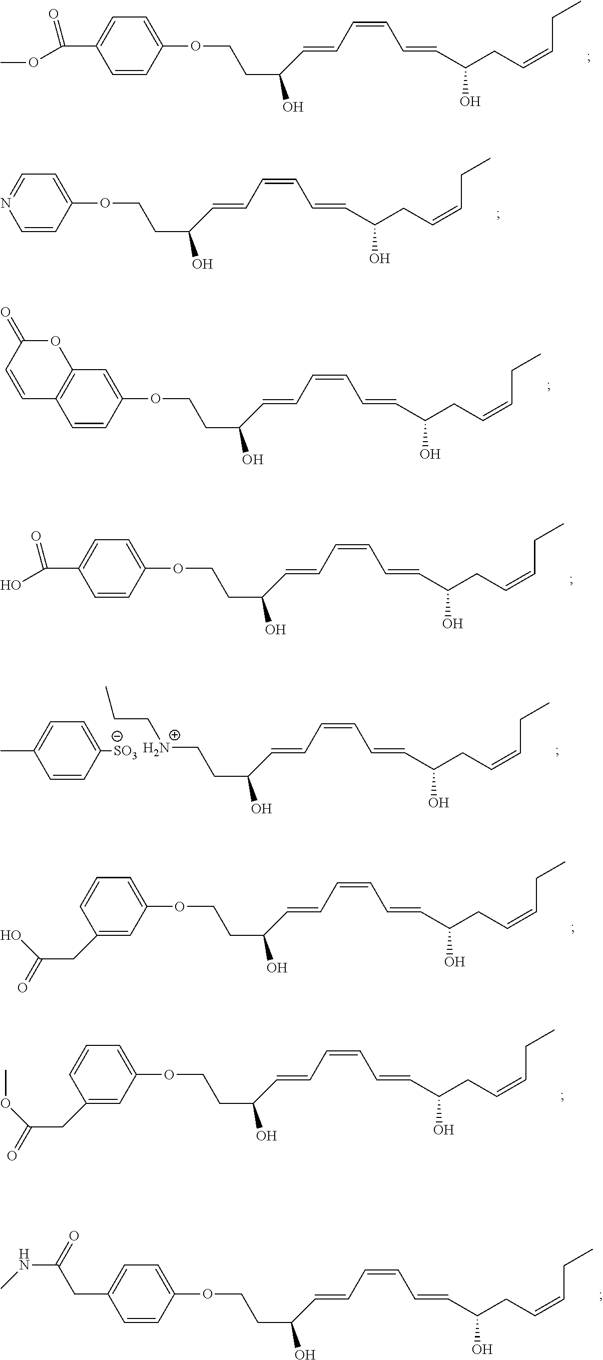

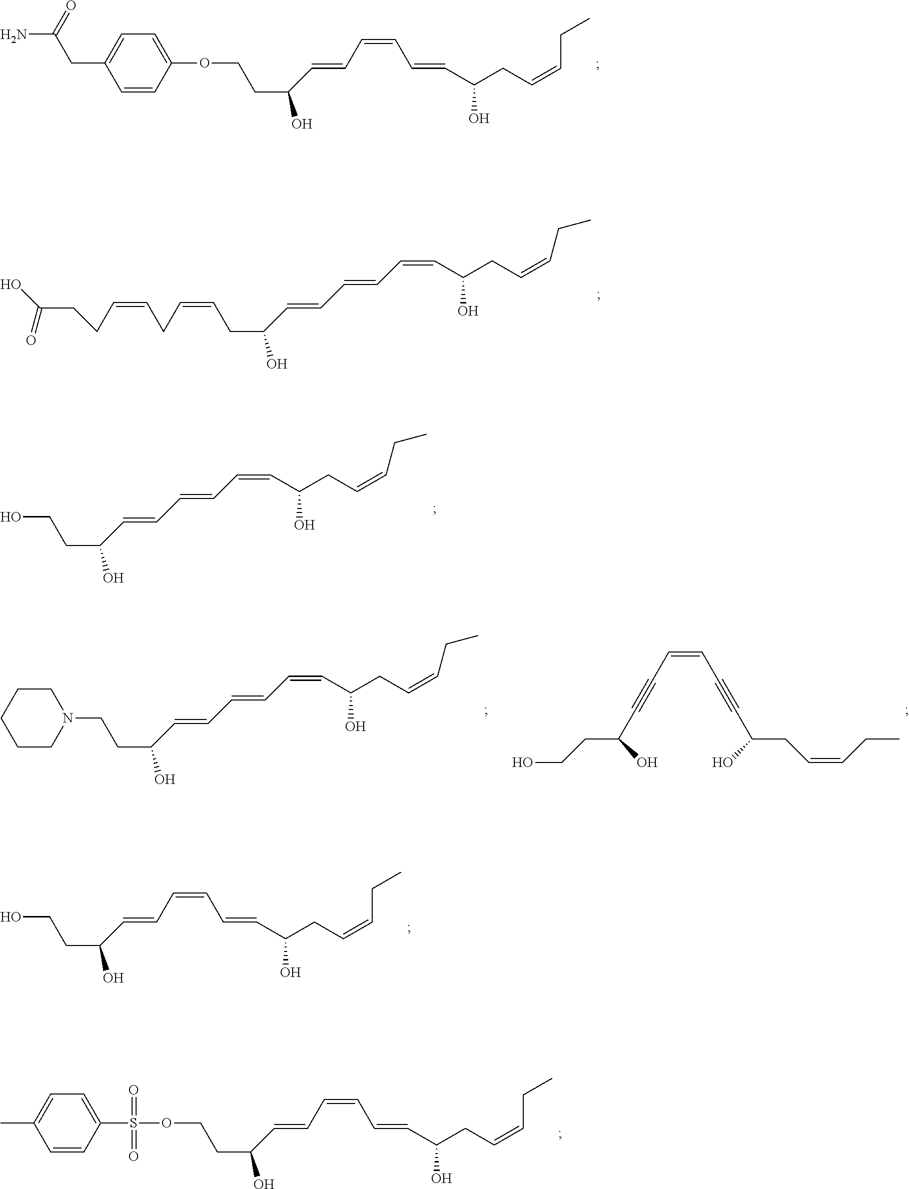

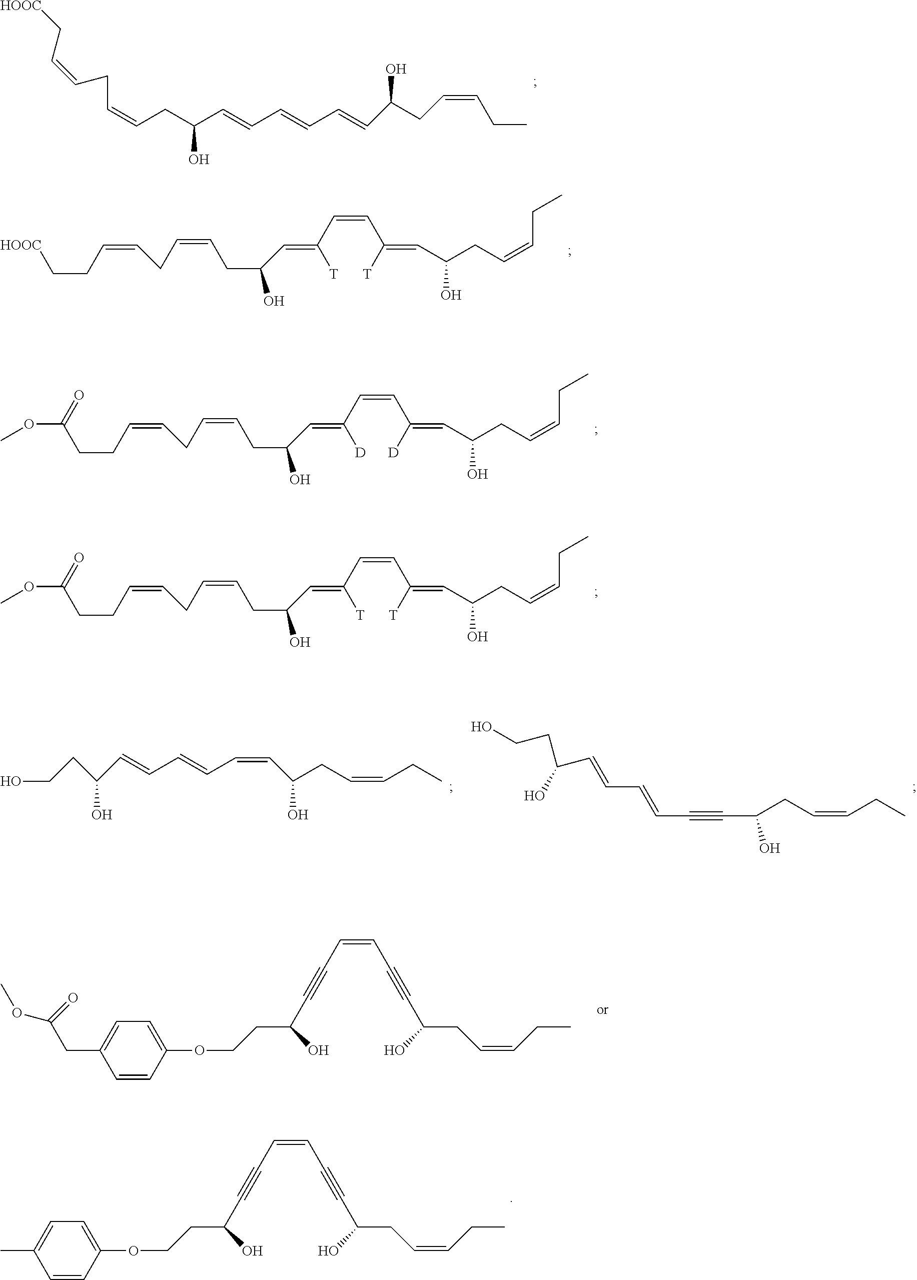

11-31. (canceled)

32. A method of preventing, delaying the onset or reducing the severity of, preventing, or reversing the progression of, or treating insulin resistance or diabetes in a subject, said method comprising administering a protectin, protectin analog, structural isomer, or a pharmaceutically acceptable salt thereof, to the subject.

33. The method of claim 32, wherein insulin resistance is diagnosed by measuring a patient's insulin level.

34. The method of claim 32, wherein diabetes is diagnosed by measuring a patient's glucose level.

35. The method of claim 32, wherein diabetes is type 2 diabetes.

36. The method of claim 32, wherein the subject suffers from obesity.

37. The method of claim 32, wherein the protectin, protectin analog, structural isomer, or a pharmaceutically acceptable salt thereof is at least one of:

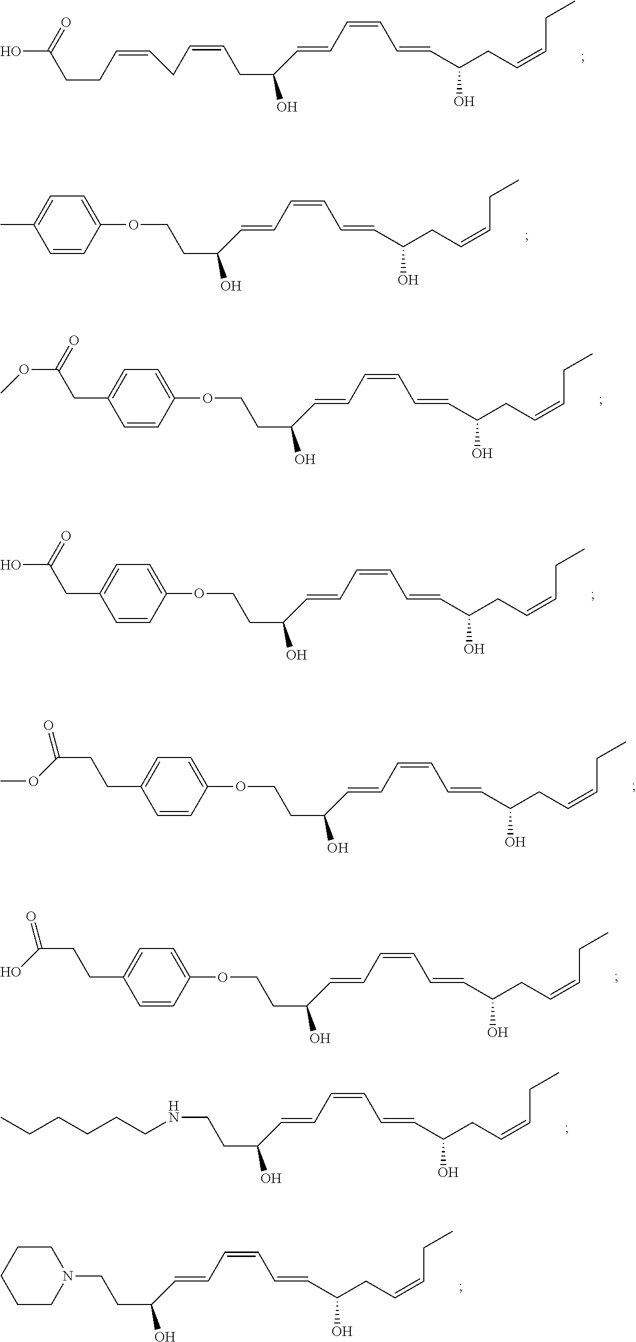

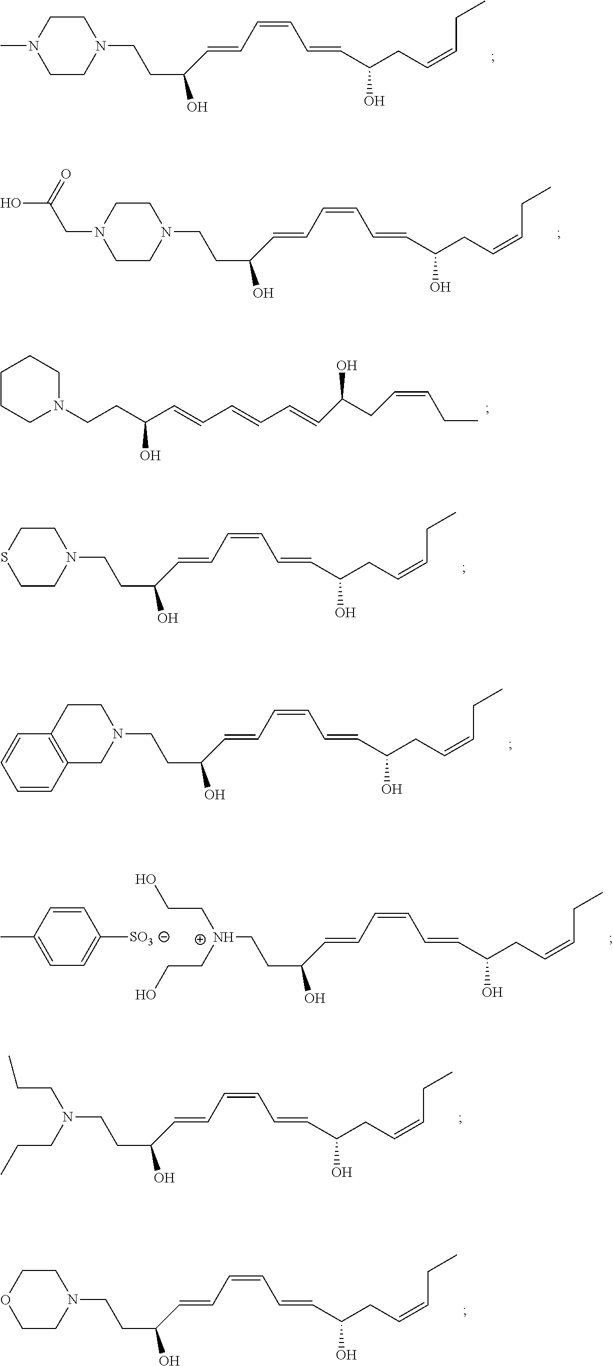

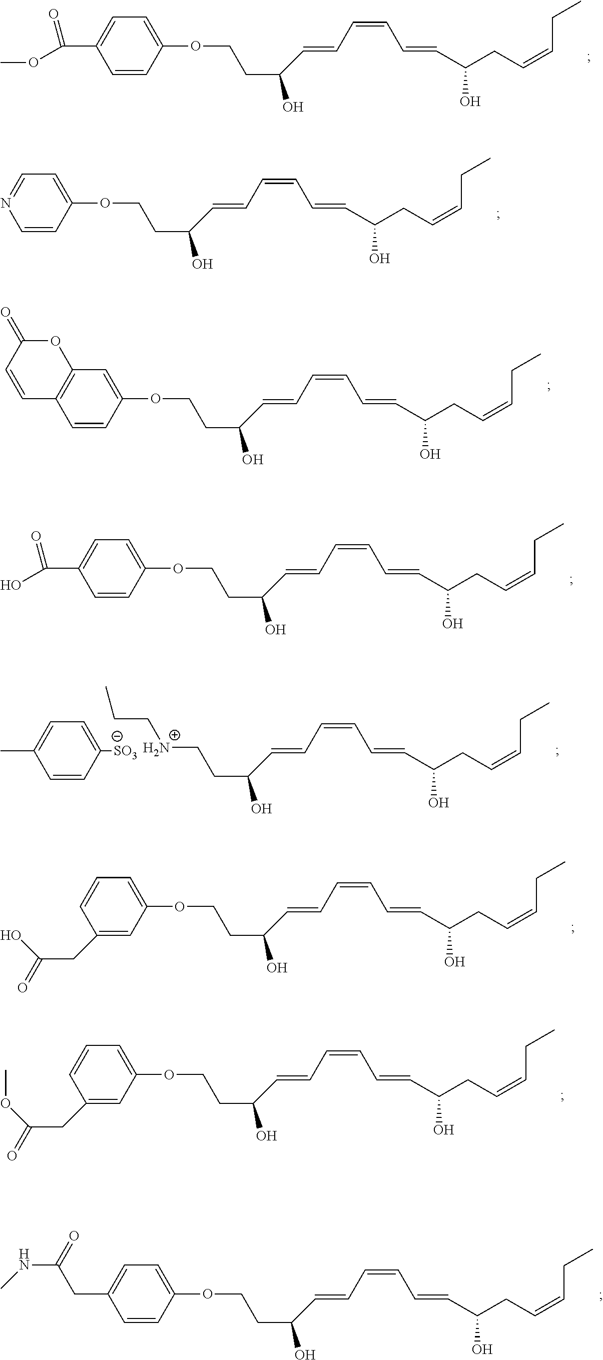

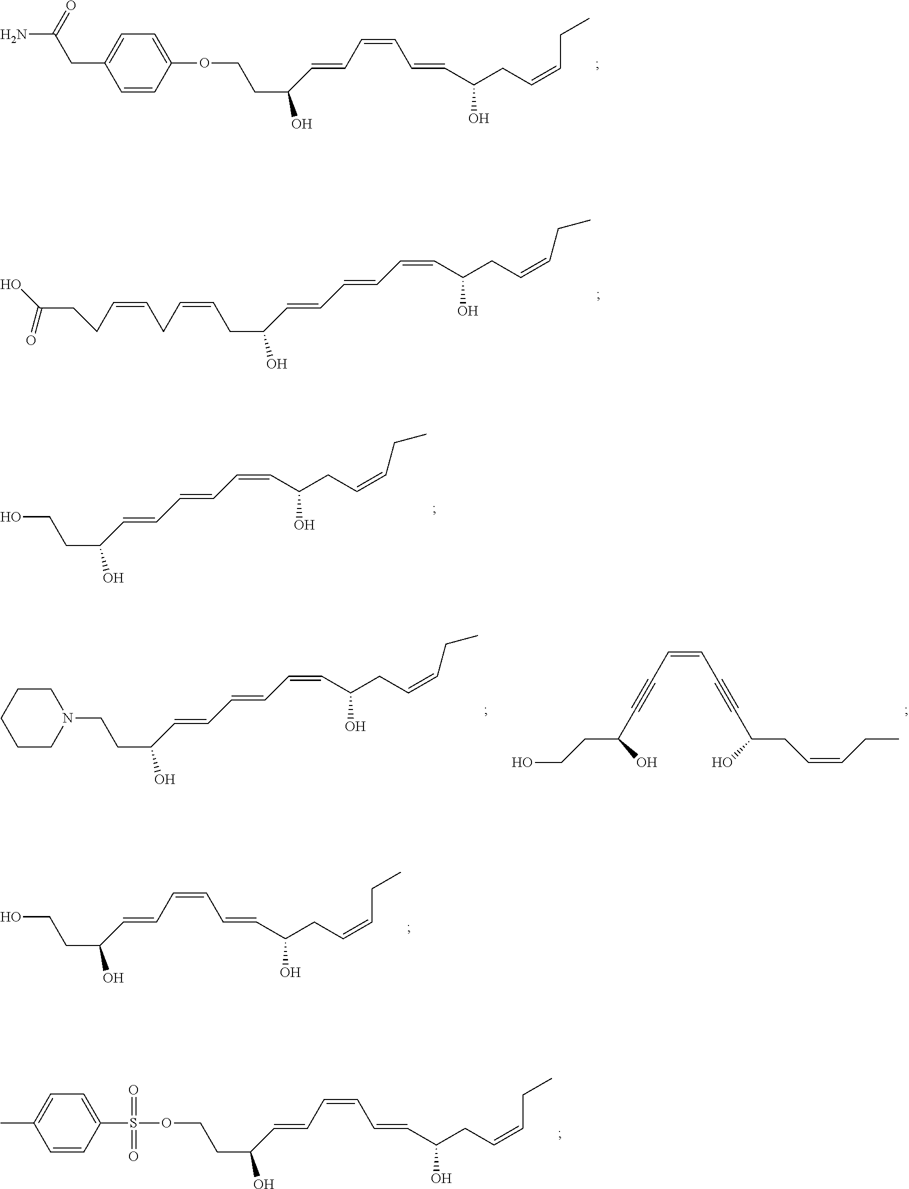

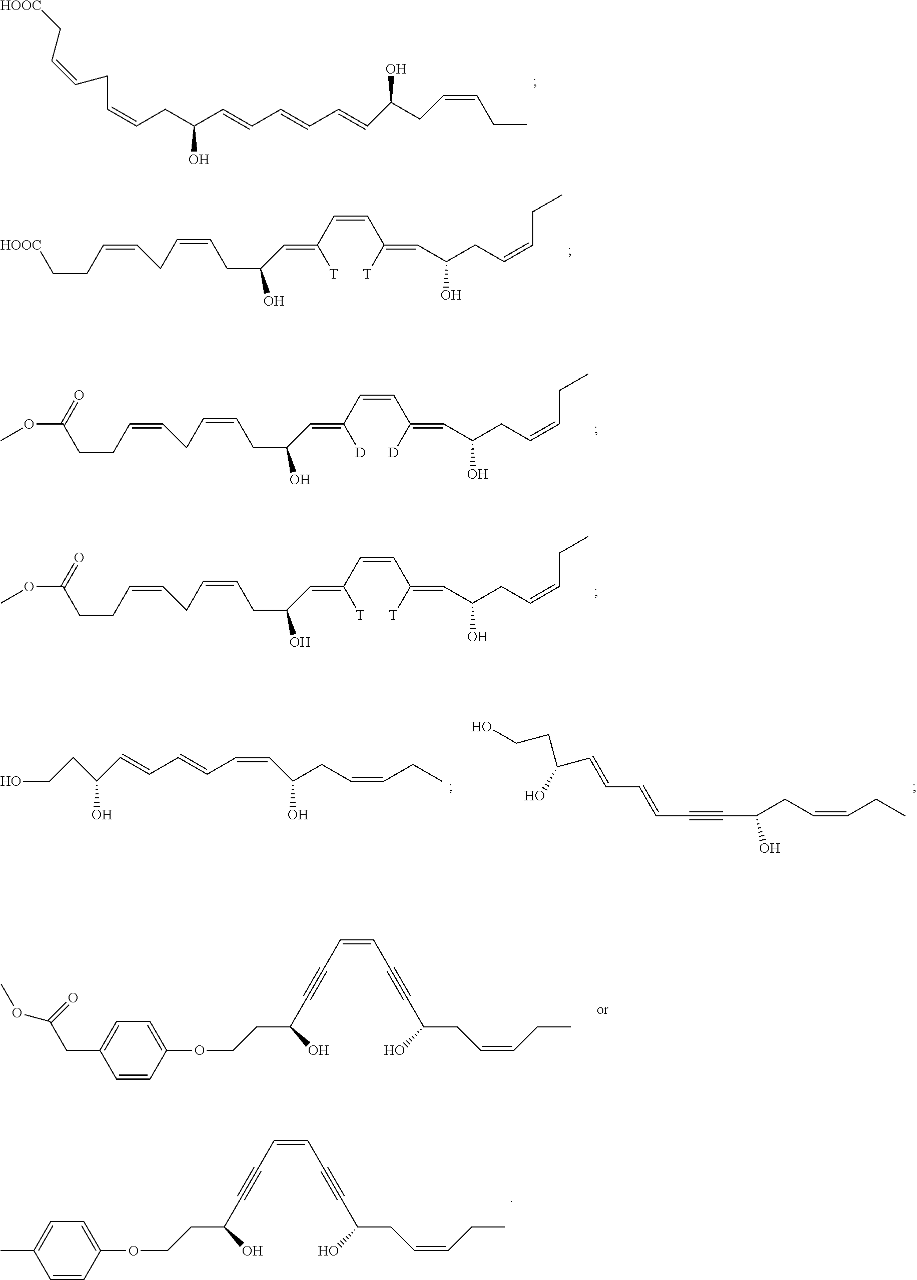

38-70. (canceled)

71. A method of preventing, delaying the onset or reducing the severity of, preventing or reversing the progression of, or treating an inflammatory disease, disorder, condition, or symptom in a subject, said method comprising administering a protectin, protectin analog, structural isomer, or a pharmaceutically acceptable salt thereof, to the subject.

72. The method of claim 71, wherein the subject suffers from obesity.

73. The method of claim 71, wherein the protectin, protectin analog, structural isomer, or a pharmaceutically acceptable salt thereof is at least one of:

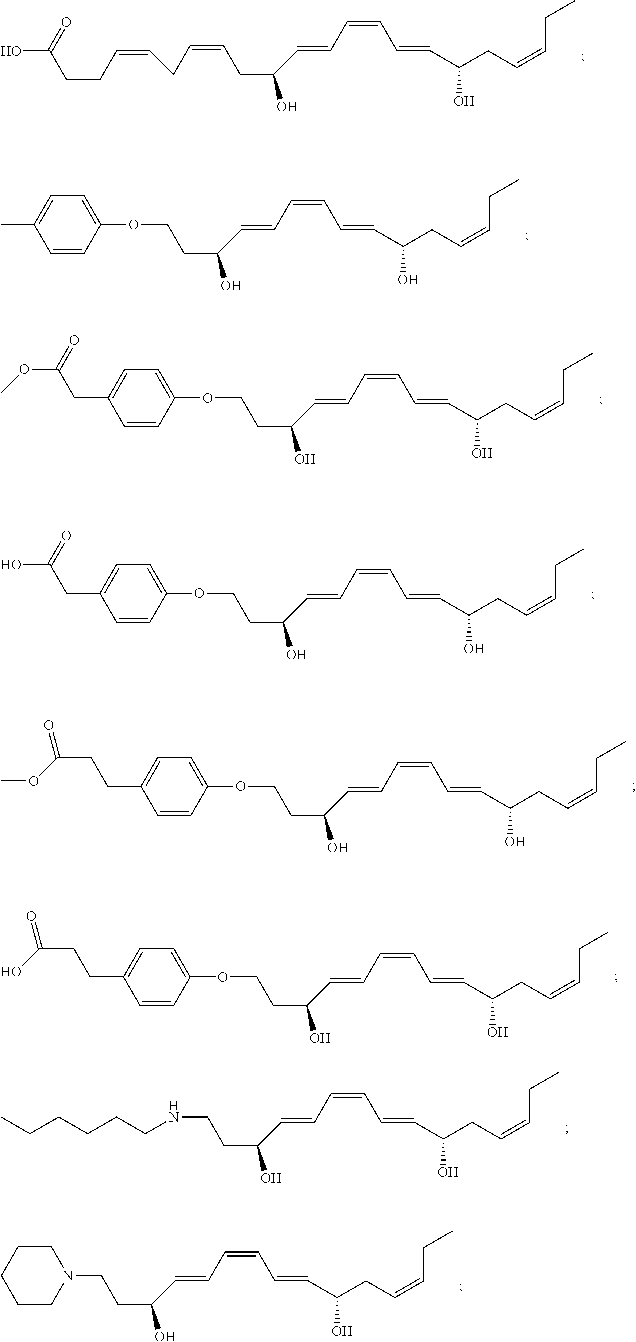

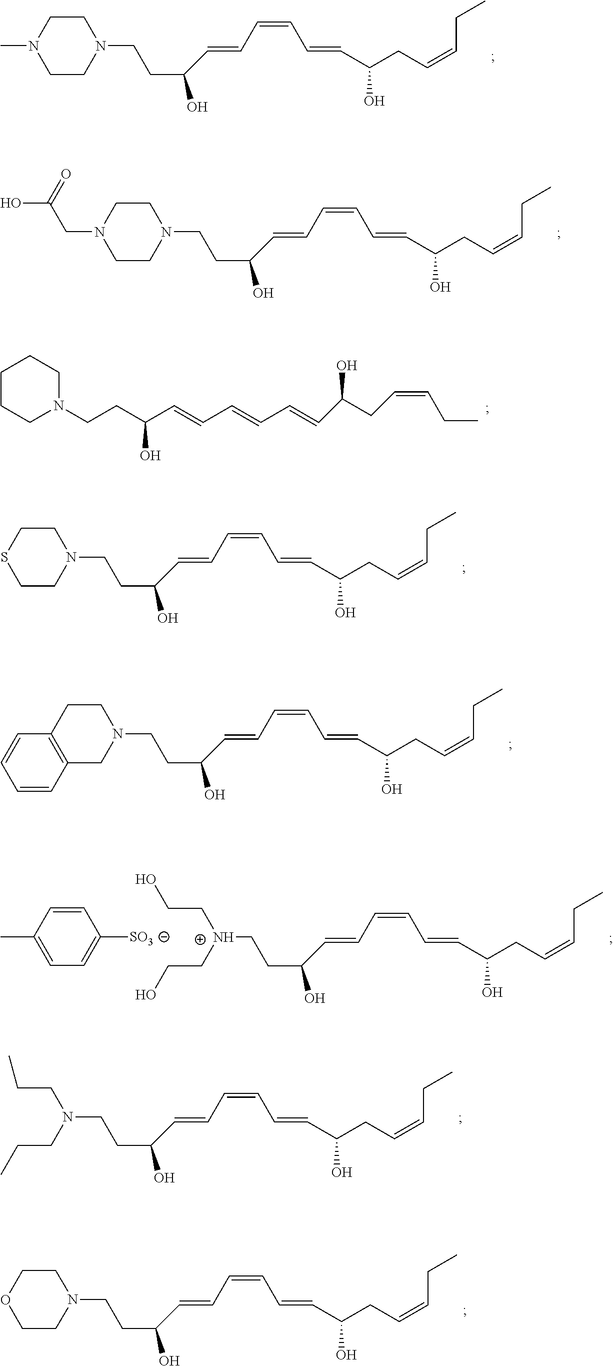

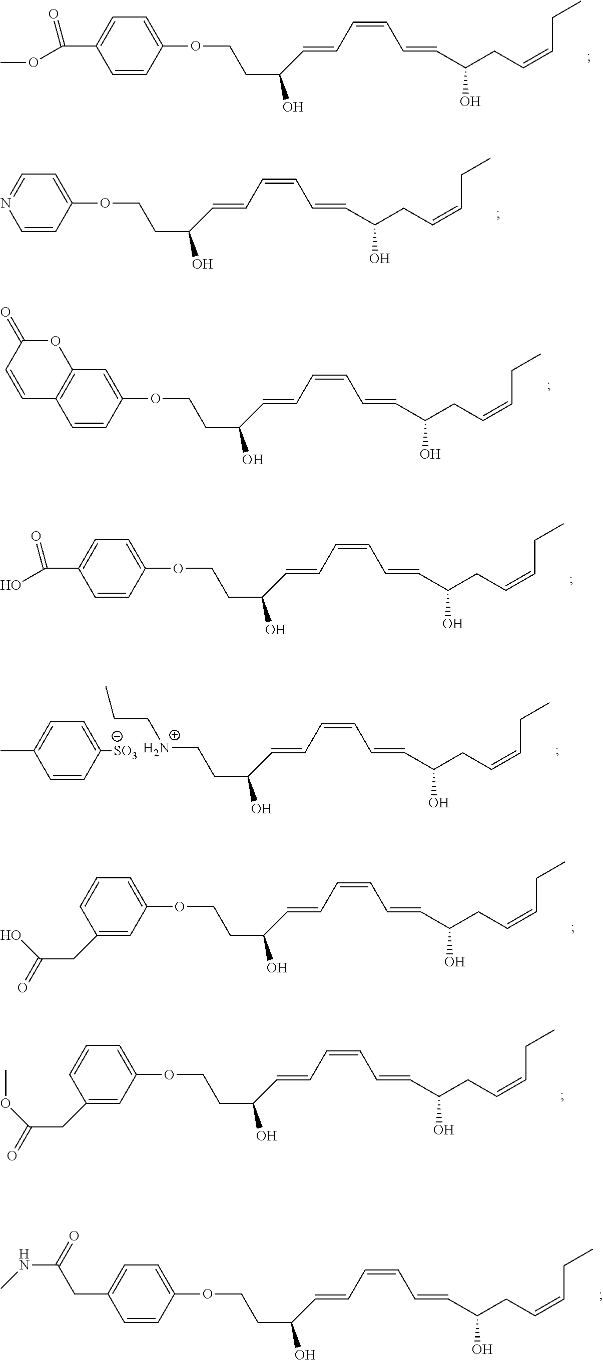

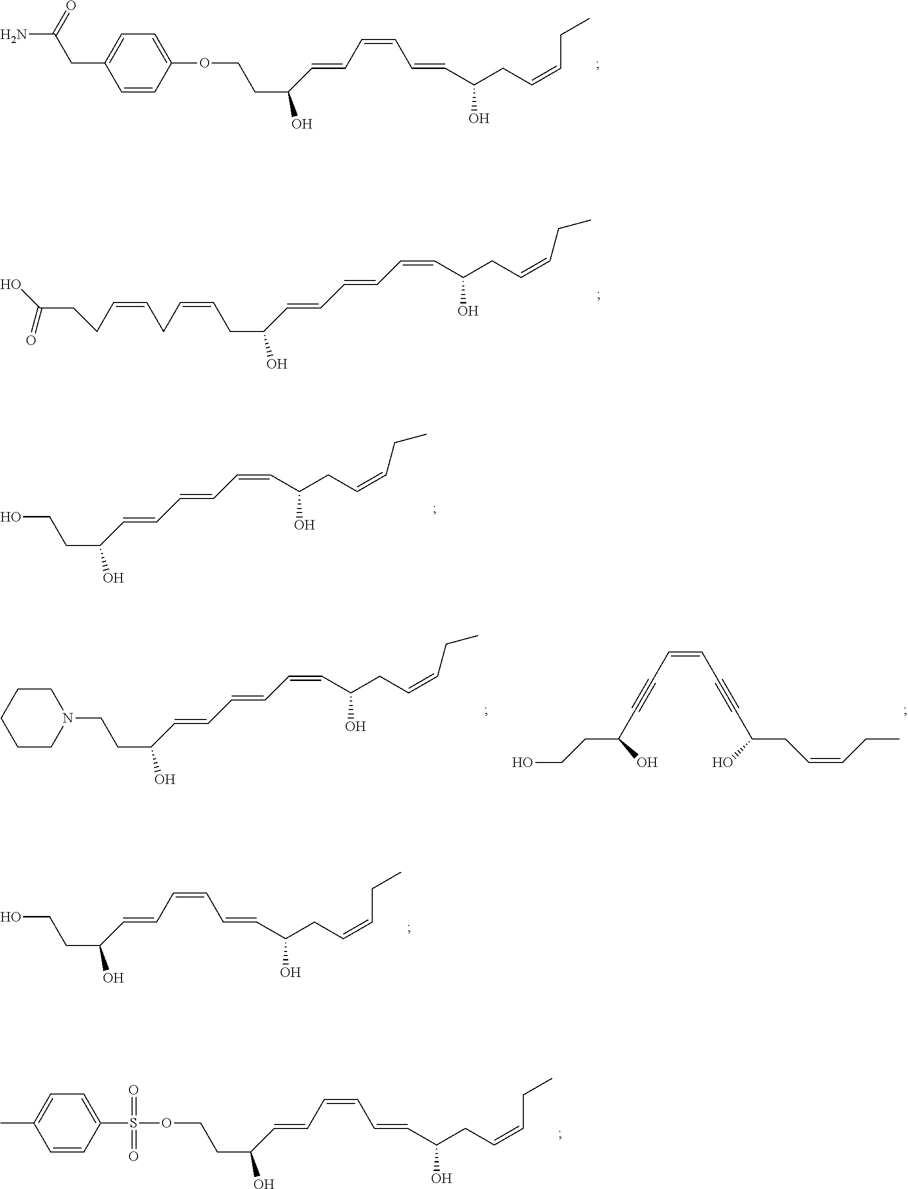

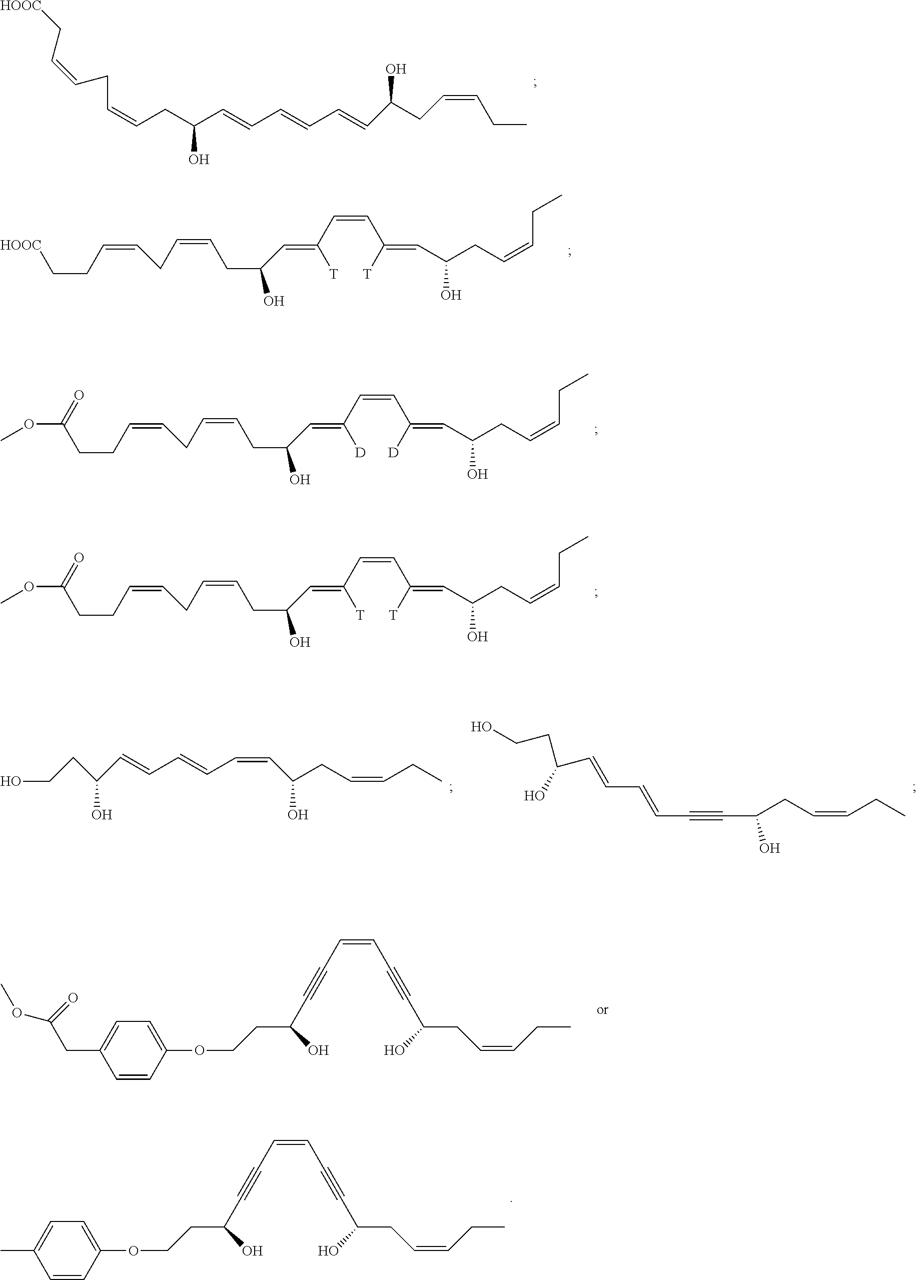

74-80. (canceled)

81. The method of claim 1, further comprising a second agent for delaying the onset or reducing the severity of, preventing, or reversing the progression of, or treating a viral infection or disease in a subject.

82. The method of claim 32, further comprising a second agent for delaying the onset or reducing the severity of, preventing, or reversing the progression of, or treating insulin resistance or diabetes in a subject.

83-84. (canceled)

85. The method of claim 71, further comprising a second agent for delaying the onset or reducing the severity of, preventing, or reversing the progression of, or treating an inflammatory disease, disorder, condition, or symptom in a subject.

86. A method of preventing, delaying the onset or reducing the severity of, preventing, or reversing the progression of, or treating an inflammatory disease, disorder, condition, or symptom that is secondary to another condition in a subject, said method comprising administering a protectin, protectin analog, structural isomer, or a pharmaceutically acceptable salt thereof, to the subject.

87. The method of claim 86, wherein the protectin, protectin analog, structural isomer, or a pharmaceutically acceptable salt thereof is at least one of:

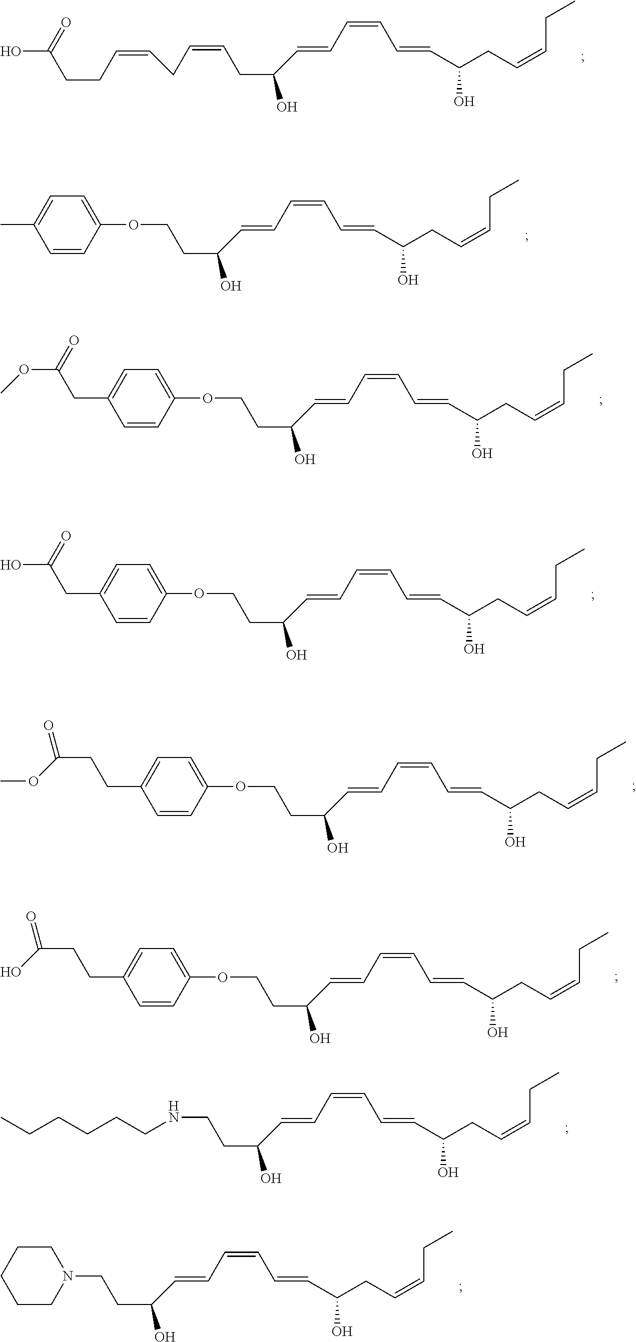

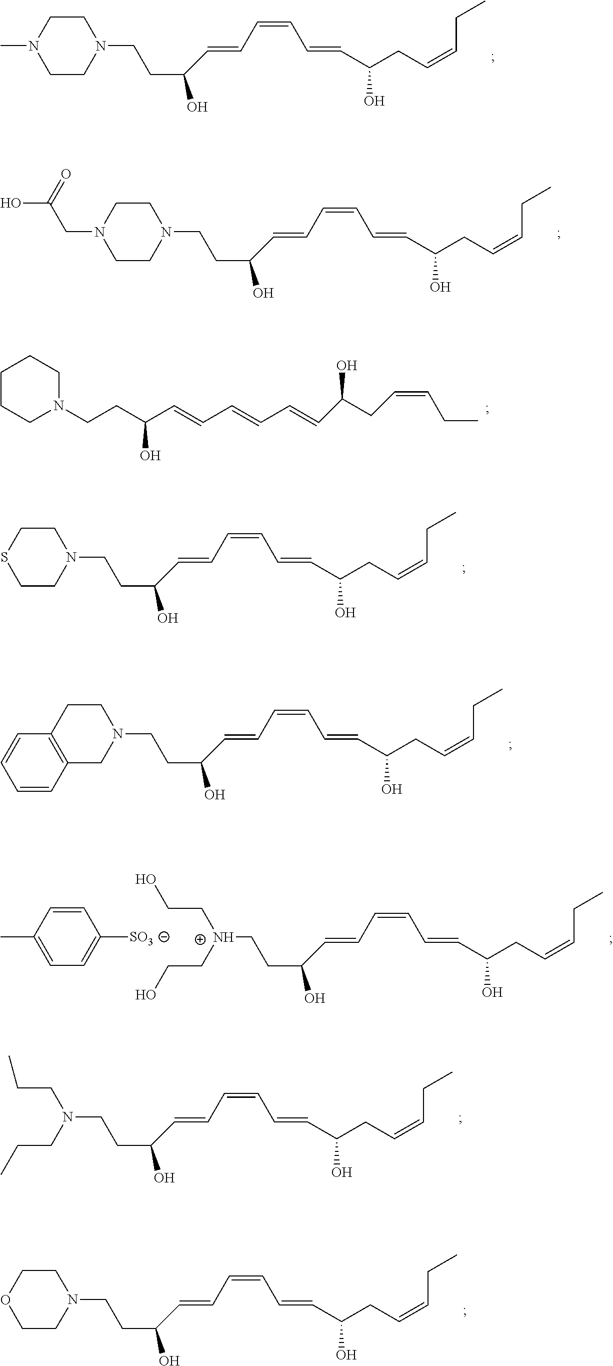

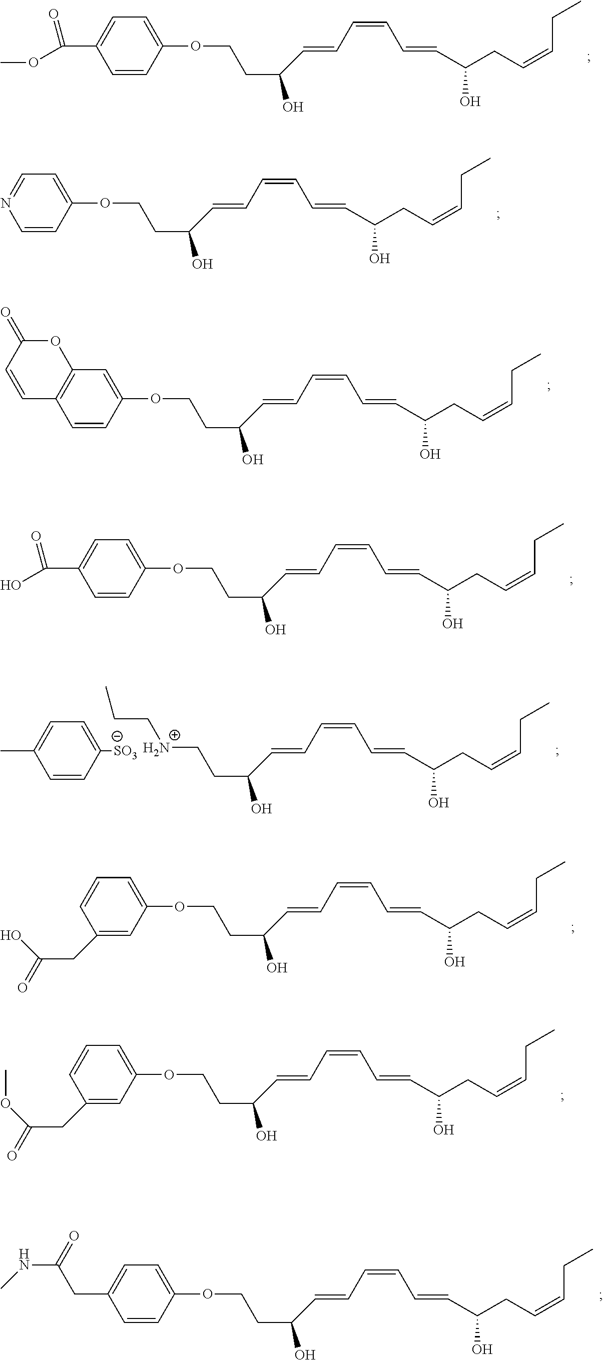

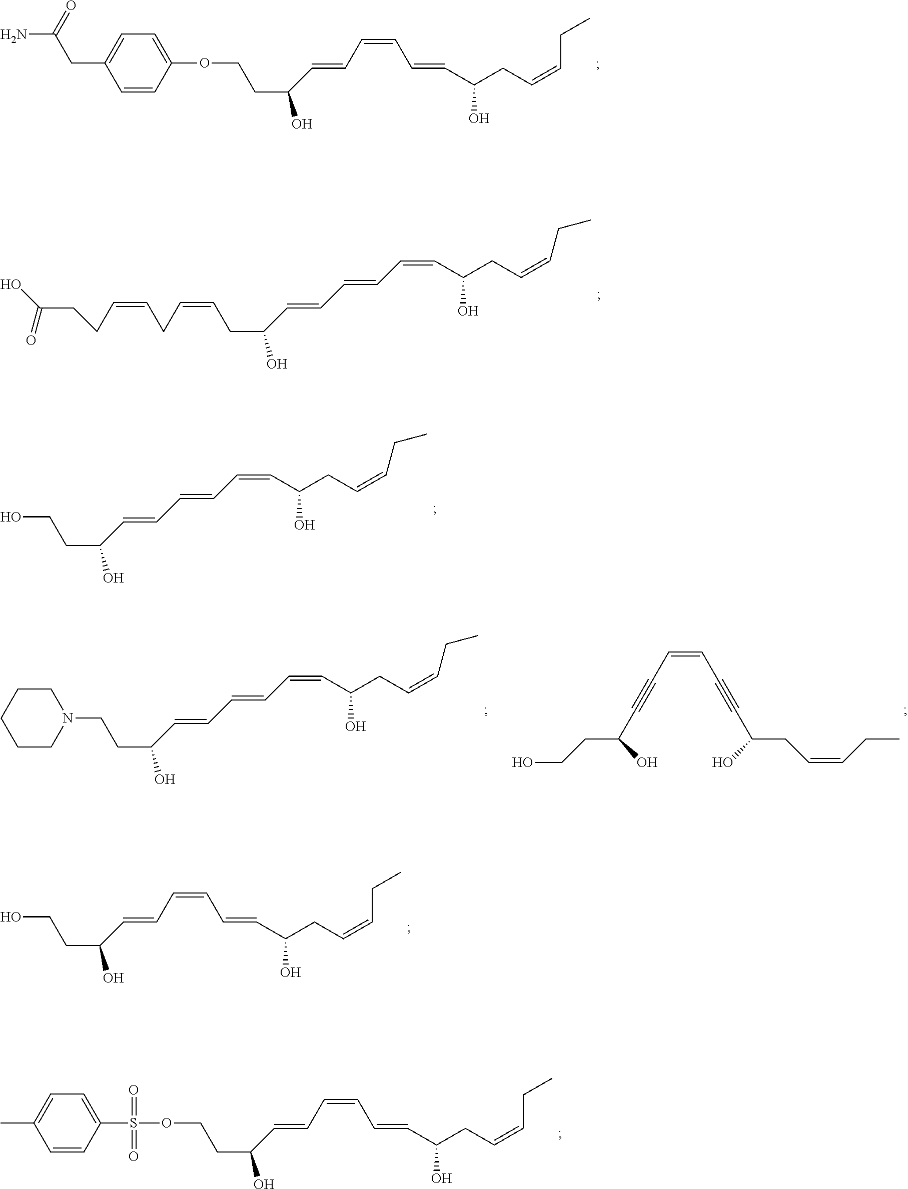

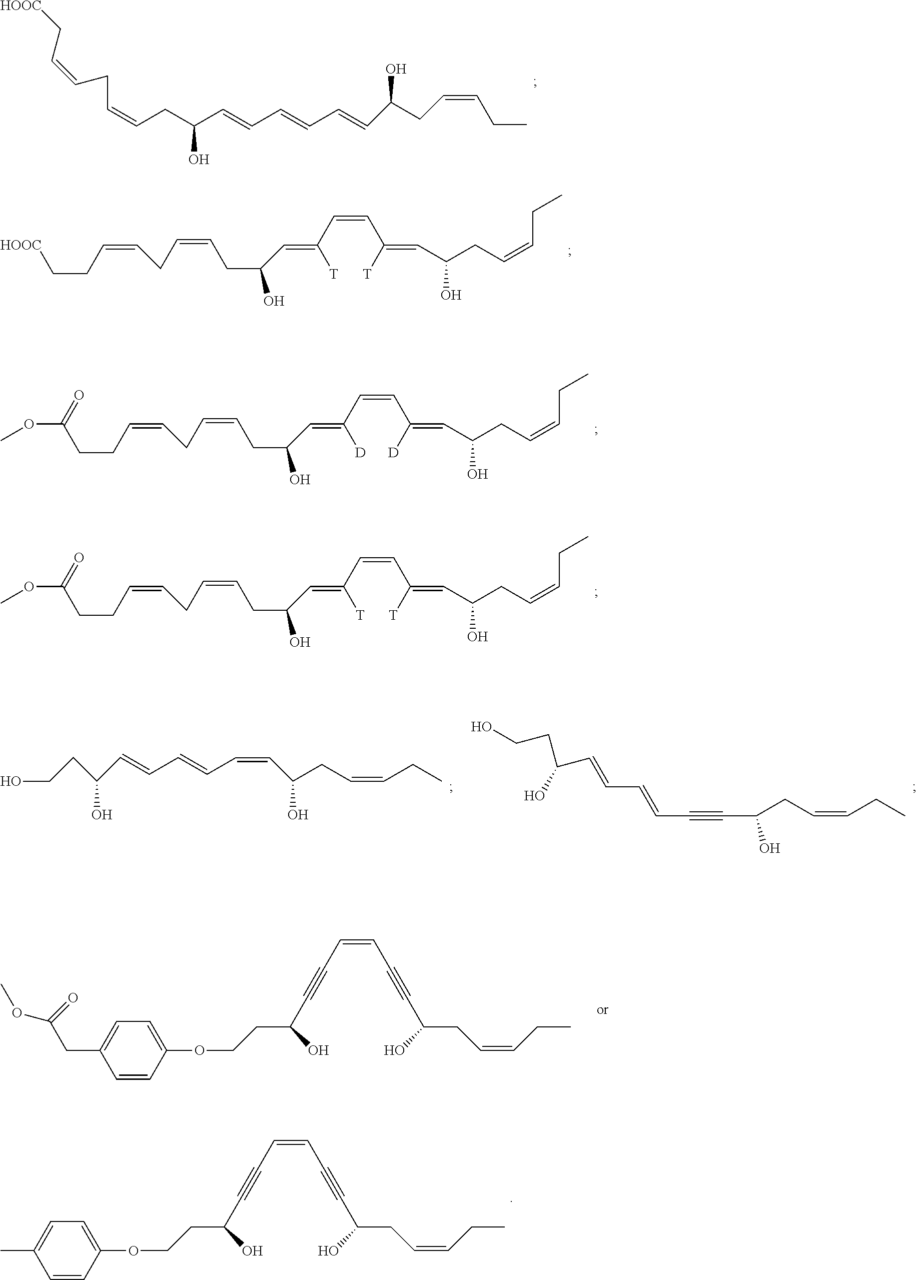

88-92. (canceled)

93. A method of preventing, delaying the onset or reducing the severity of, preventing, or reversing the progression of, or treating chronic low-level inflammation associated with chronic conditions in a subject, said method comprising administering a protectin, protectin analog, structural isomer, or a pharmaceutically acceptable salt thereof, to the subject.

94. The method of claim 93, wherein the chronic conditions comprise neurological disorders, ocular disorders, autoimmune disorders, metabolic disorders, gastrointestinal disorders, and skin disorders.

95. The method of claim 93, wherein reduction of inflammation is through concomitantly reducing the initiation phase and enhancing the resolution phase of the inflammation.

96. The method of claim 93, wherein the protectin, protectin analog, structural isomer, or a pharmaceutically acceptable salt thereof is at least one of: 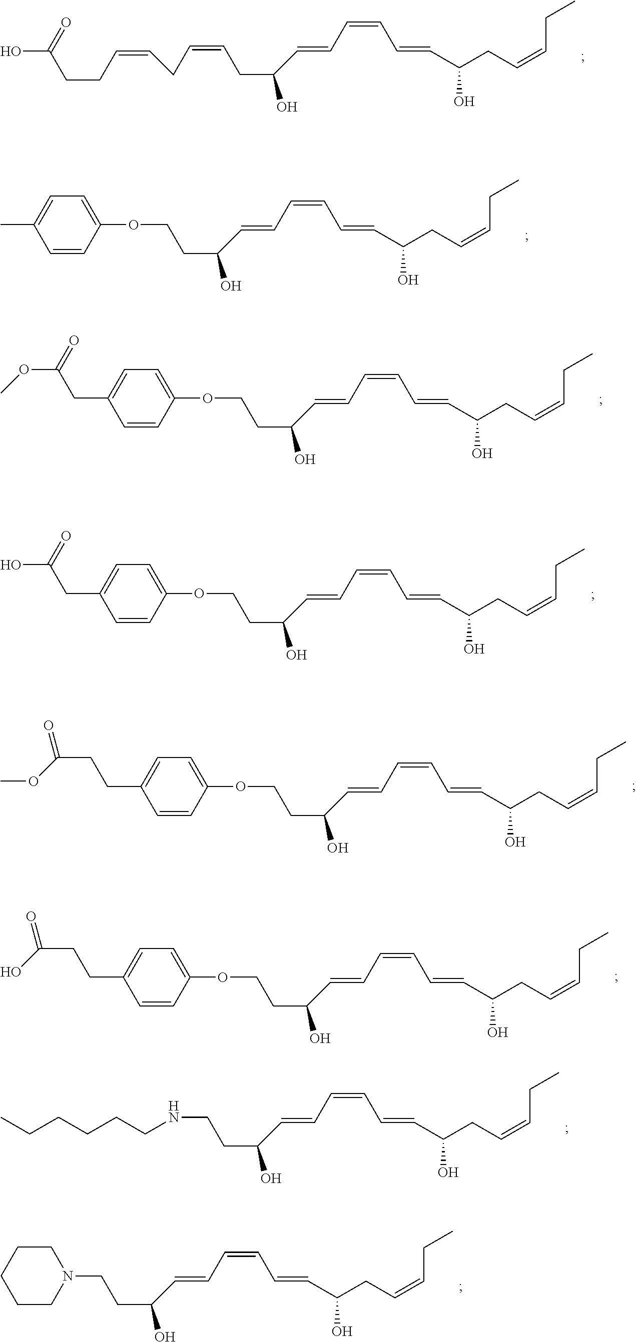 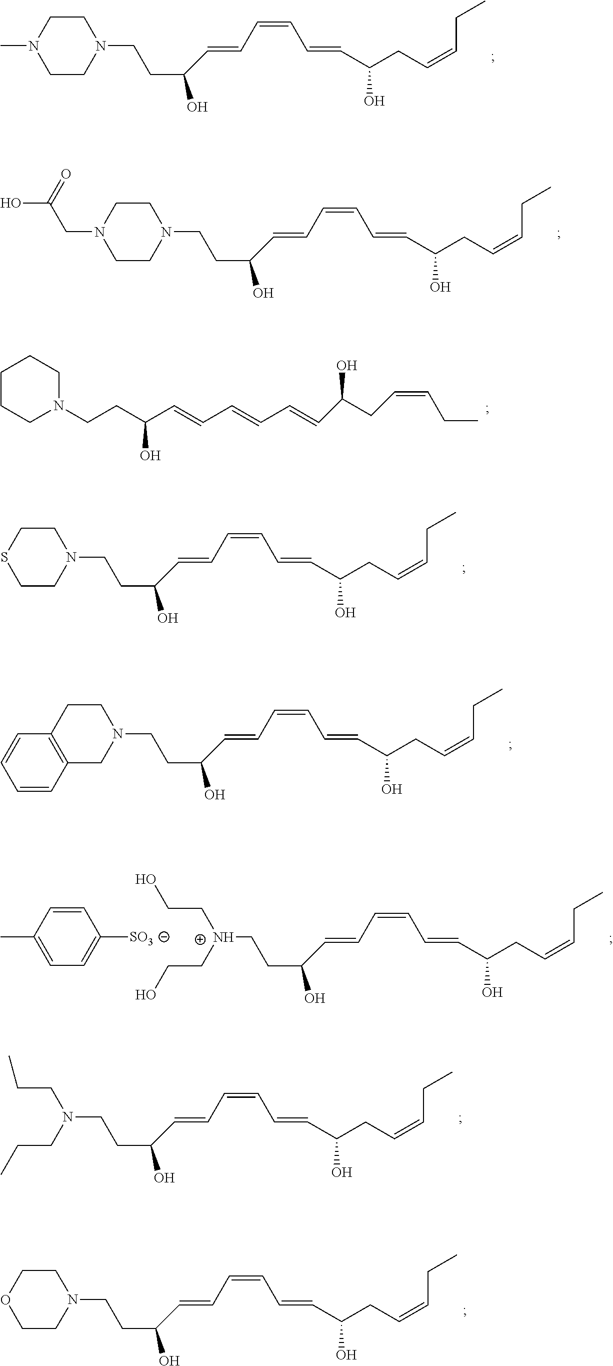 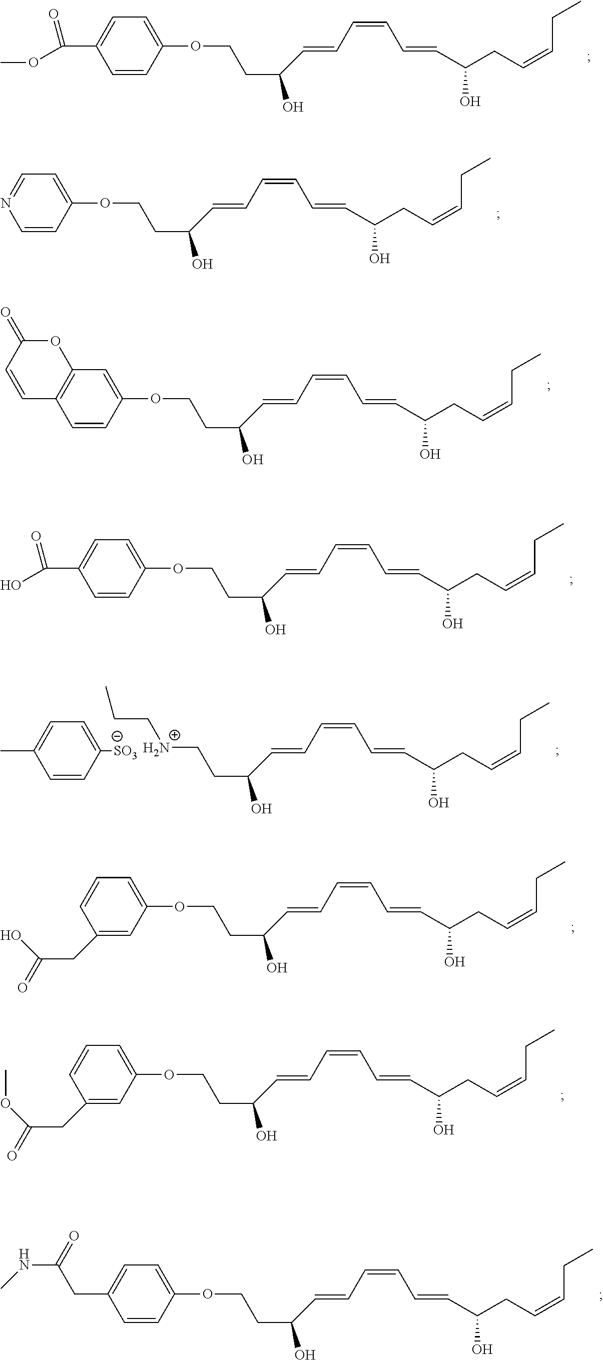 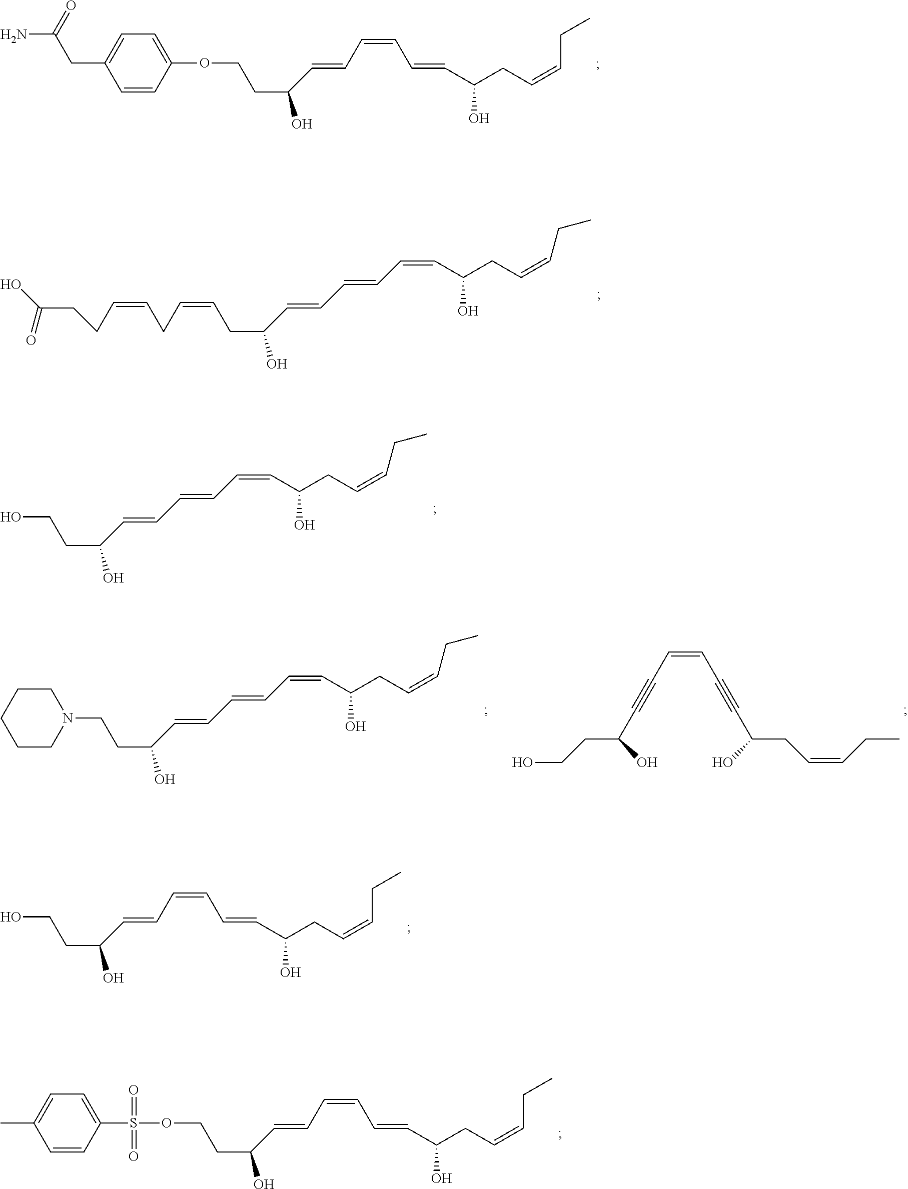 [chemical structure]
